(19) United States
(12) Patent Application Publication
(10) Pub. No.: US 2022/0118113 A1
(43) Pub. Date: Apr. 21, 2022

(54) SORTASE-LABELLED CLOSTRIDIUM NEUROTOXINS

(71) Applicant: IPSEN BIOPHARM LIMITED, Wrexham (GB)

(72) Inventors: Omar LOSS, Wrexham (GB); Mark ELLIOTT, Wrexham (GB)

(21) Appl. No.: 17/310,019

(22) PCT Filed: Jan. 16, 2020
(86) PCT No.: PCT/GB2020/050089

§ 371 Date: Jul. 11, 2021

(30) Foreign Application Priority Data
Jan. 16, 2019 (GB) 1900621.2

(51) Int. Cl. A61K 49/00; C12N 9/52; G01N 33/58

(52) U.S. Cl. CPC A61K 49/0056; C12N 9/52; C12Y 304/24069; C12Y 304/24068; G01N 33/582

(57) ABSTRACT

The present invention relates to a method for preparing a labelled polypeptide, the method comprising: a. providing a polypeptide comprising: i. a sortase acceptor site or a sortase donor site; ii. a non-cytotoxic protease or a proteolytically inactive mutant thereof; iii. a Targeting Moiety (TM) that is capable of binding to a Binding Site on a target cell; and iv. a translocation domain; b. incubating the polypeptide with: a sortase; and a labelled substrate comprising a sortase donor site or a sortase acceptor site, respectively, and a conjugated detectable label; wherein the sortase catalyses: conjugation between an amino acid of the sortase acceptor site of the polypeptide and an amino acid of the sortase donor site of the labelled substrate; or conjugation between an amino acid of the sortase acceptor site of the labelled substrate and an amino acid of the sortase donor site of the polypeptide; thereby labelling the polypeptide; and c. obtaining the labelled polypeptide. The invention also relates to polypeptides for labelling, labelled polypeptides, nucleic acids encoding said polypeptides, and methods of using and manufacturing said polypeptides.

The present invention relates to labelled polypeptides and methods for preparing and using the same.

Bacteria in the genus Clostridia produce highly potent and specific protein toxins, which can poison neurons and other cells to which they are delivered. Examples of such clostridial neurotoxins include the neurotoxins produced by C. tetani (TeNT) and by C. botulinum (BoNT) serotypes A-G, and X (see WO 2018/009903 A2), as well as those produced by C. baratii and C. butyricum.

Among the clostridial neurotoxins are some of the most potent toxins known. By way of example, botulinum neurotoxins have median lethal dose (LD₅₀) values for mice ranging from 0.5 to 5 ng/kg, depending on the serotype. Both tetanus and botulinum toxins act by inhibiting the function of affected neurons, specifically the release of neurotransmitters. While botulinum toxin acts at the neuromuscular junction and inhibits cholinergic transmission in the peripheral nervous system, tetanus toxin acts in the central nervous system.

Clostridial neurotoxins are expressed as single-chain polypeptides in Clostridium. Each clostridial neurotoxin has a catalytic light chain separated from the heavy chain (encompassing the N-terminal translocation domain and the C-terminal receptor binding domain) by an exposed region called the activation loop. During protein maturation proteolytic cleavage of the activation loop separates the light and heavy chain of the clostridial neurotoxin, which are held together by a disulphide bridge, to create fully active di-chain toxin.

Also known in the art are re-targeted clostridial neurotoxins, which may be modified to include an exogenous ligand known as a Targeting Moiety (TM). The TM is selected to provide binding specificity for a desired target cell, and as part of the re-targeting process the native binding portion of the clostridial neurotoxin (e.g. the H_{C} domain, or the H_{CC} domain) may be removed. Re-targeting technology is described, for example, in: EP-B-0689459; WO 1994/021300; EP-B-0939818; U.S. Pat. Nos. 6,461,617; 7,192,596; WO 1998/007864; EP-B-0826051; U.S. Pat. Nos. 5,989,545; 6,395,513; 6,962,703; WO 1996/033273; EP-B-0996468; U.S. Pat. No. 7,052,702; WO 1999/017806; EP-B-1107794; U.S. Pat. No. 6,632,440; WO 2000/010598; WO 2001/21213; WO 2006/059093; WO 2000/62814; WO 2000/04926; WO 1993/15766; WO 2000/61192; and WO 1999/58571; all of which are hereby incorporated by reference in their entirety.

A further variation comprises polypeptides prepared from one or more of the non-cytotoxic protease, translocation or binding domains of clostridial neurotoxins or of polypeptides with equivalent/similar functionality.

The binding, translocation, and proteolytic cleavage of SNARE proteins by clostridial neurotoxins (or other polypeptides described herein) remains poorly understood. Thus, there remains a need for an assay that allows for the visualisation of each of these stages, particularly in real-time and/or in live cells. Such an assay would facilitate the development and characterisation of clostridial neurotoxin therapeutics, especially characterisation of new BoNT therapeutics, hybrid toxins, and re-targeted clostridial neurotoxins (and variants thereof).

Furthermore, antibodies (e.g. fluorescent antibodies) used in conventional methods to visualise clostridial neurotoxins and other such polypeptides are poor, with limited specificity and/or sensitivity. Moreover, such conventional methods typically rely on fixation of cells, which can have a detrimental effect on the cellular architecture, and is not amenable to live/real-time imaging, particularly in complex biological systems such as in vivo in animals. Thus, there is a need for improved/alternative techniques.

The present invention overcomes one or more of the above-mentioned problems.

The present inventors have surprisingly found that sortase can be used to conjugate a detectable label to polypeptides of the invention (comprising a non-cytotoxic protease or a proteolytically inactive mutant thereof; a Targeting Moiety (TM) that binds to a Binding Site on a target cell; and a translocation domain) without reducing potency of the labelled polypeptide. In other words, the labelled polypeptides demonstrate similar (or improved) cell binding, translocation, and SNARE protein cleavage when compared to an equivalent unlabelled polypeptide. This was completely unexpected given that polypeptides labelled using alternative techniques (e.g. non-site specific labelling and SNAP labelling) exhibited reduced potency.

Moreover, polypeptides of the invention comprising a sortase acceptor or donor site could be easily purified and expressed, again this was surprising given that GFP tagging was associated with expression/purification difficulties, indicating that incorporation of the sortase acceptor or donor sites did not negatively influence polypeptide structure or folding.

Additionally, the methods comprising the use of sortase allowed for the production of a dual-labelled polypeptide, which also allowed visualisation of translocation events occurring within the cellular endosomes, one of the least understood aspects of clostridial neurotoxin (and re-targeted clostridial neurotoxin) trafficking. Advantageously, the present invention allows the visualisation of translocation using live imaging microscopy and will greatly contribute to the understanding of the translocation mechanisms in several cellular models and tissues.

The labelled polypeptides of the invention open new avenues for live and/or real-time monitoring of the mechanism of action of said polypeptides and remove the need for fixative products, which have a detrimental effect on the cellular architecture. Thus, the present invention allows for the visualisation of toxins in more complex biological systems such as ex vivo tissue preparations (e.g. brain slices), histopathological samples, and in vivo in animals, and will not be limited to simple cellular systems such as immortalized cell lines and neurons as per conventional techniques. The polypeptides of the present invention may therefore be used (for example) to measure dispersal of the polypeptide away from a site of administration.

In one aspect the invention provides a method for preparing a labelled polypeptide, the method comprising:
a. providing a polypeptide comprising:
   i. a sortase acceptor or donor site;
   ii. a non-cytotoxic protease or a proteolytically inactive mutant thereof;
   iii. a Targeting Moiety (TM) that is capable of binding to a Binding Site on a target cell; and
   iv. a translocation domain;
b. incubating the polypeptide with:
   a sortase; and
   a labelled substrate comprising a sortase donor or acceptor site and a conjugated detectable label;
   wherein the sortase catalyses conjugation between an amino acid of the sortase acceptor site and an amino acid of the sortase donor site, thereby labelling the polypeptide; and
c. obtaining the labelled polypeptide.

When the method of the invention comprises the use of a polypeptide comprising a sortase acceptor site, the labelled substrate comprising the conjugated detectable label (e.g. as referred to in b.) comprises a sortase donor site. Likewise, when the method of the invention comprises the use of a polypeptide comprising a sortase donor site, the labelled substrate comprising the conjugated detectable label (e.g. as referred to in b.) comprises a sortase acceptor site.

The invention thus relates to the use of a sortase acceptor site and a corresponding sortase donor site, wherein a sortase is capable of catalysing conjugation of an amino acid of the sortase acceptor site and an amino acid of the sortase donor site. Therefore, the corresponding sortase acceptor and donor sites for use in the invention are selected such that the conjugation can be performed by a sortase.

Thus, in one embodiment a method of the invention comprises:
a. providing a polypeptide comprising:
   i. a sortase acceptor site;
   ii. a non-cytotoxic protease or a proteolytically inactive mutant thereof;
   iii. a Targeting Moiety (TM) that is capable of binding to a Binding Site on a target cell; and
   iv. a translocation domain;
b. incubating the polypeptide with:
   a sortase; and
   a labelled substrate comprising a sortase donor site and a conjugated detectable label;
   wherein the sortase catalyses conjugation between an amino acid of the sortase acceptor site and an amino acid of the sortase donor site, thereby labelling the polypeptide; and
c. obtaining the labelled polypeptide.

In another embodiment a method of the invention comprises:
a. providing a polypeptide comprising:
   i. a sortase donor site;
   ii. a non-cytotoxic protease or a proteolytically inactive mutant thereof;
   iii. a Targeting Moiety (TM) that is capable of binding to a Binding Site on a target cell; and
   iv. a translocation domain;
b. incubating the polypeptide with:
   a sortase; and
   a labelled substrate comprising a sortase acceptor site and a conjugated detectable label;
   wherein the sortase catalyses conjugation between an amino acid of the sortase acceptor site and an amino acid of the sortase donor site, thereby labelling the polypeptide; and
c. obtaining the labelled polypeptide.

The present invention also provides a labelled polypeptide obtainable by a method of the invention.

In one embodiment the detectable label is conjugated at or near to the sortase acceptor or donor site of the polypeptide comprising a non-cytotoxic protease or a proteolytically inactive mutant thereof; Targeting Moiety (TM); and a translocation domain.

In one embodiment a detectable label is conjugated at the sortase acceptor or donor site, e.g. conjugated directly to an amino acid of the sortase acceptor or donor site. Alternatively, the detectable label may be conjugated C-terminal to the sortase acceptor or donor site, for example 1-50, e.g. 1-25 or 1-10 amino acids C-terminal to the sortase acceptor or donor site.

In another embodiment a detectable label is conjugated N-terminal to the sortase acceptor or donor site, for example 1-50, e.g. 1-25 or 1-10 amino acids N-terminal to the sortase acceptor or donor site.

The term “obtainable” as used herein also encompasses the term “obtained”. In one embodiment the term “obtainable” means obtained.

In a related aspect there is provided a polypeptide for labelling using a sortase, the polypeptide comprising:
i. a sortase acceptor or donor site;
ii. a non-cytotoxic protease that is capable of cleaving a protein of the exocytic fusion apparatus in a target cell or a proteolytically inactive mutant thereof;
iii. a Targeting Moiety (TM) that is capable of binding to a Binding Site on a target cell; and
iv. a translocation domain that is capable of translocating the non-cytotoxic protease from within an endosome, across the endosomal membrane and into the cytosol of the target cell;
   wherein when the polypeptide comprises a sortase donor site, the sortase donor site is located at an N-terminus of the polypeptide, and wherein when the sortase donor site comprises Gₙ or Aₙ, n is at least 2; and
      wherein the N-terminal residue of the donor site is the N-terminal residue of the polypeptide; or
      wherein the polypeptide comprises one or more amino acid residues N-terminal to the sortase donor site and a cleavable site, which when cleaved exposes the N-terminus of the sortase donor site.

In one embodiment a polypeptide for labelling using a sortase comprises:
i. a sortase donor site;
ii. a non-cytotoxic protease that is capable of cleaving a protein of the exocytic fusion apparatus in a target cell or a proteolytically inactive mutant thereof;
iii. a Targeting Moiety (TM) that is capable of binding to a Binding Site on a target cell; and
iv. a translocation domain that is capable of translocating the non-cytotoxic protease from within an endosome, across the endosomal membrane and into the cytosol of the target cell;
   wherein the sortase donor site is located at an N-terminus of the polypeptide, and wherein when the sortase donor site comprises Gₙ or Aₙ, n is at least 2; and
      wherein the N-terminal residue of the donor site is the N-terminal residue of the polypeptide.

In one embodiment a polypeptide for labelling using a sortase comprises:
i. a sortase donor site;
ii. a non-cytotoxic protease that is capable of cleaving a protein of the exocytic fusion apparatus in a target cell or a proteolytically inactive mutant thereof;
iii. a Targeting Moiety (TM) that is capable of binding to a Binding Site on a target cell; and
iv. a translocation domain that is capable of translocating the non-cytotoxic protease from within an endosome, across the endosomal membrane and into the cytosol of the target cell;
   wherein the sortase donor site is located at an N-terminus of the polypeptide, and wherein when the sortase donor site comprises Gₙ or Aₙ, n is at least 2; and
      wherein the polypeptide comprises one or more amino acid residues N-terminal to the sortase donor site and a cleavable site, which when cleaved exposes the N-terminus of the sortase donor site.

In one embodiment a polypeptide for labelling using a sortase comprises:
i. a sortase acceptor site;
ii. a non-cytotoxic protease that is capable of cleaving a protein of the exocytic fusion apparatus in a target cell or a proteolytically inactive mutant thereof;
iii. a Targeting Moiety (TM) that is capable of binding to a Binding Site on a target cell; and
iv. a translocation domain that is capable of translocating the non-cytotoxic protease from within an endosome, across the endosomal membrane and into the cytosol of the target cell.

The polypeptide is suitably used in a method of the invention.

A polypeptide of the invention may comprise a sortase acceptor site. Alternatively, said polypeptide may comprise a sortase donor site.

In a preferred embodiment, said polypeptide comprises a sortase acceptor site and a sortase donor site.

A polypeptide of the present invention may comprise a polypeptide sequence having at least 70% sequence identity to SEQ ID NO: 2. In one embodiment a polypeptide of the invention comprises a polypeptide sequence having at least 80% or 90% sequence identity to SEQ ID NO: 2. Preferably, a polypeptide of the invention comprises (more preferably consists of) a polypeptide shown as SEQ ID NO: 2.

A polypeptide of the present invention may comprise a polypeptide sequence having at least 70% sequence identity to SEQ ID NO: 4. In one embodiment a polypeptide of the invention comprises a polypeptide sequence having at least 80% or 90% sequence identity to SEQ ID NO: 4. Preferably, a polypeptide of the invention comprises (more preferably consists of) a polypeptide shown as SEQ ID NO: 4.

A polypeptide of the present invention may comprise a polypeptide sequence having at least 70% sequence identity to SEQ ID NO: 40. In one embodiment a polypeptide of the invention comprises a polypeptide sequence having at least 80% or 90% sequence identity to SEQ ID NO: 40. Preferably, a polypeptide of the invention comprises (more preferably consists of) a polypeptide shown as SEQ ID NO: 40.

A polypeptide may be encoded by a nucleic acid of the invention.

The invention also provides a labelled polypeptide, the polypeptide comprising:
i. a detectable label conjugated to the polypeptide;
ii. a non-cytotoxic protease or a proteolytically inactive mutant thereof;
iii. a Targeting Moiety (TM) that is capable of binding to a Binding Site on a target cell; and
iv. a translocation domain.

The invention also provides a labelled polypeptide, the polypeptide comprising:
i. a detectable label conjugated to the polypeptide;
ii. an amino acid sequence that comprises L(A/P/S)X(T/S/A/C)Gₙ (SEQ ID NO: 59), wherein X is any amino acid and n is at least 1, L(A/P/S)X(T/S/A/C)Aₙ (SEQ ID NO: 60), wherein X is any amino acid and n is at least 1, NPQTN (SEQ ID NO: 61), YPRTG (SEQ ID NO: 62), IPQTG (SEQ ID NO: 63), VPDTG (SEQ ID NO: 64), LPXTGS (SEQ ID NO: 65), wherein X is any amino acid, NPKTG (SEQ ID NO: 46), XPETG (SEQ ID NO: 47), LGATG (SEQ ID NO: 48), IPNTG (SEQ ID NO: 49), IPETG (SEQ ID NO: 50), NSKTA (SEQ ID NO: 51), NPQTG (SEQ ID NO: 52), NAKTN (SEQ ID NO: 53), NPQSS (SEQ ID NO: 54), LPXTX (SEQ ID NO: 55), wherein X is any amino acid, NPX₁TX₂ (SEQ ID NO: 56), wherein X₁ is Lys or Gln and X₂ is Asn, Asp or Gly, X₁PX₂X₃G (SEQ ID NO: 57), wherein X₁ is Leu, Ile, Val or Met, X₂ is any amino acid and X₃ is Ser, Thr or Ala, LPEX₁G (SEQ ID NO: 58), wherein X, is Ala, Cys or Ser, LPXS (SEQ ID NO: 66), LAXT (SEQ ID NO: 67), MPXT (SEQ ID NO: 68), MPXTG (SEQ ID NO: 69), LAXS (SEQ ID NO: 70), NPXT (SEQ ID NO: 71), NPXTG (SEQ ID NO: 72), NAXT (SEQ ID NO: 73), NAXTG (SEQ ID NO: 74), NAXS (SEQ ID NO: 75), NAXSG (SEQ ID NO: 76), LPXP (SEQ ID NO: 77), LPXPG (SEQ ID NO: 78), wherein X is any amino acid, LRXTGₙ (SEQ ID NO: 111) or LPAXGₙ (SEQ ID NO: 106), wherein X is any amino acid and n is at least 1;
iii. a non-cytotoxic protease or a proteolytically inactive mutant thereof;
iv. a Targeting Moiety (TM) that is capable of binding to a Binding Site on a target cell; and
v. a translocation domain.

The invention also provides a labelled polypeptide, the polypeptide comprising:
i. a detectable label conjugated to the polypeptide;
ii. an amino acid sequence that comprises L(A/P/S)X(T/S/A/C)Gₙ, wherein X is any amino acid and n is at least 1, L(A/P/S)X(T/S/A/C)Aₙ, wherein X is any amino acid and n is at least 1, NPQTN, YPRTG, IPQTG, VPDTG, or LPXTGS, wherein X is any amino acid;
iii. a non-cytotoxic protease or a proteolytically inactive mutant thereof;
iv. a Targeting Moiety (TM) that is capable of binding to a Binding Site on a target cell; and
v. a translocation domain.

In one embodiment a labelled polypeptide comprises:
i. a detectable label conjugated to the polypeptide;
ii. an amino acid sequence that comprises L(A/P/S)X(T/S/A/C)Gₙ, wherein X is any amino acid and n is at least 1, NPQTN, YPRTG, IPQTG, VPDTG, LPXTGS, wherein X is any amino acid, NPKTG, XPETG, LGATG, IPNTG, IPETG, NSKTA, NPQTG, NAKTN, NPQSS, LPXTX, wherein X is any amino acid, NPX₁TX₂, wherein X₁ is Lys or Gln and X₂ is Asn, Asp or Gly, X₁PX₂X₃G, wherein X, is Leu, Ile, Val or Met, X₂ is any amino acid and X₃ is Ser, Thr or Ala, LPEX₁G, wherein X₁ is Ala, Cys or Ser, LPXS, LAXT, MPXT, MPXTG, LAXS, NPXT, NPXTG, NAXT, NAXTG, NAXS, NAXSG, LPXP, LPXPG, wherein X is any amino acid, LRXTGₙ or LPAXGₙ, wherein X is any amino acid and n is at least 1;
iii. a non-cytotoxic protease or a proteolytically inactive mutant thereof;
iv. a Targeting Moiety (TM) that is capable of binding to a Binding Site on a target cell; and
v. a translocation domain.

In one embodiment a labelled polypeptide comprises:
i. a detectable label conjugated to the polypeptide;
ii. an amino acid sequence that comprises L(A/P/S)X(T/S/A/C)Gₙ, wherein X is any amino acid and n is at least 1, NPQTN, YPRTG, IPQTG, VPDTG, or LPXTGS, wherein X is any amino acid;
iii. a non-cytotoxic protease or a proteolytically inactive mutant thereof;
iv. a Targeting Moiety (TM) that is capable of binding to a Binding Site on a target cell; and
v. a translocation domain.

In one embodiment a labelled polypeptide of the invention demonstrates similar cell binding, translocation, and SNARE protein cleavage when compared to an equivalent unlabelled polypeptide. In another embodiment a labelled polypeptide demonstrates improved cell binding, translocation, and/or SNARE protein cleavage when compared to an equivalent unlabelled polypeptide. In a particularly preferred embodiment a labelled polypeptide demonstrates improved cell binding, translocation, and SNARE protein cleavage when compared to an equivalent unlabelled polypeptide. The cell binding, translocation, and/or SNARE protein cleavage may be determined using any technique known in the art and/or described herein. In one embodiment cell binding, translocation, and/or SNARE protein cleavage may be determined using a cell-based or in vivo assay. Suitable assays may include the Digit Abduction Score (DAS), the dorsal root ganglia (DRG) assay, spinal cord neuron (SCN) assay, and mouse phrenic nerve hemidiaphragm (PNHD) assay, which are routine in the art. A suitable assay may be one described in Donald et al (2018), Pharmacol Res Perspect, e00446, 1-14, which is incorporated herein by reference. Preferably, a suitable assay is the SNAP25 cleavage assay as described in Fonfria, E., S. Donald and V. A. Cadd (2016), “Botulinum neurotoxin A and an engineered derivate targeted secretion inhibitor (TSI) A enter cells via different vesicular compartments.” J Recept Signal Transduct Res 36(1): 79-88, which is incorporated herein by reference.

In one embodiment the detectable label is conjugated at or near to the amino acid sequence comprising L(A/P/S)X(T/S/A/C)Gₙ, L(A/P/S)X(T/S/A/C)Aₙ, NPQTN, YPRTG, IPQTG, VPDTG, LPXTGS, NPKTG, XPETG, LGATG, IPNTG, IPETG, NSKTA, NPQTG, NAKTN, NPQSS, LPXTX, wherein X is any amino acid, NPX₁TX₂, wherein X, is Lys or Gln and X₂ is Asn, Asp or Gly, X₁PX₂X₃G, wherein X₁ is Leu, Ile, Val or Met, X₂ is any amino acid and X₃ is Ser, Thr or Ala, LPEX₁G, wherein X, is Ala, Cys or Ser, LPXS, LAXT, MPXT, MPXTG, LAXS, NPXT, NPXTG, NAXT, NAXTG, NAXS, NAXSG, LPXP, LPXPG, wherein X is any amino acid, LRXTGₙ or LPAXGₙ, wherein X is any amino acid and n is at least 1. In one embodiment the detectable label is conjugated at or near to the amino acid sequence comprising L(A/P/S)X(T/S/A/C)Gₙ, L(A/P/S)X(T/S/A/C)Aₙ, NPQTN, YPRTG, IPQTG, VPDTG, or LPXTGS.

In one embodiment an amino acid sequence comprising L(A/P/S)X(T/S/A/C)Gₙ, L(A/P/S)X(T/S/A/C)Aₙ, NPQTN, YPRTG, IPQTG, VPDTG, LPXTGS, NPKTG, XPETG, LGATG, IPNTG, IPETG, NSKTA, NPQTG, NAKTN, NPQSS, LPXTX, wherein X is any amino acid, NPX₁TX₂, wherein X₁ is Lys or Gin and X₂ is Asn, Asp or Gly, X₁PX₂X₃G, wherein X₁ is Leu, Ile, Val or Met, X₂ is any amino acid and X₃ is Ser, Thr or Ala, LPEX₁G, wherein X₁ is Ala, Cys or Ser, LPXS, LAXT, MPXT, MPXTG, LAXS, NPXT, NPXTG, NAXT, NAXTG, NAXS, NAXSG, LPXP, LPXPG, wherein X is any amino acid, LRXTGₙ or LPAXGₙ, wherein X is any amino acid and n is at least 1, may be located C-terminal to the TM of the polypeptide. In one embodiment an amino acid sequence comprising L(A/P/S)X(T/S/A/C)Gₙ, L(A/P/S)X(T/S/A/C)Aₙ, NPQTN, YPRTG, IPQTG, VPDTG, or LPXTGS may be located C-terminal to the TM of the polypeptide. In another embodiment an amino acid sequence comprising L(A/P/S)X(T/S/A/C)Gₙ, L(A/P/S)X(T/S/A/C)Aₙ, NPQTN, YPRTG, IPQTG, VPDTG, LPXTGS, NPKTG, XPETG, LGATG, IPNTG, IPETG, NSKTA, NPQTG, NAKTN, NPQSS, LPXTX, wherein X is any amino acid, NPX₁TX₂, wherein X₁ is Lys or Gin and X₂ is Asn, Asp or Gly, X₁PX₂X₃G, wherein X, is Leu, Ile, Val or Met, X₂ is any amino acid and X₃ is Ser, Thr or Ala, LPEX₁G, wherein X, is Ala, Cys or Ser, LPXS, LAXT, MPXT, MPXTG, LAXS, NPXT, NPXTG, NAXT, NAXTG, NAXS, NAXSG, LPXP, LPXPG, wherein X is any amino acid, LRXTGₙ or LPAXGₙ, wherein X is any amino acid and n is at least 1, may be located N-terminal to the non-cytotoxic protease or proteolytically inactive mutant thereof of the polypeptide. In another embodiment an amino acid sequence comprising L(A/P/S)X(T/S/A/C)Gₙ, L(A/P/S)X(T/S/A/C)Aₙ, NPQTN, YPRTG, IPQTG, VPDTG, or LPXTGS may be located N-terminal to the non-cytotoxic protease or proteolytically inactive mutant thereof of the polypeptide.

In one embodiment a labelled polypeptide comprises two or more detectable labels, preferably a labelled polypeptide comprises two detectable labels. In preferred embodiment the detectable labels are different, e.g. differently-coloured fluorophores.

A first and second (or more) detectable label may be conjugated at or near to an amino acid sequence comprising L(A/P/S)X(T/S/A/C)Gₙ, L(A/P/S)X(T/S/A/C)Aₙ, NPQTN, YPRTG, IPQTG, VPDTG, LPXTGS, NPKTG, XPETG, LGATG, IPNTG, IPETG, NSKTA, NPQTG, NAKTN, NPQSS, LPXTX, wherein X is any amino acid, NPX₁TX₂, wherein X₁ is Lys or Gin and X₂ is Asn, Asp or Gly, X₁PX₂X₃G, wherein X₁ is Leu, Ile, Val or Met, X₂ is any amino acid and X₃ is Ser, Thr or Ala, LPEX₁G, wherein X₁ is Ala, Cys or Ser, LPXS, LAXT, MPXT, MPXTG, LAXS, NPXT, NPXTG, NAXT, NAXTG, NAXS, NAXSG, LPXP, LPXPG, wherein X is any amino acid, LRXTGₙ or LPAXGₙ, wherein X is any amino acid and n is at least 1, wherein the first and second (or more) detectable labels are conjugated at different sites on the labelled polypeptide. A first and second (or more) detectable label may be conjugated at or near to an amino acid sequence comprising L(A/P/S)X(T/S/A/C)Gₙ, L(A/P/S)X(T/S/A/C)Aₙ, NPQTN, YPRTG, IPQTG, VPDTG, or LPXTGS, wherein the first and second (or more) detectable labels are conjugated at different sites on the labelled polypeptide. For example, a first detectable label may be conjugated to an amino acid sequence located N-terminal to the non-cytotoxic protease or proteolytically inactive mutant thereof and a second detectable label may be conjugated to an amino acid sequence located C-terminal to the TM (or vice versa). Preferably the sequence of the amino acid sequence where the first and second (or more) detectable labels are conjugated are different.

In one embodiment a detectable label is conjugated at L(A/P/S)X(T/S/A/C)Gₙ, L(AP/S)X(T/S/A/C)Aₙ, NPQTN, YPRTG, IPQTG, VPDTG, LPXTGS, NPKTG, XPETG, LGATG, IPNTG, IPETG, NSKTA, NPQTG, NAKTN, NPQSS, LPXTX, wherein X is any amino acid, NPX₁TX₂, wherein X₁ is Lys or Gln and X₂ is Asn, Asp or Gly, X₁PX₂X₃G, wherein X₁ is Leu, Ile, Val or Met, X₂ is any amino acid and X₃ is Ser, Thr or Ala, LPEX₁G, wherein X₁ is Ala, Cys or Ser, LPXS, LAXT, MPXT, MPXTG, LAXS, NPXT, NPXTG, NAXT, NAXTG, NAXS, NAXSG, LPXP, LPXPG, wherein X is any amino acid, LRXTGₙ or LPAXGₙ, wherein X is any amino acid and n is at least 1. Alternatively, the detectable label may be conjugated C-terminal to L(A/P/S)X(T/S/A/C)Gₙ, L(A/P/S)X(T/S/A/C)Aₙ, NPQTN, YPRTG, IPQTG, VPDTG, LPXTGS, NPKTG, XPETG, LGATG, IPNTG, IPETG, NSKTA, NPQTG, NAKTN, NPQSS, LPXTX, wherein X is any amino acid, NPX₁TX₂, wherein X, is Lys or Gln and X₂ is Asn, Asp or Gly, X₁PX₂X₃G, wherein X₁ is Leu, Ile, Val or Met, X₂ is any amino acid and X₃ is Ser, Thr or Ala, LPEX₁G, wherein X, is Ala, Cys or Ser, LPXS, LAXT, MPXT, MPXTG, LAXS, NPXT, NPXTG, NAXT, NAXTG, NAXS, NAXSG, LPXP, LPXPG, wherein X is any amino acid, LRXTGₙ or LPAXGₙ, wherein X is any amino acid and n is at least 1, for example 1-50, e.g. 1-25 or 1-10 amino acids C-terminal to L(A/P/S)X(T/S/A/C)Gₙ, L(A/P/S)X(T/S/A/C)Aₙ, NPQTN, YPRTG, IPQTG, VPDTG, LPXTGS, NPKTG, XPETG, LGATG, IPNTG, IPETG, NSKTA, NPQTG, NAKTN, NPQSS, LPXTX, wherein X is any amino acid, NPX₁TX₂, wherein X₁ is Lys or Gln and X₂ is Asn, Asp or Gly, X₁PX₂X₃G, wherein X₁ is Leu, Ile, Val or Met, X₂ is any amino acid and X₃ is Ser, Thr or Ala, LPEX₁G, wherein X, is Ala, Cys or Ser, LPXS, LAXT, MPXT, MPXTG, LAXS, NPXT, NPXTG, NAXT, NAXTG, NAXS, NAXSG, LPXP, LPXPG, wherein X is any amino acid, LRXTGₙ or LPAXGₙ, wherein X is any amino acid and n is at least 1.

In one embodiment a detectable label is conjugated at L(A/P/S)X(T/S/A/C)Gₙ, L(A/P/S)X(T/S/A/C)Aₙ, NPQTN, YPRTG, IPQTG, VPDTG, or LPXTGS. Alternatively, the detectable label may be conjugated C-terminal to L(A/P/S)X(T/S/A/C)Gₙ, L(A/P/S)X(T/S/A/C)Aₙ, NPQTN, YPRTG, IPQTG, VPDTG, or LPXTGS, for example 1-50, e.g. 1-25 or 1-10 amino acids C-terminal to L(A/P/S)X(T/S/A/C)Gₙ, L(NP/S)X(T/S/A/C)Aₙ, NPQTN, YPRTG, IPQTG, VPDTG, or LPXTGS.

In another embodiment a detectable label is conjugated N-terminal to L(A/P/S)X(T/S/AC)Gₙ, L(A/P/S)X(T/S/A/C)Aₙ, NPQTN, YPRTG, IPQTG, VPDTG, LPXTGS, NPKTG, XPETG, LGATG, IPNTG, IPETG, NSKTA, NPQTG, NAKTN, NPQSS, LPXTX, wherein X is any amino acid, NPX₁TX₂, wherein X₁ is Lys or Gln and X₂ is Asn, Asp or Gly, X₁PX₂X₃G, wherein X, is Leu, Ile, Val or Met, X₂ is any amino acid and X₃ is Ser, Thr or Ala, LPEX₁G, wherein X, is Ala, Cys or Ser, LPXS, LAXT, MPXT, MPXTG, LAXS, NPXT, NPXTG, NAXT, NAXTG, NAXS, NAXSG, LPXP, LPXPG, wherein X is any amino acid, LRXTGₙ or LPAXGₙ, wherein X is any amino acid and n is at least 1, for example 1-50, e.g. 1-25 or 1-10 amino acids N-terminal to L(A/P/S)X(T/S/A/C)Gₙ.

In another embodiment a detectable label is conjugated N-terminal to L(A/P/S)X(T/S/A/C)Gₙ, L(A/P/S)X(T/S/A/C)Aₙ, NPQTN, YPRTG, IPQTG, VPDTG, or LPXTGS, for example 1-50, e.g. 1-25 or 1-10 amino acids N-terminal to L(A/P/S)X(T/S/A/C)Gₙ.

In embodiments where an amino acid sequence comprises L(A/P/S)X(T/S/NC)Aₙ, X is any amino acid and n may be at least 2, 3, 4, 5, 6, 7, 8, 9 or 10, such an amino acid sequence may comprise LPXTAₙ (SEQ ID NO: 102). Preferably n is 1-10, more preferably 1-4. In such embodiments the conjugated detectable label and the amino acid sequence that comprises L(A/P/S)X(T/S/A/C)Aₙ, wherein X is any amino acid and n is at least 1, indicates that the polypeptide has been successfully labelled by a sortase (e.g. from Streptococcus pyogenes).

In a particularly preferred embodiment an amino acid sequence comprises L(A/P/S)X(T/S/A/C)Gₙ, wherein X is any amino acid and n is at least 1. Such an amino acid sequence may comprise LPXSGₙ (SEQ ID NO: 103), LAXTGₙ (SEQ ID NO: 104), LPXTGₙ (SEQ ID NO: 105), LPXCGₙ (SEQ ID NO: 107), LAXSGₙ (SEQ ID NO: 108), LPXAGₙ (SEQ ID NO: 109), or LSXTGₙ (SEQ ID NO: 110). Preferably an amino acid sequence may comprise LPXSGₙ, LAXTGₙ, LPXTGₙ, or LAXSGₙ.

In one embodiment an amino acid sequence comprises LRXTGₙ, wherein X is any amino acid and n is at least 1.

In one embodiment an amino acid sequence comprises LPAXGₙ, wherein X is any amino acid and n is at least 1.

The conjugated detectable label and the amino acid sequence that comprises L(A/P/S)X(T/S/A/C)Gₙ, wherein X is any amino acid and n is at least 1, indicates that the polypeptide has been successfully labelled by a sortase. In one embodiment n may be at least 2, 3, 4, 5, 6, 7, 8, 9 or 10. Preferably n is 1-10, more preferably 1-4.

In one embodiment the detectable label is conjugated at or near to L(A/P/S)X(T/S/A/C)Gₙ.

In one embodiment a detectable label is conjugated at L(A/P/S)X(T/S/A/C)Gₙ, such as at a G amino acid residue thereof. Alternatively, the detectable label may be conjugated C-terminal to L(A/P/S)X(T/S/A/C)Gₙ, for example 1-50, e.g. 1-25 or 1-10 amino acids C-terminal to L(A/P/S)X(T/S/A/C)Gₙ.

In another embodiment a detectable label is conjugated N-terminal to L(A/P/S)X(T/S/A/C)Gₙ, for example 1-50, e.g. 1-25 or 1-10 amino acids N-terminal to L(A/P/S)X(T/S/A/C)Gₙ.

In one embodiment a detectable label is conjugated at or near an amino acid sequence LPXSGₙ, wherein n is at least 1, e.g. at least 2, 3, 4, 5, 6, 7, 8, 9 or 10. Preferably wherein n is 1-10, more preferably 1-5. The detectable label is preferably conjugated C-terminal to LPXSGₙ, e.g. to a lysine residue C-terminal to LPXSGₙ. X is any amino acid, such as E.

In one embodiment a detectable label is conjugated at or near an amino acid sequence LAXTGₙ, wherein n is at least 1, e.g. at least 2, 3, 4, 5, 6, 7, 8, 9 or 10. Preferably wherein n is 1-10, more preferably 1-4. The detectable label is preferably conjugated N-terminal to LAXTGₙ, e.g. to a histidine residue N-terminal to LAXTGₙ. X is any amino acid, such as E.

In one embodiment a first detectable label is conjugated at or near an amino acid sequence LPXSGₙ (wherein n is at least 1, e.g. at least 2, 3, 4, 5, 6, 7, 8, 9 or 10, preferably wherein n is 1-10, more preferably 1-5) and a second detectable label conjugated at or near an amino acid sequence LAXTG, (wherein n is at least 1, e.g. at least 2, 3, 4, 5, 6, 7, 8, 9 or 10, preferably wherein n is 1-10, more preferably 1-4). The first detectable label is preferably conjugated C-terminal to LPXSGₙ, e.g. to a lysine residue C-terminal to LPXSG, and the second detectable label is preferably conjugated N-terminal to LAXTGₙ, e.g. to a histidine residue N-terminal to LAXTGₙ. X is any amino acid, such as E. In one embodiment the first detectable label is located C-terminal to a TM of the polypeptide and the second detectable label is located N-terminal to a non-cytotoxic protease or proteolytically inactive mutant thereof (preferably non-cytotoxic protease) of the polypeptide.

A labelled polypeptide of the present invention may comprise a polypeptide sequence having at least 70% sequence identity to SEQ ID NO: 26. In one embodiment a labelled polypeptide of the invention comprises a polypeptide sequence having at least 80% or 90% sequence identity to SEQ ID NO: 26. Preferably, a labelled polypeptide of the invention comprises (more preferably consists of) a polypeptide shown as SEQ ID NO: 26.

A sortase described herein may be a Sortase A, Sortase B, Sortase C or Sortase D. An overview of the biological properties of sortases is provided by Mazmanian, S. K., G. Liu, H. Ton-That and O. Schneewind (1999). “Staphylococcus aureus sortase, an enzyme that anchors surface proteins to the cell wall.” Science 285(5428): 760-763 and Paterson, G. K. and T. J. Mitchell (2004). “The biology of Gram-positive sortase enzymes.” Trends Microbiol 12(2): 89-95, both of which are incorporated herein by reference.

Also encompassed by the present invention are sortase variants. Sortase variants suitably have altered specificity, such that they recognise alternative sortase sites (e.g. acceptor sites). Sortase variants are described in Dorr, B. M., H. O. Ham, C. An, E. L. Chaikof and D. R. Liu (2014). “Reprogramming the specificity of sortase enzymes.” Proc Natl Acad Sci USA 111(37): 13343-13348, Chen, I., B. M. Dorr and D. R. Liu (2011). “A general strategy for the evolution of bond-forming enzymes using yeast display.” Proc Natl Acad Sci USA 108(28): 11399-11404, Dorr, B. M., H. O. Ham, C. An, E. L. Chaikof and D. R. Liu (2014). “Reprogramming the specificity of sortase enzymes.” Proc Natl Acad Sci USA 111(37): 13343-13348, and Chen, L., J. Cohen, X. Song, A. Zhao, Z. Ye, C. J. Feulner, P. Doonan, W. Somers, L. Lin and P. R. Chen (2016). “Improved variants of SrtA for site-specific conjugation on antibodies and proteins with high efficiency.” Sci Rep 6: 31899 each of which are incorporated herein by reference. Bespoke sortase variants may be generated using the methodology described in said references. The skilled person will select the appropriate sortase donor and/or acceptor sites recognised by the sortase variant when employing said variant in the present invention. The skilled person will further recognise that said sortase donor and/or acceptor sites may vary from those presented herein.

In one embodiment, a sortase variant may comprise an evolved Staphylococcus aureus Sortase A. An evolved Sortase A may include one or more mutations relative to the sequence of SEQ ID NO: 31 described herein. For example, an evolved Sortase A may comprise one or more of the following mutations relative to the sequence of SEQ ID NO: 31: P86L, P94S, P94R, N98S, A104T, E106G, A118T, F122S, F122Y, D124G, N127S, K134R, F154R, D160N, D165A, K173E, G174S, K177E, I182V, K190E, K196T, or a combination thereof. In some embodiments, an evolved sortase is provided herein that includes 1, 2, 3, 4, 5, 6, 7, 8, 9, 10, 11, 12, 13, 14, 15, 16, 17, 18, or all 19 of these mutations. The aforementioned amino acid substitution may provide an evolved sortase that efficiently uses acceptor and/or donor sites not bound by the respective parent wild type sortase. For example, in some embodiments, an evolved sortase utilizes a sortase acceptor site having the sequence LPXTG and a donor site having an N-terminal polyglycine motif. In some embodiments, the evolved sortase utilizes an acceptor and/or donor site that is different to an acceptor and/or donor site (respectively) used by the parent sortase, e.g., a sortase acceptor site including LPXS, LAXT, LAXTG (SEQ ID NO: 116), MPXT, MPXTG, LAXS, LAXSG (SEQ ID NO: 120), NPXT, NPXTG, NAXT, NAXTG, NAXS, NAXSG, LPXP, LPXPG, or an LPXTA (SEQ ID NO: 114) motif.

Preferably the sortase is Sortase A or a variant thereof. Sortase A is a transpeptidase that recognizes a (preferably C-terminal) L(A/P/S)X(T/S/A/C)(G/A) motif of proteins to cleave between (T/S/A/C) and G/A, and subsequently transfers the acyl component to a nucleophile containing (preferably N-terminal) (oligo)glycines (where the motif is L(A/P/S)X(T/S/A/C)G) or (oligo)alanines (where the motif is L(A/P/S)X(T/S/A/C)A). In one embodiment a Sortase A may be one obtainable from Streptococcus pyogenes (e.g. SEQ ID NO: 37), said sortase recognises (inter alia) a sortase acceptor site having the sequence LPXTA, in such cases preferably the sortase acceptor site is Aₙ, wherein n is at least 1. Use of an S. pyogenes sortase is described in Antos et al (2009), J Am Chem Soc, 131, 10800-10801, which is incorporated herein by reference.

Preferably, a Sortase A may be one obtainable from Staphylococcus aureus or a variant thereof.

In one embodiment a sortase acceptor site may comprise (or consist of) L(A/P/S)X(T/S/A/C)(G/A), NPQTN, YPRTG, IPQTG, VPDTG, or LPXTGS, wherein X is any amino acid. For example, a sortase acceptor site may comprise (or consist of) L(A/P/S)X(T/S/A/C)G, NPQTN, YPRTG, IPQTG, VPDTG, or LPXTGS, wherein X is any amino acid.

In one embodiment a sortase acceptor site may comprise (or consist of) NPKTG, XPETG, LGATG, IPNTG, IPETG, NSKTA, NPQTG, NAKTN, NPQSS, LPXTX wherein X is any amino acid, NPX₁TX₂, wherein X, is Lys or Gin and X₂ is Asn, Asp or Gly, X₁PX₂X₃G, wherein X₁ is Leu, Ile, Val or Met, X₂ is any amino acid and X₃ is Ser, Thr or Ala, LPEX₁G, wherein X₁ is Ala, Cys or Ser, LPXS, LAXT, MPXT, MPXTG, LAXS, NPXT, NPXTG, NAXT, NAXTG, NAXS, NAXSG, LPXP, LPXPG, wherein X is any amino acid, LRXTG (SEQ ID NO: 123) or LPAXG (SEQ ID NO: 118), wherein X is any amino acid.

The sortase acceptor site X₁PX₂X₃G may be recognised by Sortase A. In some embodiments where a sortase acceptor site comprises (or consists of) X₁PX₂X₃G, X₂ may be Asp, Glu, Ala, Gin, Lys or Met. In some embodiments, said sortase acceptor site comprises (or consists of) LPX₁TG, where X₁ is any amino acid. In other embodiments the sortase acceptor site comprises (or consists of): LPKTG, LPATG, LPNTG, LPETG, LPNAG, LPNTA, LGATG, IPNTG, or IPETG.

The sortase acceptor site NPX₁TX₂ may be recognised by Sortase B. In some embodiments the sortase acceptor site comprises (or consists of): NPQTN, NPKTG, NSKTA, NPQTG, NAKTN, or NPQSS.

The sortase acceptor site LPXTX may be recognised by Sortase C.

In one embodiment a sortase acceptor site does not comprise (or consist of) NPKTG, XPETG, LGATG, IPNTG, IPETG, NSKTA, NPQTG, NAKTN, NPQSS, LPXTX wherein X is any amino acid, NPX₁TX₂, wherein X, is Lys or Gin and X₂ is Asn, Asp or Gly, X₁PX₂X₃G, wherein X₁ is Leu, Ile, Val or Met, X₂ is any amino acid and X₃ is Ser, Thr or Ala, LPEX₁G, wherein X₁ is Ala, Cys or Ser, LPXS, LAXT, MPXT, MPXTG, LAXS, NPXT, NPXTG, NAXT, NAXTG, NAXS, NAXSG, LPXP, LPXPG, wherein X is any amino acid, LRXTG or LPAXG wherein X is any amino acid.

In embodiments where Sortase A is used, a sortase site (e.g. acceptor or donor site) is a Sortase A site.

In a preferred embodiment a sortase acceptor site described herein may be a Sortase A site. A Sortase A consensus acceptor site may be L(A/P/S)X(T/S/A/C)(G/A), wherein X is any amino acid, such as E. However, it is preferred that the Sortase A consensus acceptor site is L(AP/S)X(T/S/A/C)G.

In one embodiment a Sortase A acceptor site comprises or is selected from LPXSG (SEQ ID NO: 115), LAXTG, LPXTG (SEQ ID NO: 117), LPAXG, LPXCG (SEQ ID NO: 119), LAXSG, LPXAG (SEQ ID NO: 121), LSXTG (SEQ ID NO: 122), LRXTG, and LPXTA. Preferably a Sortase A acceptor site may be selected from LPXSG, LAXTG, LPXTG, and LAXSG, more preferably LPXSG or LAXTG. For example, the Sortase A acceptor site may be LPESG (SEQ ID NO: 112) or LAETG (SEQ ID NO: 113) as exemplified herein.

In some embodiments a sortase acceptor site described herein is followed by one or more C-terminal amino acid residues, such as 1-50, 1-10 or preferably 1-5 (e.g. 2) amino acid residues. In some embodiments a sortase acceptor site is followed by one or more acidic amino acid residues. The acidic amino acid residue may be aspartate or glutamate.

A sortase donor site may comprise (or consist of) Gₙ, wherein n is at least 1, 2, 3, 4, 5, 6, 7, 8, 9 or 10. In one embodiment n is at least 2. Preferably n is 2-10, such as 2-5. More preferably n is 4. Such a donor site may preferably be a Sortase A site, preferably for use with a sortase A acceptor site L(A/P/S)X(T/S/A/C)G.

In some embodiments a sortase donor site may be GₙK, wherein n is at least 1 (e.g. at least 1, 2, 3, 4, 5, 6, 7, 8, 9 or 10, in one embodiment n is at least 2, and preferably n is 2-10, such as 2-5).

In one embodiment a sortase acceptor site for use in the invention comprises (or consists of) L(AP/S)X(T/S/NC)G, wherein X is any amino acid, and a sortase donor site for use in the invention comprises (or consists of) Gₙ, wherein n is at least 1, 2, 3, 4, 5, 6, 7, 8, 9 or 10.

A sortase donor site may comprise (or consist of) Aₙ, wherein n is at least 1, 2, 3, 4, 5, 6, 7, 8, 9 or 10. In one embodiment n is at least 2. Preferably n is 2-10, such as 2-5. More preferably n is 4. Such a donor site may preferably be a Sortase A site, preferably for use with a sortase A acceptor site L(A/P/S)X(T/S/A/C)A.

In one embodiment a sortase acceptor site for use in the invention comprises (or consists of) L(A/P/S)X(T/S/A/C)A, wherein X is any amino acid, and a sortase donor site for use in the invention comprises (or consists of) Aₙ, wherein n is at least 1, 2, 3, 4, 5, 6, 7, 8, 9 or 10.

In the context of sortase acceptor or donor sites X may be any amino acid, for example selected from the standard amino acids: aspartic acid, glutamic acid, arginine, lysine, histidine, asparagine, glutamine, serine, threonine, tyrosine, methionine, tryptophan, cysteine, alanine, glycine, valine, leucine, isoleucine, proline, and phenylalanine. In some embodiments X may be any amino acid except proline.

Where a non-sortase A acceptor site is employed, such as:
a Staphylococcus aureus Sortase B site: NPQTN;
a Streptococcus pneumoniae Sortase B site: YPRTG, IPQTG, or VPDTG;
a Streptococcus pyogenes Sortase B site: LPXTGS;
a Streptococcus pneumoniae Sortase C site: YPRTG, IPQTG, or VPDTG; and
a Streptococcus pneumoniae Sortase D site: YPRTG, IPQTG, or VPDTG;

the person skilled in the art will select the appropriate donor site for use with said non-sortase A acceptor site based on the teaching in the art.

Sortase B may be a catalytically active polypeptide having at least 70% sequence identity to SEQ ID NO: 32 or 34. In one embodiment Sortase B may be a catalytically active polypeptide having at least 80% or 90% sequence identity to SEQ ID NO: 32 or 34. Preferably Sortase B may be a may be a catalytically active comprising (more preferably consisting of) SEQ ID NO: 32 or 34.

Sortase C may be a catalytically active polypeptide having at least 70% sequence identity to SEQ ID NO: 35. In one embodiment Sortase C may be a catalytically active polypeptide having at least 80% or 90% sequence identity to SEQ ID NO: 35. Preferably Sortase C may be a may be a catalytically active comprising (more preferably consisting of) SEQ ID NO: 35.

Sortase D may be a catalytically active polypeptide having at least 70% sequence identity to SEQ ID NO: 36. In one embodiment Sortase D may be a catalytically active polypeptide having at least 80% or 90% sequence identity to SEQ ID NO: 36. Preferably Sortase D may be a may be a catalytically active comprising (more preferably consisting of) SEQ ID NO: 36.

The sortase acceptor site is preferably located at the C-terminus of the polypeptide. The sortase donor site is preferably located at the N-terminus of the polypeptide.

The term “located at the C-terminus” as used in this context may mean that the C-terminal residue of the acceptor site is located up to 50 amino acid residues N-terminal to the C-terminal residue of the polypeptide, for example that the C-terminal residue of the acceptor site is located 1-50, preferably 10-40 amino acid residues N-terminal to the C-terminal residue of the polypeptide. In particularly preferred embodiments the C-terminal residue of the acceptor site may be the C-terminal residue of the polypeptide.

In embodiments where there are one or more residues C-terminal to a sortase acceptor site of the polypeptide, it is preferable that said one or more residues are removed prior to the use of the polypeptide in a labelling method described herein.

The term “located at the N-terminus” as used in this context may mean that the C-terminal residue of the donor site is located up to 50 amino acid residues C-terminal to the N-terminal residue of the polypeptide, for example that the N-terminal residue of the donor site is located 1-50, preferably 1-25 amino acid residues C-terminal to the N-terminal residue of the polypeptide. In particularly preferred embodiments the N-terminal residue of the donor site may be the N-terminal residue of the polypeptide.

In embodiments where there are one or more residues N-terminal to a sortase donor site of the polypeptide, it is preferable that said one or more residues are removed prior to the use of the polypeptide in a labelling method described herein.

In one embodiment a sortase acceptor or donor site is located C-terminal to the TM of the polypeptide. In one embodiment a sortase acceptor or donor site is located N-terminal to the non-cytotoxic protease or proteolytically inactive mutant thereof.

In one embodiment a polypeptide of the invention comprises at least two sortase acceptor sites, at least two sortase donor sites, or at least one sortase acceptor site and at least one sortase donor site. Preferably a polypeptide of the invention comprises one sortase acceptor site and one sortase donor site. When labelled in a method of the invention polypeptides comprising at least two (preferably two) sites as described herein comprise at least two (preferably two) detectable labels. For such polypeptides the at least two sites are preferably different, for example one site may be a donor site and one may be an acceptor site, or alternatively where the at least two sites are the same (e.g. both donor sites or both acceptor sites) it is preferred that the sites have different amino acid sequences. This allows the use of different sortases to mediate labelling, such as sortases that recognise different acceptor sites.

In one embodiment a polypeptide of the invention comprises a sortase acceptor site located C-terminal to the TM of the polypeptide and a sortase donor site located N-terminal to the non-cytotoxic protease or proteolytically inactive mutant thereof (preferably the non-cytotoxic protease).

In one embodiment a method of labelling a polypeptide comprises a two-step labelling process. In one embodiment one of the steps comprises the use of a sortase that recognises a first sortase acceptor site of the polypeptide or labelled substrate, and a second step that comprises the use of a different sortase that recognises a different acceptor site of the polypeptide or labelled substrate. The skilled person will appreciate that should more than two different sortase acceptor sites be used, the method may comprise more than two labelling steps and the use of more than two different sortases, wherein each sortase recognises one of the different sortase acceptor sites.

Preferably a polypeptide comprises an acceptor site comprising (or consisting of) LPXSG and a donor site comprising (or consisting of) Gₙ, wherein n is 2-5. In a particularly preferred embodiment a polypeptide comprises an acceptor site comprising (or consisting of) LPESG and a donor site comprising (or consisting of) G₃.

In one embodiment a method of the invention comprises:
a. providing a polypeptide comprising a sortase acceptor site and a sortase donor site;
b. incubating the polypeptide with:
   a first sortase that recognises the sortase acceptor site; and
   a first labelled substrate comprising a sortase donor site and a conjugated detectable label;
wherein the first sortase catalyses conjunction between an amino acid of the sortase acceptor site and an amino acid of the sortase donor site, thereby labelling the polypeptide;
c. further incubating the polypeptide with:
   a second labelled substrate comprising a different sortase acceptor site and a conjugated detectable label, wherein the sortase acceptor site is different to the sortase acceptor site of the polypeptide; and
   a second sortase that recognises the different sortase acceptor site (and preferably does not recognise the sortase acceptor site of the polypeptide);
wherein the second sortase catalyses conjunction between an amino acid of the different sortase acceptor site and an amino acid of the sortase donor site, thereby further labelling the polypeptide; and
d. obtaining the labelled polypeptide.

The skilled person will appreciate that the order of steps b. and c. of the above-mentioned method can be carried out in any order.

In another embodiment a method of the invention comprises:
a. providing a polypeptide comprising a first sortase acceptor site and a second sortase acceptor site, wherein the first and second sortase acceptor sites are different;
b. incubating the polypeptide with:
   a first sortase that recognises the first sortase acceptor site (and preferably does not recognise the second sortase acceptor site); and
   a labelled substrate comprising a sortase donor site and a conjugated detectable label;
wherein the first sortase catalyses conjunction between an amino acid of the first sortase acceptor site and an amino acid of the sortase donor site, thereby labelling the polypeptide;
c. further incubating the polypeptide with:
   a second sortase that recognises the second sortase acceptor site (and preferably does not recognise the first sortase acceptor site); and
   a labelled substrate comprising a sortase donor site and a conjugated detectable label;
wherein the second sortase catalyses conjunction between an amino acid of the second sortase acceptor site and an amino acid of the sortase donor site, thereby further labelling the polypeptide; and
d. obtaining the labelled polypeptide.

The skilled person will appreciate that the order of steps b. and c. of the above-mentioned method can be carried out in any order.

In step c. the labelled substrate preferably comprises a different detectable label to the labelled substrate of step b., e.g. differently-coloured fluorophores.

In another embodiment a method of the invention comprises:
a. providing a polypeptide comprising a first sortase donor site and a second sortase donor site;
b. incubating the polypeptide with:
   a first labelled substrate comprising a first sortase acceptor site and a conjugated detectable label; and
   a first sortase that recognises the first sortase acceptor site (and preferably does not recognise the second sortase acceptor site);
wherein the first sortase catalyses conjunction between an amino acid of the first sortase acceptor site and an amino acid of the first or second sortase donor site, thereby labelling the polypeptide;
c. further incubating the polypeptide with:
   a second labelled substrate comprising a second sortase acceptor site and a conjugated detectable label, wherein the second sortase acceptor site is different to the first sortase acceptor site; and
   a second sortase that recognises the second sortase acceptor site (and does not recognise the first sortase acceptor site); and
wherein the second sortase catalyses conjunction between an amino acid of the second sortase acceptor site and an amino acid of the first or second sortase donor site, thereby further labelling the polypeptide; and
d. obtaining the labelled polypeptide.

The skilled person will appreciate that the order of steps b. and c. of the above-mentioned method can be carried out in any order.

In step c. the labelled substrate preferably comprises a different detectable label to the labelled substrate of step b., e.g. differently-coloured fluorophores.

In a preferred embodiment a method of the invention comprises:
a. providing a polypeptide comprising a sortase acceptor site comprising LPXSG, wherein X is any amino acid, and a sortase donor site comprising Gₙ, wherein n is 2-5;
b. incubating the polypeptide with:
   a first sortase that recognises the sortase acceptor site comprising LPXSG (and preferably does not recognise the sortase acceptor site comprising LAXTG); and
   a first labelled substrate comprising the sortase donor site comprising Gₙ, wherein n is 2-10 (preferably 2-5), and a conjugated detectable label;
wherein the first sortase catalyses conjunction between an amino acid of the sortase acceptor site of the polypeptide and an amino acid of the sortase donor site of the first labelled substrate, thereby labelling the polypeptide;
c. incubating the polypeptide with:
   a second labelled substrate comprising a sortase acceptor site comprising LAXTG, wherein X is any amino acid, and a conjugated detectable label; and
   a second sortase that recognises the sortase acceptor site comprising LAXTG (and preferably does not recognise the sortase acceptor site comprising LPXSG);
wherein the second sortase catalyses conjunction between an amino acid of the sortase acceptor site of the second labelled substrate and an amino acid of the sortase donor site of the polypeptide, thereby further labelling the polypeptide; and
d. obtaining the labelled polypeptide.

The skilled person will appreciate that the order of steps b. and c. of the above-mentioned method can be carried out in any order.

The detectable label conjugated to the first and second labelled substrates are preferably different, e.g. differently-coloured fluorophores.

The skilled person will appreciate where it is intended to add more than two detectable labels to a polypeptide the polypeptide can comprise more than two sites (e.g. donor or acceptor sites) and that the method can be carried out iteratively.

The term “does not recognise the sortase acceptor site” (or permutations thereof) may mean that the sortase has a lower activity (e.g. cleavage or conjugation) with a polypeptide comprising the subject sortase acceptor site when compared to the activity with the polypeptide of a sortase that recognises said site. In one embodiment the term “does not recognise the sortase acceptor site may mean that the sortase has substantially no, or no, activity (e.g. cleavage or conjugation) with a polypeptide comprising the subject sortase acceptor site when compared to the activity with the polypeptide of a sortase that recognises said site. In one embodiment the term “does not recognise the sortase acceptor site” (or permutations thereof) may mean that the sortase has a lower activity (e.g. cleavage or conjugation) with a polypeptide comprising the subject sortase acceptor site when compared to the activity of said sortase with a polypeptide comprising a sortase acceptor site recognised by the sortase. In one embodiment the term “does not recognise the sortase acceptor site may mean that the sortase has substantially no, or no, activity (e.g. cleavage or conjugation) with a polypeptide comprising the subject sortase acceptor site when compared to the activity of said sortase with a polypeptide comprising a sortase acceptor site recognised by the sortase. A sortase acceptor site recognised by the sortase may be one known in the art to be recognised by said sortase.

An incubation step of a method of the invention may be carried out under any conditions that allow successful labelling of a polypeptide using sortase. Such conditions can be determined by the skilled person using routine techniques/optimisation.

The amounts of polypeptide, sortase, and labelled substrate for use in an incubation step of a method as described herein can be determined by the skilled person using routine techniques. In one embodiment the method comprises the use of an excess of labelled substrate to polypeptide and sortase, and optionally an excess of sortase to polypeptide. In one embodiment the method comprises the use of a weight ratio of 1:2:20 of polypeptide to sortase to labelled substrate. In another embodiment the method comprises the use of a molar ratio of 1:2:20 of polypeptide to sortase to labelled substrate.

The reaction conditions for an incubation step of a method as described herein can also be determined by the skilled person using routine techniques. For example, the reaction may be carried out for at least 2, 4, 6, 8, 10 or 12 hours. Preferably the reaction may be carried out for at least 10 hours. The reaction may be carried out at 1-40 cc, such as 1-37 CC. In one embodiment the reaction may be carried out at 1-10° C., preferably 3-5° C., e.g. about 4° C. The reaction time may be adjusted dependent on the temperature used, e.g. lower temperatures may require a longer incubation time.

After an incubation step of a method of the invention, any free labelled substrate and/or sortase and/or unlabelled polypeptide may be separated from the labelled polypeptide. In one embodiment separation is achieved by way of a tag on a sortase or a labelled polypeptide, preferably a tag (e.g. His-tag) on the labelled polypeptide. The tag may be present on the labelled polypeptide but not on the unlabelled polypeptide, e.g. where the tag is present on the labelled substrate that has been conjugated to the labelled polypeptide.

In one embodiment a separation step may be employed when a polypeptide comprises two or more sites and the method comprises two or more incubation/labelling steps. The separation step may be employed after each incubation/labelling step.

In one embodiment a method of the invention comprises a first incubation and a second incubation (e.g. as detailed herein), wherein after the first incubation a first tag is used to separate the labelled polypeptide from an unlabelled polypeptide. Preferably the first tag is absent from the labelled polypeptide but present on the unlabelled polypeptide, and the unlabelled polypeptide can be removed by way of immuno-depletion. A first tag may be a Strep-tag. In one embodiment after the second incubation a second tag is used to separate the dual-labelled polypeptide from any single-labelled (or unlabelled) polypeptide. Preferably the second tag is present on the dual-labelled polypeptide but absent from the single-labelled (or unlabelled) polypeptide, and the dual-labelled polypeptide can be separated by way of immunoaffinity chromatography. A second tag may be a His-tag.

In embodiments where a polypeptide for labelling using sortase comprises a sortase donor site, the N-terminus of said site may be protected, e.g. by one or more amino acid residues N-terminal thereto. Advantageously, this may prevent circularisation of a polypeptide further comprising a sortase acceptor site. Said one or more amino acids may be removed by way of a cleavable site, such as a TEV cleavage site, thereby exposing the N-terminus of said sortase donor site. Thus, a method of the invention may comprise a step of deprotecting the N-terminus of a sortase donor, e.g. by removing one or more amino acids N-terminal thereto. A deprotection step may be carried out between a first and second incubation step.

In one embodiment where a polypeptide of the invention comprises a cleavable site (e.g. a cleavable site N-terminus to a sortase donor site), said cleavable site may be any cleavable site. In one embodiment a cleavable site may be a site that is non-native (i.e. exogenous) to a clostridial neurotoxin. In some embodiments, a cleavable site is a protease recognition site or a variant thereof with the proviso that the variant is cleavable by the relevant protease. A cleavable site may be one cleaved by Enterokinase, Factor Xa, Tobacco Etch Virus (TEV), Thrombin, PreScission, ADAM17, Human Airway Trypsin-Like Protease (HAT), Elastase, Furin, Granzyme or Caspase 2, 3, 4, 7, 9 or 10. A cleavable site may comprise a polypeptide sequence having at least 70% sequence identity to any one of SEQ ID NOs: 84, 85, 86, 87, 88, 89, 90, 91, 92, 93, 94, 95, 96, 97, 98, 99, or 100. In one embodiment a cleavable site may comprise a polypeptide sequence having at least 80% or 90% sequence identity to any one of SEQ ID NOs: 84, 85, 86, 87, 88, 89, 90, 91, 92, 93, 94, 95, 96, 97, 98, 99, or 100. In another embodiment, a cleavable site comprises (preferably consists of) a non-clostridial cleavable site with a polypeptide sequence shown as any one of SEQ ID NOs: 84, 85, 86, 87, 88, 89, 90, 91, 92, 93, 94, 95, 96, 97, 98, 99, or 100. Preferably, a cleavable site comprises (more preferably consists of) a TEV cleavage site shown as SEQ ID NO: 87.

A sortase for use in the present invention may comprise a polypeptide sequence having at least 70% sequence identity to SEQ ID NO: 14. In one embodiment a sortase for use in the invention may comprise a polypeptide having at least 80% or 90% sequence identity to SEQ ID NO: 14. Preferably, a sortase for use in the invention may comprise (more preferably consist of) a polypeptide sequence shown as SEQ ID NO: 14.

The sortase for use in the invention may be encoded by a nucleic acid sequence having at least 70% sequence identity to SEQ ID NO: 13. In one embodiment a sortase for use in the invention may be encoded by a nucleic acid sequence having at least 80% 90% sequence identity to SEQ ID NO: 13. Preferably, a sortase for use in the invention may be encoded by a nucleic acid sequence comprising (more preferably consisting of) a nucleic acid sequence shown as SEQ ID NO: 13.

A sortase for use in the present invention may comprise a polypeptide sequence having at least 70% sequence identity to SEQ ID NO: 16. In one embodiment a sortase for use in the invention may comprise a polypeptide having at least 80% or 90% sequence identity to SEQ ID NO: 16. Preferably, a sortase for use in the invention may comprise (more preferably consist of) a polypeptide sequence shown as SEQ ID NO: 16.

The sortase for use in the invention may be encoded by a nucleic acid sequence having at least 70% sequence identity to SEQ ID NO: 15. In one embodiment a sortase for use in the invention may be encoded by a nucleic acid sequence having at least 80% or 90% sequence identity to SEQ ID NO: 15. Preferably, a sortase for use in the invention may be encoded by a nucleic acid sequence comprising (more preferably consisting of) a nucleic acid sequence shown as SEQ ID NO: 15.

Sortase A may be a catalytically active polypeptide having at least 70% sequence identity to SEQ ID NO: 31, 33 or 37. In one embodiment Sortase A may be a catalytically active polypeptide having at least 80% or 90% sequence identity to SEQ ID NO: 31, 33 or 37. Preferably Sortase A may be a may be a catalytically active comprising (more preferably consisting of) SEQ ID NO: 31, 33 or 37.

The present invention may comprise the use of at least two sortases (more preferably two), e.g. wherein said sortases comprise polypeptides having at least 70% sequence identity to SEQ ID NOs: 14 and 16, respectively. In one embodiment the present invention may comprise the use of at least two sortases, wherein said sortases comprise polypeptides having at least 80% or 90% sequence identity to SEQ ID NOs: 14 and 16, respectively. Preferably, the present invention may comprise the use of at least two sortases, wherein said sortases comprise (more preferably consist of) polypeptides having SEQ ID NOs: 14 and 16, respectively.

A labelled substrate for use in the methods comprising the use of sortase is a sortase substrate, and comprises a sortase donor or acceptor site and a conjugated detectable label. Where it is intended that a labelled substrate is for labelling a polypeptide comprising a sortase acceptor site, the labelled substrate comprises a sortase donor site, and vice versa. A labelled substrate may be a peptide or polypeptide, preferably a peptide.

A labelled substrate may comprise any of the sortase donor or acceptor sites described herein. A labelled substrate may also comprise one or more tags, such as purification tags (e.g. a His-tag) to aid in purification thereof or separation from the labelled polypeptide.

In one embodiment a labelled substrate comprises a sortase donor site. An example of a labelled substrate comprising a sortase donor site is provided by SEQ ID NO: 29. Thus, in one embodiment there is provided a labelled substrate comprising a polypeptide sequence having at least 70% sequence identity to SEQ ID NO: 29. The labelled substrate may comprise a polypeptide sequence having at least 80% or 90% sequence identity to SEQ ID NO: 29. Preferably the labelled substrate comprises (more preferably consists of) a polypeptide sequence shown as SEQ ID NO: 29.

In one embodiment a labelled substrate comprises a sortase acceptor site. An example of a labelled substrate comprising a sortase acceptor site is provided by SEQ ID NO: 30. Thus, in one embodiment there is provided a labelled substrate comprising a polypeptide sequence having at least 70% sequence identity to SEQ ID NO: 30. The labelled substrate may comprise a polypeptide sequence having at least 80% or 90% sequence identity to SEQ ID NO: 30. Preferably the labelled substrate comprises (more preferably consists of) a polypeptide sequence shown as SEQ ID NO: 30.

The sortase acceptor site is preferably located at the C-terminus of the labelled substrate. The sortase donor site is preferably located at the N-terminus of the labelled substrate.

A polypeptide of the invention is preferably for use as a di-chain polypeptide wherein the two chains are joined together by way of a disulphide bond. In such embodiments, the polypeptide may comprise a sortase donor site located at the N-terminus of one or both of the two polypeptide chains. For example, a di-chain polypeptide may comprise a sortase donor site N-terminal to a non-cytotoxic protease (or proteolytically inactive mutant thereof) and/or a translocation domain thereof. In embodiments where the sortase donor site is N-terminal to a translocation domain of the polypeptide, the sortase donor site may only be accessible for use in a method of the invention once the polypeptide has been converted into a di-chain form (e.g. by proteolytic activation).

The term “located at the C-terminus” as used in this context may mean that the C-terminal residue of the acceptor site is located up to 50 amino acid residues N-terminal to the C-terminal residue of the labelled substrate, for example that the C-terminal residue of the acceptor site is located 1-50, preferably 10-40 amino acid residues N-terminal to the C-terminal residue of the labelled substrate. In particularly preferred embodiments the C-terminal residue of the acceptor site may be the C-terminal residue of the labelled substrate.

In embodiments where there are one or more residues C-terminal to a sortase acceptor site of the labelled substrate, it is preferable that said one or more residues are removed prior to the use of the labelled substrate in a labelling method described herein.

The term “located at the N-terminus” as used in this context may mean that the C-terminal residue of the donor site is located up to 50 amino acid residues C-terminal to the N-terminal residue of the labelled substrate, for example that the N-terminal residue of the donor site is located 1-50, preferably 1-25 amino acid residues C-terminal to the N-terminal residue of the labelled substrate. In particularly preferred embodiments the N-terminal residue of the donor site may be the N-terminal residue of the labelled substrate.

In embodiments where there are one or more residues N-terminal to a sortase donor site of the labelled substrate, it is preferable that said one or more residues are removed prior to the use of the labelled substrate in a labelling method described herein.

By way of proof-of-principle data, the present inventors have demonstrated that any labelling technique similar to the sortase-mediated labelling may be employed in the present invention without negatively affecting the potency (e.g. binding, translocation, and/or catalytic activity) of a polypeptide of the invention. Thus, the present invention encompasses the use of alternative enzymes that are capable of conjugating a labelled polypeptide to the polypeptide of the invention. These may be used instead of or additional to sortase (preferably in addition to, e.g. when labelling at an additional site). Enzymes that may also find utility in the present invention may include alternative transpeptidases or ligases. Thus, embodiments described herein in respect of sortases may be applied to alternative transpeptidases or ligases.

In one embodiment the present invention may comprise the use of a ligase, such as butelase 1 (or a variant thereof), which is a ligase obtainable from the plant species Clitoria ternatea and is described in Nguyen, G. K., Y. Cao, W. Wang, C. F. Liu and J. P. Tam (2015). “Site-Specific N-Terminal Labeling of Peptides and Proteins using Butelase 1 and Thiodepsipeptide.” Angew Chem Int Ed Engl 54(52): 15694-15698 and Nguyen et al (2016), Nature Protocols, 11, 10, 1977-1988, which are incorporated herein by reference. Where the invention comprises the use of a transpeptidase or ligase alternative to sortase, the labelled substrate is a substrate of said transpeptidase or ligase, respectively.

In embodiments where butelase 1 is employed, the polypeptide comprises a butelase 1 acceptor or donor site and a labelled substrate is employed comprising a butelase 1 donor or acceptor site and a conjugated detectable label. Similarly to the methods comprising the use of sortase, where the polypeptide comprises a butelase acceptor site, the labelled substrate comprising the conjugated detectable label comprises a butelase donor site (and vice versa). In such embodiments the labelled substrate is a substrate of butelase (e.g. butelase 1).

Butelase cleaves between Asn/Asp and His of a C-terminal Asn/Asp-His-Val consensus sequence and can ligate a polypeptide comprising an N-terminal amino acid sequence Xaa-(Ile/Leu/Val/Cys), wherein Xaa is any amino acid apart from proline to form a bond between Asn/Asp-Xaa-(Ile/Leu/Val/Cys). In one embodiment the butelase acceptor site comprises (or consists of) Asn/Asp-His-Val. In one embodiment the butelase donor site comprises (or consists of) Xaa-(Ile/Leu/Val/Cys), wherein Xaa is any amino acid apart from proline.

In the context of butelase sites Xaa may be selected (for example) from the standard amino acids: aspartic acid, glutamic acid, arginine, lysine, histidine, asparagine, glutamine, serine, threonine, tyrosine, methionine, tryptophan, cysteine, alanine, glycine, valine, leucine, isoleucine, and phenylalanine.

Thus, there is provided a method for preparing a labelled polypeptide, the method comprising:
a. providing a polypeptide comprising:
   i. a butelase acceptor or donor site;
   ii. a non-cytotoxic protease or a proteolytically inactive mutant thereof;
   iii. a Targeting Moiety (TM) that is capable of binding to a Binding Site on a target cell; and
   iv. a translocation domain;
b. incubating the polypeptide with:
   a butelase (e.g. butelase 1); and
   a labelled substrate comprising a butelase donor or acceptor site and a conjugated detectable label;
   wherein the butelase catalyses conjugation between an amino acid of the butelase acceptor site and an amino acid of the butelase donor site, thereby labelling the polypeptide; and
c. obtaining the labelled polypeptide.

In another aspect the invention provides a polypeptide for labelling with butelase comprising:
a butelase acceptor or donor site;
a non-cytotoxic protease that is capable of cleaving a protein of the exocytic fusion apparatus in a target cell or a proteolytically inactive mutant thereof;
a Targeting Moiety (TM) that is capable of binding to a Binding Site on a target cell; and
a translocation domain that is capable of translocating the non-cytotoxic protease from within an endosome, across the endosomal membrane and into the cytosol of the target cell;
wherein when the polypeptide comprises a butelase donor site, the butelase donor site is located at an N-terminus of the polypeptide; and
   wherein the N-terminal residue of the donor site is the N-terminal residue of the polypeptide; or
   wherein the polypeptide comprises one or more amino acid residues N-terminal to the butelase donor site and a cleavable site, which when cleaved exposes the N-terminus of the butelase donor site.

The invention also provides a labelled polypeptide, the polypeptide comprising:
i. a detectable label conjugated to the polypeptide;
ii. an amino acid sequence that comprises Asn/Asp-Xaa-(Ile/Leu/Val/Cys), wherein Xaa is any amino acid apart from proline;
iii. a non-cytotoxic protease or a proteolytically inactive mutant thereof;
iv. a Targeting Moiety (TM) that is capable of binding to a Binding Site on a target cell; and
v. a translocation domain.

A labelled polypeptide may therefore comprise a detectable label conjugated at or near to an amino acid sequence that comprises (or consists of) Asn/Asp-Xaa-(Ile/Leu/Val/Cys), wherein Xaa is any amino acid apart from proline.

In one embodiment a transpeptidase or ligase, such as butelase 1 is used in combination with sortase to obtain a polypeptide having two or more labels. Thus, in one embodiment a polypeptide of the invention may comprises at least one sortase acceptor or donor site as described herein, and at least one butelase (e.g. butelase 1) acceptor or donor site.

Butelase 1 may be a catalytically-active polypeptide comprising a polypeptide sequence having at least 70% sequence identity to SEQ ID NO: 27 or 28 (preferably SEQ ID NO: 28). In one embodiment butelase 1 may comprise a polypeptide sequence having at least 80%, 90% or 95% sequence identity to SEQ ID NO: 27 or 28 (preferably SEQ ID NO: 28). Preferably butelase 1 may comprise (more preferably consist of) a polypeptide sequence shown as SEQ ID NO: 27 or 28 (preferably SEQ ID NO: 28).

Other ligases may include PATG (SEQ ID NO: 41), PCY1 (SEQ ID NO: 42), POPB (SEQ ID NO: 43) or Butelase homologue OaAEP1b SEQ ID NOs: 44 and 45) (Harris et al (2015), Nat Commun, 6, 10199). Where said ligases have a signal peptide or other N-terminal leader sequence, said signal peptide or leader sequence is preferably removed prior to use in the present invention.

POPB as well as suitable methods for the use thereof are taught in the art. For example as described in Luo H (2014), Chemistry and Biology 21: 1610-1617, which is incorporated herein by reference.

Thus, a ligase for use in the present invention may comprise a polypeptide sequence having at least 70% sequence identity to any one of SEQ ID NOs: 41-44. In one embodiment a ligase may comprise a polypeptide sequence having at least 80%, 90% or 95% sequence identity to any one of SEQ ID NOs: 41-44. Preferably a ligase may comprise (more preferably consist of) a polypeptide sequence shown as any one of SEQ ID NOs: 41-44.

The present invention encompasses the use of any suitable detectable label known to the person skilled in the art. The detectable label may be a label that can be detected visually, by way of the label's optical properties. Such a label may be detected using fluorescent techniques, e.g. fluorescent microscopy. Thus, in a particularly preferred embodiment, a detectable label is a fluorophore. Preferably the detectable label is (or comprises) a fluorescent dye, such the HiLyte fluorescent dyes (commercially available from AnaSpec), AlexaFluor (commercially available from Thermo Fisher), Atto (commercially available from Sigma-Aldrich), Quantum Dots commercially available from Sigma-Aldrich), Janelia Fluor dyes (available from Janelia, US) amongst others. In a preferred embodiment a detectable label does not comprise a polysaccharide and/or a polyalcohol and/or a bacterial or viral polymer (e.g. polysaccharide or polypeptide).

In one aspect the invention also provides a method for assaying a polypeptide of the present invention, the method comprising:

a. contacting a target cell with the labelled polypeptide of the invention; and

b. detecting the detectable label.

Such methods may be carried out in vitro or in vivo (e.g. in a mammal, such as non-human mammal, for example a mouse). Preferably the methods are carried out in vitro. When carried out in vivo the method may comprise removing a tissue sample for ex vivo analysis.

The methods of the invention are preferably carried out using live cells/tissues, preferably in real-time. Said methods advantageously allow for determining binding, trafficking and translocation of a polypeptide of the invention.

The method may be a pulse-chase experiment or include a pulse step (e.g. comprising the use of a labelled polypeptide) and a chase step (e.g. not comprising the use of labelled polypeptide and optionally comprising the use of unlabelled polypeptide).

Detecting the detectable label allows detection of the polypeptide or a portion thereof. For example, where the polypeptide comprises a first detectable label conjugated to the non-cytotoxic protease or proteolytically inactive mutant thereof and a second detectable label conjugated to the translocation domain or TM, the method may comprise detection of both of said detectable labels.

A method of the invention may comprise detecting the presence or absence of co-localisation of two or more detectable labels. Detection can be achieved using any technique known to the person skilled in the art (e.g. FRET and related techniques). In one embodiment a method of the invention comprises detecting a change in the co-localisation of two or more detectable labels, e.g. over time. In embodiments where the polypeptide comprises a first detectable label conjugated to the non-cytotoxic protease or proteolytically inactive mutant thereof and a second detectable label conjugated to the translocation domain or TM, detecting a reduction in co-localisation of the first and second detectable labels (e.g. over time) may allow for the measurement of translocation of the non-cytotoxic protease or proteolytically inactive mutant thereof out of an endosome. The time taken for such a change in co-localisation to occur may be used to determine a translocation rate. Detecting no change (e.g. substantially no change) in co-localisation may indicate that translocation has not occurred.

The method may comprise detecting the presence of the first detectable label in the cytosol of a cell and/or the second detectable label in an endosome of a cell, which may also provide an assay of translocation. Likewise, detecting the first and second detectable label (co-localisation) in an endosome may be an indication that the polypeptide has been successfully endocytosed.

In some embodiments a method of the invention may comprise quantifying the amount of detectable label, e.g. at a particular location in a cell and/or over a particular time course.

Such quantification may be determined by detecting the intensity of a detectable label at a particular location in a cell (e.g. over time). Alternatively or additionally, quantification may be performed by determining the number or size of agglomerates comprising said detectable label present in a cell.

In one embodiment a method of the invention comprises:
i) contacting a target cell with a labelled polypeptide of the invention that is to be assessed for endosome release ability, wherein said target cell comprises a cell membrane including a Binding Site present on the outer surface of the cell membrane of said cell;
ii) incubating the labelled polypeptide with said target cell, and thereby allowing
   a) the labelled polypeptide to bind to and form a bound complex with the Binding Site present on the target cell, thereby permitting said bound complex to enter the target cell by endocytosis;
   b) one or more endosomes to form within said cell, wherein the one or more endosomes contain the labelled polypeptide; and
   c) said labelled polypeptide to enter the cytosol of the target cell by crossing the endosomal membrane of the one or more endosomes;
iii) removing excess labelled polypeptide that is not bound to the Binding Sites present on the target cells;
iv) after a predetermined period of time, detecting the amount of labelled polypeptide present in the one or more endosomes, or detecting the amount of labelled polypeptide present in the cytosol of said target cell;
v) comparing the amount of labelled polypeptide detected in step iv) with a control value, wherein said control value represents the amount of labelled polypeptide present in the one or more endosomes or the amount of labelled polypeptide present in the cytosol prior to step iv);
vi) calculating an endosome release value for the labelled polypeptide by determining the relative change in the amount of labelled polypeptide that is present within the one or more endosomes, or by determining the relative change in the amount of labelled polypeptide present in the cytosol of said target cell.

The target cell may be a eukaryotic cell such as a mammalian cell, for example a target cell described herein.

Incubation step ii) may proceed for any given time period, for example for a time period from 5 minutes to 5 days. A typical time period is 1-12 hours, for example 2-10 hours, 4-8 hours, or 6-8 hours. During this period, the target cell (i.e. the outer surface of the cell membrane) may be exposed to labelled polypeptide (typically an excess of labelled polypeptide) with the result that a ‘steady state’ is achieved in which labelled polypeptide enters and leaves the intracellular endosomes at approximately the same rate. This point in time represents an optimal time point at which to perform steps iii and/or iv).

Step iii) may involve reducing or removing the source of labelled polypeptide external to the target cell, thereby reducing the amount of (or substantially preventing) the labelled polypeptide entering the cell. Said reduction in the amount of labelled polypeptide entering the target cell, in turn, provides a change in the amount of labelled polypeptide entering the endosomes, which in turn results in a change in the amount (or rate) of labelled polypeptide leaving the endosomes and/or entering the cytosol of the target cell. It is the amount (or rate) of labelled polypeptide leaving the endosome structures that may provide in one embodiment the basis of the assay—said amount (or rate) of labelled polypeptide leaving the endosome structures may be measured by a change in the amount of labelled polypeptide present in the endosomes and/or by a change in the amount of labelled polypeptide present in the cytosol. When measuring the amount of labelled polypeptide present in the endosomes, a reduction in the amount of labelled polypeptide present is typically observed. When measuring the amount of labelled polypeptide present in the cytosol, an increase or decrease in the amount of labelled polypeptide present within the cytosol may be observed. By way of example, an increase in the amount of labelled polypeptide in the cytosol may be observed when step iii) is initiated prior to establishment of steady state endosomal transport of the labelled polypeptide. Alternatively, a decrease in the amount of labelled polypeptide in the cytosol may be observed when the rate of cellular secretion of the labelled polypeptide from the target cell exceeds the rate of endosomal transport of the labelled polypeptide from the endosomes into the cytosol.

The target cells employed in the assay may be immobilised on a surface. Immobilisation of the cells may be performed as a pre-assay step (i.e. pre-immobilization), or may be performed as part of the assay protocol. Thus, in one embodiment, the cells of the assay are pre-immobilized. Immobilisation of the target cells may be performed by any conventional means. By way of example, cells are seeded into the assay plates at high density and allowed to adhere before the assay is conducted. Alternatively, cells are seeded into assay plates and cultured for several days before use to provide a confluent monolayer. Cell attachment may be enhanced by using conventional coatings, such as poly-D-lysine coated plates.

In one embodiment, immobilisation of the target cells may be performed prior to or during step iii), thereby providing a simple means for separating said cells from free (e.g. unbound or exogenous) labelled polypeptide. Alternatively, immobilisation may be performed after step iii), for example to facilitate detection step iv).

Step iii) may include a filtering step or affinity ligand step during which the target cells are separated from excess (e.g. unbound or exogenous) labelled polypeptide. Step iii) may include a washing step in which excess (e.g. unbound or exogenous) labelled polypeptide is washed away from the target cells, for example using a conventional buffer. Excess labelled polypeptide is intended to mean labelled polypeptide that is present in the assay medium, external to the target cells, and which has not yet become bound to a Binding Site present on the surface of the target cells.

Detection of labelled polypeptide in step iv) is typically performed shortly after step iii). By way of example, a typical timeframe for step iv) is between 5 minutes and 5 hours following step iii). In one embodiment, step iv) is performed 15-240 minutes, or 30-180 minutes, or 45-150 minutes following step iii). Detection step iv) may be repeated over several time points, for example at intervals of 10 minutes or 15 minutes or 30 minutes—this will permit a rate of endosomal release to be calculated.

Detection step iv) may be performed by any conventional means. Detection of the labelled polypeptide may be based upon intracellular localisation of said labelled polypeptide.

Comparison step v) employs the use of a control value, which represents the amount of labelled polypeptide present in the endosomes and/or cytosol prior to detecting step iv). The control value is typically determined by the same means/method by which the amount of labelled polypeptide is determined in detection step iv). The control value typically represents the amount of labelled polypeptide present in the endosomes and/or cytosol during or before step iii). By way of example, the control value may represent the amount of labelled polypeptide present in the endosomes and/or cytosol during or at the end of step ii)—in one embodiment, the control value represents the amount of labelled polypeptide that is present in the endosomes and/or cytosol when a ‘steady state’ translocation rate has been established, namely when labelled polypeptide enters and leaves the intracellular endosomes at approximately the same rate.

In the foregoing embodiments the term labelled polypeptide may also encompass a portion thereof, such as a non-cytotoxic protease domain, a translocation domain, or a TM (e.g. a translocation domain and a TM). The methods may also comprise detecting two or more labels, such as a label on one portion of the polypeptide and a label on a second portion of the polypeptide.

In one embodiment a method of the invention may also comprise assaying cleavage of a protein of the exocytic fusion apparatus (e.g. a SNARE protein).

The detectable label may be detected using any suitable techniques known to the person skilled in the art. In one embodiment microscopy is used to detect the detectable label. Techniques for detecting a detectable label may include any suitable light, confocal (preferably 3D live confocal microscopy), super resolution, or single molecule imaging technique (e.g. light microscopy, confocal microscopy, super resolution microscopy or single molecule imaging). Microscopes such as STED, PALM, STORM and TIRF might be employed in methods of the invention. Such microscopy techniques are well established and of high resolution.

The term “proteolytically inactive mutant” is intended to encompass a non-cytotoxic protease mutant that exhibits significantly-reduced cleavage of proteins of the exocytic fusion apparatus in a target cell when compared to a non-mutant form thereof. Preferably, a proteolytically inactive mutant comprises a proteolytically inactive clostridial neurotoxin L-chain. In one embodiment, the proteolytically inactive mutant may comprise a L-chain of SEQ ID NOs: 38 or 40.

In one embodiment a “proteolytically inactive mutant” exhibits substantially no non-cytotoxic protease activity, preferably exhibits no non-cytotoxic protease activity. The term “substantially no non-cytotoxic protease activity” means that the proteolytically inactive mutant has less than 5% of the non-cytotoxic protease activity of a non-mutant (i.e. proteolytically active) form thereof, for example less than 2%, 1% or preferably less than 0.1% of the non-cytotoxic protease activity of a non-mutant form thereof. Non-cytotoxic protease activity can be determined in vitro by incubating a test non-cytotoxic protease mutant with a SNARE protein and comparing the amount of SNARE protein cleaved by the test non-cytotoxic protease when compared to the amount of SNARE protein cleaved by a non-mutant (i.e. proteolytically active) form thereof under the same conditions. Routine techniques, such as SDS-PAGE and Western blotting can be used to quantify the amount of SNARE protein cleaved. Suitable in vitro assays are described in WO 2019/145577 A1, which is incorporated herein by reference. Alternatively or additionally, a cell-based assay described herein may be used.

In one embodiment, the proteolytically inactive mutant may have one or more mutations that inactivate said protease activity. For example, the proteolytically inactive mutant of a non-cytotoxic protease may comprise a BoNT/A L-chain comprising a mutation of an active site residue, such as His223, Glu224, His227, Glu262, and/or Tyr366. The position numbering corresponds to the amino acid positions of SEQ ID NO: 17 and can be determined by aligning a polypeptide with SEQ ID NO: 17.

A polypeptide of the invention preferably has one or more activities associated with a clostridial neurotoxin (e.g. a botulinum neurotoxin). In other words a polypeptide of the invention may be an active neurotoxin. For example, a polypeptide of the invention may cleave a protein of the exocytic fusion apparatus in a target cell, be capable of binding to a Binding Site on a target cell and/or possess translocation activity. Preferably, a polypeptide of the invention may cleave a protein of the exocytic fusion apparatus in a target cell, be capable of binding to a Binding Site on a target cell, and possess translocation activity. Thus, preferably a polypeptide is not subjected to (and has not been subjected to) a detoxification treatment. For example, the polypeptide may not be (and may not have been) chemically inactivated and/or heat-inactivated. In one embodiment the polypeptide is not contacted with (and has not been contacted with) a crosslinking agent, more preferably the polypeptide is not contacted with (and has not been contacted with) with formaldehyde.

A polypeptide described herein preferably comprises a non-cytotoxic protease that is capable of cleaving a protein of the exocytic fusion apparatus in a target cell.

The Targeting Moiety (TM) of a polypeptide of the invention is preferably capable of binding to a Binding Site on a target cell, which Binding Site is capable of undergoing endocytosis to be incorporated into an endosome within the target cell.

The translocation domain is preferably capable of translocating the non-cytotoxic protease from within an endosome, across the endosomal membrane and into the cytosol of the target cell.

In a preferred embodiment a non-cytotoxic protease of a polypeptide described herein comprises a clostridial neurotoxin L-chain. More preferably, the clostridial neurotoxin L-chain is a botulinum neurotoxin L-chain.

In a preferred embodiment a translocation domain of a polypeptide described herein comprises a clostridial neurotoxin translocation domain. More preferably, the clostridial neurotoxin translocation domain is a botulinum neurotoxin translocation domain.

In one embodiment a polypeptide described herein lacks a functional H_{C} domain of a clostridial neurotoxin.

In an alternative embodiment, a polypeptide described herein comprises a clostridial neurotoxin binding domain (H_{C} domain) TM. More preferably, the clostridial neurotoxin binding domain (H_{C} domain) TM is a botulinum neurotoxin binding domain (H_{C} domain) TM.

Thus, in a preferred embodiment a polypeptide described herein comprises a clostridial neurotoxin L-chain, a clostridial neurotoxin translocation domain, and a non-clostridial TM.

In an equally-preferred alternative embodiment, a polypeptide described herein comprises a clostridial neurotoxin L-chain and a clostridial neurotoxin H-chain (having a clostridial neurotoxin translocation domain [H_{N}] and H_{C} domain). In such embodiments a polypeptide described herein is a clostridial neurotoxin.

More preferably, a polypeptide described herein comprises a botulinum neurotoxin L-chain, a botulinum neurotoxin translocation domain, and a non-clostridial TM.

In an equally-preferred alternative embodiment, a polypeptide described herein comprises a botulinum neurotoxin L-chain and a botulinum neurotoxin H-chain (having a botulinum neurotoxin translocation domain [H_{N}] and H_{C} domain). In such embodiments a polypeptide described herein is a botulinum neurotoxin.

Preferably the polypeptide is a botulinum neurotoxin (BoNT) further comprising the sortase acceptor and/or donor site and/or the detectable label conjugated thereto and an amino acid sequence that comprises L(A/P/S)X(T/S/A/C)Gₙ, wherein X is any amino acid and n is at least 1, L(A/P/S)X(T/S/A/C)Aₙ, wherein X is any amino acid and n is at least 1, NPQTN, YPRTG, IPQTG, VPDTG, LPXTGS, wherein X is any amino acid, NPKTG, XPETG, LGATG, IPNTG, IPETG, NSKTA, NPQTG, NAKTN, NPQSS, LPXTX, wherein X is any amino acid, NPX₁TX₂, wherein X₁ is Lys or Gln and X₂ is Asn, Asp or Gly, X₁PX₂X₃G, wherein X₁ is Leu, Ile, Val or Met, X₂ is any amino acid and X₃ is Ser, Thr or Ala, LPEX₁G, wherein X₁ is Ala, Cys or Ser, LPXS, LAXT, MPXT, MPXTG, LAXS, NPXT, NPXTG, NAXT, NAXTG, NAXS, NAXSG, LPXP, LPXPG, wherein X is any amino acid, LRXTGₙ or LPAXGₙ, wherein X is any amino acid and n is at least 1 (more preferably L(A/P/S)X(T/S/A/C)Gₙ, wherein X is any amino acid and n is at least 1). The BoNT may be one or more selected from BoNT/A, BoNT/B, BoNT/C, BoNT/D, BoNT/E, BoNT/F, BoNT/G or BoNT/X. Also encompassed are variants thereof comprising a proteolytically inactive mutant of the non-cytotoxic protease.

Preferably the polypeptide is a botulinum neurotoxin (BoNT) further comprising the sortase acceptor and/or donor site and/or the detectable label conjugated thereto and an amino acid sequence that comprises L(A/P/S)X(T/S/A/C)Gₙ, wherein X is any amino acid and n is at least 1, L(A/P/S)X(T/S/A/C)Aₙ, wherein X is any amino acid and n is at least 1, NPQTN, YPRTG, IPQTG, VPDTG, or LPXTGS, wherein X is any amino acid (more preferably L(A/P/S)X(T/S/A/C)Gₙ, wherein X is any amino acid and n is at least 1). The BoNT may be one or more selected from BoNT/A, BoNT/B, BoNT/C, BoNT/D, BoNT/E, BoNT/F, BoNT/G or BoNT/X. Also encompassed are variants thereof comprising a proteolytically inactive mutant of the non-cytotoxic protease.

Alternatively, the polypeptide may be a tetanus neurotoxin (TeNT) further comprising the sortase acceptor and/or donor site and/or the detectable label conjugated thereto and an amino acid sequence that comprises L(A/P/S)X(T/S/A/C)Gₙ, wherein X is any amino acid and n is at least 1, L(A/P/S)X(T/S/A/C)Aₙ, wherein X is any amino acid and n is at least 1, NPQTN, YPRTG, IPQTG, VPDTG, LPXTGS, wherein X is any amino acid, NPKTG, XPETG, LGATG, IPNTG, IPETG, NSKTA, NPQTG, NAKTN, NPQSS, LPXTX, wherein X is any amino acid, NPX₁TX₂, wherein X₁ is Lys or Gln and X₂ is Asn, Asp or Gly, X₁PX₂X₃G, wherein X₁ is Leu, Ile, Val or Met, X₂ is any amino acid and X₃ is Ser, Thr or Ala, LPEX₁G, wherein X₁ is Ala, Cys or Ser, LPXS, LAXT, MPXT, MPXTG, LAXS, NPXT, NPXTG, NAXT, NAXTG, NAXS, NAXSG, LPXP, LPXPG, wherein X is any amino acid, LRXTGₙ or LPAXGₙ, wherein X is any amino acid and n is at least 1 (more preferably L(A/P/S)X(T/S/A/C)Gₙ, wherein X is any amino acid and n is at least 1). Also encompassed are variants thereof comprising a proteolytically inactive mutant of the non-cytotoxic protease.

Alternatively, the polypeptide may be a tetanus neurotoxin (TeNT) further comprising the sortase acceptor and/or donor site and/or the detectable label conjugated thereto and an amino acid sequence that comprises L(A/P/S)X(T/S/A/C)Gₙ, wherein X is any amino acid and n is at least 1, L(A/P/S)X(T/S/A/C)Aₙ, wherein X is any amino acid and n is at least 1, NPQTN, YPRTG, IPQTG, VPDTG, or LPXTGS, wherein X is any amino acid (more preferably L(A/P/S)X(T/S/A/C)Gₙ, wherein X is any amino acid and n is at least 1). Also encompassed are variants thereof comprising a proteolytically inactive mutant of the non-cytotoxic protease.

Representative polypeptide sequences for BoNT/A, BoNT/B, BoNT/C, BoNT/D, BoNT/E, BoNT/F, BoNT/G, BoNT/X, and TeNT are described herein as SEQ ID NOs 17-25, respectively. Said polypeptide sequences can be modified to include a sortase acceptor or donor site for use in the present invention.

A polypeptide of the invention may be a polypeptide comprising the sortase acceptor and/or donor site and/or the detectable label conjugated thereto and an amino acid sequence that comprises L(A/P/S)X(T/S/A/C)Gₙ, wherein X is any amino acid and n is at least 1, L(A/P/S)X(T/S/A/C)Aₙ, wherein X is any amino acid and n is at least 1, NPQTN, YPRTG, IPQTG, VPDTG, LPXTGS, wherein X is any amino acid, NPKTG, XPETG, LGATG, IPNTG, IPETG, NSKTA, NPQTG, NAKTN, NPQSS, LPXTX, wherein X is any amino acid, NPX₁TX₂, wherein X₁ is Lys or Gln and X₂ is Asn, Asp or Gly, X₁PX₂X₃G, wherein X₁ is Leu, Ile, Val or Met, X₂ is any amino acid and X₃ is Ser, Thr or Ala, LPEX₁G, wherein X₁ is Ala, Cys or Ser, LPXS, LAXT, MPXT, MPXTG, LAXS, NPXT, NPXTG, NAXT, NAXTG, NAXS, NAXSG, LPXP, LPXPG, wherein X is any amino acid, LRXTGₙ or LPAXGₙ, wherein X is any amino acid and n is at least 1 (more preferably L(A/P/S)X(T/S/A/C)Gₙ, wherein X is any amino acid and n is at least 1) and wherein the polypeptide further comprises a polypeptide sequence having at least 70% sequence identity to any of SEQ ID NOs 17-25. In one embodiment a polypeptide of the invention may be a polypeptide comprising the sortase acceptor and/or donor site and/or the detectable label conjugated thereto and an amino acid sequence that comprises L(A/P/S)X(T/S/A/C)Gₙ, wherein X is any amino acid and n is at least 1, L(A/P/S)X(T/S/A/C)Aₙ, wherein X is any amino acid and n is at least 1, NPQTN, YPRTG, IPQTG, VPDTG, LPXTGS, wherein X is any amino acid, NPKTG, XPETG, LGATG, IPNTG, IPETG, NSKTA, NPQTG, NAKTN, NPQSS, LPXTX, wherein X is any amino acid, NPX₁TX₂, wherein X₁ is Lys or Gin and X₂ is Asn, Asp or Gly, X₁PX₂X₃G, wherein X₁ is Leu, Ile, Val or Met, X₂ is any amino acid and X₃ is Ser, Thr or Ala, LPEX₁G, wherein X₁ is Ala, Cys or Ser, LPXS, LAXT, MPXT, MPXTG, LAXS, NPXT, NPXTG, NAXT, NAXTG, NAXS, NAXSG, LPXP, LPXPG, wherein X is any amino acid, LRXTGₙ or LPAXGₙ, wherein X is any amino acid and n is at least 1 (more preferably L(A/P/S)X(T/S/A/C)Gₙ, wherein X is any amino acid and n is at least 1) and wherein the polypeptide further comprises a polypeptide sequence having at least 80% or 90% sequence identity to any of SEQ ID NOs 17-25. Preferably a polypeptide of the invention may be a polypeptide comprising the sortase acceptor and/or donor site and/or the detectable label conjugated thereto and an amino acid sequence that comprises L(A/P/S)X(T/S/A/C)Gₙ, wherein X is any amino acid and n is at least 1, L(A/P/S)X(T/S/A/C)Aₙ, wherein X is any amino acid and n is at least 1, NPQTN, YPRTG, IPQTG, VPDTG, LPXTGS, wherein X is any amino acid, NPKTG, XPETG, LGATG, IPNTG, IPETG, NSKTA, NPQTG, NAKTN, NPQSS, LPXTX, wherein X is any amino acid, NPX₁TX₂, wherein X, is Lys or Gin and X₂ is Asn, Asp or Gly, X₁PX₂X₃G, wherein X₁ is Leu, Ile, Val or Met, X₂ is any amino acid and X₃ is Ser, Thr or Ala, LPEX₁G, wherein X₁ is Ala, Cys or Ser, LPXS, LAXT, MPXT, MPXTG, LAXS, NPXT, NPXTG, NAXT, NAXTG, NAXS, NAXSG, LPXP, LPXPG, wherein X is any amino acid, LRXTGₙ or LPAXGₙ, wherein X is any amino acid and n is at least 1 (more preferably L(A/P/S)X(T/S/A/C)Gₙ, wherein X is any amino acid and n is at least 1) and wherein the polypeptide further comprises a polypeptide comprising (more preferably consisting of) any of SEQ ID NOs 17-25.

A polypeptide of the invention may be a polypeptide comprising the sortase acceptor and/or donor site and/or the detectable label conjugated thereto and an amino acid sequence that comprises L(A/P/S)X(T/S/A/C)Gₙ, wherein X is any amino acid and n is at least 1, L(A/P/S)X(T/S/A/C)Aₙ, wherein X is any amino acid and n is at least 1, NPQTN, YPRTG, IPQTG, VPDTG, or LPXTGS, wherein X is any amino acid (more preferably L(A/P/S)X(T/S/A/C)Gₙ, wherein X is any amino acid and n is at least 1) and wherein the polypeptide further comprises a polypeptide sequence having at least 70% sequence identity to any of SEQ ID NOs 17-25. In one embodiment a polypeptide of the invention may be a polypeptide comprising the sortase acceptor and/or donor site and/or the detectable label conjugated thereto and an amino acid sequence that comprises L(A/P/S)X(T/S/A/C)Gₙ, wherein X is any amino acid and n is at least 1, L(A/P/S)X(T/S/A/C)Aₙ, wherein X is any amino acid and n is at least 1, NPQTN, YPRTG, IPQTG, VPDTG, or LPXTGS, wherein X is any amino acid (more preferably L(A/P/S)X(T/S/A/C)Gₙ, wherein X is any amino acid and n is at least 1) and wherein the polypeptide further comprises a polypeptide sequence having at least 80% or 90% sequence identity to any of SEQ ID NOs 17-25. Preferably a polypeptide of the invention may be a polypeptide comprising the sortase acceptor and/or donor site and/or the detectable label conjugated thereto and an amino acid sequence that comprises L(A/P/S)X(T/S/A/C)Gₙ, wherein X is any amino acid and n is at least 1, L(A/P/S)X(T/S/A/C)Aₙ, wherein X is any amino acid and n is at least 1, NPQTN, YPRTG, IPQTG, VPDTG, or LPXTGS, wherein X is any amino acid (more preferably L(A/P/S)X(T/S/A/C)Gₙ, wherein X is any amino acid and n is at least 1) and wherein the polypeptide further comprises a polypeptide comprising (more preferably consisting of) any of SEQ ID NOs 17-25.

Alternatively, a polypeptide of the invention may be a polypeptide comprising the sortase acceptor and/or donor site and/or the detectable label conjugated thereto and an amino acid sequence that comprises L(A/P/S)X(T/S/A/C)Gₙ, wherein X is any amino acid and n is at least 1, L(A/P/S)X(T/S/A/C)Aₙ, wherein X is any amino acid and n is at least 1, NPQTN, YPRTG, IPQTG, VPDTG, LPXTGS, wherein X is any amino acid, NPKTG, XPETG, LGATG, IPNTG, IPETG, NSKTA, NPQTG, NAKTN, NPQSS, LPXTX, wherein X is any amino acid, NPX₁TX₂, wherein X₁ is Lys or Gln and X₂ is Asn, Asp or Gly, X₁PX₂X₃G, wherein X₁ is Leu, Ile, Val or Met, X₂ is any amino acid and X₃ is Ser, Thr or Ala, LPEX₁G, wherein X₁ is Ala, Cys or Ser, LPXS, LAXT, MPXT, MPXTG, LAXS, NPXT, NPXTG, NAXT, NAXTG, NAXS, NAXSG, LPXP, LPXPG, wherein X is any amino acid, LRXTGₙ or LPAXGₙ, wherein X is any amino acid and n is at least 1 (more preferably L(A/P/S)X(T/S/A/C)Gₙ, wherein X is any amino acid and n is at least 1) and wherein the polypeptide further comprises a polypeptide sequence having at least 70% sequence identity to SEQ ID NO: 38. In one embodiment a polypeptide of the invention may be a polypeptide comprising the sortase acceptor and/or donor site and/or the detectable label conjugated thereto and an amino acid sequence that comprises L(A/P/S)X(T/S/A/C)Gₙ, wherein X is any amino acid and n is at least 1, L(A/P/S)X(T/S/A/C)Aₙ, wherein X is any amino acid and n is at least 1, NPQTN, YPRTG, IPQTG, VPDTG, LPXTGS, wherein X is any amino acid, NPKTG, XPETG, LGATG, IPNTG, IPETG, NSKTA, NPQTG, NAKTN, NPQSS, LPXTX, wherein X is any amino acid, NPX₁TX₂, wherein X, is Lys or Gln and X₂ is Asn, Asp or Gly, X₁PX₂X₃G, wherein X₁ is Leu, Ile, Val or Met, X₂ is any amino acid and X₃ is Ser, Thr or Ala, LPEX₁G, wherein X₁ is Ala, Cys or Ser, LPXS, LAXT, MPXT, MPXTG, LAXS, NPXT, NPXTG, NAXT, NAXTG, NAXS, NAXSG, LPXP, LPXPG, wherein X is any amino acid, LRXTGₙ or LPAXGₙ, wherein X is any amino acid and n is at least 1 (more preferably L(A/P/S)X(T/S/A/C)Gₙ, wherein X is any amino acid and n is at least 1) and wherein the polypeptide further comprises a polypeptide sequence having at least 80% or 90% sequence identity to SEQ ID NO: 38. Preferably a polypeptide of the invention may be a polypeptide comprising the sortase acceptor and/or donor site and/or the detectable label conjugated thereto and an amino acid sequence that comprises L(A/P/S)X(T/S/A/C)Gₙ, wherein X is any amino acid and n is at least 1, L(A/P/S)X(T/S/A/C)Aₙ, wherein X is any amino acid and n is at least 1, NPQTN, YPRTG, IPQTG, VPDTG, LPXTGS, wherein X is any amino acid, NPKTG, XPETG, LGATG, IPNTG, IPETG, NSKTA, NPQTG, NAKTN, NPQSS, LPXTX, wherein X is any amino acid, NPX₁TX₂, wherein X₁ is Lys or Gin and X₂ is Asn, Asp or Gly, X₁PX₂X₃G, wherein X₁ is Leu, Ile, Val or Met, X₂ is any amino acid and X₃ is Ser, Thr or Ala, LPEX₁G, wherein X₁ is Ala, Cys or Ser, LPXS, LAXT, MPXT, MPXTG, LAXS, NPXT, NPXTG, NAXT, NAXTG, NAXS, NAXSG, LPXP, LPXPG, wherein X is any amino acid, LRXTGₙ or LPAXGₙ, wherein X is any amino acid and n is at least 1 (more preferably L(A/P/S)X(T/S/A/C)Gₙ, wherein X is any amino acid and n is at least 1) and wherein the polypeptide further comprises a polypeptide comprising (more preferably consisting of) SEQ ID NO: 38.

Alternatively, a polypeptide of the invention may be a polypeptide comprising the sortase acceptor and/or donor site and/or the detectable label conjugated thereto and an amino acid sequence that comprises L(A/P/S)X(T/S/A/C)Gₙ, wherein X is any amino acid and n is at least 1, L(A/P/S)X(T/S/A/C)Aₙ, wherein X is any amino acid and n is at least 1, NPQTN, YPRTG, IPQTG, VPDTG, or LPXTGS, wherein X is any amino acid (more preferably L(A/P/S)X(T/S/A/C)Gₙ, wherein X is any amino acid and n is at least 1) and wherein the polypeptide further comprises a polypeptide sequence having at least 70% sequence identity to SEQ ID NO: 38. In one embodiment a polypeptide of the invention may be a polypeptide comprising the sortase acceptor and/or donor site and/or the detectable label conjugated thereto and an amino acid sequence that comprises L(A/P/S)X(T/S/A/C)Gₙ, wherein X is any amino acid and n is at least 1, L(A/P/S)X(T/S/A/C)Aₙ, wherein X is any amino acid and n is at least 1, NPQTN, YPRTG, IPQTG, VPDTG, or LPXTGS, wherein X is any amino acid (more preferably L(A/P/S)X(T/S/A/C)Gₙ, wherein X is any amino acid and n is at least 1) and wherein the polypeptide further comprises a polypeptide sequence having at least 80% or 90% sequence identity to SEQ ID NO: 38. Preferably a polypeptide of the invention may be a polypeptide comprising the sortase acceptor and/or donor site and/or the detectable label conjugated thereto and an amino acid sequence that comprises L(A/P/S)X(T/S/A/C)Gₙ, wherein X is any amino acid and n is at least 1, L(A/P/S)X(T/S/A/C)Aₙ, wherein X is any amino acid and n is at least 1, NPQTN, YPRTG, IPQTG, VPDTG, or LPXTGS, wherein X is any amino acid (more preferably L(A/P/S)X(T/S/A/C)Gₙ, wherein X is any amino acid and n is at least 1) and wherein the polypeptide further comprises a polypeptide comprising (more preferably consisting of) SEQ ID NO: 38.

Polypeptides described herein (or the nucleotide sequences encoding the same) may comprise one or more tags (e.g. purification tags), such as a His-tag or Strep-tag. It is intended that the present invention also encompasses polypeptide sequences (and nucleotide sequences encoding the same) where the tag is removed, e.g. before use thereof. The polypeptide may also comprise one or more cleavage sites, such as a TEV cleavage site, to facilitate removal of a tag.

The present invention is suitable for application to many different varieties of clostridial neurotoxin. Thus, in the context of the present invention, the term “clostridial neurotoxin” embraces toxins produced by C. botulinum (botulinum neurotoxin serotypes A, B, C1, D, E, F, G, H, and X), C. tetani (tetanus neurotoxin), C. butyricum (botulinum neurotoxin serotype E), and C. barati (botulinum neurotoxin serotype F), as well as modified clostridial neurotoxins or derivatives derived from any of the foregoing. The term “clostridial neurotoxin” also embraces botulinum neurotoxin serotype H. Preferably the clostridial neurotoxin is not BoNT/C1.

Botulinum neurotoxin (BoNT) is produced by C. botulinum in the form of a large protein complex, consisting of BoNT itself complexed to a number of accessory proteins. There are at present nine different classes of botulinum neurotoxin, namely: botulinum neurotoxin serotypes A, B, C1, D, E, F, G, H, and X all of which share similar structures and modes of action. Different BoNT serotypes can be distinguished based on inactivation by specific neutralising anti-sera, with such classification by serotype correlating with percentage sequence identity at the amino acid level. BoNT proteins of a given serotype are further divided into different subtypes on the basis of amino acid percentage sequence identity.

BoNTs are absorbed in the gastrointestinal tract, and, after entering the general circulation, bind to the presynaptic membrane of cholinergic nerve terminals and prevent the release of their neurotransmitter acetylcholine. BoNT/B, BoNT/D, BoNT/F and BoNT/G cleave synaptobrevin/vesicle-associated membrane protein (VAMP); BoNT/C1, BoNT/A and BoNT/E cleave the synaptosomal-associated protein of 25 kDa (SNAP-25); and BoNT/C1 cleaves syntaxin. BoNT/X has been found to cleave SNAP-25, VAMP1, VAMP2, VAMP3, VAMP4, VAMP5, Ykt6, and syntaxin 1.

Tetanus toxin is produced in a single serotype by C. tetani. C. butyricum produces BoNT/E, while C. baratii produces BoNT/F.

The term “clostridial neurotoxin” is also intended to embrace modified clostridial neurotoxins and derivatives thereof, including but not limited to those described below. A modified clostridial neurotoxin or derivative may contain one or more amino acids that has been modified as compared to the native (unmodified) form of the clostridial neurotoxin, or may contain one or more inserted amino acids that are not present in the native (unmodified) form of the clostridial neurotoxin. By way of example, a modified clostridial neurotoxin may have modified amino acid sequences in one or more domains relative to the native (unmodified) clostridial neurotoxin sequence. Such modifications may modify functional aspects of the toxin, for example biological activity or persistence. Thus, in one embodiment, the polypeptide of the invention is a modified clostridial neurotoxin, or an modified clostridial neurotoxin derivative, or a clostridial neurotoxin derivative.

A modified clostridial neurotoxin may have one or more modifications in the amino acid sequence of the heavy chain (such as a modified H_{C} domain), wherein said modified heavy chain binds to target nerve cells with a higher or lower affinity than the native (unmodified) clostridial neurotoxin. Such modifications in the H_{C} domain can include modifying residues in the ganglioside binding site of the H_{C} domain or in the protein (SV2 or synaptotagmin) binding site that alter binding to the ganglioside receptor and/or the protein receptor of the target nerve cell. Examples of such modified clostridial neurotoxins are described in WO 2006/027207 and WO 2006/114308, both of which are hereby incorporated by reference in their entirety.

A modified clostridial neurotoxin may have one or more modifications in the amino acid sequence of the light chain, for example modifications in the substrate binding or catalytic domain which may alter or modify the SNARE protein specificity of the modified L-chain. Examples of such modified clostridial neurotoxins are described in WO 2010/120766 and US 2011/0318385, both of which are hereby incorporated by reference in their entirety.

A modified clostridial neurotoxin may comprise one or more modifications that increases or decreases the biological activity and/or the biological persistence of the modified clostridial neurotoxin. For example, a modified clostridial neurotoxin may comprise a leucine- or tyrosine-based motif, wherein said motif increases or decreases the biological activity and/or the biological persistence of the modified clostridial neurotoxin. Suitable leucine-based motifs include xDxxxLL (SEQ ID NO: 79), xExxxLL (SEQ ID NO: 80), xExxxIL (SEQ ID NO: 81), and xExxxLM (SEQ ID NO: 82) (wherein x is any amino acid). Suitable tyrosine-based motifs include Y-x-x-Hy (SEQ ID NO: 83) (wherein Hy is a hydrophobic amino acid). Examples of modified clostridial neurotoxins comprising leucine- and tyrosine-based motifs are described in WO 2002/08268, which is hereby incorporated by reference in its entirety.

The term “clostridial neurotoxin” is intended to embrace hybrid and chimeric clostridial neurotoxins. A hybrid clostridial neurotoxin comprises at least a portion of a light chain from one clostridial neurotoxin or subtype thereof, and at least a portion of a heavy chain from another clostridial neurotoxin or clostridial neurotoxin subtype. In one embodiment the hybrid clostridial neurotoxin may contain the entire light chain of a light chain from one clostridial neurotoxin subtype and the heavy chain from another clostridial neurotoxin subtype. In another embodiment, a chimeric clostridial neurotoxin may contain a portion (e.g. the binding domain) of the heavy chain of one clostridial neurotoxin subtype, with another portion of the heavy chain being from another clostridial neurotoxin subtype. Similarly or alternatively, the therapeutic element may comprise light chain portions from different clostridial neurotoxins. Such hybrid or chimeric clostridial neurotoxins are useful, for example, as a means of delivering the therapeutic benefits of such clostridial neurotoxins to patients who are immunologically resistant to a given clostridial neurotoxin subtype, to patients who may have a lower than average concentration of receptors to a given clostridial neurotoxin heavy chain binding domain, or to patients who may have a protease-resistant variant of the membrane or vesicle toxin substrate (e.g., SNAP-25, VAMP and syntaxin). Hybrid and chimeric clostridial neurotoxins are described in U.S. Pat. No. 8,071,110, which publication is hereby incorporated by reference in its entirety. Thus, in one embodiment, the engineered clostridial neurotoxin of the invention is an engineered hybrid clostridial neurotoxin, or an engineered chimeric clostridial neurotoxin.

The term “clostridial neurotoxin” is also intended to embrace newly discovered botulinum neurotoxin protein family members expressed by non-clostridial microorganisms, such as the Enterococcus encoded toxin which has closest sequence identity to BoNT/X, the Weissella oryzae encoded toxin called BoNT/Wo (NCBI Ref Seq: WP_027699549.1), which cleaves VAMP2 at W89-W90, the Enterococcus faecium encoded toxin (GenBank: OTO22244.1), which cleaves VAMP2 and SNAP25, and the Chryseobacterium pipero encoded toxin (NCBI Ref.Seq: WP_034687872.1).

The ‘bioactive’ component of the polypeptides of the present invention is provided by a non-cytotoxic protease. This distinct group of proteases act by proteolytically-cleaving intracellular transport proteins known as SNARE proteins (e.g. SNAP-25, VAMP, or Syntaxin)—see Gerald K (2002) “Cell and Molecular Biology” (4th edition) John Wiley & Sons, Inc. The acronym SNARE derives from the term Soluble NSF Attachment Receptor, where NSF means N-ethylmaleimide-Sensitive Factor. SNARE proteins are integral to intracellular vesicle formation, and thus to secretion of molecules via vesicle transport from a cell. Accordingly, once delivered to a desired target cell, the non-cytotoxic protease is capable of inhibiting cellular secretion from the target cell.

Non-cytotoxic proteases are a discrete class of molecules that do not kill cells; instead, they act by inhibiting cellular processes other than protein synthesis. Non-cytotoxic proteases are produced as part of a larger toxin molecule by a variety of plants, and by a variety of microorganisms such as Clostridium sp. and Neisseria sp.

Clostridial neurotoxins represent a major group of non-cytotoxic toxin molecules, and comprise two polypeptide chains joined together by a disulphide bond. The two chains are termed the heavy chain (H-chain), which has a molecular mass of approximately 100 kDa, and the light chain (L-chain), which has a molecular mass of approximately 50 kDa. It is the L-chain, which possesses a protease function and exhibits a high substrate specificity for vesicle and/or plasma membrane associated (SNARE) proteins involved in the exocytic process (eg. synaptobrevin, syntaxin or SNAP-25). These substrates are important components of the neurosecretory machinery.

Neisseria sp., most importantly from the species N. gonorrhoeae, and Streptococcus sp., most importantly from the species S. pneumoniae, produce functionally similar non-cytotoxic toxin molecules. An example of such a non-cytotoxic protease is IgA protease (see WO99/58571, which is hereby incorporated in its entirety by reference thereto). Thus, the non-cytotoxic protease of the present invention is preferably a clostridial neurotoxin protease or an IgA protease.

Turning now to the Targeting Moiety (TM) component of the present invention, it is this component that binds the polypeptide of the present invention to a target cell.

Thus, a TM of the present invention binds to a receptor on a target cell. By way of example, a TM of the present invention may bind to a receptor on a neuronal cell, such as a receptor on a sensory or motor neuron. Alternatively, a TM of the present invention may bind to an EGF receptor. In one embodiment a target cell is a neuronal cell, such as a motor or sensory neuron. In another embodiment a target cell is a cell expressing an EGF receptor. However, the person skilled in the art can select a peptide TM for targeting a target cell of choice based on the presence of a Binding Site (e.g. cell-surface receptor) for said peptide on the target cell.

In one embodiment a polypeptide of the invention may comprise a TM comprising one or more of the following peptides: a growth hormone releasing hormone (GHRH) peptide, a somatostatin peptide, a cortistatin peptide, a ghrelin peptide, a bombesin peptide, a urotensin peptide, melanin-concentrating hormone peptide, a KISS-1 peptide, a gonadotropin-releasing hormone (GnRH) peptide, or a prolactin-releasing peptide. Said TMs and polypeptides comprising the same are described in WO 2009/150469, which is incorporated herein by reference.

In one embodiment a polypeptide of the invention may comprise a TM comprising one or more of the following peptides a leptin peptide, an insulin-like growth factor (IGF) peptide, a transforming growth factor (TGF) peptide, a VIP-glucagon-GRF-secretin superfamily peptide, a PACAP peptide, a vasoactive intestinal peptide (VIP), an orexin peptide, an interleukin peptide, a nerve growth factor (NGF) peptide, a vascular endothelial growth factor (VEGF) peptide, a thyroid hormone peptide, an oestrogen peptide, an ErbB peptide, an epidermal growth factor (EGF) peptide, an EGF and TGF-α chimera peptide, an amphiregulin peptide, a betacellulin peptide, an epigen peptide, an epiregulin peptide, a heparin-binding EGF (HB-EGF) peptide, a bombesin peptide, a urotensin peptide, a melanin-concentrating hormone (MCH) peptide, a a Kisspeptin-10 peptide, a Kisspeptin-54 peptide, a corticotropin-releasing hormone peptide, a urocortin 1 peptide, or a urocortin 2 peptide. Said TMs and polypeptides comprising the same are described in WO2009/150470, which is incorporated herein by reference.

In another embodiment a polypeptide of the invention may comprise a TM comprising one or more of the following: thyroid stimulating hormone, (TSH); TSH receptor antibodies; antibodies to the islet-specific monosialoganglioside, GM2-1; insulin, insulin-like growth factor and antibodies to the receptors of both; TSH releasing hormone (protirelin) and antibodies to its receptor; FSH/LH releasing hormone (gonadorelin) and antibodies to its receptor; corticotrophin releasing hormone (CRH) and antibodies to its receptor; and ACTH and antibodies to its receptor. Said TMs and polypeptides comprising the same are described in WO 01/21213, which is incorporated herein by reference.

The polypeptides of the present invention may comprise 3 principal components: a non-cytotoxic protease or proteolytically inactive mutant thereof; a TM; and a translocation domain.

The general technology associated with the preparation of such fusion proteins is often referred to as re-targeted toxin technology. By way of exemplification, we refer to: WO94/21300; WO96/33273; WO98/07864; WO00/10598; WO01/21213; WO06/059093; WO00/62814; WO00/04926; WO93/15766; WO00/61192; and WO99/58571. All of these publications are herein incorporated by reference thereto.

In more detail, the TM component of the present invention may be fused to either the protease component or the translocation component of the present invention. Said fusion is preferably by way of a covalent bond, for example either a direct covalent bond or via a spacer/linker molecule. The protease component and the translocation component are preferably linked together via a covalent bond, for example either a direct covalent bond or via a spacer/linker molecule. Suitable spacer/linked molecules are well known in the art, and typically comprise an amino acid-based sequence of between 5 and 40, preferably between 10 and 30 amino acid residues in length.

In use, the polypeptides have a di-chain conformation, wherein the protease component and the translocation component are linked together, preferably via a disulphide bond.

Thus, the polypeptides and labelled polypeptides of the invention may be in a single-chain form or a di-chain form, preferably in a di-chain form.

The polypeptides of the present invention may be prepared by conventional chemical conjugation techniques, which are well known to a skilled person. By way of example, reference is made to Hermanson, G. T. (1996), Bioconjugate techniques, Academic Press, and to Wong, S. S. (1991), Chemistry of protein conjugation and cross-linking, CRC Press, Nagy et al., PNAS 95 p 1794-99 (1998). Further detailed methodologies for attaching synthetic TMs to a polypeptide of the present invention are provided in, for example, EP0257742. The above-mentioned conjugation publications are herein incorporated by reference thereto.

Alternatively, the polypeptides may be prepared by recombinant preparation of a single polypeptide fusion protein (see, for example, WO98/07864). This technique is based on the in vivo bacterial mechanism by which native clostridial neurotoxin (i.e. holotoxin) is prepared, and results in a fusion protein having the following ‘simplified’ structural arrangement:

NH₂-[protease component]-[translocation component]-[TM]-COOH

According to WO98/07864, the TM is placed towards the C-terminal end of the fusion protein. The fusion protein is then activated by treatment with a protease, which cleaves at a site between the protease component and the translocation component. A di-chain protein is thus produced, comprising the protease component as a single polypeptide chain covalently attached (via a disulphide bridge) to another single polypeptide chain containing the translocation component plus TM.

Alternatively, according to WO06/059093, the TM component of the fusion protein is located towards the middle of the linear fusion protein sequence, between the protease cleavage site and the translocation component. This ensures that the TM is attached to the translocation domain (i.e. as occurs with native clostridial holotoxin), though in this case the two components are reversed in order vis-à-vis native holotoxin. Subsequent cleavage at the protease cleavage site exposes the N-terminal portion of the TM, and provides the di-chain polypeptide fusion protein.

The above-mentioned protease cleavage sequence(s) may be introduced (and/or any inherent cleavage sequence removed) at the DNA level by conventional means, such as by site-directed mutagenesis. Screening to confirm the presence of cleavage sequences may be performed manually or with the assistance of computer software (e.g. the MapDraw program by DNASTAR, Inc.). Whilst any protease cleavage site may be employed (ie. clostridial, or non-clostridial), the following are preferred:

| | |
|---|---|
| Enterokinase | (DDDDK↓, SEQ ID NO: 84) |
| Factor Xa | (IEGR↓/IDGR↓, SEQ ID NOs: 85 and 86) |
| TEV(Tobacco | (ENLYFQ↓G, SEQ ID NO: 87) |
| Etch virus) | |
| Thrombin | (LVPR↓GS, SEQ ID NO: 88) |
| PreScission | (LEVLFQ↓GP, SEQ ID NO: 89). |

Additional protease cleavage sites include recognition sequences that are cleaved by a non-cytotoxic protease, for example by a clostridial neurotoxin. These include the SNARE (eg. SNAP-25, syntaxin, VAMP) protein recognition sequences that are cleaved by non-cytotoxic proteases such as clostridial neurotoxins. Particular examples are provided in US2007/0166332, which is hereby incorporated in its entirety by reference thereto.

Also embraced by the term protease cleavage site is an intein, which is a self-cleaving sequence. The self-splicing reaction is controllable, for example by varying the concentration of reducing agent present. The above-mentioned ‘activation’ cleavage sites may also be employed as a ‘destructive’ cleavage site (discussed below) should one be incorporated into a polypeptide of the present invention.

In a preferred embodiment, the fusion protein of the present invention may comprise one or more N-terminal and/or C-terminal located purification tags. Whilst any purification tag may be employed, the following are preferred:
His-tag (e.g. 6× histidine), preferably as a C-terminal and/or N-terminal tag
MBP-tag (maltose binding protein), preferably as an N-terminal tag
GST-tag (glutathione-S-transferase), preferably as an N-terminal tag
His-MBP-tag, preferably as an N-terminal tag
GST-MBP-tag, preferably as an N-terminal tag
Thioredoxin-tag, preferably as an N-terminal tag
CBD-tag (Chitin Binding Domain), preferably as an N-terminal tag.

One or more peptide spacer/linker molecules may be included in the fusion protein. For example, a peptide spacer may be employed between a purification tag and the rest of the fusion protein molecule.

In one aspect the invention provides a method for manufacturing a polypeptide for labelling using a sortase, the method comprising:
a. providing a nucleic acid sequence encoding a polypeptide, wherein the polypeptide comprises:
   i. a non-cytotoxic protease or a proteolytically inactive mutant thereof;
   ii. a Targeting Moiety (TM) that is capable of binding to a Binding Site on a target cell; and
   iii. a translocation domain; and
b. introducing a sortase acceptor or donor site into said nucleic acid, thereby producing a modified nucleic acid that encodes a polypeptide comprising a sortase acceptor or donor site.

Introduction of a sortase acceptor or donor site can be achieved by any modifications/methods known to the person skilled in the art, e.g. by way of substitution, insertion or deletion of sequences encoding amino acid residues in the resultant polypeptide. By way of example, modifications may be introduced by modification of a nucleic acid sequence using standard molecular cloning techniques, for example by site-directed mutagenesis where short strands of DNA (oligonucleotides) coding for the desired amino acid(s) are used to replace the original coding sequence using a polymerase enzyme, or by inserting/deleting parts of the gene with various enzymes (e.g., ligases and restriction endonucleases). Alternatively a modified gene sequence can be chemically synthesised.

Preferably the method further comprises expressing the modified nucleic acid in a host cell. More preferably, the method further comprises expressing the modified nucleic acid in a host cell and obtaining the expressed polypeptide. The polypeptide may be activated using a method described herein.

The invention also extends to a polypeptide obtainable by a method of the invention.

The term “obtaining” as used in the context of “obtaining the labelled polypeptide” or “obtaining the expressed polypeptide” may mean isolating the polypeptide. Isolating can be achieved by any purification methods, such as chromatographic or immunoaffinity methods known to the person skilled in the art.

The nucleic acid for use in the methods of manufacturing may be a nucleic acid encoding a polypeptide described herein. For example, such a nucleic acid may encode a polypeptide having at least 70% sequence identity to any one of SEQ ID NOs: 6, 8, 17-25 or 38. In one embodiment a nucleic acid may encode a polypeptide having at least 80% or 90% sequence identity to any one of SEQ ID NOs: 6, 8, 17-25 or 38. Preferably a nucleic acid may encode a polypeptide comprising (more preferably consisting of) any one of SEQ ID NOs: 6, 8, 17-25 or 38.

The nucleic acid for use in the methods of manufacturing may be a nucleic acid comprising a nucleic acid sequence having at least 70% sequence identity to any one of SEQ ID NO: 5 or 7. In one embodiment a nucleic acid may be a nucleic acid comprising a nucleic acid sequence having at least 80% or 90% sequence identity to any one of SEQ ID NO: 5 or 7. Preferably a nucleic acid may comprise (more preferably consist of) SEQ ID NO: 5 or 7.

Thus, the present invention provides a nucleic acid (e.g. DNA) sequence (e.g. modified nucleic acid) encoding a polypeptide of the invention. Said nucleic acid may be included in the form of a vector, such as a plasmid, which may optionally include one or more of an origin of replication, a nucleic acid integration site, a promoter, a terminator, and a ribosome binding site.

A nucleic acid (e.g. modified nucleic acid) of the present invention may comprise a nucleic acid sequence having at least 70% sequence identity to SEQ ID NOs: 1, 3 or 39. In one embodiment a nucleic acid of the present invention may comprise a nucleic acid sequence having at least 80% or 90% sequence identity to SEQ ID NOs: 1, 3 or 39. Preferably, a nucleic acid of the present invention comprises (more preferably consists of) a nucleic acid sequence shown as SEQ ID NOs: 1, 3 or 39.

A nucleic acid (e.g. modified nucleic acid) of the present invention may be one that encodes a polypeptide having at least 70% sequence identity to SEQ ID NOs: 2, 4 or 40. In one embodiment a nucleic acid of the present invention may be one that encodes a polypeptide having at least 80% or 90% sequence identity to SEQ ID NOs: 2, 4 or 40. Preferably, a nucleic acid of the present invention may be one that encodes a polypeptide comprising (more preferably consisting of) SEQ ID NOs: 2, 4 or 40.

The present invention also encompasses a host cell comprising a nucleic or vector of the invention.

The present invention also includes a method for expressing the above-described nucleic acid sequence in a host cell, in particular in E. coli or via a baculovirus expression system.

The present invention also includes a method for activating a polypeptide of the present invention, said method comprising contacting the polypeptide with a protease (e.g. FXa) that cleaves the polypeptide at a recognition site (cleavage site, such as a FXa site) located between the non-cytotoxic protease component and the translocation component, thereby converting the polypeptide into a di-chain polypeptide wherein the non-cytotoxic protease and translocation components are joined together by a disulphide bond. In a preferred embodiment, the recognition site is not native to a naturally-occurring clostridial neurotoxin and/or to a naturally-occurring IgA protease.

The polypeptides of the present invention may be further modified to reduce or prevent unwanted side-effects associated with dispersal into non-targeted areas. According to this embodiment, the polypeptide comprises a destructive cleavage site. The destructive cleavage site is distinct from the ‘activation’ site (i.e. di-chain formation), and is cleavable by a second protease and not by the non-cytotoxic protease. Moreover, when so cleaved at the destructive cleavage site by the second protease, the polypeptide has reduced potency (e.g. reduced binding ability to the intended target cell, reduced translocation activity and/or reduced non-cytotoxic protease activity). For completeness, any of the ‘destructive’ cleavage sites of the present invention may be separately employed as an ‘activation’ site in a polypeptide of the present invention.

Thus, according to this embodiment, the present invention provides a polypeptide that can be controllably inactivated and/or destroyed at an off-site location.

In a preferred embodiment, the destructive cleavage site is recognised and cleaved by a second protease (i.e. a destructive protease) selected from a circulating protease (e.g. an extracellular protease, such as a serum protease or a protease of the blood clotting cascade), a tissue-associated protease (e.g. a matrix metalloprotease (MMP), such as an MMP of muscle), and an intracellular protease (preferably a protease that is absent from the target cell).

Thus, in use, should a polypeptide of the present invention become dispersed away from its intended target cell and/or be taken up by a non-target cell, the polypeptide will become inactivated by cleavage of the destructive cleavage site (by the second protease).

In one embodiment, the destructive cleavage site is recognised and cleaved by a second protease that is present within an off-site cell-type. In this embodiment, the off-site cell and the target cell are preferably different cell types. Alternatively (or in addition), the destructive cleavage site is recognised and cleaved by a second protease that is present at an off-site location (e.g. distal to the target cell). Accordingly, when destructive cleavage occurs extracellularly, the target cell and the off-site cell may be either the same or different cell-types. In this regard, the target cell and the off-site cell may each possess a receptor to which the same polypeptide of the invention binds.

The destructive cleavage site of the present invention provides for inactivation/destruction of the polypeptide when the polypeptide is in or at an off-site location. In this regard, cleavage at the destructive cleavage site minimises the potency of the polypeptide (when compared with an identical polypeptide lacking the same destructive cleavage site, or possessing the same destructive site but in an uncleaved form). By way of example, reduced potency includes: reduced binding (to a mammalian cell receptor) and/or reduced translocation (across the endosomal membrane of a mammalian cell in the direction of the cytosol), and/or reduced SNARE protein cleavage.

When selecting destructive cleavage site(s) in the context of the present invention, it is preferred that the destructive cleavage site(s) are not substrates for any proteases that may be separately used for post-translational modification of the polypeptide of the present invention as part of its manufacturing process. In this regard, the non-cytotoxic proteases of the present invention typically employ a protease activation event (via a separate ‘activation’ protease cleavage site, which is structurally distinct from the destructive cleavage site of the present invention). The purpose of the activation cleavage site is to cleave a peptide bond between the non-cytotoxic protease and the translocation or the binding components of the polypeptide of the present invention, thereby providing an ‘activated’ di-chain polypeptide wherein said two components are linked together via a di-sulfide bond.

Thus, to help ensure that the destructive cleavage site(s) of the polypeptides of the present invention do not adversely affect the ‘activation’ cleavage site and subsequent di-sulfide bond formation, the former are preferably introduced into polypeptide of the present invention at a position of at least 20, at least 30, at least 40, at least 50, and more preferably at least 60, at least 70, at least 80 (contiguous) amino acid residues away from the ‘activation’ cleavage site.

The destructive cleavage site(s) and the activation cleavage site are preferably exogenous (i.e. engineered/artificial) with regard to the native components of the polypeptide. In other words, said cleavage sites are preferably not inherent to the corresponding native components of the polypeptide. By way of example, a protease or translocation component based on BoNT/A L-chain or H-chain (respectively) may be engineered according to the present invention to include a cleavage site. Said cleavage site would not, however, be present in the corresponding BoNT native L-chain or H-chain. Similarly, when the Targeting Moiety component of the polypeptide is engineered to include a protease cleavage site, said cleavage site would not be present in the corresponding native sequence of the corresponding Targeting Moiety.

In a preferred embodiment of the present invention, the destructive cleavage site(s) and the ‘activation’ cleavage site are not cleaved by the same protease. In one embodiment, the two cleavage sites differ from one another in that at least one, more preferably at least two, particularly preferably at least three, and most preferably at least four of the tolerated amino acids within the respective recognition sequences is/are different.

By way of example, in the case of a polypeptide chimera containing a Factor Xa ‘activation’ site between clostridial L-chain and H_{N} components, it is preferred to employ a destructive cleavage site that is a site other than a Factor Xa site, which may be inserted elsewhere in the L-chain and/or H_{N} and/or TM component(s). In this scenario, the polypeptide may be modified to accommodate an alternative ‘activation’ site between the L-chain and H_{N} components (for example, an enterokinase cleavage site), in which case a separate Factor Xa cleavage site may be incorporated elsewhere into the polypeptide as the destructive cleavage site. Alternatively, the existing Factor Xa ‘activation’ site between the L-chain and H_{N} components may be retained, and an alternative cleavage site such as a thrombin cleavage site incorporated as the destructive cleavage site.

When identifying suitable sites within the primary sequence of any of the components of the present invention for inclusion of cleavage site(s), it is preferable to select a primary sequence that closely matches with the proposed cleavage site that is to be inserted. By doing so, minimal structural changes are introduced into the polypeptide. By way of example, cleavage sites typically comprise at least 3 contiguous amino acid residues. Thus, in a preferred embodiment, a cleavage site is selected that already possesses (in the correct position(s)) at least one, preferably at least two of the amino acid residues that are required in order to introduce the new cleavage site. By way of example, in one embodiment, the Caspase 3 cleavage site (DMQD) may be introduced. In this regard, a preferred insertion position is identified that already includes a primary sequence selected from, for example, Dxxx, xMxx, xxQx, xxxD, DMxx, DxQx, DxxD, xMQx, xMxD, xxQD, DMQx, xMQD, DxQD, and DMxD.

Similarly, it is preferred to introduce the cleavage sites into surface exposed regions. Within surface exposed regions, existing loop regions are preferred.

In a preferred embodiment of the present invention, the destructive cleavage site(s) are introduced at one or more of the following position(s), which are based on the primary amino acid sequence of BoNT/A. Whilst the insertion positions are identified (for convenience) by reference to BoNT/A, the primary amino acid sequences of alternative protease domains and/or translocation domains may be readily aligned with said BoNT/A positions.

For the protease component, one or more of the following positions is preferred: 27-31, 56-63, 73-75, 78-81, 99-105, 120-124, 137-144, 161-165, 169-173, 187-194, 202-214, 237-241, 243-250, 300-304, 323-335, 375-382, 391-400, and 413-423. The above numbering preferably starts from the N-terminus of the protease component of the present invention.

In a preferred embodiment, the destructive cleavage site(s) are located at a position greater than 8 amino acid residues, preferably greater than 10 amino acid residues, more preferably greater than 25 amino acid residues, particularly preferably greater than 50 amino acid residues from the N-terminus of the protease component. Similarly, in a preferred embodiment, the destructive cleavage site(s) are located at a position greater than 20 amino acid residues, preferably greater than 30 amino acid residues, more preferably greater than 40 amino acid residues, particularly preferably greater than 50 amino acid residues from the C-terminus of the protease component.

For the translocation component, one or more of the following positions is preferred: 474-479, 483-495, 507-543, 557-567, 576-580, 618-631, 643-650, 669-677, 751-767, 823-834, 845-859. The above numbering preferably acknowledges a starting position of 449 for the N-terminus of the translocation domain component of the present invention, and an ending position of 871 for the C-terminus of the translocation domain component.

In a preferred embodiment, the destructive cleavage site(s) are located at a position greater than 10 amino acid residues, preferably greater than 25 amino acid residues, more preferably greater than 40 amino acid residues, particularly preferably greater than 50 amino acid residues from the N-terminus of the translocation component. Similarly, in a preferred embodiment, the destructive cleavage site(s) are located at a position greater than 10 amino acid residues, preferably greater than 25 amino acid residues, more preferably greater than 40 amino acid residues, particularly preferably greater than 50 amino acid residues from the C-terminus of the translocation component.

In a preferred embodiment, the destructive cleavage site(s) are located at a position greater than 10 amino acid residues, preferably greater than 25 amino acid residues, more preferably greater than 40 amino acid residues, particularly preferably greater than 50 amino acid residues from the N-terminus of the TM component. Similarly, in a preferred embodiment, the destructive cleavage site(s) are located at a position greater than 10 amino acid residues, preferably greater than 25 amino acid residues, more preferably greater than 40 amino acid residues, particularly preferably greater than 50 amino acid residues from the C-terminus of the TM component.

The polypeptide of the present invention may include one or more (e.g. two, three, four, five or more) destructive protease cleavage sites. Where more than one destructive cleavage site is included, each cleavage site may be the same or different. In this regard, use of more than one destructive cleavage site provides improved off-site inactivation. Similarly, use of two or more different destructive cleavage sites provides additional design flexibility.

The destructive cleavage site(s) may be engineered into any of the following component(s) of the polypeptide: the non-cytotoxic protease component; the translocation component; the Targeting Moiety; or the spacer peptide (if present). In this regard, the destructive cleavage site(s) are chosen to ensure minimal adverse effect on the potency of the polypeptide (for example by having minimal effect on the targeting/binding regions and/or translocation domain, and/or on the non-cytotoxic protease domain) whilst ensuring that the polypeptide is labile away from its target site/target cell.

Preferred destructive cleavage sites (plus the corresponding second proteases) are listed in the Table immediately below. The listed cleavage sites are purely illustrative and are not intended to be limiting to the present invention.

| | Destructive | | | | | | | |
|---|---|---|---|---|---|---|---|---|
| | cleavage site | Tolerated recognition sequence variance | | | | | | |
| Second | recognition | P4-P3-P2-P1-▾-P1′-P2′-P3′ | | | | | | |
| protease | sequence | P4 | P3 | P2 | P1 | P1′ | P2′ | P3′ |
| Thrombin | LVPR▾GS (SEQ | A, F, G, | A, F, G, | P | R | Not D | Not D | — |
| | ID NO: 88) | I, L, T, | I, L, T, | | | or E | or E | |
| | | V or M | V, W or A | | | | | |
| Thrombin | GR▾G | | | G | R | G | | |
| Factor Xa | IEGR▾ (SEQ | A, F, G, | D or E | G | R | — | — | — |
| | ID NO: 85) | I, L, T, | | | | | | |
| | | V or M | | | | | | |
| ADAM17 | PLAQA▾VRSSS | | | | | | | |
| | (SEQ ID NO: 90) | | | | | | | |
| Human | SKGR▾SLIGRV | | | | | | | |
| airway | (SEQ ID NO: 91) | | | | | | | |
| trypsin-like | | | | | | | | |
| protease | | | | | | | | |
| (HAT) | | | | | | | | |
| ACE | | — | — | — | — | Not P | Not D | N/A |
| (peptidyl- | | | | | | | or E | |
| dipeptidase A) | | | | | | | | |
| Elastase | MEA▾VTY | M, R | E | A, H | V, T | V, T, H | Y | — |
| (leukocyte) | (SEQ ID NO: 92) | | | | | | | |
| Furin | RXR/KR▾ | R | X | R or K | R | | | |
| | (SEQ ID NO: 93) | | | | | | | |
| Granzyme | IEPD▾ | I | E | P | D | — | — | — |
| | (SEQ ID NO: 94) | | | | | | | |
| Caspase 1 | | F, W, Y, | — | H, A, T | D | Not P, E.D. | — | — |
| | | L | | | | Q.K or R | | |
| Caspase 2 | DVAD▾ | D | V | A | D | Not P, E.D. | — | — |
| | (SEQ ID NO: 95) | | | | | Q.K or R | | |
| Caspase 3 | DMQD▾ | D | M | Q | D | Not P, E.D. | — | — |
| | (SEQ ID NO: 96) | | | | | Q.K or R | | |
| Caspase 4 | LEVD▾ | L | E | V | D | Not P, E.D. | — | — |
| | (SEQ ID NO: 97) | | | | | Q.K or R | | |
| Caspase 5 | | L or W | E | H | D | — | — | — |
| Caspase 6 | | V | E | H or I | D | Not P, E.D. | — | — |
| | | | | | | Q.K or R | | |
| Caspase 7 | DEVD▾ | D | E | V | D | Not P, E.D. | — | — |
| | (SEQ ID NO: 98) | | | | | Q.K or R | | |
| Caspase 8 | | I or L | E | T | D | Not P, E.D. | — | — |
| | | | | | | Q.K or R | | |
| Caspase 9 | LEHD▾ | L | E | H | D | — | — | — |
| | (SEQ ID NO: 99) | | | | | | | |
| Caspase 10 | IEHD▾ | I | E | H | D | — | — | — |
| | (SEQ ID NO: 100) | | | | | | | |

Matrix metalloproteases (MMPs) are a preferred group of destructive proteases in the context of the present invention. Within this group, ADAM17 (EC 3.4.24.86, also known as TACE), is preferred and cleaves a variety of membrane-anchored, cell-surface proteins to “shed” the extracellular domains. Additional, preferred MMPs include adamalysins, serralysins, and astacins.

Another group of preferred destructive proteases is a mammalian blood protease, such as Thrombin, Coagulation Factor VIIa, Coagulation Factor IXa, Coagulation Factor Xa, Coagulation Factor XIa, Coagulation Factor XIIa, Kallikrein, Protein C, and MBP-associated serine protease.

In one embodiment of the present invention, said destructive cleavage site comprises a recognition sequence having at least 3 or 4, preferably 5 or 6, more preferably 6 or 7, and particularly preferably at least 8 contiguous amino acid residues. In this regard, the longer (in terms of contiguous amino acid residues) the recognition sequence, the less likely non-specific cleavage of the destructive site will occur via an unintended second protease.

It is preferred that the destructive cleavage site of the present invention is introduced into the protease component and/or the Targeting Moiety and/or into the translocation component and/or into the spacer peptide. Of these four components, the protease component is preferred. Accordingly, the polypeptide may be rapidly inactivated by direct destruction of the non-cytotoxic protease and/or binding and/or translocation components.

The polypeptides of the invention may be formulated as part of a pharmaceutical composition, comprising a polypeptide, together with at least one component selected from a pharmaceutically acceptable carrier, excipient, adjuvant, propellant and/or salt.

The polypeptides of the present invention may be formulated for oral, parenteral, continuous infusion, implant, inhalation or topical application. Compositions suitable for injection may be in the form of solutions, suspensions or emulsions, or dry powders which are dissolved or suspended in a suitable vehicle prior to use.

Local delivery means may include an aerosol, or other spray (e.g. a nebuliser). In this regard, an aerosol formulation of a polypeptide enables delivery to the lungs and/or other nasal and/or bronchial or airway passages.

The preferred route of administration is selected from: systemic (e.g. iv), laparoscopic and/or localised injection (for example, transsphenoidal injection directly into a tumour).

In the case of formulations for injection, it is optional to include a pharmaceutically active substance to assist retention at or reduce removal of the polypeptide from the site of administration. One example of such a pharmaceutically active substance is a vasoconstrictor such as adrenaline. Such a formulation confers the advantage of increasing the residence time of polypeptide following administration and thus increasing and/or enhancing its effect.

The dosage ranges for administration of the polypeptides of the present invention are those to produce the desired therapeutic effect. It will be appreciated that the dosage range required depends on the precise nature of the polypeptide or composition, the route of administration, the nature of the formulation, the age of the patient, the nature, extent or severity of the patient's condition, contraindications, if any, and the judgement of the attending physician. Variations in these dosage levels can be adjusted using standard empirical routines for optimisation.

Suitable daily dosages (per kg weight of patient) are in the range 0.0001-1 mg/kg, preferably 0.0001-0.5 mg/kg, more preferably 0.002-0.5 mg/kg, and particularly preferably 0.004-0.5 mg/kg. The unit dosage can vary from less than 1 microgram to 30 mg, but typically will be in the region of 0.01 to 1 mg per dose, which may be administered daily or preferably less frequently, such as weekly or six monthly.

A particularly preferred dosing regimen is based on 2.5 ng of polypeptide as the 1× dose. In this regard, preferred dosages are in the range 1×-100× (i.e. 2.5-250 ng).

Fluid dosage forms are typically prepared utilising the polypeptide and a pyrogen-free sterile vehicle. The polypeptide, depending on the vehicle and concentration used, can be either dissolved or suspended in the vehicle. In preparing solutions the polypeptide can be dissolved in the vehicle, the solution being made isotonic if necessary by addition of sodium chloride and sterilised by filtration through a sterile filter using aseptic techniques before filling into suitable sterile vials or ampoules and sealing. Alternatively, if solution stability is adequate, the solution in its sealed containers may be sterilised by autoclaving. Advantageously additives such as buffering, solubilising, stabilising, preservative or bactericidal, suspending or emulsifying agents and or local anaesthetic agents may be dissolved in the vehicle.

Dry powders, which are dissolved or suspended in a suitable vehicle prior to use, may be prepared by filling pre-sterilised ingredients into a sterile container using aseptic technique in a sterile area. Alternatively the ingredients may be dissolved into suitable containers using aseptic technique in a sterile area. The product is then freeze dried and the containers are sealed aseptically.

Parenteral suspensions, suitable for intramuscular, subcutaneous or intradermal injection, are prepared in substantially the same manner, except that the sterile components are suspended in the sterile vehicle, instead of being dissolved and sterilisation cannot be accomplished by filtration. The components may be isolated in a sterile state or alternatively it may be sterilised after isolation, e.g. by gamma irradiation.

Advantageously, a suspending agent for example polyvinylpyrrolidone is included in the composition/s to facilitate uniform distribution of the components.

Targeting Moiety (TM) means any chemical structure that functionally interacts with a Binding Site to cause a physical association between the polypeptide of the invention and the surface of a target cell (typically a mammalian cell, especially a human cell). The term TM embraces any molecule (ie. a naturally occurring molecule, or a chemically/physically modified variant thereof) that is capable of binding to a Binding Site on the target cell, which Binding Site is preferably capable of internalisation (eg. endosome formation)—also referred to as receptor-mediated endocytosis. The TM may possess an endosomal membrane translocation function, in which case separate TM and Translocation Domain components need not be present in an agent of the present invention. Throughout the preceding description, specific TMs have been described. Reference to said TMs is merely exemplary, and the present invention embraces all variants and derivatives thereof, which possess a basic binding (i.e. targeting) ability to a Binding Site on a target cell, preferably wherein the Binding Site is capable of internalisation.

The TM of the present invention binds (preferably specifically binds) to the target cell in question. The term “specifically binds” preferably means that a given TM binds to the target cell with a binding affinity (Ka) of 10⁶M⁻¹ or greater, preferably 10⁷M⁻¹ or greater, or 10⁸M⁻¹ or greater, or 10⁹ M⁻¹ or greater. The TMs of the present invention (when in a free form, namely when separate from any protease and/or translocation component), preferably demonstrate a binding affinity (IC₅₀) for the target receptor in question in the region of 0.05-18 nM.

The TM of the present invention is preferably not wheat germ agglutinin (WGA).

Reference to TM in the present specification embraces fragments and variants thereof, which retain the ability to bind to the target cell in question. By way of example, a variant may have at least 80%, preferably at least 90%, more preferably at least 95%, and most preferably at least 97 or at least 99% amino acid sequence homology with the reference TM—the latter is any TM sequence recited in the present application. Thus, a variant may include one or more analogues of an amino acid (e.g. an unnatural amino acid), or a substituted linkage. Also, by way of example, the term fragment, when used in relation to a TM, means a peptide having at least five, preferably at least ten, more preferably at least twenty, and most preferably at least twenty five amino acid residues of the reference TM. The term fragment also relates to the above-mentioned variants. Thus, by way of example, a fragment of the present invention may comprise a peptide sequence having at least 7, 10, 14, 17, 20, 25, 28, 29, or 30 amino acids, wherein the peptide sequence has at least 80% sequence homology over a corresponding peptide sequence (of contiguous) amino acids of the reference peptide.

The TM may comprise a longer amino acid sequence, for example, at least 30 or 35 amino acid residues, or at least 40 or 45 amino acid residues, so long as the TM is able to bind to a target cell.

It is routine to confirm that a TM binds to the selected target cell. For example, a simple radioactive displacement experiment may be employed in which tissue or cells representative of a target cell are exposed to labelled (eg. tritiated) TM in the presence of an excess of unlabelled TM. In such an experiment, the relative proportions of non-specific and specific binding may be assessed, thereby allowing confirmation that the TM binds to the target cell. Optionally, the assay may include one or more binding antagonists, and the assay may further comprise observing a loss of TM binding. Examples of this type of experiment can be found in Hulme, E. C. (1990), Receptor-binding studies, a brief outline, pp. 303-311, In Receptor biochemistry, A Practical Approach, Ed. E. C. Hulme, Oxford University Press.

In some embodiments, the polypeptides of the present invention lack a functional H_{C} domain of a clostridial neurotoxin. Accordingly, said polypeptides are not able to bind rat synaptosomal membranes (via a clostridial H_{C} component) in binding assays as described in Shone et al. (1985) Eur. J. Biochem. 151, 75-82. In a preferred embodiment, the polypeptides preferably lack the last 50 C-terminal amino acids of a clostridial neurotoxin holotoxin. In another embodiment, the polypeptides preferably lack the last 100, preferably the last 150, more preferably the last 200, particularly preferably the last 250, and most preferably the last 300 C-terminal amino acid residues of a clostridial neurotoxin holotoxin. Alternatively, the H_{C} binding activity may be negated/reduced by mutagenesis—by way of example, referring to BoNT/A for convenience, modification of one or two amino acid residue mutations (W1266 to L and Y1267 to F) in the ganglioside binding pocket causes the H_{C} region to lose its receptor binding function. Analogous mutations may be made to non-serotype A clostridial peptide components, e.g. a construct based on botulinum B with mutations (W1262 to L and Y1263 to F) or botulinum E (W1224 to L and Y1225 to F). Other mutations to the active site achieve the same ablation of H_{C} receptor binding activity, e.g. Y1267S in botulinum type A toxin and the corresponding highly conserved residue in the other clostridial neurotoxins. Details of this and other mutations are described in Rummel et al (2004) (Molecular Microbiol. 51:631-634), which is hereby incorporated by reference thereto.

In another embodiment, the polypeptides of the present invention lack a functional H_{C} domain of a clostridial neurotoxin and also lack any functionally equivalent TM. Accordingly, said polypeptides lack the natural binding function of a clostridial neurotoxin and are not able to bind rat synaptosomal membranes (via a clostridial H_{C} component, or via any functionally equivalent TM) in binding assays as described in Shone et al. (1985) Eur. J. Biochem. 151, 75-82.

The H_{C} peptide of a native clostridial neurotoxin comprises approximately 400-440 amino acid residues, and consists of two functionally distinct domains of approximately 25 kDa each, namely the N-terminal region (commonly referred to as the H_{CN} peptide or domain) and the C-terminal region (commonly referred to as the H_{CC} peptide or domain). This fact is confirmed by the following publications, each of which is herein incorporated in its entirety by reference thereto: Umland TC (1997) Nat. Struct. Biol. 4: 788-792; Herreros J (2000) Biochem. J. 347: 199-204; Halpern J (1993) J. Biol. Chem. 268: 15, pp. 11188-11192; Rummel A (2007) PNAS 104: 359-364; Lacey DB (1998) Nat. Struct. Biol. 5: 898-902; Knapp (1998) Am. Cryst. Assoc. Abstract Papers 25: 90; Swaminathan and Eswaramoorthy (2000) Nat. Struct. Biol. 7: 1751-1759; and Rummel A (2004) Mol. Microbiol. 51(3), 631-643. Moreover, it has been well documented that the C-terminal region (H_{CC}), which constitutes the C-terminal 160-200 amino acid residues, is responsible for binding of a clostridial neurotoxin to its natural cell receptors, namely to nerve terminals at the neuromuscular junction—this fact is also confirmed by the above publications. Thus, reference throughout this specification to a clostridial heavy-chain lacking a functional heavy chain H_{C} peptide (or domain) such that the heavy-chain is incapable of binding to cell surface receptors to which a native clostridial neurotoxin binds means that the clostridial heavy-chain simply lacks a functional H_{CC} peptide. In other words, the H_{CC} peptide region is either partially or wholly deleted, or otherwise modified (e.g. through conventional chemical or proteolytic treatment) to inactivate its native binding ability for nerve terminals at the neuromuscular junction.

Thus, in one embodiment, a clostridial H_{N} peptide of the present invention lacks part of a C-terminal peptide portion (H_{CC}) of a clostridial neurotoxin and thus lacks the H_{C} binding function of native clostridial neurotoxin. By way of example, in one embodiment, the C-terminally extended clostridial H_{N} peptide lacks the C-terminal 40 amino acid residues, or the C-terminal 60 amino acid residues, or the C-terminal 80 amino acid residues, or the C-terminal 100 amino acid residues, or the C-terminal 120 amino acid residues, or the C-terminal 140 amino acid residues, or the C-terminal 150 amino acid residues, or the C-terminal 160 amino acid residues of a clostridial neurotoxin heavy-chain. In another embodiment, the clostridial H_{N} peptide of the present invention lacks the entire C-terminal peptide portion (H_{CC}) of a clostridial neurotoxin and thus lacks the H_{C} binding function of native clostridial neurotoxin. By way of example, in one embodiment, the clostridial H_{N} peptide lacks the C-terminal 165 amino acid residues, or the C-terminal 170 amino acid residues, or the C-terminal 175 amino acid residues, or the C-terminal 180 amino acid residues, or the C-terminal 185 amino acid residues, or the C-terminal 190 amino acid residues, or the C-terminal 195 amino acid residues of a clostridial neurotoxin heavy-chain. By way of further example, the clostridial H_{N} peptide of the present invention lacks a clostridial H_{CC} reference sequence selected from the group consisting of:
Botulinum type A neurotoxin—amino acid residues (Y1111-L1296)
Botulinum type B neurotoxin—amino acid residues (Y1098-E1291)
Botulinum type C neurotoxin—amino acid residues (Y1112-E1291)
Botulinum type D neurotoxin—amino acid residues (Y1099-E1276)
Botulinum type E neurotoxin—amino acid residues (Y1086-K1252)
Botulinum type F neurotoxin—amino acid residues (Y1106-E1274)
Botulinum type G neurotoxin—amino acid residues (Y1106-E1297)
Tetanus neurotoxin—amino acid residues (Y1128-D1315).

The above-identified reference sequences should be considered a guide as slight variations may occur according to sub-serotypes.

The protease of the present invention embraces all non-cytotoxic proteases that are capable of cleaving one or more proteins of the exocytic fusion apparatus in eukaryotic cells.

The protease of the present invention is preferably a bacterial protease (or fragment thereof). More preferably the bacterial protease is selected from the genera Clostridium or Neisseria/Streptococcus (e.g. a clostridial L-chain, or a neisserial IgA protease preferably from N. gonorrhoeae or S. pneumoniae).

The present invention also embraces variant non-cytotoxic proteases (ie. variants of naturally-occurring protease molecules), so long as the variant proteases still demonstrate the requisite protease activity. By way of example, a variant may have at least 70%, preferably at least 80%, more preferably at least 90%, and most preferably at least 95 or at least 98% amino acid sequence homology with a reference protease sequence. Thus, the term variant includes non-cytotic proteases having enhanced (or decreased) endopeptidase activity—particular mention here is made to the increased K_{cat}/Kₘ of BoNT/A mutants 0161A, E54A, and K165L see Ahmed, S. A. (2008) Protein J. DOI 10.1007/s10930-007-9118-8, which is incorporated by reference thereto. The term fragment, when used in relation to a protease, typically means a peptide having at least 150, preferably at least 200, more preferably at least 250, and most preferably at least 300 amino acid residues of the reference protease. As with the TM ‘fragment’ component (discussed above), protease ‘fragments’ of the present invention embrace fragments of variant proteases based on a reference sequence.

The protease of the present invention preferably demonstrates a serine or metalloprotease activity (e.g. endopeptidase activity). The protease is preferably specific for a SNARE protein (e.g. SNAP-25, synaptobrevin/VAMP, or syntaxin).

Particular mention is made to the protease domains of neurotoxins, for example the protease domains of bacterial neurotoxins. Thus, the present invention embraces the use of neurotoxin domains, which occur in nature, as well as recombinantly prepared versions of said naturally-occurring neurotoxins.

Exemplary neurotoxins are produced by clostridia, and the term clostridial neurotoxin embraces neurotoxins produced by C. tetani (TeNT), and by C. botulinum (BoNT) serotypes A-G, as well as the closely related BoNT-like neurotoxins produced by C. baratii and C. butyricum. The above-mentioned abbreviations are used throughout the present specification. For example, the nomenclature BoNT/A denotes the source of neurotoxin as BoNT (serotype A). Corresponding nomenclature applies to other BoNT serotypes.

BoNTs are the most potent toxins known, with median lethal dose (LD50) values for mice ranging from 0.5 to 5 ng/kg depending on the serotype. BoNTs are adsorbed in the gastrointestinal tract, and, after entering the general circulation, bind to the presynaptic membrane of cholinergic nerve terminals and prevent the release of their neurotransmitter acetylcholine. BoNT/B, BoNT/D, BoNT/F and BoNT/G cleave synaptobrevin/vesicle-associated membrane protein (VAMP); BoNT/C, BoNT/A and BoNT/E cleave the synaptosomal-associated protein of 25 kDa (SNAP-25); and BoNT/C cleaves syntaxin.

BoNTs share a common structure, being di-chain proteins of ˜150 kDa, consisting of a heavy chain (H-chain) of ˜100 kDa covalently joined by a single disulfide bond to a light chain (L-chain) of ˜50 kDa. The H-chain consists of two domains, each of ˜50 kDa. The C-terminal domain (H_{C}) is required for the high-affinity neuronal binding, whereas the N-terminal domain (H_{N}) is proposed to be involved in membrane translocation. The L-chain is a zinc-dependent metalloprotease responsible for the cleavage of the substrate SNARE protein.

The term L-chain fragment means a component of the L-chain of a neurotoxin, which fragment demonstrates a metalloprotease activity and is capable of proteolytically cleaving a vesicle and/or plasma membrane associated protein involved in cellular exocytosis.

Examples of suitable protease (reference) sequences include:
Botulinum type A neurotoxin—amino acid residues (1-448)
Botulinum type B neurotoxin—amino acid residues (1-440)
Botulinum type C neurotoxin—amino acid residues (1-441)
Botulinum type D neurotoxin—amino acid residues (1-445)
Botulinum type E neurotoxin—amino acid residues (1-422)
Botulinum type F neurotoxin—amino acid residues (1-439)
Botulinum type G neurotoxin—amino acid residues (1-441)
Tetanus neurotoxin—amino acid residues (1-457)
IgA protease—amino acid residues (1-959)* *Pohlner, J. et al. (1987). Nature 325, pp. 458-462, which is hereby incorporated by reference thereto.

For recently-identified BoNT/X, the L-chain has been reported as corresponding to amino acids 1-439 thereof, with the L-chain boundary potentially varying by approximately 25 amino acids (e.g. 1-414 or 1-464).

The above-identified reference sequence should be considered a guide as slight variations may occur according to sub-serotypes. By way of example, US 2007/0166332 (hereby incorporated by reference thereto) cites slightly different clostridial sequences:
Botulinum type A neurotoxin—amino acid residues (M1-K448)
Botulinum type B neurotoxin—amino acid residues (M1-K441)
Botulinum type C neurotoxin—amino acid residues (M1-K449)
Botulinum type D neurotoxin—amino acid residues (M1-R445)
Botulinum type E neurotoxin—amino acid residues (M1-R422)
Botulinum type F neurotoxin—amino acid residues (M1-K439)
Botulinum type G neurotoxin—amino acid residues (M1-K446)
Tetanus neurotoxin—amino acid residues (M1-A457)

A variety of clostridial toxin fragments comprising the light chain can be useful in aspects of the present invention with the proviso that these light chain fragments can specifically target the core components of the neurotransmitter release apparatus and thus participate in executing the overall cellular mechanism whereby a clostridial toxin proteolytically cleaves a substrate. The light chains of clostridial toxins are approximately 420-460 amino acids in length and comprise an enzymatic domain. Research has shown that the entire length of a clostridial toxin light chain is not necessary for the enzymatic activity of the enzymatic domain. As a non-limiting example, the first eight amino acids of the BoNT/A light chain are not required for enzymatic activity. As another non-limiting example, the first eight amino acids of the TeNT light chain are not required for enzymatic activity. Likewise, the carboxyl-terminus of the light chain is not necessary for activity. As a non-limiting example, the last 32 amino acids of the BoNT/A light chain (residues 417-448) are not required for enzymatic activity. As another non-limiting example, the last 31 amino acids of the TeNT light chain (residues 427-457) are not required for enzymatic activity. Thus, aspects of this embodiment can include clostridial toxin light chains comprising an enzymatic domain having a length of, for example, at least 350 amino acids, at least 375 amino acids, at least 400 amino acids, at least 425 amino acids and at least 450 amino acids. Other aspects of this embodiment can include clostridial toxin light chains comprising an enzymatic domain having a length of, for example, at most 350 amino acids, at most 375 amino acids, at most 400 amino acids, at most 425 amino acids and at most 450 amino acids.

The non-cytotoxic protease component of the present invention preferably comprises a BoNT/A, BoNT/B, BoNT/C, BoNT/D, BoNT/E, BoNT/F, BoNT/G or BoNT/X serotype L-chain (or fragment or variant thereof).

The polypeptides of the present invention, especially the protease component thereof, may be PEGylated—this may help to increase stability, for example duration of action of the protease component. PEGylation is particularly preferred when the protease comprises a BoNT/A, B or C₁ protease. PEGylation preferably includes the addition of PEG to the N-terminus of the protease component. By way of example, the N-terminus of a protease may be extended with one or more amino acid (e.g. cysteine) residues, which may be the same or different. One or more of said amino acid residues may have its own PEG molecule attached (e.g. covalently attached) thereto. An example of this technology is described in WO2007/104567, which is incorporated in its entirety by reference thereto.

A Translocation Domain is a molecule that enables translocation of a protease into a target cell such that a functional expression of protease activity occurs within the cytosol of the target cell. Whether any molecule (e.g. a protein or peptide) possesses the requisite translocation function of the present invention may be confirmed by any one of a number of conventional assays.

For example, Shone C. (1987) describes an in vitro assay employing liposomes, which are challenged with a test molecule. Presence of the requisite translocation function is confirmed by release from the liposomes of K⁺ and/or labelled NAD, which may be readily monitored [see Shone C. (1987) Eur. J. Biochem; vol. 167(1): pp. 175-180].

A further example is provided by Blaustein R. (1987), which describes a simple in vitro assay employing planar phospholipid bilayer membranes. The membranes are challenged with a test molecule and the requisite translocation function is confirmed by an increase in conductance across said membranes [see Blaustein (1987) FEBS Letts; vol. 226, no. 1: pp. 115-120].

Additional methodology to enable assessment of membrane fusion and thus identification of Translocation Domains suitable for use in the present invention are provided by Methods in Enzymology Vol 220 and 221, Membrane Fusion Techniques, Parts A and B, Academic Press 1993.

The present invention also embraces variant translocation domains, preferably so long as the variant domains still demonstrate the requisite translocation activity. By way of example, a variant may have at least 70%, preferably at least 80%, more preferably at least 90%, and most preferably at least 95% or at least 98% amino acid sequence homology with a reference translocation domain. The term fragment, when used in relation to a translocation domain, means a peptide having at least 20, preferably at least 40, more preferably at least 80, and most preferably at least 100 amino acid residues of the reference translocation domain. In the case of a clostridial translocation domain, the fragment preferably has at least 100, preferably at least 150, more preferably at least 200, and most preferably at least 250 amino acid residues of the reference translocation domain (eg. H_{N} domain). As with the TM ‘fragment’ component (discussed above), translocation ‘fragments’ of the present invention embrace fragments of variant translocation domains based on the reference sequences.

The Translocation Domain is preferably capable of formation of ion-permeable pores in lipid membranes under conditions of low pH. Preferably it has been found to use only those portions of the protein molecule capable of pore-formation within the endosomal membrane.

The Translocation Domain may be obtained from a microbial protein source, in particular from a bacterial or viral protein source. Hence, in one embodiment, the Translocation Domain is a translocating domain of an enzyme, such as a bacterial toxin or viral protein.

It is well documented that certain domains of bacterial toxin molecules are capable of forming such pores. It is also known that certain translocation domains of virally expressed membrane fusion proteins are capable of forming such pores. Such domains may be employed in the present invention.

The Translocation Domain may be of a clostridial origin, such as the H_{N} domain (or a functional component thereof). H_{N} means a portion or fragment of the H-chain of a clostridial neurotoxin approximately equivalent to the amino-terminal half of the H-chain, or the domain corresponding to that fragment in the intact H-chain. The H-chain may lack the natural binding function of the H_{C} component of the H-chain. In some embodiments, the H_{C} function may be removed by deletion of the H_{C} amino acid sequence (either at the DNA synthesis level, or at the post-synthesis level by nuclease or protease treatment). Alternatively, in some embodiments the H_{C} function may be inactivated by chemical or biological treatment. Thus, in some embodiments the H-chain is incapable of binding to the Binding Site on a target cell to which native clostridial neurotoxin (i.e. holotoxin) binds.

Examples of suitable (reference) Translocation Domains include:
Botulinum type A neurotoxin—amino acid residues (449-871)
Botulinum type B neurotoxin—amino acid residues (441-858)
Botulinum type C neurotoxin—amino acid residues (442-866)
Botulinum type D neurotoxin—amino acid residues (446-862)
Botulinum type E neurotoxin—amino acid residues (423-845)
Botulinum type F neurotoxin—amino acid residues (440-864)
Botulinum type G neurotoxin—amino acid residues (442-863)
Tetanus neurotoxin—amino acid residues (458-879)

The above-identified reference sequence should be considered a guide as slight variations may occur according to sub-serotypes. By way of example, US 2007/0166332 (hereby incorporated by reference thereto) cites slightly different clostridial sequences:
Botulinum type A neurotoxin—amino acid residues (A449-K871)
Botulinum type B neurotoxin—amino acid residues (A442-S858)
Botulinum type C neurotoxin—amino acid residues (T450-N866)
Botulinum type D neurotoxin—amino acid residues (D446-N862)
Botulinum type E neurotoxin—amino acid residues (K423-K845)
Botulinum type F neurotoxin—amino acid residues (A440-K864)
Botulinum type G neurotoxin—amino acid residues (S447-S863)
Tetanus neurotoxin—amino acid residues (S458-V879)

In the context of the present invention, a variety of Clostridial toxin H_{N} regions comprising a translocation domain can be useful in aspects of the present invention preferably with the proviso that these active fragments can facilitate the release of a non-cytotoxic protease (e.g. a clostridial L-chain) from intracellular vesicles into the cytoplasm of the target cell and thus participate in executing the overall cellular mechanism whereby a clostridial toxin proteolytically cleaves a substrate. The H_{N} regions from the heavy chains of Clostridial toxins are approximately 410-430 amino acids in length and comprise a translocation domain. Research has shown that the entire length of a H_{N} region from a Clostridial toxin heavy chain is not necessary for the translocating activity of the translocation domain. Thus, aspects of this embodiment can include clostridial toxin H_{N} regions comprising a translocation domain having a length of, for example, at least 350 amino acids, at least 375 amino acids, at least 400 amino acids and at least 425 amino acids. Other aspects of this embodiment can include clostridial toxin H_{N} regions comprising translocation domain having a length of, for example, at most 350 amino acids, at most 375 amino acids, at most 400 amino acids and at most 425 amino acids.

For further details on the genetic basis of toxin production in Clostridium botulinum and C. tetani, we refer to Henderson et al (1997) in The Clostridia: Molecular Biology and Pathogenesis, Academic press.

The term H_{N} embraces naturally-occurring neurotoxin H_{N} portions, and modified H_{N} portions having amino acid sequences that do not occur in nature and/or synthetic amino acid residues, preferably so long as the modified H_{N} portions still demonstrate the above-mentioned translocation function.

Alternatively, the Translocation Domain may be of a non-clostridial origin. Examples of non-clostridial (reference) Translocation Domain origins include, but not be restricted to, the translocation domain of diphtheria toxin [O'Keefe et al., Proc. Natl. Acad. Sci. USA (1992) 89, 6202-6206; Silverman et al., J. Biol. Chem. (1993) 269, 22524-22532; and London, E. (1992) Biochem. Biophys. Acta., 1112, pp. 25-51], the translocation domain of Pseudomonas exotoxin type A [Prior et al. Biochemistry (1992) 31, 3555-3559], the translocation domains of anthrax toxin [Blanke et al. Proc. Natl. Acad. Sci. USA (1996) 93, 8437-8442], a variety of fusogenic or hydrophobic peptides of translocating function [Plank et al. J. Biol. Chem. (1994) 269, 12918-12924; and Wagner et al (1992) PNAS, 89, pp. 7934-7938], and amphiphilic peptides [Murata et al (1992) Biochem., 31, pp. 1986-1992]. The Translocation Domain may mirror the Translocation Domain present in a naturally-occurring protein, or may include amino acid variations preferably so long as the variations do not destroy the translocating ability of the Translocation Domain.

Particular examples of viral (reference) Translocation Domains suitable for use in the present invention include certain translocating domains of virally expressed membrane fusion proteins. For example, Wagner et al. (1992) and Murata et al. (1992) describe the translocation (i.e. membrane fusion and vesiculation) function of a number of fusogenic and amphiphilic peptides derived from the N-terminal region of influenza virus haemagglutinin. Other virally expressed membrane fusion proteins known to have the desired translocating activity are a translocating domain of a fusogenic peptide of Semliki Forest Virus (SFV), a translocating domain of vesicular stomatitis virus (VSV) glycoprotein G, a translocating domain of SER virus F protein and a translocating domain of Foamy virus envelope glycoprotein. Virally encoded Aspike proteins have particular application in the context of the present invention, for example, the E1 protein of SFV and the G protein of the G protein of VSV.

Use of the (reference) Translocation Domains listed in Table (below) includes use of sequence variants thereof. A variant may comprise one or more conservative nucleic acid substitutions and/or nucleic acid deletions or insertions, preferably with the proviso that the variant possesses the requisite translocating function. A variant may also comprise one or more amino acid substitutions and/or amino acid deletions or insertions, preferably so long as the variant possesses the requisite translocating function.

| Translocation | | |
|---|---|---|
| Domain source | Amino acid residues | References |
| Diphtheria toxin | 194-380 | Silverman et al., 1994, J. Biol. |
| | | Chem. 269, 22524-22532 |
| | | London E., 1992, Biochem. |
| | | Biophys. Acta., 1113, 25-51 |
| Domain II of | 405-613 | Prior et al., 1992, Biochemistry 31, |
| pseudomonas | | 3555-3559 |
| exotoxin | | Kihara & Pastan, 1994, Bioconj |
| | | Chem. 5, 532-538 |
| Influenza virus | GLFGAIAGFIENGWEGMIDGWYG | Plank et al., 1994, J. Biol. Chem. |
| haemagglutinin | (SEQ ID NO: 101), and | 269, 12918-12924 |
| | Variants thereof | Wagner et al., 1992, PNAS, 89, |
| | | 7934-7938 |
| | | Murata et al., 1992, Biochemistry |
| | | 31, 1986-1992 |
| Semliki Forest virus | Translocation domain | Kielian et al., 1996, J Cell Biol. |
| fusogenic protein | | 134(4), 863-872 |
| Vesicular Stomatitis | 118-139 | Yao et al., 2003, Virology 310(2), |
| virus glycoprotein G | | 319-332 |
| SER virus F protein | Translocation domain | Seth et al., 2003, J Virol 77(11) |
| | | 6520-6527 |
| Foamy virus envelope | Translocation domain | Picard-Maureau et al., 2003, J |
| glycoprotein | | Virol. 77(8), 4722-4730 |

Examples of clostridial neurotoxin H_{C} domain reference sequences include:
BoNT/A—N872-L1296
BoNT/B—E859-E1291
BoNT/C1—N867-E1291
BoNT/D—S863-E1276
BoNT/E—R846-K1252
BoNT/F—K865-E1274
BoNT/G—N864-E1297
TeNT—I880-D1315

For recently-identified BoNT/X, the H_{C} domain has been reported as corresponding to amino acids 893-1306 thereof, with the domain boundary potentially varying by approximately 25 amino acids (e.g. 868-1306 or 918-1306).

The polypeptides of the present invention may further comprise a translocation facilitating domain. Said domain facilitates delivery of the non-cytotoxic protease into the cytosol of the target cell and are described, for example, in WO 08/008803 and WO 08/008805, each of which is herein incorporated by reference thereto.

By way of example, suitable translocation facilitating domains include an enveloped virus fusogenic peptide domain, for example, suitable fusogenic peptide domains include influenzavirus fusogenic peptide domain (eg. influenza A virus fusogenic peptide domain of 23 amino acids), alphavirus fusogenic peptide domain (eg. Semliki Forest virus fusogenic peptide domain of 26 amino acids), vesiculovirus fusogenic peptide domain (eg. vesicular stomatitis virus fusogenic peptide domain of 21 amino acids), respirovirus fusogenic peptide domain (eg. Sendai virus fusogenic peptide domain of 25 amino acids), morbiliivirus fusogenic peptide domain (eg. Canine distemper virus fusogenic peptide domain of 25 amino acids), avulavirus fusogenic peptide domain (eg. Newcastle disease virus fusogenic peptide domain of 25 amino acids), henipavirus fusogenic peptide domain (eg. Hendra virus fusogenic peptide domain of 25 amino acids), metapneumovirus fusogenic peptide domain (eg. Human metapneumovirus fusogenic peptide domain of 25 amino acids) or spumavirus fusogenic peptide domain such as simian foamy virus fusogenic peptide domain; or fragments or variants thereof.

By way of further example, a translocation facilitating domain may comprise a Clostridial toxin H_{CN} domain or a fragment or variant thereof. In more detail, a Clostridial toxin H_{CN} translocation facilitating domain may have a length of at least 200 amino acids, at least 225 amino acids, at least 250 amino acids, at least 275 amino acids. In this regard, a Clostridial toxin H_{CN} translocation facilitating domain preferably has a length of at most 200 amino acids, at most 225 amino acids, at most 250 amino acids, or at most 275 amino acids. Specific (reference) examples include:
Botulinum type A neurotoxin—amino acid residues (872-1110)
Botulinum type B neurotoxin—amino acid residues (859-1097)
Botulinum type C neurotoxin—amino acid residues (867-1111)
Botulinum type D neurotoxin—amino acid residues (863-1098)
Botulinum type E neurotoxin—amino acid residues (846-1085)
Botulinum type F neurotoxin—amino acid residues (865-1105)
Botulinum type G neurotoxin—amino acid residues (864-1105)
Tetanus neurotoxin—amino acid residues (880-1127)

The above sequence positions may vary a little according to serotype/sub-type, and further examples of suitable (reference) Clostridial toxin H_{CN} domains include:
Botulinum type A neurotoxin—amino acid residues (874-1110)
Botulinum type B neurotoxin—amino acid residues (861-1097)
Botulinum type C neurotoxin—amino acid residues (869-1111)
Botulinum type D neurotoxin—amino acid residues (865-1098)
Botulinum type E neurotoxin—amino acid residues (848-1085)
Botulinum type F neurotoxin—amino acid residues (867-1105)
Botulinum type G neurotoxin—amino acid residues (866-1105)
Tetanus neurotoxin—amino acid residues (882-1127)

Any of the above-described facilitating domains may be combined with any of the previously described translocation domain peptides that are suitable for use in the present invention. Thus, by way of example, a non-clostridial facilitating domain may be combined with non-clostridial translocation domain peptide or with clostridial translocation domain peptide. Alternatively, a Clostridial toxin H_{CN} translocation facilitating domain may be combined with a non-clostridial translocation domain peptide. Alternatively, a Clostridial toxin H_{CN} facilitating domain may be combined or with a clostridial translocation domain peptide, examples of which include:
Botulinum type A neurotoxin—amino acid residues (449-1110)
Botulinum type B neurotoxin—amino acid residues (442-1097)
Botulinum type C neurotoxin—amino acid residues (450-1111)
Botulinum type D neurotoxin—amino acid residues (446-1098)
Botulinum type E neurotoxin—amino acid residues (423-1085)
Botulinum type F neurotoxin—amino acid residues (440-1105)
Botulinum type G neurotoxin—amino acid residues (447-1105)
Tetanus neurotoxin—amino acid residues (458-1127)

Embodiments related to the various methods of the invention are intended to be applied equally to other methods, the polypeptides, e.g. polypeptides suitable for labelling or labelled polypeptides, the nucleic acids, and vice versa.

Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics:1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes). The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

ALIGNMENT SCORES FOR DETERMINING SEQUENCE IDENTITY

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | −8 | | | | | | | | | | | |
| I | −1 | −3 | −3 | −3 | −1 | −3 | −3 | −4 | −4 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −1 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{\begin{matrix}[ length of the longer sequence plus the \\ number of gaps introduced into the longer \\ sequence in order to align the two sequence ]\end{matrix}} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

Conservative Amino Acid Substitutions

Basic: arginine
lysine
histidine

Acidic: glutamic acid
aspartic acid

Polar: glutamine
asparagine

Hydrophobic: leucine
isoleucine
valine

Aromatic: phenylalanine
tryptophan
tyrosine

Small: glycine
alanine
serine
threonine
methionine

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a polypeptide” includes a plurality of such candidate agents and reference to “the polypeptide” includes reference to one or more polypeptides and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

### BRIEF DESCRIPTION OF THE DRAWINGS

Embodiments of the invention will now be described, by way of example only, with reference to the following Figures and Examples.

FIG. 1 shows a schematic representation of the dual-labelling strategy of liganded polypeptides. The protein contains a SrtA recognition site at the C-terminal followed by a Strep-tag. At the N-terminal the protein contains a stretch of glycine protected by TEV cleavage site. A peptide containing a stretch of glycine attached to a fluorophore of choice and a second peptide containing the SrtA recognition site and 6 His tag (HT) were also generated. The two different SrtA enzymes allow site-specific labelling of fluorophores of different colours at the N- and C-termini.

FIG. 2 shows a SNAP-25 cleavage assay of unlabelled, single and dual-labelled polypeptides. A. SNAP-25 cleavage in cortical neurons by 3, 10, 30, 100, 300 and 1000 nM unlabelled EGF-liganded polypeptide, TxRed labelled EGF-polypeptide, SNAP594-labelled EGF-liganded polypeptide, single SrtA-mediated labelled EGF-liganded polypeptide and dual SrtA-labelled EGF-liganded polypeptide. As a control a polypeptide without the ligand (unliganded) was used for all concentrations. Exposure to the polypeptides was performed for 24 h. B. SNAP-25 cleavage in cortical neurons by 3, 10, 30, 100, 300 and 1000 nM unlabelled nociceptin-liganded polypeptide and dual SrtA-mediated labelled nociceptin-polypeptide. As a control a polypeptide without the ligand (unliganded) was used for all concentrations. Exposure to the polypeptides was performed for 24 h.

FIG. 3 shows live confocal imaging of dual-labelled EGF-liganded polypeptide. A. Snapshot of confocal live imaging recording of A549 cells treated with an EGF-liganded polypeptide labelled with HF555 at the N-terminal and HF488 at the C-terminal. The images (right) are snapshots of the boxed area shown on large image (left) taken at different intervals starting from 0.5 minutes after addition of the protein. Formation of the agglomerates characteristic of this polypeptide can be seen from 3 minutes onwards. B. Snapshot of confocal live imaging recording of A549 cells treated with an EGF-liganded polypeptide labelled with HF555 at the N-terminal and HF488 at the C-terminal. The images (right) are snapshots of the boxed area shown on large image (left) taken at different intervals starting from 30 minutes after addition of the protein. Disappearance of the agglomerates can be seen from 45 minutes onwards.

FIG. 4 shows a schematic representation of a dual-labelled full length proteolytically inactivate mutant of BoNT/A1, referred to as BoNT/A(0). The sortase donor and acceptor sites and protocol are the same as those of FIG. 1.

FIG. 5 shows SDS-PAGE analysis of a dual-labelled proteolytically inactivated BoNT/A (BoNT/A(0)) imaged using fluorescence (left) and Coomassie staining (right). Lanes 1 and 4 show the protein ladder, lanes 2 and 5 non-reduced dual-labelled BoNT/A(0) and lanes 3 and 6 show reduced dual-labelled (L-chain bottom and H-chain top) BoNT/A(0).

FIG. 6 shows timelapse single molecule TIRF microscopy images of single labelled BoNT/A(0) recorded at 5 second intervals. The white arrow shows the moving single molecule throughout time in seconds.

### SEQUENCE LISTING

Where an initial Met amino acid residue or a corresponding initial codon is indicated in any of the following SEQ ID NOs, said residue/codon is optional. In the event of any differences between the sequences described in the description and those of the ST.25 Sequence Listing, the sequences in the description shall prevail.

SEQ ID NO: 1—Nucleotide sequence of EGF-liganded (EGF TM) polypeptide with dual-labelling SrtA sites

SEQ ID NO: 2—Polypeptide sequence of EGF-liganded (EGF TM) polypeptide with dual-labelling SrtA sites

SEQ ID NO: 3—Nucleotide sequence of nociceptin-liganded (nociceptin TM) polypeptide with dual-labelling SrtA sites

SEQ ID NO: 4—Polypeptide sequence of nociceptin-liganded (nociceptin TM) polypeptide with dual-labelling SrtA sites

SEQ ID NO: 5—Nucleotide sequence of EGF-liganded (EGF TM) polypeptide

SEQ ID NO: 6—Polypeptide sequence of EGF-liganded (EGF TM) polypeptide

SEQ ID NO: 7—Nucleotide sequence of nociceptin-liganded (nociceptin TM) polypeptide

SEQ ID NO: 8—Polypeptide sequence of nociceptin-liganded (nociceptin TM) polypeptide

SEQ ID NO: 9—Nucleotide sequence of EGF-liganded polypeptide GFP-tagged

SEQ ID NO: 10—Polypeptide sequence of EGF-liganded polypeptide GFP-tagged

SEQ ID NO: 11—Nucleotide sequence of EGF-liganded polypeptide SNAP tagged

SEQ ID NO: 12—Polypeptide sequence of EGF-liganded polypeptide SNAP tagged

SEQ ID NO: 13—Nucleotide sequence of Sortase A (LPESG-targeting)

SEQ ID NO: 14—Polypeptide sequence of Sortase A (LPESG-targeting)

SEQ ID NO: 15—Nucleotide sequence of Sortase A (LAETG-targeting)

SEQ ID NO: 16—Polypeptide sequence of Sortase A (LAETG-targeting)

SEQ ID NO: 17—BoNT/A—UniProt P10845

SEQ ID NO: 18—BoNT/B—UniProt P10844

SEQ ID NO: 19—BoNT/C—UniProt P18640

SEQ ID NO: 20—BoNT/D—UniProt P19321

SEQ ID NO: 21—BoNT/E—UniProt Q00496

SEQ ID NO: 22—BoNT/F—UniProt A7GBG3

SEQ ID NO: 23—BoNT/G—UniProt Q60393

SEQ ID NO: 24—Polypeptide Sequence of BoNT/X

SEQ ID NO: 25—TeNT—UniProt P04958

SEQ ID NO: 26—Polypeptide sequence of labelled EGF TM polypeptide

SEQ ID NO: 27—Polypeptide sequence of C. ternatea butelase 1 (plus signal peptide)

SEQ ID NO: 28—Polypeptide sequence of C. ternatea butelase 1 (minus signal peptide)

SEQ ID NO: 29—Peptide with conjugated detectable label and sortase donor site

SEQ ID NO: 30—Peptide with conjugated detectable label and sortase acceptor site

SEQ ID NO: 31—Polypeptide sequence of Staphylococcus aureus Sortase A

SEQ ID NO: 32—Polypeptide sequence of Staphylococcus aureus Sortase B

SEQ ID NO: 33—Polypeptide sequence of Streptococcus pneumoniae Sortase A

SEQ ID NO: 34—Polypeptide sequence of Streptococcus pneumoniae Sortase B

SEQ ID NO: 35—Polypeptide sequence of Streptococcus pneumoniae Sortase C

SEQ ID NO: 36—Polypeptide sequence of Streptococcus pneumoniae Sortase D

SEQ ID NO: 37—Polypeptide sequence of Streptococcus pyogenes Sortase A

SEQ ID NO: 38—Polypeptide sequence of proteolytically inactive mutant BoNT/A(0)

SEQ ID NO: 39—Nucleotide sequence of full length proteolytically inactive mutant BoNT/A(0) with dual-labelling SrtA sites

SEQ ID NO: 40—Polypeptide sequence of full length proteolytically inactive mutant BoNT/A(O) with dual-labelling SrtA sites

SEQ ID NO: 41—Polypeptide sequence of Prochloron didemni PATG

SEQ ID NO: 42—Polypeptide sequence of Saponaria vaccaria PCY1

SEQ ID NO: 43—Polypeptide sequence of Galerina marginata POPB

SEQ ID NO: 44—Polypeptide sequence of Oldenlandia affinis Butelase homologue OaAEP1b (plus signal peptide)

SEQ ID NO: 45—Polypeptide sequence of Oldenlandia affinis Butelase homologue OaAEP1b (minus signal peptide)

Nucleotide sequence of EGF−liganded polypeptide with dual−labelling
SrtA sites
SEQ ID NO: 1
TGGCGAATGGGACGCGCCCTGTAGCGGCGCATTAAGCGCGGCGGGTGTGGTGGTTACGCGCAGCGTGA
CCGCTACACTTGCCAGCGCCCTAGCGCCCGCTCCTTTCGCTTTCTTCCCTTCCTTTCTCGCCACGTTC
GCCGGCTTTCCCCGTCAAGCTCTAAATCGGGGGCTCCCTTTAGGGTTCCGATTTAGTGCTTTACGGCA
CCTCGACCCCAAAAAACTTGATTAGGGTGATGGTTCACGTAGTGGGCCATCGCCCTGATAGACGGTTT
TTCGCCCTTTGACGTTGGAGTCCACGTTCTTTAATAGTGGACTCTTGTTCCAAACTGGAACAACACTC
AACCCTATCTCGGTCTATTCTTTTGATTTATAAGGGATTTTGCCGATTTCGGCCTATTGGTTAAAAAA
TGAGCTGATTTAACAAAAATTTAACGCGAATTTTAACAAAATATTAACGCTTACAATTTAGGTGGCAC
TTTTCGGGGAAATGTGCGCGGAACCCCTATTTGTTTATTTTTCTAAATACATTCAAATATGTATCCGC
TCATGAATTAATTCTTAGAAAAACTCATCGAGCATCAAATGAAACTGCAATTTATTCATATCAGGATT
ATCAATACCATATTTTTGAAAAAGCCGTTTCTGTAATGAAGGAGAAAACTCACCGAGGCAGTTCCATA
GGATGGCAAGATCCTGGTATCGGTCTGCGATTCCGACTCGTCCAACATCAATACAACCTATTAATTTC
CCCTCGTCAAAAATAAGGTTATCAAGTGAGAAATCACCATGAGTGACGACTGAATCCGGTGAGAATGG
CAAAAGTTTATGCATTTCTTTCCAGACTTGTTCAACAGGCCAGCCATTACGCTCGTCATCAAAATCAC
TCGCATCAACCAAACCGTTATTCATTCGTGATTGCGCCTGAGCGAGACGAAATACGCGATCGCTGTTA
AAAGGACAATTACAAACAGGAATCGAATGCAACCGGCGCAGGAACACTGCCAGCGCATCAACAATATT
TTCACCTGAATCAGGATATTCTTCTAATACCTGGAATGCTGTTTTCCCGGGGATCGCAGTGGTGAGTA
ACCATGCATCATCAGGAGTACGGATAAAATGCTTGATGGTCGGAAGAGGCATAAATTCCGTCAGCCAG
TTTAGTCTGACCATCTCATCTGTAACATCATTGGCAACGCTACCTTTGCCATGTTTCAGAAACAACTC
TGGCGCATCGGGCTTCCCATACAATCGATAGATTGTCGCACCTGATTGCCCGACATTATCGCGAGCCC
ATTTATACCCATATAAATCAGCATCCATGTTGGAATTTAATCGCGGCCTAGAGCAAGACGTTTCCCGT
TGAATATGGCTCATAACACCCCTTGTATTACTGTTTATGTAAGCAGACAGTTTTATTGTTCATGACCA
AAATCCCTTAACGTGAGTTTTCGTTCCACTGAGCGTCAGACCCCGTAGAAAAGATCAAAGGATCTTCT
TGAGATCCTTTTTTTCTGCGCGTAATCTGCTGCTTGCAAACAAAAAAACCACCGCTACCAGCGGTGGT
TTGTTTGCCGGATCAAGAGCTACCAACTCTTTTTCCGAAGGTAACTGGCTTCAGCAGAGCGCAGATAC
CAAATACTGTCCTTCTAGTGTAGCCGTAGTTAGGCCACCACTTCAAGAACTCTGTAGCACCGCCTACA
TACCTCGCTCTGCTAATCCTGTTACCAGTGGCTGCTGCCAGTGGCGATAAGTCGTGTCTTACCGGGTT
GGACTCAAGACGATAGTTACCGGATAAGGCGCAGCGGTCGGGCTGAACGGGGGGTTCGTGCACACAGC
CCAGCTTGGAGCGAACGACCTACACCGAACTGAGATACCTACAGCGTGAGCTATGAGAAAGCGCCACG
CTTCCCGAAGGGAGAAAGGCGGACAGGTATCCGGTAAGCGGCAGGGTCGGAACAGGAGAGCGCACGAG
GGAGCTTCCAGGGGGAAACGCCTGGTATCTTTATAGTCCTGTCGGGTTTCGCCACCTCTGACTTGAGC
GTCGATTTTTGTGATGCTCGTCAGGGGGGCGGAGCCTATGGAAAAACGCCAGCAACGCGGCCTTTTTA
CGGTTCCTGGCCTTTTGCTGGCCTTTTGCTCACATCGGCGATAATGGCCTGCTTCTCGCCGAAACGTT
TGGTGGCGGGACCAGTGACGAAGGCTTGAGCGAGGGCGTGCAAGATTCCGAATACCGCAAGCGACAGG
CCGATCATCGTCGCGCTCCAGCGAAAGCGGTCCTCGCCGAAAATGACCCAGAGCGCTGCCGGCACCTG
TCCTACGAGTTGCATGATAAAGAAGACAGTCATAAGTGCGGCGACGATAGTCATGCCCCGCGCCCACC
GGAAGGAGCTGACTGGGTTGAAGGCTCTCAAGGGCATCGGTCGAGATCCCGGTGCCTAATGAGTGAGC
TAACTTACATTAATTGCGTTGCGCTCACTGCCCGCTTTCCAGTCGGGAAACCTGTCGTGCCAGCTGCA
TTAATGAATCGGCCAACGCGCGGGGAGAGGCGGTTTGCGTATTGGGCGCCAGGGTGGTTTTTCTTTTC
ACCAGTGAGACGGGCAACAGCTGATTGCCCTTCACCGCCTGGCCCTGAGAGAGTTGCAGCAAGCGGTC
CACGCTGGTTTGCCCCAGCAGGCGAAAATCCTGTTTGATGGTGGTTAACGGCGGGATATAACATGAGC
TGTCTTCGGTATCGTCGTATCCCACTACCGAGATATCCGCACCAACGCGCAGCCCGGACTCGGTAATG
GCGCGCATTGCGCCCAGCGCCATCTGATCGTTGGCAACCAGCATCGCAGTGGGAACGATGCCCTCATT
CAGCATTTGCATGGTTTGTTGAAAACCGGACATGGCACTCCAGTCGCCTTCCCGTTCCGCTATCGGCT
GAATTTGATTGCGAGTGAGATATTTATGCCAGCCAGCCAGACGCAGACGCGCCGAGACAGAACTTAAT
GGGCCCGCTAACAGCGCGATTTGCTGGTGACCCAATGCGACCAGATGCTCCACGCCCAGTCGCGTACC
GTCTTCATGGGAGAAAATAATACTGTTGATGGGTGTCTGGTCAGAGACATCAAGAAATAACGCCGGAA
CATTAGTGCAGGCAGCTTCCACAGCAATGGCATCCTGGTCATCCAGCGGATAGTTAATGATCAGCCCA
CTGACGCGTTGCGCGAGAAGATTGTGCACCGCCGCTTTACAGGCTTCGACGCCGCTTCGTTCTACCAT
CGACAGGACCACGCTGGCACCCAGTTGATCGGCGCGAGATTTAATCGCCGCGACAATTTGCGACGGCG
CGTGCAGGGCCAGACTGGAGGTGGCAACGCCAATCAGCAACGACTGTTTGCCCGCCAGTTGTTGTGCC
ACGCGGTTGGGAATGTAATTCAGCTCCGCCATCGCCGCTTCCACTTTTTCCCGCGTTTTCGCAGAAAC
GTGGCTGGCCTGGTTCACCACGCGGGAAACGGTCTGATAAGAGACACCGGCATACTCTGCGACATCGT
ATAACGTTACTGGTTTCACATTCACCACCCTGAATTGACTCTCTTCCGGGCGCTATCATGCCATACCG
CGAAAGGTTTTGCGCCATTCGATGGTGTCCGGGATCTCGACGCTCTCCCTTATGCGACTCCTGCATTA
GGAAGCAGCCCAGTAGTAGGTTGAGGCCGTTGAGCACCGCCGCCGCAAGGAATGGTGCATGCAAGGAG
ATGGCGCCCAACAGTCCCCCGGCCACGGGGCCTGCCACCATACCCACGCCGAAACAAGCGCTCATGAG
CCCGAAGTGGCGAGCCCGATCTTCCCCATCGGTGATGTCGGCGATATAGGCGCCAGCAACCGCACCTG
TGGCGCCGGTGATGCCGGCCACGATGCGTCCGGCGTAGAGGATCGAGATCTCGATCCCGCGAAATTAA
TACGACTCACTATAGGGGAATTGTGAGCGGATAACAATTCCCCTCAAGAAATAATTTTGTTTAACTTT
AAGAAGGAGATATACATATgggatccatgGAGAACCTGTATTTTCAGGGCGGCGGTGGCAGCGGCGGC
AGCGGCGGCAGCcctttcgttaacaaacagttcaactataaagacccagttaacggtgttgacattgc
ttacatcaaaatcccgaacgctggccagatgcagccggtaaaggcattcaaaatccacaacaaaatct
gggttatcccggaacgtgatacctttactaacccggaagaaggtgacctgaacccgccaccggaagcg
aaacaggtgccggtatcttactatgactccacctacctgtctaccgataacgaaaaggacaactacct
gaaaggtgttactaaactgttcgagcgtatttactccaccgacctgggccgtatgctgctgactagca
tcgttcgcggtatcccgttctggggcggttctaccatcgataccgaactgaaagtaatcgacactaac
tgcatcaacgttattcagccggacggttcctatcgttccgaagaactgaacctggtgatcatcggccc
gtctgctgatatcatccagttcgagtgtaagagctttggtcacgaagttctgaacctcacccgtaacg
gctacggttccactcagtacatccgtttctctccggacttcaccttcggttttgaagaatccctggaa
gtagacacgaacccactgctgggcgctggtaaattcgcaactgatcctgcggttaccctggctcacga
actgattcatgcaggccaccgcctgtacggtatcgccatcaatccgaaccgtgtcttcaaagttaaca
ccaacgcgtattacgagatgtccggtctggaagttagcttcgaagaactgcgtacttttggcggtcac
gacgctaaattcatcgactctctgcaagaaaacgagttccgtctgtactactataacaagttcaaaga
tatcgcatccaccctgaacaaagcgaaatccatcgtgggtaccactgcttctctccagtacatgaaga
acgtttttaaagaaaaatacctgctcagcgaagacacctccggcaaattctctgtagacaagttgaaa
ttcgataaactttacaaaatgctgactgaaatttacaccgaagacaacttcgttaagttctttaaagt
tctgaaccgcaaaacctatctgaacttcgacaaggcagtattcaaaatcaacatcgtgccgaaagtta
actacactatctacgatggtttcaacctgcgtaacaccaacctggctgctaattttaacggccagaac
acggaaatcaacaacatgaacttcacaaaactgaaaaacttcactggtctgttcgagttttacaagct
gctgtgcgtcgacggcatcattacctccaaaactaaatctctgatagaaggtagaaacaaagcgctga
acctgcagtgtatcaaggttaacaactgggatttattcttcagcccgagtgaagacaacttcaccaac
gacctgaacaaaggtgaagaaatcacctcagatactaacatcgaagcagccgaagaaaacatctcgct
agacctgatccagcagtactacctgacctttaatttcgacaacgagccggaaaacatttctatcgaaa
acctgagctctgatatcatcggccagctggaactgatgccgaacatcgaacgtttcccaaacggtaaa
aagtacgagctggacaaatataccatgttccactacctgcgcgcgcaggaatttgaacacggcaaatc
ccgtatcgcactgactaactccgttaacgaagctctgctcaacccgtcccgtgtatacaccttcttct
ctagcgactacgtgaaaaaggtcaacaaagcgactgaagctgcaatgttcttgggttgggttgaacag
cttgtttatgattttaccgacgagacgtccgaagtatctactaccgacaaaattgcggatatcactat
catcatcccgtacatcggtccggctctgaacattggcaacatgctgtacaaagacgacttcgttggcg
cactgatcttctccggtgcggtgatcctgctggagttcatcccggaaatcgccatcccggtactgggc
acctttgctctggtttcttacattgcaaacaaggttctgactgtacaaaccatcgacaacgcgctgag
caaacgtaacgaaaaatgggatgaagtttacaaatatatcgtgaccaactggctggctaaggttaata
ctcagatcgacctcatccgcaaaaaaatgaaagaagcactggaaaaccaggcggaagctaccaaggca
atcattaactaccagtacaaccagtacaccgaggaagaaaaaaacaacatcaacttcaacatcgacga
tctgtcctctaaactgaacgaatccatcaacaaagctatgatcaacatcaacaagttcctgaaccagt
gctctgtaagctatctgatgaactccatgatcccgtacggtgttaaacgtctggaggacttcgatgcg
tctctgaaagacgccctgctgaaatacatttacgacaaccgtggcactctgatcggtcaggttgatcg
tctgaaggacaaagtgaacaataccttatcgaccgacatcccttttcagctcagtaaatatgtcgata
accaacgccttttgtccactctagaaggcggTGGCGGTAGCGGTGGCGGTGGCAGCGGCGGTGGCGGT
AGCGCACTAGacAACAGCGACCCTAAATGCCCACTgAGTCATGAAGGATACTGCCTTAATGATGGTGT
TTGTATGTACATAGGAACATTGGACCGTTATGCTTGCAATTGTGTAGTGGGCTATGTCGGGGAAAGGT
GTCAATATCGAGATCTCAAGCTGGCAGAGTTAAGAgggctagaagcaGGCGGCAGCGGCGGCGGCAGC
GGCCTGCCCGAAAGCGGTGGCGGATCTGCTTGGTCTCACCCGCAGTTCGAAAAAGGTGGTGGTTCTGG
TGGTGGTTCTGGTGGTTCTGCTTGGTCTCACCCGCAGTTCGAAAAAtaatgaAAGCTTGCGGCCGCAC
TCGAGCACCACCACCACCACCACTGAGATCCGGCTGCTAACAAAGCCCGAAAGGAAGCTGAGTTGGCT
GCTGCCACCGCTGAGCAATAACTAGCATAACCCCTTGGGGCCTCTAAACGGGTCTTGAGGGGTTTTTT
GCTGAAAGGAGGAACTATATCCGGAT
Polypeptide sequence of EGF-liganded polypeptide with dual-labelling
SrtA sites
SEQ ID NO: 2
MENLYFQGGGGSGGSGGSPFVNKQFKYKDPVNGVDIAYIKIPNAGQMQPVKAFKIHKKIWVIPERDTF
TNPEEGDLNPPPEAKQVPVSYYDSTYLSTDNEKDNYLKGVTKLFERIYSTDLGRMLLTSIVRGIPFWG
GSTIDTELKVIDTNCINVIQPDGSYRSEELNLVIIGPSADIIQFECKSFGHEVLNLTRNGYGSTQYIR
FSPDFTFGFEESLEVDTNPLLGAGKFATDPAVTLAHELIHAGHRLYGIAINPNRVFKVNTNAYYEMSG
LEVSFEELRTFGGHDAKFIDSLQENEFRLYYYNKFKDIASTLNKAKSIVGTTASLQYMKNVFKEKYLL
SEDTSGKFSVDKLKFDKLYKMLTEIYTEDNFVKFFKVLNRKTYLNFDKAVFKINIVPKVNYTIYDGFN
LRNTNLAANFNGQNTEINNMNFTKLKNFTGLFEFYKLLCVDGIITSKTKSLIEGRNKALNLQCIKVNN
WDLFFSPSEDNFTNDLNKGEEITSDTNIEAAEENISLDLIQQYYLTFNFDNEPENISIENLSSDIIGQ
LELMPNIERFPNGKKYELDKYTMFHYLRAQEFEHGKSRIALTNSVNEALLNPSRVYTFFSSDYVKKVN
KATEAAMFLGWVEQLVYDFTDETSEVSTTDKIADITIIIPYIGPALNIGNMLYKDDFVGALIFSGAVI
LLEFIPEIAIPVLGTFALVSYIANKVLTVQTIDNALSKRNEKWDEVYKYIVTNWLAKVNTQIDLIRKK
MKEALENQAEATKAIINYQYNQYTEEEKNNINFNIDDLSSKLNESINKAMININKFLNQCSVSYLMNS
MIPYGVKRLEDFDASLKDALLKYIYDMRGTLIGQvDRLKDKVNNTLSTDIPFQLSKYVDNQRLLSTLE
GGGGSGGGGSGGGGSALDNSDPKCPLSHEGYCLNDGVCMYIGTLDRYACNCWGYVGERCQYRDLKLA
ELRGLEAGGSGGGSGLPESGGGSAWSHPQFEKGGGSGGGSGGSAWSHPQFEK
Nucleotide sequence of nociceptin-liganded polypeptide with dual-
labelling SrtA sites
SEQ ID NO: 3
TGGCGAATGGGACGCGCCCTGTAGCGGCGCATTAAGCGCGGCGGGTGTGGTGGTTACGCGCAGCGTGA
CCGCTACACTTGCCAGCGCCCTAGCGCCCGCTCCTTTCGCTTTCTTCCCTTCCTTTCTCGCCACGTTC
GCCGGCTTTCCCCGTCAAGCTCTAAATCGGGGGCTCCCTTTAGGGTTCCGATTTAGTGCTTTACGGCA
CCTCGACCCCAAAAAACTTGATTAGGGTGATGGTTCACGTAGTGGGCCATCGCCCTGATAGACGGTTT
TTCGCCCTTTGACGTTGGAGTCCACGTTCTTTAATAGTGGACTCTTGTTCCAAACTGGAACAACACTC
AACCCTATCTCGGTCTATTCTTTTGATTTATAAGGGATTTTGCCGATTTCGGCCTATTGGTTAAAAAA
TGAGCTGATTTAACAAAAATTTAACGCGAATTTTAACAAAATATTAACGCTTACAATTTAGGTGGCAC
TTTTCGGGGAAATGTGCGCGGAACCCCTATTTGTTTATTTTTCTAAATACATTCAAATATGTATCCGC
TCATGAATTAATTCTTAGAAAAACTCATCGAGCATCAAATGAAACTGCAATTTATTCATATCAGGATT
ATCAATACCATATTTTTGAAAAAGCCGTTTCTGTAATGAAGGAGAAAACTCACCGAGGCAGTTCCATA
GGATGGCAAGATCCTGGTATCGGTCTGCGATTCCGACTCGTCCAACATCAATACAACCTATTAATTTC
CCCTCGTCAAAAATAAGGTTATCAAGTGAGAAATCACCATGAGTGACGACTGAATCCGGTGAGAATGG
CAAAAGTTTATGCATTTCTTTCCAGACTTGTTCAACAGGCCAGCCATTACGCTCGTCATCAAAATCAC
TCGCATCAACCAAACCGTTATTCATTCGTGATTGCGCCTGAGCGAGACGAAATACGCGATCGCTGTTA
AAAGGACAATTACAAACAGGAATCGAATGCAACCGGCGCAGGAACACTGCCAGCGCATCAACAATATT
TTCACCTGAATCAGGATATTCTTCTAATACCTGGAATGCTGTTTTCCCGGGGATCGCAGTGGTGAGTA
ACCATGCATCATCAGGAGTACGGATAAAATGCTTGATGGTCGGAAGAGGCATAAATTCCGTCAGCCAG
TTTAGTCTGACCATCTCATCTGTAACATCATTGGCAACGCTACCTTTGCCATGTTTCAGAAACAACTC
TGGCGCATCGGGCTTCCCATACAATCGATAGATTGTCGCACCTGATTGCCCGACATTATCGCGAGCCC
ATTTATACCCATATAAATCAGCATCCATGTTGGAATTTAATCGCGGCCTAGAGCAAGACGTTTCCCGT
TGAATATGGCTCATAACACCCCTTGTATTACTGTTTATGTAAGCAGACAGTTTTATTGTTCATGACCA
AAATCCCTTAACGTGAGTTTTCGTTCCACTGAGCGTCAGACCCCGTAGAAAAGATCAAAGGATCTTCT
TGAGATCCTTTTTTTCTGCGCGTAATCTGCTGCTTGCAAACAAAAAAACCACCGCTACCAGCGGTGGT
TTGTTTGCCGGATCAAGAGCTACCAACTCTTTTTCCGAAGGTAACTGGCTTCAGCAGAGCGCAGATAC
CAAATACTGTCCTTCTAGTGTAGCCGTAGTTAGGCCACCACTTCAAGAACTCTGTAGCACCGCCTACA
TACCTCGCTCTGCTAATCCTGTTACCAGTGGCTGCTGCCAGTGGCGATAAGTCGTGTCTTACCGGGTT
GGACTCAAGACGATAGTTACCGGATAAGGCGCAGCGGTCGGGCTGAACGGGGGGTTCGTGCACACAGC
CCAGCTTGGAGCGAACGACCTACACCGAACTGAGATACCTACAGCGTGAGCTATGAGAAAGCGCCACG
CTTCCCGAAGGGAGAAAGGCGGACAGGTATCCGGTAAGCGGCAGGGTCGGAACAGGAGAGCGCACGAG
GGAGCTTCCAGGGGGAAACGCCTGGTATCTTTATAGTCCTGTCGGGTTTCGCCACCTCTGACTTGAGC
GTCGATTTTTGTGATGCTCGTCAGGGGGGCGGAGCCTATGGAAAAACGCCAGCAACGCGGCCTTTTTA
CGGTTCCTGGCCTTTTGCTGGCCTTTTGCTCACATCGGCGATAATGGCCTGCTTCTCGCCGAAACGTT
TGGTGGCGGGACCAGTGACGAAGGCTTGAGCGAGGGCGTGCAAGATTCCGAATACCGCAAGCGACAGG
CCGATCATCGTCGCGCTCCAGCGAAAGCGGTCCTCGCCGAAAATGACCCAGAGCGCTGCCGGCACCTG
TCCTACGAGTTGCATGATAAAGAAGACAGTCATAAGTGCGGCGACGATAGTCATGCCCCGCGCCCACC
GGAAGGAGCTGACTGGGTTGAAGGCTCTCAAGGGCATCGGTCGAGATCCCGGTGCCTAATGAGTGAGC
TAACTTACATTAATTGCGTTGCGCTCACTGCCCGCTTTCCAGTCGGGAAACCTGTCGTGCCAGCTGCA
TTAATGAATCGGCCAACGCGCGGGGAGAGGCGGTTTGCGTATTGGGCGCCAGGGTGGTTTTTCTTTTC
ACCAGTGAGACGGGCAACAGCTGATTGCCCTTCACCGCCTGGCCCTGAGAGAGTTGCAGCAAGCGGTC
CACGCTGGTTTGCCCCAGCAGGCGAAAATCCTGTTTGATGGTGGTTAACGGCGGGATATAACATGAGC
TGTCTTCGGTATCGTCGTATCCCACTACCGAGATATCCGCACCAACGCGCAGCCCGGACTCGGTAATG
GCGCGCATTGCGCCCAGCGCCATCTGATCGTTGGCAACCAGCATCGCAGTGGGAACGATGCCCTCATT
CAGCATTTGCATGGTTTGTTGAAAACCGGACATGGCACTCCAGTCGCCTTCCCGTTCCGCTATCGGCT
GAATTTGATTGCGAGTGAGATATTTATGCCAGCCAGCCAGACGCAGACGCGCCGAGACAGAACTTAAT
GGGCCCGCTAACAGCGCGATTTGCTGGTGACCCAATGCGACCAGATGCTCCACGCCCAGTCGCGTACC
GTCTTCATGGGAGAAAATAATACTGTTGATGGGTGTCTGGTCAGAGACATCAAGAAATAACGCCGGAA
CATTAGTGCAGGCAGCTTCCACAGCAATGGCATCCTGGTCATCCAGCGGATAGTTAATGATCAGCCCA
CTGACGCGTTGCGCGAGAAGATTGTGCACCGCCGCTTTACAGGCTTCGACGCCGCTTCGTTCTACCAT
CGACACCACCACGCTGGCACCCAGTTGATCGGCGCGAGATTTAATCGCCGCGACAATTTGCGACGGCG
CGTGCAGGGCCAGACTGGAGGTGGCAACGCCAATCAGCAACGACTGTTTGCCCGCCAGTTGTTGTGCC
ACGCGGTTGGGAATGTAATTCAGCTCCGCCATCGCCGCTTCCACTTTTTCCCGCGTTTTCGCAGAAAC
GTGGCTGGCCTGGTTCACCACGCGGGAAACGGTCTGATAAGAGACACCGGCATACTCTGCGACATCGT
ATAACGTTACTGGTTTCACATTCACCACCCTGAATTGACTCTCTTCCGGGCGCTATCATGCCATACCG
CGAAAGGTTTTGCGCCATTCGATGGTGTCCGGGATCTCGACGCTCTCCCTTATGCGACTCCTGCATTA
GGAAGCAGCCCAGTAGTAGGTTGAGGCCGTTGAGCACCGCCGCCGCAAGGAATGGTGCATGCAAGGAG
ATGGCGCCCAACAGTCCCCCGGCCACGGGGCCTGCCACCATACCCACGCCGAAACAAGCGCTCATGAG
CCCGAAGTGGCGAGCCCGATCTTCCCCATCGGTGATGTCGGCGATATAGGCGCCAGCAACCGCACCTG
TGGCGCCGGTGATGCCGGCCACGATGCGTCCGGCGTAGAGGATCGAGATCTCGATCCCGCGAAATTAA
TACGACTCACTATAGGGGAATTGTGAGCGGATAACAATTCCCCTCAAGAAATAATTTTGTTTAACTTT
AAGAAGGAGATATACATatgGAGAACCTGTATTTTCAGGGCGGCGGTGGCAGCGGCGGCAGCGGCGGC
AGCGGCAGCATGcctTTTGTGAACAAACAGTTCAACTATAAGGATCCGGTTAATGGTGTGGATATCGC
CTATATCAAAATTCCGAATGCAGGTCAGATGCAGCCGGTTAAAGCCTTTAAAATCCATAACAAAATTT
GGGTGATTCCGGAACGTGATACCTTTACCAATCCGGAAGAAGGTGATCTGAATCCGCCTCCGGAAGCA
AAACAGGTTCCGGTTAGCTATTATGATAGCACCTATCTGAGCACCGATAACGAGAAAGATAACTATCT
GAAAGGTGTGACCAAACTGTTTGAACGCATTTATAGTACCGATCTGGGTCGTATGCTGCTGACCAGCA
TTGTTCGTGGTATTCCGTTTTGGGGTGGTAGCACCATTGATACCGAACTGAAAGTTATTGACACCAAC
TGCATTAATGTGATTCAGCCGGATGGTAGCTATCGTAGCGAAGAACTGAATCTGGTTATTATTGGTCC
GAGCGCAGATATCATTCAGTTTGAATGTAAATCCTTTGGCCACGAAGTTCTGAATCTGACCCGTAATG
GTTATGGTAGTACCCAGTATATTCGTTTCAGTCCGGATTTTACCTTTGGCTTTGAAGAAAGCCTGGAA
GTTGATACAAATCCGCTGTTAGGTGCAGGTAAATTTGCAACCGATCCGGCAGTTACCCTGGCACATGA
ACTGATTCATGCCGGTCATCGTCTGTATGGTATTGCAATTAATCCGAACCGTGTGTTCAAAGTGAATA
CCAACGCATATTATGAAATGAGCGGTCTGGAAGTGTCATTTGAAGAACTGCGTACCTTTGGTGGTCAT
GATGCCAAATTTATCGATAGCCTGCAAGAAAATGAATTTCGCCTGTACTACTATAACAAATTCAAGGA
TATTGCGAGCACCCTGAATAAAGCCAAAAGCATTGTTGGCACCACCGCAAGCCTGCAGTATATGAAAA
ATGTGTTTAAAGAAAAATATCTGCTGAGCGAAGATACCAGCGGTAAATTTAGCGTTGACAAACTGAAA
TTCGATAAACTGTACAAGATGCTGACCGAGATTTATACCGAAGATAACTTCGTGAAGTTTTTCAAAGT
GCTGAACCGCAAAACCTACCTGAACTTTGATAAAGCCGTGTTCAAAATCAACATCGTGCCGAAAGTGA
ACTATACCATCTATGATGGTTTTAACCTGCGCAATACCAATCTGGCAGCAAACTTTAATGGTCAGAAC
ACCGAAATCAACAACATGAACTTTACCAAACTGAAGAACTTCACCGGTCTGTTCGAATTTTACAAACT
GCTGTGTGTGGATGGCATTATTACCAGCAAAACCAAATCCGATGATGACGATAAATTCGGTGGTTTTA
CCGGTGCACGTAAAAGCGCACGTAAACGTAAAAATCAGGCACTGGCAGGCGGTGGTGGTAGCGGTGGC
GGTGGTTCAGGTGGTGGTGGCTCAGCACTGGTTCTGCAGTGTATTAAAGTTAATAACTGGGACCTGTT
TTTTAGCCCGAGCGAGGATAATTTCACCAACGATCTGAACAAAGGCGAAGAAATTACCAGCGATACCA
ATATTGAAGCAGCCGAAGAAAACATTAGCCTGGATCTGATTCAGCAGTATTATCTGACCTTCAACTTC
GATAATGAGCCGGAAAATATCAGCATTGAAAACCTGAGCAGCGATATTATTGGCCAGCTGGAkCTGAT
GCCGAATATTGAACGTTTTCCGAACGGCAAAAAATACGAGCTGGATAAATACACCATGTTCCATTATC
TGCGTGCCCAAGAATTTGAACATGGTAAAAGCCGTATTGCACTGACCAATAGCGTTAATGAAGCACTG
CTGAACCCGAGCCGTGTTTATACCTTTTTTAGCAGCGATTACGTGAAAAAGGTTAACAAAGCAACCGA
AGCAGCCATGTTTTTAGGTTGGGTTGAACAGCTGGTTTATGATTTCACCGATGAAACCAGCGAAGTTA
GCACCACCGATAAAATTGCAGATATTACCATCATCATCCCGTATATCGGTCCGGCACTGAATATTGGC
AATATGCTGTATAAAGACGATTTTGTGGGTGCCCTGATCTTTAGCGGTGCAGTTATTCTGCTGGAATT
TATTCCGGAAATTGCCATTCCGGTTCTGGGCACCTTTGCACTGGTGAGCTATATTGCAAATAAAGTTC
TGACCGTGCAGACCATCGATAATGCACTGAGCAAACGTAACGAAAAATGGGATGAAGTGTACAAGTAT
ATCGTGACCAATTGGCTGGCAAAAGTTAACACCCAGATTGACCTGATTCGCAAGAAGATGAAAGAAGC
ACTGGAAAACCAGGCAGAAGCAACCAAAGCCATTATTAACTATCAGTACAACCAGTACACCGAAGAAG
AGAAGAATAACATCAACTTCAACATCGATGATCTGAGCAGCAAGCTGAATGAAAGCATCAACAAAGCC
ATGATCAACATTAACAAATTTCTGAATCAGTGCAGCGTGAGCTATCTGATGAATAGCATGATTCCGTA
TGGTGTGAAACGTCTGGAAGATTTTGATGCAAGCCTGAAAGATGCCCTGCTGAAATATATCTATGATA
ATCGTGGCACCCTGATTGGTCAGGTTGATCGTCTGAAAGATAAAGTGAACAACACCCTGAGTACCGAT
ATTCCTTTTCAGCTGAGCAAATATGTGGATAATCAGCGTCTGCTGAGTACCCTGGATGGCGGCAGCGG
CGGCGGCAGCGGCCTGCCCGAAAGCGGTGGCGGATCTGCTTGGTCTCACCCGCAGTTCGAAAAAGGTG
GTGGTTCTGGTGGTGGTTCTGGTGGTTCTGCTTGGTCTCACCCGCAGTTCGAAAAAtaatgaAAGCTT
GCGGCCGCACTCGAGCACCACCACCACCACCACTGAGATCCGGCTGCTAACAAAGCCCGAAAGGAAGC
TGAGTTGGCTGCTGCCACCGCTGAGCAATAACTAGCATAACCCCTTGGGGCCTCTAAACGGGTCTTGA
GGGGTTTTTTGCTGAAAGGAGGAACTATATCCGGAT
Polypeptide sequence of nociceptin-liganded polypeptide with dual-
labelling SrtA sites
SEQ ID NO: 4
MENLYFQGGGGSGGSGGSGSMPFVNKQFNYKDPVNGVDIAYIKIPNAGQMQPVKAFKIHNKIWVIPER
DTFTNPEEGDLNPPPEAKQVPVSYYDSTYLSTDNEKDNYLKGVTKLFERIYSTDLGRMLLTSIVRGIP
FWGGSTIDTELKVIDTNCINVIQPDGSYRSEELNLVIIGPSADIIQFECKSFGHEVLNLTRNGYGSTQ
YIRFSPDFTFGFEESLEVDTNPLLGAGKFATDPAVTLAHELIHAGHRLYGIAINPNRVFKVNTNAYYE
MSGLEVSFEELRTFGGHDAKFIDSLQENEFRLYYYNKFKDIASTLNKAKSIVGTTASLQYMKNVFKEK
YLLSFDTSGKFSVDKLKFDKLYKMLTEIYTEDNFVKFFKVLNRKTYLNFDKAVFKINIVPKVNYTIYD
GFKLRNTNLAANFNGQNTEINNMNFTKLKNFTGLFEFYKLLCVDGIITSKTKSDDDDKFGGFTGARKS
ARKRKNQALAGGGGSGGGGSGGGGSALVLQCIKVNNWDLFFSPSEDNFTNDLNKGEEITSDTNIEAAE
ENISLDLIQQYYLTFNFDNEPENISIENLSSDIIGQLELMPNIERFPNGKKYELDKYTMFHYLRAQEF
EHGKSRIALTNSVNEALLNPSRVYTFFSSDYVKKVNKATEAAMFLGWVEQLVYDFTDETSEVSTTDKI
ADITIIIPYIGPALNIGNMLYKDDFVGALIFSGAVILLEFIFEIAIPVLGTFALVSYIANKVLTVQTI
DNALSKRNEKWDEVYKYIVTNWLAKVNTQIDLIRKKMKEALENQAEATKAIINYQYNQYTEEEKNNIM
FNIDDLSSKLNESINKAMININKFLNQCSVSYLMNSMIPYGVKRLEDFDASLKDALLKYIYDNRGTLI
GQVDRLKDKVNNTLSTDIPFQLSKYVDNQRLLSTLDGGSGGGSGLPESGGGSAWSHPQFEKGGG
Nucleotide sequence of EGF-liganded polypeptide
SEQ ID NO: 5
TGGCGAATGGGACGCGCCCTGTAGCGGCGCATTAAGCGCGGCGGGTGTGGTGGTTACGCGCAGCGTGA
CCGCTACACTTGCCAGCGCCCTAGCGCCCGCTCCTTTCGCTTTCTTCCCTTCCTTTCTCGCCACGTTC
GCCGGCTTTCCCCGTCAAGCTCTAAATCGGGGGCTCCCTTTAGGGTTCCGATTTAGTGCTTTACGGCA
CCTCGACCCCAAAAAACTTGATTAGGGTGATGGTTCACGTAGTGGGCCATCGCCCTGATAGACGGTTT
TTCGCCCTTTGACGTTGGAGTCCACGTTCTTTAATAGTGGACTCTTGTTCCAAACTGGAACAACACTC
AACCCTATCTCGGTCTATTCTTTTGATTTATAAGGGATTTTGCCGATTTCGGCCTATTGGTTAAAAAA
TGAGCTGATTTAACAAAAATTTAACGCGAATTTTAACAAAATATTAACGCTTACAATTTAGGTGGCAC
TTTTCGGGGAAATGTGCGCGGAACCCCTATTTGTTTATTTTTCTAAATACATTCAAATATGTATCCGC
TCATGAATTAATTCTTAGAAAAACTCATCGAGCATCAAATGAAACTGCAATTTATTCATATCAGGATT
ATCAATACCATATTTTTGAAAAAGCCGTTTCTGTAATGAAGGAGAAAACTCACCGAGGCAGTTCCATA
GGATGGCAAGATCCTGGTATCGGTCTGCGATTCCGACTCGTCCAACATCAATACAACCTATTAATTTC
CCCTCGTCAAAAATAAGGTTATCAAGTGAGAAATCACCATGAGTGACGACTGAATCCGGTGAGAATGG
CAAAAGTTTATGCATTTCTTTCCAGACTTGTTCAACAGGCCAGCCATTACGCTCGTCATCAAAATCAC
TCGCATCAACCAAACCGTTATTCATTCGTGATTGCGCCTGAGCGAGACGAAATACGCGATCGCTGTTA
AAAGGACAATTACAAACAGGAATCGAATGCAACCGGCGCAGGAACACTGCCAGCGCATCAACAATATT
TTCACCTGAATCAGGATATTCTTCTAATACCTGGAATGCTGTTTTCCCGGGGATCGCAGTGGTGAGTA
ACCATGCATCATCAGGAGTACGGATAAAATGCTTGATGGTCGGAAGAGGCATAAATTCCGTCAGCCAG
TTTAGTCTGACCATCTCATCTGTAACATCATTGGCAACGCTACCTTTGCCATGTTTCAGAAACAACTC
TGGCGCATCGGGCTTCCCATACAATCGATAGATTGTCGCACCTGATTGCCCGACATTATCGCGAGCCC
ATTTATACCCATATAAATCAGCATCCATGTTGGAATTTAATCGCGGCCTAGAGCAAGACGTTTCCCGT
TGAATATGGCTCATAACACCCCTTGTATTACTGTTTATGTAAGCAGACAGTTTTATTGTTCATGACCA
AAATCCCTTAACGTGAGTTTTCGTTCCACTGAGCGTCAGACCCCGTAGAAAAGATCAAAGGATCTTCT
TGAGATCCTTTTTTTCTGCGCGTAATCTGCTGCTTGCAAACAAAAAAACCACCGCTACCAGCGGTGGT
TTGTTTGCCGGATCAAGAGCTACCAACTCTTTTTCCGAAGGTAACTGGCTTCAGCAGAGCGCAGATAC
CAAATACTGTCCTTCTAGTGTAGCCGTAGTTAGGCCACCACTTCAAGAACTCTGTAGCACCGCCTACA
TACCTCGCTCTGCTAATCCTGTTACCAGTGGCTGCTGCCAGTGGCGATAAGTCGTGTCTTACCGGGTT
GGACTCAAGACGATAGTTACCGGATAAGGCGCAGCGGTCGGGCTGAACGGGGGGTTCGTGCACACAGC
CCAGCTTGGAGCGAACGACCTACACCGAACTGAGATACCTACAGCGTGAGCTATGAGAAAGCGCCACG
CTTCCCGAAGGGAGAAAGGCGGACAGGTATCCGGTAAGCGGCAGGGTCGGAACAGGAGAGCGCACGAG
GGAGCTTCCAGGGGGAAACGCCTGGTATCTTTATAGTCCTGTCGGGTTTCGCCACCTCTGACTTGAGC
GTCGATTTTTGTGATGCTCGTCAGGGGGGCGGAGCCTATGGAAAAACGCCAGCAACGCGGCCTTTTTA
CGGTTCCTGGCCTTTTGCTGGCCTTTTGCTCACATCGGCGATAATGGCCTGCTTCTCGCCGAAACGTT
TGGTGGCGGGACCAGTGACGAAGGCTTGAGCGAGGGCGTGCAAGATTCCGAATACCGCAAGCGACAGG
CCGATCATCGTCGCGCTCCAGCGAAAGCGGTCCTCGCCGAAAATGACCCAGAGCGCTGCCGGCACCTG
TCCTACGAGTTGCATGATAAAGAAGACAGTCATAAGTGCGGCGACGATAGTCATGCCCCGCGCCCACC
GGAAGGAGCTGACTGGGTTGAAGGCTCTCAAGGGCATCGGTCGAGATCCCGGTGCCTAATGAGTGAGC
TAACTTACATTAATTGCGTTGCGCTCACTGCCCGCTTTCCAGTCGGGAAACCTGTCGTGCCAGCTGCA
TTAATGAATCGGCCAACGCGCGGGGAGAGGCGGTTTGCGTATTGGGCGCCAGGGTGGTTTTTCTTTTC
ACCAGTGAGACGGGCAACAGCTGATTGCCCTTCACCGCCTGGCCCTGAGAGAGTTGCAGCAAGCGGTC
CACGCTGGTTTGCCCCAGCAGGCGAAAATCCTGTTTGATGGTGGTTAACGGCGGGATATAACATGAGC
TGTCTTCGGTATCGTCGTATCCCACTACCGAGATATCCGCACCAACGCGCAGCCCGGACTCGGTAATG
GCGCGCATTGCGCCCAGCGCCATCTGATCGTTGGCAACCAGCATCGCAGTGGGAACGATGCCCTCATT
CAGGATTTGCATGGTTTGTTGAAAACCGGACATGGCACTCCAGTCGCCTTCCCGTTCCGCTATCGGCT
GAATTTGATTGCGAGTGAGATATTTATGCCAGCCAGCCAGACGCAGACGCGCCGAGACAGAACTTAAT
GGGCCCGCTAACAGCGCGATTTGCTGGTGACCCAATGCGACCAGATGCTCCACGCCCAGTCGCGTACC
GTCTTCATGGGAGAAAATAATACTGTTGATGGGTGTCTGGTCAGAGACATCAAGAAATAACGCCGGAA
CATTAGTGCAGGCAGCTTCCACAGCAATGGCATCCTGGTCATCCAGCGGATAGTTAATGATCAGCCCA
CTGACGCGTTGCGCGAGAAGATTGTGCACCGCCGCTTTACAGGCTTCGACGCCGCTTCGTTCTACCAT
CGACACCACCACGCTGGCACCCAGTTGATCGGCGCGAGATTTAATCGCCGCGACAATTTGCGACGGCG
CGTGCAGGGCCAGACTGGAGGTGGCAACGCCAATCAGCAACGACTGTTTGCCCGCCAGTTGTTGTGCC
ACGCGGTTGGGAATGTAATTCAGCTCCGCCATCGCCGCTTCCACTTTTTCCCGCGTTTTCGCAGAAAC
GTGGCTGGCCTGGTTCACCACGCGGGAAACGGTCTGATAAGAGACACCGGCATACTCTGCGACATCGT
ATAACGTTACTGGTTTCACATTCACCACCCTGAATTGACTCTCTTCCGGGCGCTATCATGCCATACCG
CGAAAGGTTTTGCGCCATTCGATGGTGTCCGGGATCTCGACGCTCTCCCTTATGCGACTCCTGCATTA
GGAAGCAGCCCAGTAGTAGGTTGAGGCCGTTGAGCACCGCCGCCGCAAGGAATGGTGCATGCAAGGAG
ATGGCGCCCAACAGTCCCCCGGCCACGGGGCCTGCCACCATACCCACGCCGAAACAAGCGCTCATGAG
CCCGAAGTGGCGAGCCCGATCTTCCCCATCGGTGATGTCGGCGATATAGGCGCCAGCAACCGCACCTG
TGGCGCCGGTGATGCCGGCCACGATGCGTCCGGCGTAGAGGATCGAGATCTCGATCCCGCGAAATTAA
TACGACTCACTATAGGGGAATTGTGAGCGGATAACAATTCCCCTCAAGAAATAATTTTGTTTAACTTT
AAGAAGGAGATATACATATgggatccatggagttcgttaacaaacagttcaactataaagacccagtt
aacggtgttgacattgcttacatcaaaatcccgaacgctggccagatgcagccggtaaaggcattcaa
aatccacaacaaaatctgggttatcccggaacgtgatacctttactaacccggaagaaggtgacctga
acccgccaccggaagcgaaacaggtgccggtatcttactatgactccacctacctgtctaccgataac
gaaaaggacaactacctgaaaggtgttactaaactgttcgagcgtatttactccaccgacctgggccg
tatgctgctgactagcatcgttcgcggtatcccgttctggggcggttctaccatcgataccgaactga
aagtaatcgacactaactgcatcaacgttattcagccggacggttcctatcgttccgaagaactgaac
ctggtgatcatcggcccgtctgctgatatcatccagttcgagtgtaagagctttggtcacgaagttct
gaacctcacccgtaacggctacggttccactcagtacatccgtttctctccggacttcaccttcggtt
ttgaagaatccctggaagtagacacgaacccactgctgggcgctggtaaattcgcaactgatcctgcg
gttaccctggctcacgaactgattcatgcaggccaccgcctgtacggtatcgccatcaatccgaaccg
tgtcttcaaagttaacaccaacgcgtattacgagatgtccggtctggaagttagcttcgaagaactgc
gtacttttggcggtcacgacgctaaattcatcgactctctgcaagaaaacgagttccgtctgtactac
tataacaagttcaaagatatcgcatccaccctgaacaaagcgaaatccatcgtgggtaccactgcttc
tctccagtacatgaagaacgtttttaaagaaaaatacctgctcagcgaagacacctccggcaaattct
ctgtagacaagttgaaattcgataaactttacaaaatgctgactgaaatttacaccgaagacaacttc
gttaagttctttaaagttctgaaccgcaaaacctatctgaacttcgacaaggcagtattcaaaatcaa
catcgtgccgaaagttaactacactatctacgatggtttcaacctgcgtaacaccaacctggctgcta
attttaacggccagaacacggaaatcaacaacatgaacttcacaaaactgaaaaacttcactggtctg
ttcgagttttacaagctgctgtgcgtcgacggcatcattacctccaaaactaaatctctgatagaagg
tagaaacaaagcgctgaacctgcagtgtatcaaggttaacaactgggatttattcttcagcccgagtg
aagacaacttcaccaacgacctgaacaaaggtgaagaaatcacctcagatactaacatcgaagcagcc
gaagaaaacatctcgctggacctgatccagcagtactacctgacctttaatttcgacaacgagccgga
aaacatttctatcgaaaacctgagctctgatatcatcggccagctggaactgatgccgaacatcgaac
gtttcccaaacggtaaaaagtacgagctggacaaatataccatgttccactacctgcgcgcgcaggaa
tttgaacacggcaaatcccgtatcgcactgactaactccgttaacgaagctctgctcaacccgtcccg
tgtatacaccttcttctctagcgactacgtgaaaaaggtcaacaaagcgactgaagctgcaatgttct
tgggttgggttgaacagcttgtttatgattttaccgacgagacgtccgaagtatctactaccgacaaa
attgcggatatcactatcatcatcccgtacatcggtccggctctgaacattggcaacatgctgtacaa
agacgacttcgttggcgcactgatcttctccggtgcggtgatcctgctggagttcatcccggaaatcg
ccatcccggtactaggcacctttgctctggtttcttacattgcaaacaaggttctgactgtacaaacc
atcgacaacgcgctgagcaaacgtaacgaaaaatgggatgaagtttacaaatatatcgtgaccaactg
gctggctaaggttaatactcagatcgacctcatccgcaaaaaaatgaaagaagcactggaaaaccagg
cggaagctaccaaggcaatcattaactaccagtacaaccagtacaccgaggaagaaaaaaacaacatc
aacttcaacatcgacgatctgtcctctaaactgaacgaatccatcaacaaagctatgatcaacatcaa
caagttcctgaaccagtgctctgtaagctatctgatgaactccatgatcccgtacggtgttaaacgtc
tggaggacttcgatgcgtctctgaaagacgccctgctgaaatacatttacgacaaccgtggcactctg
atcggtcaggttgatcgtctgaaggacaaagtgaacaataccttatcgaccgacatcccttttcagct
cagtaaatatgtcgataaccaacgccttttgtccactctagaaggcggTGGCGGTAGCGGTGGCGGTG
GCAGCGGCGGTGGCGGTAGCGCACTAGacAACAGCGACCCTAAATGCCCACTgAGTCATGAAGGATAC
TGCCTTAATGATGGTGTTTGTATGTACATAGGAACATTGGACCGTTATGCTTGCAATTGTGTAGTGGG
CTATGTCGGGGAAAGGTGTCAATATCGAGATCTCAAGCTGGCAGAGTTAAGAgggctagaagcaCACC
ATCATCACcaccatcaccatcaccattaatgaAAGCTTGCGGCCGCACTCGAGCACCACCACCACCAC
CACTGAGATCCGGCTGCTAACAAAGCCCGAAAGGAAGCTGAGTTGGCTGCTGCCACCGCTGAGCAATA
ACTAGCATAACCCCTTGGGGCCTCTAAACGGGTCTTGAGGGGTTTTTTGCTGAAAGGAGGAACTATAT
CCGGAT
Polypeptide sequence of EGF-liganded polypeptide
SEQ ID NO: 6
MEFVNKQFNYKDPVNGVDIAYIKIPNAGQMQPVKAFKIHNKIWVIPERDTFTNPEEGDLNPPPEAKQV
PVSYYDSTYLSTDNEKDNYLKGVTKLFERIYSTDLGRMLLTSIVRGIPFWGGSTIDTELKVIDTKCIN
VIQPDGSYRSEELNLVIIGPSADIIQFECKSFGHEVLNLTRNGYGSTQYIRFSPDFTFGFEESLEVDT
NPLLGAGKFATDPAVTLAHELIHAGHRLYGIAINPNRVFKVNTNAYYEMSGLEVSFEELRTFGGHDAK
FIDSLQENEFRLYYYNKFKDIASTLNKAKSIVGTTASLQYMKNVFKEKYLLSEDTSGKFSVDKLKFDK
LYKMLTEIYTEDNFVKFFKVLNRKTYLNFDKAVFKINIVPKVNYTIYDGFNLRNTNLAANFNGQNTEI
NNMNFTKLKNFTGLFEFYKLLCVDGIITSKTKSLIEGRNKALNLQCIKVNNWDLFFSPSEDNFTNDLN
KGEEITSDTNIEAAEENISLDLIQQYYLTFNFDNEPENISIENLSSDIIGQLELMPNIERFPNGKKYE
LDKYTMFHYLRAQEFEHGKSRIALTNSVNEALLNPSRVYTFFSSDYVKKVNKATEAAMFLGWVEQLVY
DFTDETSEVSTTDKIADITIIIPYIGPALNIGNMLYKDDFVGALIFSGAVILLEFIPEIAIPVLGTFA
LVSYIANKVLTVQTIDNALSKRNEKWDEVYKYIVTNWLAKVNTQIDLIRKKMKEALENQAEATKAIIN
YQYNQYTEEEKNNINFNIDDLSSKLNESINKAMININKFLNQCSVSYLMNSMIPYGVKRLEDFDASLK
DALLKYIYDNRGTLIGQVDRLKDKVNNTLSTDIPFQLSKYVDNQRLLSTLEGGGGSGGGGSGGGGSAL
DNSDPKCPLSHEGYCLNDGVCMYIGTLDRYACNCVVGYVGERCQYRDLKLAELRGLEAHHHHHHHHHH
Nucleotide sequence of nociceptin-liganded polypeptide
SEQ ID NO: 7
TGGCGAATGGGACGCGCCCTGTAGCGGCGCATTAAGCGCGGCGGGTGTGGTGGTTACGCGCAGCGTGA
CCGCTACACTTGCCAGCGCCCTAGCGCCCGCTCCTTTCGCTTTCTTCCCTTCCTTTCTCGCCACGTTC
GCCGGCTTTCCCCGTCAAGCTCTAAATCGGGGGCTCCCTTTAGGGTTCCGATTTAGTGCTTTACGGCA
CCTCGACCCCAAAAAACTTGATTAGGGTGATGGTTCACGTAGTGGGCCATCGCCCTGATAGACGGTTT
TTCGCCCTTTGACGTTGGAGTCCACGTTCTTTAATAGTGGACTCTTGTTCCAAACTGGAACAACACTC
AACCCTATCTCGGTCTATTCTTTTGATTTATAAGGGATTTTGCCGATTTCGGCCTATTGGTTAAAAAA
TGAGCTGATTTAACAAAAATTTAACGCGAATTTTAACAAAATATTAACGCTTACAATTTAGGTGGCAC
TTTTCGGGGAAATGTGCGCGGAACCCCTATTTGTTTATTTTTCTAAATACATTCAAATATGTATCCGC
TCATGAATTAATTCTTAGAAAAACTCATCGAGCATCAAATGAAACTGCAATTTATTCATATCAGGATT
ATCAATACCATATTTTTGAAAAAGCCGTTTCTGTAATGAAGGAGAAAACTCACCGAGGCAGTTCCATA
GGATGGCAAGATCCTGGTATCGGTCTGCGATTCCGACTCGTCCAACATCAATACAACCTATTAATTTC
CCCTCGTCAAAAATAAGGTTATCAAGTGAGAAATCACCATGAGTGACGACTGAATCCGGTGAGAATGG
CAAAAGTTTATGCATTTCTTTCCAGACTTGTTCAACAGGCCAGCCATTACGCTCGTCATCAAAATCAC
TCGCATCAACCAAACCGTTATTCATTCGTGATTGCGCCTGAGCGAGACGAAATACGCGATCGCTGTTA
AAAGGACAATTACAAACAGGAATCGAATGCAACCGGCGCAGGAACACTGCCAGCGCATCAACAATATT
TTCACCTGAATCAGGATATTCTTCTAATACCTGGAATGCTGTTTTCCCGGGGATCGCAGTGGTGAGTA
ACCATGCATCATCAGGAGTACGGATAAAATGCTTGATGGTCGGAAGAGGCATAAATTCCGTCAGCCAG
TTTAGTCTGACCATCTCATCTGTAACATCATTGGCAACGCTACCTTTGCCATGTTTCAGAAACAACTC
TGGCGCATCGGGCTTCCCATACAATCGATAGATTGTCGCACCTGATTGCCCGACATTATCGCGAGCCC
ATTTATACCCATATAAATCAGCATCCATGTTGGAATTTAATCGCGGCCTAGAGCAAGACGTTTCCCGT
TGAATATGGCTCATAACACCCCTTGTATTACTGTTTATGTAAGCAGACAGTTTTATTGTTCATGACCA
AAATCCCTTAACGTGAGTTTTCGTTCCACTGAGCGTCAGACCCCGTAGAAAAGATCAAAGGATCTTCT
TGAGATCCTTTTTTTCTGCGCGTAATCTGCTGCTTGCAAACAAAAAAACCACCGCTACCAGCGGTGGT
TTGTTTGCCGGATCAAGAGCTACCAACTCTTTTTCCGAAGGTAACTGGCTTCAGCAGAGCGCAGATAC
CAAATACTGTCCTTCTAGTGTAGCCGTAGTTAGGCCACCACTTCAAGAACTCTGTAGCACCGCCTACA
TACCTCGCTCTGCTAATCCTGTTACCAGTGGCTGCTGCCAGTGGCGATAAGTCGTGTCTTACCGGGTT
GGACTCAAGACGATAGTTACCGGATAAGGCGCAGCGGTCGGGCTGAACGGGGGGTTCGTGCACACAGC
CCAGCTTGGAGCGAACGACCTACACCGAACTGAGATACCTACAGCGTGAGCTATGAGAAAGCGCCACG
CTTCCCGAAGGGAGAAAGGCGGACAGGTATCCGGTAAGCGGCAGGGTCGGAACAGGAGAGCGCACGAG
GGAGCTTCCAGGGGGAAACGCCTGGTATCTTTATAGTCCTGTCGGGTTTCGCCACCTCTGACTTGAGC
GTCGATTTTTGTGATGCTCGTCAGGGGGGCGGAGCCTATGGAAAAACGCCAGCAACGCGGCCTTTTTA
CGGTTCCTGGCCTTTTGCTGGCCTTTTGCTCACATCGGCGATAATGGCCTGCTTCTCGCCGAAACGTT
TGGTGGCGGGACCAGTGACGAAGGCTTGAGCGAGGGCGTGCAAGATTCCGAATACCGCAAGCGACAGG
CCGATCATCGTCGCGCTCCAGCGAAAGCGGTCCTCGCCGAAAATGACCCAGAGCGCTGCCGGCACCTG
TCCTACGAGTTGCATGATAAAGAAGACAGTCATAAGTGCGGCGACGATAGTCATGCCCCGCGCCCACC
GGAAGGAGCTGACTGGGTTGAAGGCTCTCAAGGGCATCGGTCGAGATCCCGGTGCCTAATGAGTGAGC
TAACTTACATTAATTGCGTTGCGCTGACTGCCCGCTTTCCAGTCGGGAAAGCTGTCGTGCCAGCTGCA
TTAATGAATGGGCCAACGCGCGGGGAGAGGCGGTTTGCGTATTGGGCGCCAGGGTGGTTTTTCTTTTC
ACCAGTGAGACGGGCAACAGCTGATTGCCCTTCACCGCCTGGCCCTGAGAGAGTTGCAGCAAGCGGTC
CACGCTGGTTTGCCCCAGCAGGCGAAAATCCTGTTTGATGGTGGTTAACGGCGGGATATAACATGAGC
TGTCTTCGGTATCGTCGTATCCCACTACCGAGATATCCGCACCAACGCGCAGCCCGGACTCGGTAATG
GCGCGCATTGCGCCCAGCGCCATCTGATCGTTGGCAACCAGCATCGCAGTGGGAACGATGCCCTCATT
CAGCATTTGCATGGTTTGTTGAAAACCGGACATGGCACTCCAGTCGCCTTCCCGTTCCGCTATCGGCT
GAATTTGATTGCGAGTGAGATATTTATGCCAGCCAGCCAGACGCAGACGCGCCGAGACAGAACTTAAT
GGGCCCGCTAACAGCGCGATTTGCTGGTGACCCAATGCGACCAGATGCTCCACGCCCAGTCGCGTACC
GTCTTCATGGGAGAAAATAATACTGTTGATGGGTGTCTGGTCAGAGACATCAAGAAATAACGCCGGAA
CATTAGTGCAGGCAGCTTCCACAGCAATGGCATCCTGGTCATCCAGCGGATAGTTAATGATCAGCCCA
CTGACGCGTTGCGCGAGAAGATTGTGCACCGCCGCTTTACAGGCTTCGACGCCGCTTCGTTCTACCAT
CGACACCACCACGCTGGCACCCAGTTGATCGGCGCGAGATTTAATCGCCGCGACAATTTGCGACGGCG
CGTGCAGGGCCAGACTGGAGGTGGCAACGCCAATCAGCAACGACTGTTTGCCCGCCAGTTGTTGTGCC
ACGCGGTTGGGAATGTAATTCAGCTCCGCCATCGCCGCTTCCACTTTTTCCCGCGTTTTCGCAGAAAC
GTGGCTGGCCTGGTTCACCACGCGGGAAACGGTCTGATAAGAGACACCGGCATACTCTGCGACATCGT
ATAACGTTACTGGTTTCACATTCACCACCCTGAATTGACTCTCTTCCGGGCGCTATCATGCCATACCG
CGAAAGGTTTTGCGCCATTCGATGGTGTCCGGGATCTCGACGCTCTCCCTTATGCGACTCCTGCATTA
GGAAGCAGCCCAGTAGTAGGTTGAGGCCGTTGAGCACCGCCGCCGCAAGGAATGGTGCATGCAAGGAG
ATGGCGCCCAACAGTCCCCCGGCCACGGGGCCTGCCACCATACCCACGCCGAAACAAGCGCTCATGAG
CCCGAAGTGGCGAGCCCGATCTTCCCCATCGGTGATGTCGGCGATATAGGCGCCAGCAACCGCACCTG
TGGCGCCGGTGATGCCGGCCACGATGCGTCCGGCGTAGAGGATCGAGATCTCGATCCCGCGAAATTAA
TACGACTCACTATAGGGGAATTGTGAGCGGATAACAATTCCCCTCAAGAAATAATTTTGTTTAACTTT
AAGAAGGAGATATACATATGGGCAGCATGGAATTTGTGAACAAACAGTTCAACTATAAGGATCCGGTT
AATGGTGTGGATATCGCCTATATCAAAATTCCGAATGCAGGTCAGATGCAGCCGGTTAAAGCCTTTAA
TATTGCAAATAAAGTTCTGACCGTGCAGACCATCGATAATGCACTGAGCAAACGTAACGAAAAATGGG
ATGAAGTGTACAAGTATATCGTGACCAATTGGCTGGCAAAAGTTAACACCCAGATTGACCTGATTCGC
AAGAAGATGAAAGAAGCACTGGAAAACCAGGCAGAAGCAACCAAAGCCATTATTAACTATCAGTACAA
CCAGTACACCGAAGAAGAGAAGAATAACATCAACTTCAACATCGATGATCTGAGCAGCAAGCTGAATG
AAAGCATCAACAAAGCCATGATCAACATTAACAAATTTCTGAATCAGTGCAGCGTGAGCTATCTGATG
AATAGCATGATTCCGTATGGTGTGAAACGTCTGGAAGATTTTGATGCAAGCCTGAAAGATGCCCTGCT
GAAATATATCTATGATAATCGTGGCACCCTGATTGGTCAGGTTGATCGTCTGAAAGATAAAGTGAACA
ACACCCTGAGTACCGATATTCCTTTTCAGCTGAGCAAATATGTGGATAATCAGCGTCTGCTGAGTACC
CTGGATCATCATCACCATCACCACTAAAAGCTTGCGGCCGCACTCGAGCACCACCACCACCACCACTG
AGATCCGGCTGCTAACAAAGCCCGAAAGGAAGCTGAGTTGGCTGCTGCCACCGCTGAGCAATAACTAG
CATAACCCCTTGGGGCCTCTAAACGGGTCTTGAGGGGTTTTTTGCTGAAAGGAGGAACTATATCCGGA
T
Polypeptide sequence of nociceptin-liganded polypeptide
SEQ ID NO: 8
MGSMEFVNKQFNYKDPVNGVDIAYIKIPNAGQMQPVKAFKIHNKIWVIPERDTETNPEEGDLNPPPEA
KQVPVSYYDSTYLSTDNEKDNYLKGVTKLFERIYSTDLGRMLLTSIVRGIPFWGGSTIDTELKVIDTN
CINVIQPDGSYRSEELNLVTIGPSADIIQFECKSFGHEVLNLTRNGYGSTQYIRFSPDFTFGFEESLE
VDTNPLLGAGKFATDPAVTLAHELIHAGHRLYGIAINPNRVFKVNTNAYYEMSGLEVSFEELRTFGGH
DAKFIDSLQENEFRLYYYNKFKDIASTLNKAKSIVGTTASLQYMKNVFKEKYLLSEDTSGKFSVDKLK
FDKLYKMLTEIYTEDNEVKFFKVLNRKTYLNFDKAVFKINIVPKVNYTIYDGFNLRNTNLAANFNGQN
TEINNMNFTKLKNFTGLFEFYKLLCVDGIITSKTKSDDDDKFGGFTGARKSARKRKNQALAGGGGSGG
GGSGGGGSALVLQCIKVNNWDLFFSPSEDNFTNDLNKGEEITSDTNIEAAEENISLDLIQQYYLTFNF
DNEPENISIENLSSDIIGQLELMPNIERFPNGKKYELDKYTMFHYLRAQEFEHGKSRIALTNSVNEAL
LNPSRVYTFFSSDYVKKVNKATEAAMFLGWVEQLVYDFTDETSEVSTTDKIADITIIIPYIGPALNIG
NMLYKDDFVGALIFSGAVILLEFIPEIAIPVLGTFALVSYIANKVLTVQTIDNALSKRNEKWDEVYKY
IVTNWLAKVNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDKVNNTLSTD
IPFQLSKYVDNQRLLSTLDHHHHHH
Nucleotide sequence of EGF-liganded polypeptide GFP-tagged
SEQ ID NO: 9
TGGCGAATGGGACGCGCCCTGTAGCGGCGCATTAAGCGCGGCGGGTGTGGTGGTTACGCGCAGCGTGA
CCGCTACACTTGCCAGCGCCCTAGCGCCCGCTCCTTTCGCTTTCTTCCCTTCCTTTCTCGCCACGTTC
GCCGGCTTTCCCCGTCAAGCTCTAAATCGGGGGCTCCCTTTAGGGTTCCGATTTAGTGCTTTACGGCA
CCTCGACCCCAAAAAACTTGATTAGGGTGATGGTTCACGTAGTGGGCCATCGCCCTGATAGACGGTTT
TTCGCCCTTTGACGTTGGAGTCCACGTTCTTTAATAGTGGACTCTTGTTCCAAACTGGAACAACACTC
AACCCTATCTCGGTCTATTCTTTTGATTTATAAGGGATTTTGCCGATTTCGGCCTATTGGTTAAAAAA
TGAGCTGATTTAACAAAAATTTAACGCGAATTTTAACAAAATATTAACGCTTACAATTTAGGTGGCAC
TTTTCGGGGAAATGTGCGCGGAACCCCTATTTGTTTATTTTTCTAAATACATTCAAATATGTATCCGC
TCATGAATTAATTCTTAGAAAAACTCATCGAGCATCAAATGAAACTGCAATTTATTCATATCAGGATT
ATCAATACCATATTTTTGAAAAAGCCGTTTCTGTAATGAAGGAGAAAACTCACCGAGGCAGTTCCATA
GGATGGCAAGATCCTGGTATCGGTCTGCGATTCCGACTCGTCCAACATCAATACAACCTATTAATTTC
CCCTCGTCAAAAATAAGGTTATCAAGTGAGAAATCACCATGAGTGACGACTGAATCCGGTGAGAATGG
CAAAAGTTTATGCATTTCTTTCCAGACTTGTTCAACAGGCCAGCCATTACGCTCGTCATCAAAATCAC
TCGCATCAACCAAACCGTTATTCATTCGTGATTGCGCCTGAGCGAGACGAAATACGCGATCGCTGTTA
AAAGGACAATTACAAACAGGAATCGAATGCAACCGGCGCAGGAACACTGCCAGCGCATCAACAATATT
TTCACCTGAATCAGGATATTCTTCTAATACCTGGAATGCTGTTTTCCCGGGGATCGCAGTGGTGAGTA
ACCATGCATCATCAGGAGTACGGATAAAATGCTTGATGGTCGGAAGAGGCATAAATTCCGTCAGCCAG
TGGCGCATCGGGCTTCCCATACAATCGATAGATTGTCGCACCTGATTGCCCGACATTATCGCGAGCCC
ATTTATACCCATATAAATCAGCATCCATGTTGGAATTTAATCGCGGCCTAGAGCAAGACGTTTCCCGT
TGAATATGGCTCATAACACCCCTTGTATTACTGTTTATGTAAGCAGACAGTTTTATTGTTCATGACCA
AAATCCCTTAACGTGAGTTTTCGTTCCACTGAGCGTCAGACCCCGTAGAAAAGATCAAAGGATCTTCT
TGAGATCCTTTTTTTCTGCGCGTAATCTGCTGCTTGCAAACAAAAAAACCACCGCTACCAGCGGTGGT
TTGTTTGCCGGATCAAGAGCTACCAACTCTTTTTCCGAAGGTAACTGGCTTCAGCAGAGCGCAGATAC
CAAATACTGTCCTTCTAGTGTAGCCGTAGTTAGGCCACCACTTCAAGAACTCTGTAGCACCGCCTACA
TACCTCGCTCTGCTAATCCTGTTACCAGTGGCTGCTGCCAGTGGCGATAAGTCGTGTCTTACCGGGTT
GGACTCAAGACGATAGTTACCGGATAAGGCGCAGCGGTCGGGCTGAACGGGGGGTTCGTGCACACAGC
CCAGCTTGGAGCGAACGACCTACACCGAACTGAGATACCTACAGCGTGAGCTATGAGAAAGCGCCACG
CTTCCCGAAGGGAGAAAGGCGGACAGGTATCCGGTAAGCGGCAGGGTCGGAACAGGAGAGCGCACGAG
GGAGCTTCCAGGGGGAAACGCCTGGTATCTTTATAGTCCTGTCGGGTTTCGCCACCTCTGACTTGAGC
GTCGATTTTTGTGATGCTCGTCAGGGGGGCGGAGCCTATGGAAAAACGCCAGCAACGCGGCCTTTTTA
CGGTTCCTGGCCTTTTGCTGGCCTTTTGCTCACATCGGCGATAATGGCCTGCTTCTCGCCGAAACGTT
TGGTGGCGGGACCAGTGACGAAGGCTTGAGCGAGGGCGTGCAAGATTCCGAATACCGCAAGCGACAGG
CCGATCATCGTCGCGCTCCAGCGAAAGCGGTCCTCGCCGAAAATGACCCAGAGCGCTGCCGGCACCTG
TCCTACGAGTTGCATGATAAAGAAGACAGTCATAAGTGCGGCGACGATAGTCATGCCCCGCGCCCACC
GGAAGGAGCTGACTGGGTTGAAGGCTCTCAAGGGCATCGGTCGAGATCCCGGTGCCTAATGAGTGAGC
TAACTTACATTAATTGCGTTGCGCTCACTGCCCGCTTTCCAGTCGGGAAACCTGTCGTGCCAGCTGCA
TTAATGAATCGGCCAACGCGCGGGGAGAGGCGGTTTGCGTATTGGGCGCCAGGGTGGTTTTTCTTTTC
ACCAGTGAGACGGGCAACAGCTGATTGCCCTTCACCGCCTGGCCCTGAGAGAGTTGCAGCAAGCGGTC
CACGCTGGTTTGCCCCAGCAGGCGAAAATCCTGTTTGATGGTGGTTAACGGCGGGATATAACATGAGC
TGTCTTCGGTATCGTCGTATCCCACTACCGAGATATCCGCACCAACGCGCAGCCCGGACTCGGTAATG
GCGCGCATTGCGCCCAGCGCCATCTGATCGTTGGCAACCAGCATCGCAGTGGGAACGATGCCCTCATT
CAGCATTTGCATGGTTTGTTGAAAACCGGACATGGCACTCCAGTCGCCTTCCCGTTCCGCTATCGGCT
GAATTTGATTGCGAGTGAGATATTTATGCCAGCCAGCCAGACGCAGACGCGCCGAGACAGAACTTAAT
GGGCCCGCTAACAGCGCGATTTGCTGGTGACCCAATGCGACCAGATGCTCCACGCCCAGTCGCGTACC
GTCTTCATGGGAGAAAATAATACTGTTGATGGGTGTCTGGTCAGAGACATCAAGAAATAACGCCGGAA
CATTAGTGCAGGCAGCTTCCACAGCAATGGCATCCTGGTCATCCAGCGGATAGTTAATGATCAGCCCA
CTGACGCGTTGCGCGAGAAGATTGTGCACCGCCGCTTTACAGGCTTCGACGCCGCTTCGTTCTACCAT
CGACACCACCACGCTGGCACCCAGTTGATCGGCGCGAGATTTAATCGCCGCGACAATTTGCGACGGCG
CGTGCAGGGCCAGACTGGAGGTGGCAACGCCAATCAGCAACGACTGTTTGCCCGCCAGTTGTTGTGCC
ACGCGGTTGGGAATGTAATTCAGCTCCGCCATCGCCGCTTCCACTTTTTCCCGCGTTTTCGCAGAAAC
GTGGCTGGCCTGGTTCACCACGCGGGAAACGGTCTGATAAGAGACACCGGCATACTCTGCGACATCGT
ATAACGTTACTGGTTTCACATTCACCACCCTGAATTGACTCTCTTCCGGGCGCTATCATGCCATACCG
CGAAAGGTTTTGCGCCATTCGATGGTGTCCGGGATCTCGACGCTCTCCCTTATGCGACTCCTGCATTA
GGAAGCAGCCCAGTAGTAGGTTGAGGCCGTTGAGCACCGCCGCCGCAAGGAATGGTGCATGCAAGGAG
ATGGCGCCCAACAGTCCCCCGGCCACGGGGCCTGCCACCATACCCACGCCGAAACAAGCGCTCATGAG
CCCGAAGTGGCGAGCCCGATCTTCCCCATCGGTGATGTCGGCGATATAGGCGCCAGCAACCGCACCTG
TGGCGCCGGTGATGCCGGCCACGATGCGTCCGGCGTAGAGGATCGAGATCTCGATCCCGCGAAATTAA
TACGACTCACTATAGGGGAATTGTGAGCGGATAACAATTCCCCTCAAGAAATAATTTTGTTTAACTTT
AAGAAGGAGATATACATATgATGGTGAGCAAGGGCGAGGAGCTGTTCACCGGGGTGGTGCCCATCCTG
GTCGAGCTGGACGGCGACGTAAACGGCCACAAGTTCAGCGTGTCCGGCGAGGGCGAGGGCGATGCCAC
CTACGGCAAGCTGACCCTGAAGTTCATCTGCACCACCGGCAAGCTGCCCGTGCCCTGGCCCACCCTCG
TGACCACCCTGACCTACGGCGTGCAGTGCTTCAGCCGCTACCCCGACCACATGAAGCAGCACGACTTC
TTCAAGTCCGCCATGCCCGAAGGCTACGTCCAGGAGCGCACCATCTTCTTCAAGGACGACGGCAACTA
CAAGACCCGCGCCGAGGTGAAGTTCGAGGGCGACACCCTGGTGAACCGCATCGAGCTGAAGGGCATCG
ACTTCAAGGAGGACGGCAACATCCTGGGGCACAAGCTGGAGTACAACTACAACAGCCACAACGTCTAT
ATCATGGCCGACAAGCAGAAGAACGGCATCAAGGTGAACTTCAAGATCCGCCACAACATCGAGGACGG
CAGCGTGCAGCTCGCCGACCACTACCAGCAGAACACCCCCATCGGCGACGGCCCCGTGCTGCTGCCCG
ACAACCACTACCTGAGCACCCAGTCCGCCCTGAGCAAAGACCCCAACGAGAAGCGCGATCACATGGTC
CTGCTGGAGTTCGTGACCGCCGCCGGGATCACTCACGGCATGGACGAGCTGTACAAGGGCGGCAGCGG
CGGCGGCAGCGGCGGCggatccatggagttcgttaacaaacagttcaactataaagacccagttaacg
gtgttgacattgcttacatcaaaatcccgaacgctggccagatgcagccggtaaaggcattcaaaatc
cacaacaaaatctgggttatcccggaacgtgatacctttactaacccggaagaaggtgacctgaaccc
gccaccggaagcgaaacaggtgccggtatcttactatgactccacctacctgtctaccgataacgaaa
aggacaactacctgaaaggtgttactaaactgttcgagcgtatttactccaccgacctgggccgtatg
ctgctgactagcatcgttcgcggtatcccgttctggggcggttctaccatcgataccgaactgaaagt
aatcgacactaactgcatcaacgttattcagccggacggttcctatcgttccgaagaactgaacctgg
tgatcatcggcccgtctgctgatatcatccagttcgagtgtaagagctttggtcacgaagttctgaac
ctcacccgtaacggctacggttccactcagtacatccgtttctctccggacttcaccttcggttttga
agaatccctggaagtagacacgaacccactgctgggcgctggtaaattcgcaactgatcctgcggtta
ccctggctcacgaactgattcatgcaggccaccgcctgtacggtatcgccatcaatccgaaccgtgtc
ttcaaagttaacaccaacgcgtattacgagatgtccggtctggaagttagcttcgaagaactgcgtac
ttttggcggtcacgacgctaaattcatcgactctctgcaagaaaacgagttccgtctgtactactata
acaagttcaaagatatcgcatccaccctgaacaaagcgaaatccatcgtgggtaccactgcttctctc
cagtacatgaagaacgtttttaaagaaaaatacctgctcagcgaagacacctccggcaaattctctgt
agacaagttgaaattcgataaactttacaaaatgctgactgaaatttacaccgaagacaacttcgtta
agttctttaaagttctgaaccgcaaaacctatctgaacttcgacaaggcagtattcaaaatcaacatc
gtgccgaaagttaactacactatctacgatggtttcaacctgcgtaacaccaacctggctgctaattt
taacggccagaacacggaaatcaacaacatgaacttcacaaaactgaaaaacttcactggtctgttcg
agttttacaagctgctgtgcgtcgacggcatcattacctccaaaactaaatctctgatagaaggtaga
aacaaagcgctgaacctgcagtgtatcaaggttaacaactgggatttattcttcagcccgagtgaaga
caacttcaccaacgacctgaacaaaggtgaagaaatcacctcagatactaacatcgaagcagccgaag
aaaacatctcgctggacctgatccagcagtactacctgacctttaatttcgacaacgagccggaaaac
atttctatcgaaaacctgagctctgatatcatcggccagctggaactgatgccaaacatcgaacgttt
cccaaacggtaaaaagtacgagctggacaaatataccatgttccactacctgcgcgcgcaggaatttg
aacacggcaaatcccgtatcgcactgactaactccgttaacgaagctctgctcaacccgtcccgtgta
tacaccttcttctctagcgactacgtgaaaaaggtcaacaaagcgactgaagctgcaatgttcttggg
ttgggttgaacagcttgtttatgattttaccgacgagacgtccgaagtatctactaccgacaaaattg
cggatatcactatcatcatcccgtacatcggtccggctctgaacattggcaacatgctgtacaaagac
gacttcgttggcgcactgatcttctccggtgcggtgatcctgctggsgttcatcccggaaatcgccat
cccggtactgggcacctttgctctggtttcttacattgcaaacaaggttctgactgtacaaaccatcg
acaacgcgctgagcaaacgtaacgaaaaatgggatgaagtttacaaatatatcgtgaccaactggctg
gctaaggttaatactcagatcgacctcatccgcaaaaaaatgaaagaagcactggaaaaccaggcgga
agctaccaaggcaatcattaactaccagtacaaccagtacaccgaggaagaaaaaaacaacatcaact
tcaacatcgacgatctgtcctctaaactgaacgaatccatcaacaaagctatgatcaacatcaacaag
ttcctgaaccagtgctctgtaagctatctgatgaactccatgatcccgtacggtgttaaacgtctgga
ggacttcgatgcgtctctgaaagacgccctgctgaaatacatttacgacaaccgtggcactctgatcg
gtcaggttgatcgtctgaaggacaaagtgaacaataccttatcgaccgacatcccttttcagctcagt
aaatatgtcgataaccaacgccttttgtccactctagaaggcggTGGCGGTAGCGGTGGCGGTGGCAG
CGGCGGTGGCGGTAGCGCACTAGacAACAGCGACCCTAAATGCCCACTaAGTCATGAAGGATACTGCC
TTAATGATGGTGTTTGTATGTACATAGGAACATTGGACCGTTATGCTTGCAATTGTGTAGTGGGCTAT
GTCGGGGAAAGGTGTCAATATCGAGATCTCAAGCTGGCAGAGTTAAGAgggctagaagcaCACCATCA
TCACcaccatcaccatcaccattaatgaAAGCTTGCGGCCGCACTCGAGCACCACCACCACCACCACT
GAGATCCGGCTGCTAACAAAGCCCGAAAGGAAGCTGAGTTGGCTGCTGCCACCGCTGAGCAATAACTA
GCATAACCCCTTGGGGCCTCTAAACGGGTCTTGAGGGGTTTTTTGCTGAAAGGAGGAACTATATCCGG
AT
Polypeptide sequence of EGF-liganded polypeptide GFP-tagged
SEQ ID NO: 10
MVSKGEELFTGVVPILVELDGDVNGHKFSVSGEGEGDATYGKLTLKFICTTGKLPVPWPTLVTTLTYG
VQCFSRYPDHMKQHDFFKSAMPEGYVQERTIFFKDDGNYKTRAEVKFEGDTLVNRIELKGIDFKEDGN
ILGHKLEYNYNSHNVYIMADKQKNGIKVNFKIRHNIEDGSVQLADHYQQNTFIGDGPVLLPDNHYLST
QSALSKDPNEKRDHMVLLEFVTAAGITHGMDELYKGGSGGGSGGGSMEFVNKQFNYKDPVNGVDIAYI
KIPNAGQMQPVKAFKIHNKIWVTPERDTFTNPEEGDLNPPPEAKQVPVSYYDSTYLSTDNEKDNYLKG
VTKLFERIYSTDLGRMLLTSIVRGIPFWGGSTIDTELKVIDTNCINVIQPDGSYRSEELNLVIIGPSA
DIIQFECKSFGHEVLNLTRNGYGSTQYIRFSPDFTFGFEESLEVDTNPLLGAGKFATDPAVTLAHELI
HAGHRLYGIAINPNRVFKVNTNAYYEMSGLEVSFEELRTFGGHDAKFIDSLQENEFRLYYYNKFKDIA
STLNKAKSIVGTTASLQYMKNVFKEKYLLSEDTSGKFSVDKLKFDKLYKMLTEIYTEDNFVKFFKVLN
RKTYLNFDKAVFKINIVPKVNYTIYDGFNLRNTNLAANFNGQNTEINNMNFTKLKNFTGLFEFYKLLC
VDGIITSKTKSLIEGRNKALNLQCIKVNNWDLFFSPSEDNFTNDLNKGEEITSDTNIEAAEENISLDL
IQQYYLTFNFDNEPENISIENLSSDIIGQLELMPMIERFPNGKKYELDKYTMFKYLRAQEFEHGKSRI
ALTNSVNEALLNPSRVYTFFSSDYVKKVNKATEAAMFLGWVEQLVYDFTDETSEVSTTDKIADITIII
PYIGPALNIGNMLYKDDFVGALIFSGAVILLEFIPEIAIPVLGTFALVSYIANKVLTVQTIDNALSKR
NEKWDEVYKYIVTNWLAKVNTQIDLIRKKMKEALENQAEATKAIINYQYNQYTEEEKNNINFNIDDLS
SKLNESINKAMININKFLNQCSVSYLMNSMIPYGVKRLEDFDASLKBALLKYIYDNRGTLIGQVDRLK
DKVNNTLSTDIPFQLSKYVDNQRLLSTLEGGGGSGGGGSGGGGSALDNSDPKCPLSHEGYCLNDGVCM
YIGTLDRYACNCWGYVGERCQYRDLKLAELRGLEAHHHHHHHHHH
Nucleotide sequence of EGF-liganded polypeptide SNAP tagged
SEQ ID NO: 11
TGGCGAATGGGACGCGCCCTGTAGCGGCGCATTAAGCGCGGCGGGTGTGGTGGTTACGCGCAGCGTGA
CCGCTACACTTGCCAGCGCCCTAGCGCCCGCTCCTTTCGCTTTCTTCCCTTCCTTTCTCGCCACGTTC
GCCGGCTTTCCCCGTCAAGCTCTAAATCGGGGGCTCCCTTTAGGGTTCCGATTTAGTGCTTTACGGCA
CCTCGACCCCAAAAAACTTGATTAGGGTGATGGTTCACGTAGTGGGCCATCGCCCTGATAGACGGTTT
TTCGCCCTTTGACGTTGGAGTCCACGTTCTTTAATAGTGGACTCTTGTTCCAAACTGGAACAACACTC
AACCCTATCTCGGTCTATTCTTTTGATTTATAAGGGATTTTGCCGATTTCGGCCTATTGGTTAAAAAA
TGAGCTGATTTAACAAAAATTTAACGCGAATTTTAACAAAATATTAACGCTTACAATTTAGGTGGCAC
TTTTCGGGGAAATGTGCGCGGAACCCCTATTTGTTTATTTTTCTAAATACATTCAAATATGTATCCGC
TCATGAATTAATTCTTAGAAAAACTCATCGAGCATCAAATGAAACTGCAATTTATTCATATCAGGATT
ATCAATACCATATTTTTGAAAAAGCCGTTTCTGTAATGAAGGAGAAAACTCACCGAGGCAGTTCCATA
GGATGGCAAGATCCTGGTATCGGTCTGCGATTCCGACTCGTCCAACATCAATACAACCTATTAATTTC
CCCTCGTCAAAAATAAGGTTATCAAGTGAGAAATCACCATGAGTGACGACTGAATCCGGTGAGAATGG
CAAAAGTTTATGCATTTCTTTCCAGACTTGTTCAACAGGCCAGCCATTACGCTCGTCATCAAAATCAC
TCGCATCAACCAAACCGTTATTCATTCGTGATTGCGCCTGAGCGAGACGAAATACGCGATCGCTGTTA
aaaggacaattacaaacaggaatcgaatgcaaccggcgcaggaacactgccagcgcatcaacaatatt
TTCACCTGAATCAGGATATTCTTCTAATACCTGGAATGCTGTTTTCCCGGGGATCGCAGTGGTGAGTA
ACCATGCATCATCAGGAGTACGGATAAAATGCTTGATGGTCGGAAGAGGCATAAATTCCGTCAGCCAG
TTTAGTCTGACCATCTCATCTGTAACATCATTGGCAACGCTACCTTTGCCATGTTTCAGAAACAACTC
TGGCGCATCGGGCTTCCCATACAATCGATAGATTGTCGCACCTGATTGCCCGACATTATCGCGAGCCC
ATTTATACCCATATAAATCAGCATCCATGTTGGAATTTAATCGCGGCCTAGAGCAAGACGTTTCCCGT
TGAATATGGCTCATAACACCCCTTGTATTACTGTTTATGTAAGCAGACAGTTTTATTGTTCATGACCA
AAATCCCTTAACGTGAGTTTTCGTTCCACTGAGCGTCAGACCCCGTAGAAAAGATCAAAGGATCTTCT
TGAGATCCTTTTTTTCTGCGCGTAATCTGCTGCTTGCAAACAAAAAAACCACCGCTACCAGCGGTGGT
TTGTTTGCCGGATCAAGAGCTACCAACTCTTTTTCCGAAGGTAACTGGCTTCAGCAGAGCGCAGATAC
CAAATACTGTCCTTCTAGTGTAGCCGTAGTTAGGCCACCACTTCAAGAACTCTGTAGCACCGCCTACA
TACCTCGCTCTGCTAATCCTGTTACCAGTGGCTGCTGCCAGTGGCGATAAGTCGTGTCTTACCGGGTT
GGACTCAAGACGATAGTTACCGGATAAGGCGCAGCGGTCGGGCTGAACGGGGGGTTCGTGCACACAGC
CCAGCTTGGAGCGAACGACCTACACCGAACTGAGATACCTACAGCGTGAGCTATGAGAAAGCGCCACG
CTTCCCGAAGGGAGAAAGGCGGACAGGTATCCGGTAAGCGGCAGGGTCGGAACAGGAGAGCGCACGAG
GGAGCTTCCAGGGGGAAACGCCTGGTATCTTTATAGTCCTGTCGGGTTTCGCCACCTCTGACTTGAGC
GTCGATTTTTGTGATGCTCGTCAGGGGGGCGGAGCCTATGGAAAAACGCCAGCAACGCGGCCTTTTTA
CGGTTCCTGGCCTTTTGCTGGCCTTTTGCTCACATCGGCGATAATGGCCTGCTTCTCGCCGAAACGTT
TGGTGGCGGGACCAGTGACGAAGGCTTGAGCGAGGGCGTGCAAGATTCCGAATACCGCAAGCGACAGG
CCGATGATCGTCGCGCTCCAGCGAAAGCGGTCCTCGCCGAAAATGACCCAGAGCGCTGCCGGCACCTG
TCCTACGAGTTGCATGATAAAGAAGACAGTCATAAGTGCGGCGACGATAGTCATGCCCCGCGCCCACC
GGAAGGAGCTGACTGGGTTGAAGGCTCTCAAGGGCATCGGTCGAGATCCCGGTGCCTAATGAGTGAGC
TAACTTACATTAATTGCGTTGCGCTCACTGCCCGCTTTCCAGTCGGGAAACCTGTCGTGCCAGCTGCA
TTAATGAATCGGCCAACGCGCGGGGAGAGGCGGTTTGCGTATTGGGCGCCAGGGTGGTTTTTCTTTTC
ACCAGTGAGACGGGCAACAGCTGATTGCCCTTCACCGCCTGGCCCTGAGAGAGTTGCAGCAAGCGGTC
CACGCTGGTTTGCCCCAGCAGGCGAAAATCCTGTTTGATGGTGGTTAACGGCGGGATATAACATGAGC
TGTCTTCGGTATCGTCGTATCCCACTACCGAGATATCCGCACCAACGCGCAGCCCGGACTCGGTAATG
GCGCGCATTGCGCCCAGCGCCATCTGATCGTTGGCAACCAGCATCGCAGTGGGAACGATGCCCTCATT
CAGCATTTGCATGGTTTGTTGAAAACCGGACATGGCACTCCAGTCGCCTTCCCGTTCCGCTATCGGCT
GAATTTGATTGCGAGTGAGATATTTATGCCAGCCAGCCAGACGCAGACGCGCCGAGACAGAACTTAAT
GGGCCCGCTAACAGCGCGATTTGCTGGTGACCCAATGCGACCAGATGCTCCACGCCCAGTCGCGTACC
GTCTTCATGGGAGAAAATAATACTGTTGATGGGTGTCTGGTCAGAGACATCAAGAAATAACGCCGGAA
cattagtgcaggcagcttccacagcaatggcatcctggtcatccagcggatagttaatgatcagccca
CTGACGCGTTGCGCGAGAAGATTGTGCACCGCCGCTTTACAGGCTTCGACGCCGCTTCGTTCTACCAT
CGACACCACCACGCTGGCACCCAGTTGATCGGCGCGAGATTTAATCGCCGCGACAATTTGCGACGGCG
CGTGCAGGGCCAGACTGGAGGTGGCAACGCCAATCAGCAACGACTGTTTGCCCGCCAGTTGTTGTGCC
ACGCGGTTGGGAATGTAATTCAGCTCCGCCATCGCCGCTTCCACTTTTTCCCGCGTTTTCGCAGAAAC
GTGGCTGGCCTGGTTCACCACGCGGGAAACGGTCTGATAAGAGACACCGGCATACTCTGCGACATCGT
ATAACGTTACTGGTTTCACATTCACCACCCTGAATTGACTCTCTTCCGGGCGCTATCATGCCATACCG
CGAAAGGTTTTGCGCCATTCGATGGTGTCCGGGATCTCGACGCTCTCCCTTATGCGACTCCTGCATTA
GGAAGCAGCCCAGTAGTAGGTTGAGGCCGTTGAGCACCGCCGCCGCAAGGAATGGTGCATGCAAGGAG
ATGGCGCCCAACAGTCCCCCGGCCACGGGGCCTGCCACCATACCCACGCCGAAACAAGCGCTCATGAG
CCCGAAGTGGCGAGCCCGATCTTCCCCATCGGTGATGTCGGCGATATAGGCGCCAGCAACCGCACCTG
TGGCGCCGGTGATGCCGGCCACGATGCGTCCGGCGTAGAGGATCGAGATCTCGATCCCGCGAAATTAA
TACGACTCACTATAGGGGAATTGTGAGCGGATAACAATTCCCCTCAAGAAATAATTTTGTTTAACTTT
AAGAAGGAGATATACATATgATGGACAAAGACTGCGAAATGAAGCGCACCACCCTGGATAGCCCTCTG
GGCAAGCTGGAACTGTCTGGGTGCGAACAGGGCCTGCACCGTATCATCTTCCTGGGCAAAGGAACATC
TGCCGCCGACGCCGTGGAAGTGCCTGCCCCAGCCGCCGTGCTGGGCGGACCAGAGCCACTGATGCAGG
CCACCGCCTGGCTCAACGCCTACTTTCACCAGCCTGAGGCCATCGAGGAGTTCCCTGTGCCAGCCCTG
CACCACCCAGTGTTCCAGCAGGAGAGCTTTACCCGCCAGGTGCTGTGGAAACTGCTGAAAGTGGTGAA
GTTCGGAGAGGTCATCAGCTACAGCCACCTGGCCGCCCTGGCCGGCAATCCCGCCGCCACCGCCGCCG
TGAAAACCGCCCTGAGCGGAAATCCCGTGCCCATTCTGATCCCCTGCCACCGGGTGGTGCAGGGCGAC
CTGGACGTGGGGGGCTACGAGGGCGGGCTCGCCGTGAAAGAGTGGCTGCTGGCCCACGAGGGCCACAG
ACTGGGCAAGCCTGGGCTGGGTGGCGGCAGCGGCGGCGGCAGCGGCGGCggatccatggagttcgtta
acaaacagttcaactataaagacccagttaacggtgttgacattgcttacatcaaaatcccgaacgct
ggccagatgcagccggtaaaggcattcaaaatccacaacaaaatctgggttatcccggaacgtgatac
ctttactaacccggaagaaggtgacctgaacccgccaccggaagcgaaacaggtgccggtatcttact
atgactccacctacctgtctaccgataacgaaaaggacaactacctgaaaggtgttactaaactgttc
gagcgtatttactccaccgacctgggccgtatgctgctgactagcatcgttcgcggtatcccgttctg
gggcggttctaccatcgataccgaactgaaagtaatcgacactaactgcatcaacgttattcagccgg
acggttcctatcgttccgaagaactgaacctggtgatcatcggcccgtctgctgatatcatccagttc
gagtgtaagagctttggtcacgaagttctgaacctcacccgtaacggctacggttccactcagtacat
ccgtttctctccggacttcaccttcggttttgaagaatccctggaagtagacacgaacccactgctgg
gcgctggtaaattcgcaactgatcctgcggttaccctggctcacgaactgattcatgcaggccaccgc
ctgtacggtatcgccatcaatccgaaccgtgtcttcaaagttaacaccaacgcgtattacgagatgtc
cggtctggaagttagcttcgaagaactgcgtacttttggcggtcacgacgctaaattcatcgactctc
tgcaagaaaacgagttccgtctgtactactataacaagttcaaagatatcgcatccaccctgaacaaa
gcgaaatccatcgtgggtaccactgcttctctccagtacatgaagaacgtttttaaagaaaaatacct
gctcagcgaagacacctccggcaaattctctgtagacaagttgaaattcgataaactttacaaaatgc
tgactgaaatttacaccgaagacaacttcgttaagttctttaaagttctgaaccgcaaaacctatctg
aacttcgacaaggcagtattcaaaatcaacatcgtgccgaaagttaactacactatctacgatggttt
caacctgcgtaacaccaacctggctgctaattttaacggccagaacacggaaatcaacaacatgaact
tcacaaaactgaaaaacttcactggtctgttcgagttttacaagctgctgtgcgtcgacggcatcatt
acctccaaaactaaatctctgatagaaggtagaaacaaagcgctgaacctgcagtgtatcaaggttaa
caactgggatttattcttcagcccgagtgaagacaacttcaccaacgacctgaacaaaggtgaagaaa
tcacctcagatactaacatcgaagcagccgaagaaaacatctcgctggacctgatccagcagtactac
ctgacctttaatttcgacaacgagccggaaaacatttctatcgaaaacctgagctctgatatcatcgg
ccagctggaactgatgccgaacatcgaacgtttcccaaacggtaaaaagtacgagctggacaaatata
ccatgttccactacctgcgcgcgcaggaatttgaacacggcaaatcccgtatcgcactgactaactcc
gttaacgaagctctgctcaacccgtcccgtgtatacaccttcttctctagcgactacgtgaaaaaggt
caacaaagcgactgaagctgcaatgttcttgggttgggttgaacagcttgtttatgattttaccgacg
agacgtccgaagtatctactaccgacaaaattgcggatatcactatcatcatcccgtacatcggtccg
gctctgaacattggcaacatgctgtacaaagacgacttcgttggcgcactgatcttctccggtgcggt
gatcctgctggagttcatcccggaaatcgccatcccggtactgggcacctttgctctggtttcttaca
ttgcaaacaaggttctgactgtacaaaccatcgacaacgcgctgagcaaacgtaacgaaaaatgggat
gaagtttacaaatatatcgtgaccaactggctggctaaggttaatactcagatcgacctcatccgcaa
aaaaatgaaagaagcactggaaaaccaggcggaagctaccaaggcaatcattaactaccagtacaacc
agtacaccgaggaagaaaaaaacaacatcaacttcaacatcgacgatctgtcctctaaactgaacgaa
tccatcaacaaagctatgatcaacatcaacaagttcctgaaccagtgctctgtaagctatctgatgaa
ctccatgatcccgtacggtgttaaacgtctggaggacttcgatgcgtctctgaaagacgccctgctga
aatacatttacgacaaccgtggcactctgatcggtcaggttgatcgtctgaaggacaaagtgaacaat
accttatcgaccgacatcccttttcagctcagtaaatatgtcgataaccaacgccttttgtccactct
agaaggcggTGGCGGTAGCGGTGGCGGTGGCAGCGGCGGTGGCGGTAGCGCACTAGacAACAGCGACC
CTAAATGCCCACTaAGTCATGAAGGATACTGCCTTAATGATGGTGTTTGTATGTACATAGGAACATTG
GACCGTTATGCTTGCAATTGTGTAGTGGGCTATGTCGGGGAAAGGTGTCAATATCGAGATCTCAAGCT
GGCAGAGTTAAGAgggctagaagcaCACCATCATCACcaccatcaccatcaccattaatgaAAGCTTG
CGGCCGCACTCGAGCACCACCACCACCACCACTGAGATCCGGCTGCTAACAAAGCCCGAAAGGAAGCT
GAGTTGGCTGCTGCCACCGCTGAGCAATAACTAGCATAACCCCTTGGGGCCTCTAAACGGGTCTTGAG
GGGTTTTTTGCTGAAAGGAGGAACTATATCCGGAT
Polypeptide sequence of EGF-liganded polypeptide SNAP tagged
SEQ ID NO: 12
MDKDCEMKRTTLDSPLGKLELSGCEQGLHRIIFLGKGTSAADAVEVPAPAAVLGGPEPLMQATAWLNA
YFHQPEAIEEFPVPALHHPVFQQESFTRQVLWKLLKVVKFGEVISYSHLAALAGNPAATAAVKTALSG
NPVPILIPCHRVVQGDLDVGGYEGGLAVKEWLLAHEGHRLGKPGLGGGSGGGSGGGSMEFVNKQFNYK
DPVNGVDIAYIKIPNAGQMQPVKAFKIHNKIWVIPERDTFTNPEEGDLNPPPEAKQVPVSYYDSTYLS
TDKSKDNYLKGVTKLFERIYSTDLGRMLLTSIVRGIPFWGGSTIDTELKVIDTNCINVIQPDGSYRSE
ELNLVIIGPSADIIQFECKSFGHEVLNLTRNGYGSTQYIRFSPDFTFGFEESLEVDTNPLLGAGKFAT
DPAVTLAHELIHAGHRLYGIAINPNRVFKVNTNAYYEMSGLEVSFEELRTFGGHDAKFIDSLQENEFR
LYYYNKFKDIASTLNKAKSIVGTTASLQYMKNVFKEKYLLSEDTSGKFSVDKLKFDKLYKMLTEIYTE
DNFVKFFKVLNRKTYLNFDKAVFKINIVPKVNYTIYDGFNLRNTNLAANFNGQNTEINNMNFTKLKNF
TGLFEFYKLLCVDGIITSKTKSLIEGRNKALNLQCIKVNNWDLFFSPSEDNFTNDLNKGEEITSDTNI
EAAEENISLDLIQQYYLTFNFDNEPENISIENLSSDIIGQLELMPNIERFPNGKKYELDKYTMFHYLR
AQEFEHGKSRIALTNSVNEALLNPSRVYTFFSSDYVKKVNKATEAAMFLGWVEQLVYDFTDETSEVST
TDKIADITIIIPYIGPALNIGNMLYKDDFVGALIFSGAVILLEFIPEIAIPVLGTFALVSYIANKVLT
VQTIDNALSKRNEKWDEVYKYIVTKWLAKVNTQIDLIRKKMKEALEMQAEATKAIINYQYNQYTEEEK
NNINFNIDDLSSKLNESINKAMININKFLNQCSVSYLMNSMIPYGVKRLEDFDASLKDALLKYIYDNR
GTLIGQVDRLKDKVNNTLSTDIPFQLSKYVDNQRLLSTLEGGGGSGGGGSGGGGSALDNSDPKCPLSH
EGYCLNDGVCMYIGTLDRYACNCVVGYVGERCQYRDLKLAELRGLEAHHHHHHHHHH
Nucleotide sequence of Sortase A (LPESG-targeting)
SEQ ID NO: 13
TGGCGAATGGGACGCGCCCTGTAGCGGCGCATTAAGCGCGGCGGGTGTGGTGGTTACGCGCAGCGTGA
CCGCTACACTTGCCAGCGCCCTAGCGCCCGCTCCTTTCGCTTTCTTCCCTTCCTTTCTCGCCACGTTC
GCCGGCTTTCCCCGTCAAGCTCTAAATCGGGGGCTCCCTTTAGGGTTCCGATTTAGTGCTTTACGGCA
CCTCGACCCCAAAAAACTTGATTAGGGTGATGGTTCACGTAGTGGGCCATCGCCCTGATAGACGGTTT
TTCGCCCTTTGACGTTGGAGTCCACGTTCTTTAATAGTGGACTCTTGTTCCAAACTGGAACAACACTC
AACCCTATCTCGGTCTATTCTTTTGATTTATAAGGGATTTTGCCGATTTCGGCCTATTGGTTAAAAAA
TGAGCTGATTTAACAAAAATTTAACGCGAATTTTAACAAAATATTAACGCTTACAATTTAGGTGGCAC
TTTTCGGGGAAATGTGCGCGGAACCCCTATTTGTTTATTTTTCTAAATACATTCAAATATGTATCCGC
TCATGAATTAATTCTTAGAAAAACTCATCGAGCATCAAATGAAACTGCAATTTATTCATATCAGGATT
ATCAATACCATATTTTTGAAAAAGCCGTTTCTGTAATGAAGGAGAAAACTCACCGAGGCAGTTCCATA
GGATGGCAAGATCCTGGTATCGGTCTGCGATTCCGACTCGTCCAACATCAATACAACCTATTAATTTC
CCCTCGTCAAAAATAAGGTTATCAAGTGAGAAATCACCATGAGTGACGACTGAATCCGGTGAGAATGG
CAAAAGTTTATGCATTTCTTTCCAGACTTGTTCAACAGGCCAGCCATTACGCTCGTCATCAAAATCAC
TCGCATCAACCAAACCGTTATTCATTCGTGATTGCGCCTGAGCGAGACGAAATACGCGATCGCTGTTA
AAAGGACAATTACAAACAGGAATCGAATGCAACCGGCGGAGGAACACTGCCAGCGCATCAAGAATATT
TTCACCTGAATCAGGATATTCTTCTAATACCTGGAATGCTGTTTTCCCGGGGATCGCAGTGGTGAGTA
ACCATGCATCATCAGGAGTACGGATAAAATGCTTGATGGTCGGAAGAGGCATAAATTCCGTCAGCCAG
TTTAGTCTGACCATCTCATCTGTAACATCATTGGCAACGCTACCTTTGCCATGTTTCAGAAACAACTC
TGGCGCATCGGGCTTCCCATACAATCGATAGATTGTCGCACCTGATTGCCCGACATTATCGCGAGCCC
ATTTATACCCATATAAATCAGCATCCATGTTGGAATTTAATCGCGGCCTAGAGCAAGACGTTTCCCGT
TGAATATGGCTCATAACACCCCTTGTATTACTGTTTATGTAAGCAGACAGTTTTATTGTTCATGACCA
AAATCCCTTAACGTGAGTTTTCGTTCCACTGAGCGTCAGACCCCGTAGAAAAGATCAAAGGATCTTCT
TGAGATCCTTTTTTTCTGCGCGTAATCTGCTGCTTGCAAACAAAAAAACCACCGCTACCAGCGGTGGT
TTGTTTGCCGGATCAAGAGCTACCAACTCTTTTTCCGAAGGTAACTGGCTTCAGCAGAGCGCAGATAC
CAAATACTGTCCTTCTAGTGTAGCCGTAGTTAGGCCACCACTTCAAGAACTCTGTAGCACCGCCTACA
TACCTCGCTCTGCTAATCCTGTTACCAGTGGCTGCTGCCAGTGGCGATAAGTCGTGTCTTACCGGGTT
GGACTCAAGACGATAGTTACCGGATAAGGCGCAGCGGTCGGGCTGAACGGGGGGTTCGTGCACACAGC
CCAGCTTGGAGCGAACGACCTACACCGAACTGAGATACCTACAGCGTGAGCTATGAGAAAGCGCCACG
CTTCCCGAAGGGAGAAAGGCGGACAGGTATCCGGTAAGCGGCAGGGTCGGAACAGGAGAGCGCACGAG
GGAGCTTCCAGGGGGAAACGCCTGGTATCTTTATAGTCCTGTCGGGTTTCGCCACCTCTGACTTGAGC
GTCGATTTTTGTGATGCTCGTCAGGGGGGCGGAGCCTATGGAAAAACGCCAGCAACGCGGCCTTTTTA
CGGTTCCTGGCCTTTTGCTGGCCTTTTGCTCACATCGGCGATAATGGCCTGCTTCTCGCCGAAACGTT
TGGTGGCGGGACCAGTGACGAAGGCTTGAGCGAGGGCGTGCAAGATTCCGAATACCGCAAGCGACAGG
CCGATCATCGTCGCGCTCCAGCGAAAGCGGTCCTCGCCGAAAATGACCCAGAGCGCTGCCGGCACCTG
TCCTACGAGTTGCATGATAAAGAAGACAGTCATAAGTGCGGCGACGATAGTCATGCCCCGCGCCCACC
GGAAGGAGCTGACTGGGTTGAAGGCTCTCAAGGGCATCGGTCGAGATCCCGGTGCCTAATGAGTGAGC
TAACTTACATTAATTGCGTTGCGCTCACTGCCCGCTTTCCAGTCGGGAAACCTGTCGTGCCAGCTGCA
TTAATGAATCGGCCAACGCGCGGGGAGAGGCGGTTTGCGTATTGGGCGCCAGGGTGGTTTTTCTTTTC
ACCAGTGAGACGGGCAACAGCTGATTGCCCTTCACCGCCTGGCCCTGAGAGAGTTGCAGCAAGCGGTC
CACGCTGGTTTGCCCCAGCAGGCGAAAATCCTGTTTGATGGTGGTTAACGGCGGGATATAACATGAGC
TGTCTTCGGTATCGTCGTATCCCACTACCGAGATATCCGCACCAACGCGCAGCCCGGACTCGGTAATG
GCGCGCATTGCGCCCAGCGCCATCTGATCGTTGGCAACCAGCATCGCAGTGGGAACGATGCCCTCATT
CAGCATTTGCATGGTTTGTTGAAAACCGGACATGGCACTCCAGTCGCCTTCCCGTTCCGCTATCGGCT
GAATTTGATTGCGAGTGAGATATTTATGCCAGCCAGCCAGACGCAGACGCGCCGAGACAGAACTTAAT
GGGCCCGCTAACAGCGCGATTTGCTGGTGACCCAATGCGACCAGATGCTCCACGCCCAGTCGCGTACC
GTCTTCATGGGAGAAAATAATACTGTTGATGGGTGTCTGGTCAGAGACATCAAGAAATAACGCCGGAA
CATTAGTGCAGGCAGCTTCCACAGCAATGGCATCCTGGTCATCCAGCGGATAGTTAATGATCAGCCCA
CTGACGCGTTGCGCGAGAAGATTGTGCACCGCCGCTTTACAGGCTTCGACGCCGCTTCGTTCTACCAT
CGACACCACCACGCTGGCACCCAGTTGATCGGCGCGAGATTTAATCGCCGCGACAATTTGCGACGGCG
CGTGCAGGGCCAGACTGGAGGTGGCAACGCCAATCAGCAACGACTGTTTGCCCGCCAGTTGTTGTGCC
ACGCGGTTGGGAATGTAATTCAGCTCCGCCATCGCCGCTTCCACTTTTTCCCGCGTTTTCGCAGAAAC
GTGGCTGGCCTGGTTCACCACGCGGGAAACGGTCTGATAAGAGACACCGGCATACTCTGCGACATCGT
ATAACGTTACTGGTTTCACATTCACCACCCTGAATTGACTCTCTTCCGGGCGCTATCATGCCATACCG
CGAAAGGTTTTGCGCCATTCGATGGTGTCCGGGATCTCGACGCTCTCCCTTATGCGACTCCTGCATTA
GGAAGCAGCCCAGTAGTAGGTTGAGGCCGTTGAGCACCGCCGCCGCAAGGAATGGTGCATGCAAGGAG
ATGGCGCCCAACAGTCCCCCGGCCACGGGGCCTGCCACCATACCCACGCCGAAACAAGCGCTCATGAG
CCCGAAGTGGCGAGCCCGATCTTCCCCATCGGTGATGTCGGCGATATAGGCGCCAGCAACCGCACCTG
TGGCGCCGGTGATGCCGGCCACGATGCGTCCGGCGTAGAGGATCGAGATCTCGATCCCGCGAAATTAA
TACGACTCACTATAGGGGAATTGTGAGCGGATAACAATTCCCCTCAAGAAATAATTTTGTTTAACTTT
AAGAAGGAGATATCATATGCAGGCAAAACCGCAGATTCCGAAAGATAAAAGCAAAGTGGCAGGCTATA
TTGAAATTCCGGATGCCGATATTAAAGAACCGGTTTATCCGGGTCCTGCAACACGTGAACAGCTGGAT
CGTGGTGTTTGTTTTGTTGAAGAAAATGAGAGCCTGGATGATCAGAACATTAGCATTACCGGTCATAC
CGCAATTGATCGTCCGAATTATCAGTTTACCAATCTGCGTGCAGCCAAACCGGGTAGCATGGTTTATC
TGAAAGTTGGTAATGAAACCCGCATCTACAAAATGACCAGCATTCGTAATGTTAAACCGACCGCAGTT
GGTGTTCTGGATGAACAAAAAGGTAAAGATAAACAGCTGACCCTGGTTACCTGTGATGATTATAACTT
TGAAACCGGTGTTTGGGAAACGCGCAAAATCTTTGTTGCAACCGAAGTTAAACATCACCATCACCACC
ATCATCATCACCATTAAAAGCTTGCGGCCGCACTCGAGCACCACCACCACCACCACTGAGATCCGGCT
GCTAACAAAGCCCGAAAGGAAGCTGAGTTGGCTGCTGCCACCGCTGAGCAATAACTAGCATAACCCCT
TGGGGCCTCTAAACGGGTCTTGAGGGGTTTTTTGCTGAAAGGAGGAACTATATCCGGAT
Polypeptide sequence of Sortase A (LPESG-targeting)
SEQ ID NO: 14
MQAKPQIPKDKSKVAGYIEIPDADIKEPVYPGPATREQLDRGVCFVEENESLDDQNISITGHTAIDRP
NYQFTNLRAAKPGSMVYLKVGNETRIYKMTSIRNVKPTAVGVLDEQKGKDKQLTLVTCDDYNFETGVW
ETRKIFVATEVKHHHKHHHHHH
Nucleotide sequence of Sortase A (LAETG-targeting)
SEQ ID NO: 15
TGGCGAATGGGACGCGCCCTGTAGCGGCGCATTAAGCGCGGCGGGTGTGGTGGTTACGCGCAGCGTGA
CCGCTACACTTGCCAGCGCCCTAGCGCCCGCTCCTTTCGCTTTCTTCCCTTCCTTTCTCGCCACGTTC
GCCGGCTTTCCCCGTCAAGCTCTAAATCGGGGGCTCCCTTTAGGGTTCCGATTTAGTGCTTTACGGCA
CCTCGACCCCAAAAAACTTGATTAGGGTGATGGTTCACGTAGTGGGCCATCGCCCTGATAGACGGTTT
TTCGCCCTTTGACGTTGGAGTCCACGTTCTTTAATAGTGGACTCTTGTTCCAAACTGGAACAACACTC
AACCCTATCTCGGTCTATTCTTTTGATTTATAAGGGATTTTGCCGATTTCGGCCTATTGGTTAAAAAA
TGAGCTGATTTAACAAAAATTTAACGCGAATTTTAACAAAATATTAACGCTTACAATTTAGGTGGCAC
TTTTCGGGGAAATGTGCGCGGAACCCCTATTTGTTTATTTTTCTAAATACATTCAAATATGTATCCGC
TCATGAATTAATTCTTAGAAAAACTCATCGAGCATCAAATGAAACTGCAATTTATTCATATCAGGATT
ATCAATACCATATTTTTGAAAAAGCCGTTTCTGTAATGAAGGAGAAAACTCACCGAGGCAGTTCCATA
GGATGGCAAGATCCTGGTATCGGTCTGCGATTCCGACTCGTCCAACATCAATACAACCTATTAATTTC
CCCTCGTCAAAAATAAGGTTATCAAGTGAGAAATCACCATGAGTGACGACTGAATCCGGTGAGAATGG
CAAAAGTTTATGCATTTCTTTCCAGACTTGTTCAACAGGCCAGCCATTACGCTCGTCATCAAAATCAC
TCGCATCAACCAAACCGTTATTCATTCGTGATTGCGCCTGAGCGAGACGAAATACGCGATCGCTGTTA
AAAGGACAATTACAAACAGGAATCGAATGCAACCGGCGCAGGAACACTGCCAGCGCATCAACAATATT
TTCACCTGAATCAGGATATTCTTCTAATACCTGGAATGCTGTTTTCCCGGGGATCGCAGTGGTGAGTA
ACCATGCATCATCAGGAGTACGGATAAAATGCTTGATGGTCGGAAGAGGCATAAATTCCGTCAGCCAG
TTTAGTCTGACCATCTCATCTGTAACATCATTGGCAACGCTACCTTTGCCATGTTTCAGAAACAACTC
TGGCGCATCGGGCTTCCCATACAATCGATAGATTGTCGCACCTGATTGCCCGACATTATCGCGAGCCC
ATTTATACCCATATAAATCAGCATCCATGTTGGAATTTAATCGCGGCCTAGAGCAAGACGTTTCCCGT
TGAATATGGCTCATAACACCCCTTGTATTACTGTTTATGTAAGCAGACAGTTTTATTGTTCATGACCA
AAATCCCTTAACGTGAGTTTTCGTTCCACTGAGCGTCAGACCCCGTAGAAAAGATCAAAGGATCTTCT
TGAGATCCTTTTTTTCTGCGCGTAATCTGCTGCTTGCAAACAAAAAAACCACCGCTACCAGCGGTGGT
TTGTTTGCCGGATCAAGAGCTACCAACTCTTTTTCCGAAGGTAACTGGCTTCAGCAGAGCGCAGATAC
CAAATACTGTCCTTCTAGTGTAGCCGTAGTTAGGCCACCACTTCAAGAACTCTGTAGCACCGCCTACA
TACCTCGCTCTGCTAATCCTGTTACCAGTGGCTGCTGCCAGTGGCGATAAGTCGTGTCTTACCGGGTT
GGACTCAAGACGATAGTTACCGGATAAGGCGCAGCGGTCGGGCTGAACGGGGGGTTCGTGCACACAGC
CCAGCTTGGAGCGAACGACCTACACCGAACTGAGATACCTACAGCGTGAGCTATGAGAAAGCGCCACG
CTTCCCGAAGGGAGAAAGGCGGACAGGTATCCGGTAAGCGGCAGGGTCGGAACAGGAGAGCGCACGAG
GGAGCTTCCAGGGGGAAACGCCTGGTATCTTTATAGTCCTGTCGGGTTTCGCCACCTCTGACTTGAGC
GTCGATTTTTGTGATGCTCGTCAGGGGGGCGGAGCCTATGGAAAAACGCCAGCAACGCGGCCTTTTTA
CGGTTCCTGGCCTTTTGCTGGCCTTTTGCTCACATCGGCGATAATGGCCTGCTTCTCGCCGAAACGTT
TGGTGGCGGGACCAGTGACGAAGGCTTGAGCGAGGGCGTGCAAGATTCCGAATACCGCAAGCGACAGG
CCGATCATCGTCGCGCTCCAGCGAAAGCGGTCCTCGCCGAAAATGACCCAGAGCGCTGCCGGCACCTG
TCCTACGAGTTGCATGATAAAGAAGACAGTCATAAGTGCGGCGACGATAGTCATGCCCCGCGCCCACC
GGAAGGAGCTGACTGGGTTGAAGGCTCTCAAGGGCATCGGTCGAGATCCCGGTGCCTAATGAGTGAGC
TAACTTACATTAATTGCGTTGCGCTCACTGCCCGCTTTCCAGTCGGGAAACCTGTCGTGCCAGCTGCA
TTAATGAATCGGCCAACGCGCGGGGAGAGGCGGTTTGCGTATTGGGCGCCAGGGTGGTTTTTCTTTTC
ACCAGTGAGACGGGCAACAGCTGATTGCCCTTCACCGCCTGGCCCTGAGAGAGTTGCAGCAAGCGGTC
CACGCTGGTTTGCCCCAGCAGGCGAAAATCCTGTTTGATGGTGGTTAACGGCGGGATATAACATGAGC
TGTCTTCGGTATCGTCGTATCCCACTACCGAGATATCCGCACCAACGCGCAGCCCGGACTCGGTAATG
GCGCGCATTGCGCCCAGCGCCATCTGATCGTTGGCAACCAGCATCGCAGTGGGAACGATGCCCTCATT
CAGCATTTGCATGGTTTGTTGAAAACCGGACATGGCACTCCAGTCGCCTTCCCGTTCCGCTATCGGCT
GAATTTGATTGCGAGTGAGATATTTATGCCAGCCAGCCAGACGCAGACGCGCCGAGACAGAACTTAAT
GGGCCCGCTAACAGCGCGATTTGCTGGTGACCCAATGCGACCAGATGCTCCACGCCCAGTCGCGTACC
GTCTTCATGGGAGAAAATAATACTGTTGATGGGTGTCTGGTCAGAGACATCAAGAAATAACGCCGGAA
CATTAGTGCAGGCAGCTTCCACAGCAATGGCATCCTGGTCATCCAGCGGATAGTTAATGATCAGCCCA
CTGACGCGTTGCGCGAGAAGATTGTGCACCGCCGCTTTACAGGCTTCGACGCCGCTTCGTTCTACCAT
CGACACCACCACGCTGGCACCCAGTTGATCGGCGCGAGATTTAATCGCCGCGACAATTTGCGACGGCG
CGTGCAGGGCCAGACTGGAGGTGGCAACGCCAATCAGCAACGACTGTTTGCCCGCCAGTTGTTGTGCC
ACGCGGTTGGGAATGTAATTCAGCTCCGCCATCGCCGCTTCCACTTTTTCCCGCGTTTTCGCAGAAAC
GTGGCTGGCCTGGTTCACCACGCGGGAAACGGTCTGATAAGAGACACCGGCATACTCTGCGACATCGT
ATAACGTTACTGGTTTCACATTCACCACCCTGAATTGACTCTCTTCCGGGCGCTATCATGCCATACCG
CGAAAGGTTTTGCGCCATTCGATGGTGTCCGGGATCTCGACGCTCTCCCTTATGCGACTCCTGCATTA
GGAAGCAGCCCAGTAGTAGGTTGAGGCCGTTGAGCACCGCCGCCGCAAGGAATGGTGCATGCAAGGAG
ATGGCGCCCAACAGTCCCCCGGCCACGGGGCCTGCCACCATACCCACGCCGAAACAAGCGCTCATGAG
CCCGAAGTGGCGAGCCCGATCTTCCCCATCGGTGATGTCGGCGATATAGGCGCCAGCAACCGCACCTG
TGGCGCCGGTGATGCCGGCCACGATGCGTCCGGCGTAGAGGATCGAGATCTCGATCCCGCGAAATTAA
TACGACTCACTATAGGGGAATTGTGAGCGGATAACAATTCCCCTCAAGAAATAATTTTGTTTAACTTT
AAGAAGGAGATATACATATGCAGGCAAAACCGCAGATTCCGAAAGATAAAAGCAAAGTGGCAGGCTAT
ATTGAAATTCCGGATGCCGATATTAAAGAACCGGTTTATCCGGGTCCTGCAACACGTGAACAGCTGAA
TCGTGGTGTTTGTTTTCACGATGAAAATGAGAGCCTGGATGATCAGAATATTAGCATTGCAGGCCATA
CCTTTATTGATCGTCCGAATTATCAGTTCACCAATCTGAAAGCAGCAAAACCGGGTAGCATGGTTTAT
TTCAAAGTTGGTAATGAAACCCGCATCTACAAAATGACCAGCATTCGTAAAGTTCATCCGAATGCAGT
TGGTGTTCTGGATGAACAAGAAGGCAAAGATAAACAGCTGACCCTGGTTACCTGTGATGATTATAACG
AAGAAACCGGTGTTTGGGAAAGCCGTAAAATCTTTGTTGCAACCGAAGTGAAACATCATCACCACCAT
CACCATCATCATCACTAAAAGCTTGCGGCCGCACTCGAGCACCACCACCACCACCACTGAGATCCGGC
TGCTAACAAAGCCCGAAAGGAAGCTGAGTTGGCTGCTGCCACCGCTGAGCAATAACTAGCATAACCCC
TTGGGGCCTCTAAACGGGTCTTGAGGGGTTTTTTGCTGAAAGGAGGAACTATATCCGGAT
Polypeptide sequence of Sortase A (LAETG-targeting)
SEQ ID NO: 16
MQAKPQIPKDKSKVAGYIEIPDADIKEPVYPGPATREQLKRGVCFHDENESLDDQNISIAGHTFIDRP
NYQFTNLKAAKPGSMVYFKVGNETRIYKMTSIRKVHPNAVGVLDEQEGKDKQLTLVTCDDYNEETGVW
ESRKIFVATEVKHHHHHHHHHH
BoNT/A-UniProt P10845
SEQ ID NO: 17
MPFVNKQFNYKDPVNGVDIAYIKIPNVGQMQPVKAFKIHNKIWVIPERDTFTNPEEGDLNPPPEAKQV
PVSYYDSTYLSTDNEKDNYLKGVTKLFERIYSTDLGRMLLTSIVRGIPFWGGSTIDTELKVIDTNCIN
VIQPDGSYRSEELNLVIIGPSADIIQFECKSFGHEVLNLTRNGYGSTQYIRFSPDFTFGFEESLEVDT
NPLLGAGKFATDPAVTLAHELIHAGHRLYGIAINPNRVFKVNTNAYYEMSGLEVSFEELRTFGGHDAK
FIDSLQENEFRLYYYNKFKDIASTLNKAKSIVGTTASLQYMKNVFKEKYLLSEDTSGKFSVDKLKFDK
LYKMLTEIYTEDNFVKFFKVLNRKTYLNFDKAVFKINIVPKVNYTIYDGFNLRNTNLAANFNGQNTEI
NNMNFTKLKNFTGLFEFYKLLCVRGIITSKTKSLDKGYNKALNDLCIKVNNWDLFFSPSEDNFTNDLN
KGEEITSDTNIEAAEENISLDLIQQYYLTFNFDNEPENISIENLSSDIIGQLELMPNIERFPNGKKYE
LDKYTMFHYLRAQEFEHGKSRIALTNSVNEALLNPSRVYTFFSSDYVKKVNKATEAAMFLGWVEQLVY
DFTDETSEVSTTDKIADITIIIPYIGPALNIGNMLYKDDFVGALIFSGAVILLEFIPEIAIPVLGTFA
LVSYIANKVLTVQTIDNALSKRNEKWDEVYKYIVTNWLAKVNTQIDLIRKKMKEALENQAEATKAIIN
YQYNQYTEEEKNNINFNIDDLSSKLNESINKAMININKFLNQCSVSYLMNSMIPYGVKRLEDFDASLK
DALLKYIYDNRGTLIGQVDRLKDKVNNTLSTDIPFQLSKYVDNQRLLSTFTEYIKNIINTSILNLRYE
SNHLIDLSRYASKINIGSKVNFDPIDKNQIQLFKLESSKIEVILKNAIVYNSMYENFSTSFWIRIPKY
FNSISLNNEYTIINCMENNSGWKVSLNYGEIIWTLQDTQEIKQRVVFKYSQMINISDYINRWIFVTIT
NNPANNSKIYINGRLIDQKPISNLGNIHASNNIMFKLDGCRDTHRYIWIKYFNLFDKELNEKEIKDLY
DNQSNSGILKDFWGDYLQYDKPYYMLNLYDPNKYVDVNNVGIRGYMYLKGPRGSVMTTNIYLNSSLYR
GTKFIIKKYASGNKDNIVRNNDRVYINVVVKNKEYRLATNASQAGVEKILSALEIPDVGNLSQVVVMK
SKNDQGITNKCKMNLQDNNGNDIGFIGFHQFNNIAKLVASNWYNRQIERSSRTLGCSWEFIPVDDGWG
ERPL
BoNT/B-UniProt P10844
SEQ ID NO: 18
MPVTINNFNYNDPIDNNNIIMMEPPFARGTGRYYKAFKITDRIWIIPERYTFGYKPEDFNKSSGIFNR
DVCEYYDPDYLNTNDKKKIFLQTMIKLFNRIKSKPLGEKLLEMIIMGIPYLGDRRVPLEEFNTNIASV
TVNKLISNPGEVERKKGIFANLIIFGPGPVLNENETIDIGIQNHFASREGFGGIMQMKFCPEYVSVFN
NVQENKGASIFNRRGYFSDPALILMHELIKVLHGLYGIKVDDLPIVPNEKKFFMQSTDAIQAEELYTF
GGQDPSIITPSTDKSIYDKVLQNFRGIVDRLNKVLVCISDPNININIYKNKFKDKYKFVEDSEGKYSI
DVESFDKLYKSLMFGFTETNIAENYKIKTRASYFSDSLPPVKIKKLLDNEIYTIEEGFNISDKDMEKE
YRGQNKAINKQAYEEISKEHLAVYKIQMCKSVKAPGICIDVDNEDLFFIADKNSFSDDLSKNERIEYN
TQSNYIENDFPINELILDTDLISKIELPSENTESLTDFNVDVPVYEKQFAIKKIFTDEMTIFQYLYSQ
TFPLDIRDISLTSSFDDALLFSNKVYSFFSMDYIKTANKVVEAGLFAGWVKQIVNDFVIEANKSNTMD
KIADISLIVPYIGLALNVGNETAKGNFENAFEIAGASILLEFIPELLIPVVGAFLLESYIDNKNKIIK
TIDNALTKRNEKWSDMYGLIVAQWLSTVNTQFYTIKEGMYKALNYQAQALEEIIKYRYNIYSEKEKSN
INIDFNDINSKLKEGINQAIDNINNFINGCSVSYLMKKMIPLAVEKLLDFDNTLKKNLLNYIDENKLY
LIGSAEYEKSKVNKYLKTIMPFDLSIYTNDTILIEMFNKYNSEILNNIILNLRYKDNNLIDLSGYGAK
VEVYDGVELNDKNQFKLTSSANSKIRVTQNQNIIFNSVFLDFSVSFWIRIPKYKNDGIQNYIHNEYTI
INCMKNNSGWKISIRGNRIIWTLIDINGKTKSVFFEYNIREDISEYINRWFFVTITNNLNNAKIYING
KLESNTDIKDIREVIANGEIIFKLDGDIDRTQFIWMKYFSIFNTELSQSNIEERYKIQSYSEYLKDFW
GNPLMYNKEYYMFNAGNKNSYIKLKKDSPVGEILTRSKYNQNSKYINYRDLYIGEKFIIRRKSNSQSI
NDDIVRKEDYIYLDFFNLNQEWRVYTYKYFKKEEEKLFLAPISDSDEFYNTIQIKEYDEQPTYSCQLL
FKKDEESTDEIGLIGIHRFYESGIVFEEYKDYFCISKWYLKEVKRKPYNLKLGCNWQFIPKDEGWTE
BoNT/C-UniProt P18640
SEQ ID NO: 19
MPITINNFNYSDPVDNKNILYLDTHLNTLANEPEKAFRITGNIWVIPDRFSRNSNPNLNKPPRVTSPK
SGYYDPNYLSTDSDKDPFLKEIIKLFKRINSREIGEELIYRLSTDIPFPGNNNTPINTFDFDVDFNSV
DVKTRQGNNWVKTGSINPSVIITGPRENIIDPETSTFKLTNNTFAAQEGFGALSIISISPRFMLTYSN
ATNDVGEGRFSKSEFCMDPILILMHELNHAMHNLYGIAIPNDQTISSVTSNIFYSQYNVKLEYAEIYA
FGGPTIDLIPKSARKYFEEKALDYYRSIAKRLNSITTANPSSFNKYIGEYKQKLIRKYRFVVESSGEV
TVNRNKFVELYNELTQIFTEFNYAKIYNVQNRKIYLSNVYTPVTANILDDNVYDIQNGFNIPKSNLNV
LFMGQNLSRNPALRKVNPENMLYLFTKFCHKAIDGRSLYNKTLDCRELLVKNTDLPFIGDISDVKTDI
FLRKDINEETEVIYYPDNVSVDQVILSKNTSEHGQLDLLYFSIDSESEILPGENQVFYDNRTQNVDYL
NSYYYLESQKLSDNVEDFTFTRSIEEALDNSAKVYTYFPTLANKVNAGVQGGLFLMWANDVVEDFTTN
ILRKDTLDKISDVSAIIPYIGPALNISMSVRRGNFTEAFAVTGVTILLEAFPEFTIPALGAFVIYSKV
QERNEIIKTIDNCLEQRIKRWKDSYEWMMGTWLSRIITQFNNISYQMYDSLNYQAGAIKAKIDLEYKK
YSGSDKENIKSQVENLKNSLDVKISEAMNNINKFIRECSVTYLFKNMLPKVIDELNEFDRNTKAKLIN
LIDSHNIILVGEVDKLKAKVNNSFQNTIPFNIFSYTNNSLLKDIINEYFNNINDSKILSLQNRKNTLV
DTSGYNAEVSEEGDVQLNPIFPFDFKLGSSGEDRGKVIVTQNENIVYNSMYESFSISFWIRINKWVSN
LPGYTIIDSVKNNSGWSIGIISNFLVFTLKQNEDSEQSINFSYDISNNAPGYNKWFFVTVTNNMMGNM
KIYINGKLIDTIKVKELTGINFSKTITFEINKIPDTGLITSDSDNINMWIRDFYIFAKELDGKDINIL
FNSLQYTNVVKDYWGNDLRYNKEYYMVNIDYLNRYMYANSRQIVFNTRRNNNDFNEGYKIIIKRIRGN
TNDTRVRGGDILYFDMTINNKAYNLFMKNETMYADNHSTEDIYAIGLREQTKDINDNIIFQIQPMNNT
YYYASQIFKSNFNGENISGICSIGTYRFRLGGDWYRHNYLVPTVKQGNYASLLESTSTHWGFVPVSE
BoNT/D-UniProt P19321
SEQ ID NO: 20
MTWPVKDFNYSDPVNDNDILYLRIPQNKLITTPVKAFMITQNIWVIPERFSSDTNPSLSKPPRPTSKY
QSYYDPSYLSTDEQKDTFLKGIIKLFKRINERDIGKKLINYLVVGSPFMGDSSTPEDTFDFTRHTTNI
AVEKFENGSWKVTNIITPSVLIFGPLPNILDYTASLTLQGQQSNPSFEGFGTLSILKVAPEFLLTFSD
VTSNQSSAVLGKSIFCMDPVIALMHELTHSLHQLYGINIPSDKRIRPQVSEGFFSQDGPNVQFEELYT
FGGLDVEIIPQIERSQLREKALGHYKDIAKRLNNINKTIPSSWISNIDKYKKIFSEKYNFDKDNTGNF
VVNIDKFNSLYSDLTNVMSEVVYSSQYNVKNRTHYFSRHYLPVFANILDDNIYTIRDGFNLTNKGFNI
ENSGQNIERNPALQKLSSESVVDLFTKVCLRLTKNSRDDSTCIKVKNNRLPYVADKDSISQEIFENKI
ITDETNVQNYSDKFSLDESILDGQVPINPEIVDPLLPNVNMEPLNLPGEEIVFYDDITKYVDYLNSYY
YLESQKLSNNVENITLTTSVEEALGYSNKIYTFLPSLAEKVNKGVQAGLFLNWANEVVEDFTTNIMKK
DTLDKISDVSVIIPYIGPALNIGNSALRGNFNQAFATAGVAFLLEGFPEFTIPALGVFTFYSSIQERE
KIIKTIENCLEQRVKRWKDSYQWMVSKWLSRITTQFNHINYQMYDSLSYQADAIKAKIDLEYKKYSGS
DKENIKSQVENLKNSLDVKISEAMNNINKFIRECSVTYLFKNMLPKVIDELNKFDLRTKTELINLIDS
HNIILVGEVDRLKAKVNESFENTMPFNIFSYTNNSLLKDIINEYFNSINDSKILSLQNKKNALVDTSG
YNAEVRVGDMVQLNTIYTNDFKLSSSGDKIIVNLNNNILYSAIYENSSVSFWIKISKDLTNSHNEYTI
INSIEQNSGWKLCIRNGNIEWILQDVNRKYKSLIFDYSESLSHTGYTNKWFFVTITNNIMGYMKLYIN
GELKQSQKIEDLDEVKLDKTIVFGIDENIDENQMLWIRDFNIFSKELSNEDINIVYEGQILRNVIKDY
WGKPLKFDTEYYIINDNYIDRYIAPESNVLVLVQYPDRSKLYTGNPITIKSVSDKNPYSRILNGDNII
LHMLYNSRKYMIIRDTDTIYATQGGECSQNCVYALKLQSNLGNYGIGIFSIKNIVSKNKYCSQIFSSF
RENTMLLADIYKPWRFSFKNAYTPVAVTNYETKLLSTSSFWKFISRDPGWVE
BoNT/E-UniProt Q00496
SEQ ID NO: 21
MPKINSFNYNDPVNDRTILYIKPGGCQEFYKSFNIMKNIWIIPERNVIGTTPQDFHPPTSLKNGDSSY
YDPNYLQSDEEKDRFLKIVTKIFNRINNWLSGGILLEELSKANPYLGNDNTPDNQFHIGDASAVEIKF
SNGSQDILLPNVIIMGAEPDLFETNSSNISLRNNYMPSNHRFGSIAIVTFSPEYSFRFNDNCMNEFIQ
DPALTLMHELIHSLHGLYGAKGITTKYTITQKQNPLITNIRGTNIEEFLTFGGTDLNIITSAQSNDIY
TNLLADYKKIASKLSKVQVSNPLLNPYKDVFEAKYGLDKDASGIYSVNINKFNDIFKKLYSFTEFDLR
TKFQVKCRQTYIGQYKYFKLSNLLNDSIYNISEGYNINNLKVNFRGQNANLNPRIITPITGRGLVKKI
IRFCKNIVSVKGIRKSICIEINNGELFFVASENSYNDDNINTPKEIDDTVTSNNNYENDLDQVILNFN
SESAPGLSDEKLNLTIQNDAYIPKYDSNGTSDIEQHDVNELNVFFYLDAQKVPEGENNVNLTSSIDTA
LLEQPKIYTFFSSEFINNVNKPVQAALFVSWIQQVLVDFTTEANQKSTVDKIADISIVVFYIGLALNI
GNEAQKGNFKDALELLGAGILLEFEPELLIPTILVFTIKSFLGSSDNKNKVIKAINNALKERDEKWKE
VYSFIVSNWMTKINTQFNKRKEQMYQALQNQVNAIKTIIESKYNSYTLEEKNELTNKYDIKQIENELN
QKVSIAMNNIDRFLTESSISYLMKIINEVKINKLREYDEMVKTYLLMYIIQHGSILGESQQELNSMVT
DTLNNSIPFKLSSYTDDKILISYFNKFFKRIKSSSVLNMRYKNDKYVDTSGYDSNININGDVYKYPTN
KNQFGIYNDKLSEVNISQNDYIIYDNKYKNFSISFWVRIPNYDNKIVNVNNEYTIINCMRDNNSGWKV
SLNHNEIIWTFEDNRGINQKLAFNYGNANGISDYINKWIFVTITNDRLGDSKLYINGNLIDQKSILNL
GNIHVSDMILFKIVNCSYTRYIGIRYFNIFDKELDETEIQTLYSNEPNTNILKDFWGNYLLYDKEYYL
LNVXKPNNFIDRRKDSTLSINNIRSTILLANRLYSGIKVKIQRVNNSSTNDNLVRKNDQVYIKFVASK
THLFPLYADTATTNKEKTIKISSSGNRFNQVVVMNSVGNCTMNFKNNNGNNIGLLGFKADTVVASTWY
YTHMRDHTNSNGCFWNFISEEHGWQEK
BoNT/F-UniProt A7GBG3
SEQ ID NO: 22
MPVVINSFNYNDPVNDDTILYMQIPYSEKSKKYYKAFEIMRNVWIIPERNTIGTDPSDFDPPASLENG
SSAYYDPNYLTTDAEKDRYLKTTIKLFKRINSNPAGEVLLQEISYAKPYLGNEHTPINEFHPVTRTTS
VNIKSSTNVKSSIILNLLVLGAGPDIFENSSYPVRKLMDSGGVYDPSNDGFGSINIVTFSPEYEYTFN
DISGGYNSSTESFIADPAISLAHELIHALHGLYGARGVTYKETIKVKQAPLMIAEKPIRLEEFLTFGG
QDLNIITSAMKEKIYNNLLANYEKIATRLSRVNSAPPEYDINEYKDYFQWKYGLDKNADGSYTVNENK
FNEIYKKLYSFTEIDLANKFKVKCRNTYFIKYGFLKVPNLLDDDIYTVSEGFNIGKLAVNNRGQNIKL
NPKIIDSIPDKGLVEKIVKFCKSVIPRKGTKAPPRLCIRVNNRELFFVASESSYNENDINTPKEIDDT
TNLNNNYRNNLDEVILDYNSETIPQISNQTLNTLVQDDSYVPRYDSNGTSEIEEHNVVDLNVFFYLHA
QKVPEGETNISLTSSIDTALSEESQVYTFFSSEFINTINKPVHAALFISWINQVIRDFTTEATQKSTF
DKIADISLVVPYVGLALNIGNEVQKENFKEAFELLGAGILLEEVPELLIPTILVFTIKSFIGSSENKN
KIIKAINNSLMERETKWKEIYSWIVSNWLTRINTQFNKRKEQMYQALQNQVDAIKTVIEYKYNNYTSD
ERNRLESEYNINNIREELNKKVSLAMENIERFITESSIFYLMKLINEAKVSKLREYDEGVKEYLLDYI
SEHRSILGNSVQELNDLVTSTLNNSIPFELSSYTHDKILILYFNKLYKKIKDNSILDMRYENNKFIDI
SGYGSNISINGDVYIYSTNRNQFGIYSSKPSEVNIAQNNDIIYNGRYQNFSISFWVRIPKYFNKVNLN
NEYTIIDCIRNKNSGWKISLNYNKIIWTLQDTAGNKQKLVFNYTQMISISDYINKWIFVTITNNRLGN
SRIYINGNLIDEKSISNLGDIHVSDNILFKIVGCNDTRYVGIRYFKVFDTELGKTEIETLYSDEPDPS
ILKDFWGNYLLYNKRYYLLNLLRTDKSITQNSNFLNINQQRGVYQKPNIFSNTRLYTGVEVIIRKNGS
TDISNTDNFVRKNDLAYINVVDRDVEYRLYADISIAKPEKIIKLIRTSNSNNSLGQIIVMDSIGNNCT
MNFQNNNGGNIGLLGFHSNNLVASSWYYNNIRKNTSSNGCFWSFISKEHGWQEN
BoNT/G-UniProt Q60393
SEQ ID NO: 23
MPVNIKXFNYNDPINNDDIIMMEPFNDPGPGTYYKAFRIIDRIWIVPERFTYGFQPDQFNASTGVFSK
DVYEYYDPTYLKTDAEKDKFLKTMIKLFNRINSKPSGQRLLDMIVDAIPYLGNASTPPDKFAANVANV
SINKKIIQPGAEDQIKGLMTNLIIFGPGPVLSDNFTDSMIMNGHSPISEGFGARMMIRFCPSCLNVFN
NVQENKDTSIFSRRAYFADPALTLMHELIHVLHGLYGIKISNLPITPNTKEFFMQHSDPVQAEELYTF
GGHDPSVISPSTDMNIYNKALQNFQDIANRLNIVSSAQGSGIDISLYKQIYKNKYDFVEDPNGKYSVD
KDKFDKLYKALMFGFTETNLAGEYGIKTRYSYFSEYLPPIKTEKLLDNTIYTQNEGFNIASKNLKTEF
NGQNKAVNKEAYEEISLEKLVIYRIAMCKPVMYKNTGKSEQCIIVNNEDLFFIANKDSFSKDLAKAET
IAYNTQNNTIENNFSIDQLILDNDLSSGIDLPNENTEPFTNFDDIDIPVYIKQSALKKIFVDGDSLFE
YLHAQTFPSNIENLQLTNSLNDALRNNNKVYTFFSTNLVEKANTVVGASLFVNWVKGVIDDFTSESTQ
KSTIDKVSDVSIIIPYIGPALNVGNETAKENFKNAFEIGGAAILMEFIPELIVPIVGFFTLESYVGNK
GHIIMTISNALKKRDQKWTDMYGLIVSQWLSTVNTQFYTIKERMYNALNNQSQAIEKIIEDQYNRYSE
EDKMNINIDFNDIDFKLNQSINLAINNIDDFINQCSISYLMNRMIPLAVKKLKDFDDNLKRDLLEYID
TNELYLLDEVNILKSKVNRHLKDSIPFDLSLYTKDTILIQVFNNYISNISSNAILSLSYRGGRLIDSS
GYGATMNVGSDVIFNDIGNGQFKLNNSENSNITAHQSKFVVYDSMFDNFSINFWVRTPKYNNNDIQTY
LQNEYTIISCIKNDSGWKVSIKGNRIIWTLIDVNAKSKSIFFEYSIKDKISDYIKKWFSITITNDRLG
NANIYINGSLKKSEKILNLDRINSSNDIDFKLINCTDTTKFVWIKDFNIFGRELNATEVSSLYWIQSS
TNTLKDFWGKPLRYDTQYYLFNQGMQNIYIKYFSKASMGETAPRTNFNNAAINYQNLYLGLRFIIKKA
SNSRNINNDNIVREGDYIYLNIDNISDESYRVYVLVNSKEIQTQLFLAPINDDPTFYDVLQIKKYYEK
TTYNCQILCEKDTKTFGLFGIGKFVKDYGYVWDTYDNYFCISQWYLRRISEMINKLRLGCNWQFIPVD
EGWTE
Polypeptide Sequence of BoNT/X
SEQ ID NO: 24
MKLSINKFNYNDPIDGINVITMRPPRHSDKINKGKGPFKAFQVIKNIWIVPERYNFTNNTNDLNIPSE
PIMEADAIYNPNYLNTPSEKDEFLQGVIKVLERIKSKPEGEKLLELISSSIPLPLVSNGALTLSDNET
IAYQENNNIVSNLQANLVIYGPGPDIANNATYGLYSTPISNGEGTLSEVSFSPFYLKPFDESYGNYRS
LVNIVNKFVKREFAPDPASTLMHELVHVTHNLYGISNRNFYYNFDTGKIETSRQQNSLIFEELLTFGG
IDSKAISSLIIKKIIETAKNNYTTLISERLNTVTVENDLLKYIKNKIPVQGRLGNFKLDTAEFEKKLN
TILFVLNESNLAQRFSILVRKHYLKERPIDPIYVNILDDNSYSTLEGFNISSQGSNDFQGQLLESSYF
EKIESNALRAFIKICPRNGLLYNAIYRNSKNYLNNIDLEDKKTTSKTNVSYPCSLLNGCIEVENKDLF
LISNKDSLNDINLSEEKIKPETTVFFKDKLPPQDITLSNYDFTEANSIPSISQQNILERNEELYEPIR
NSLFEIKTIYVDKLTTFHFLEAQNIDESIDSSKIRVELTDSVDEALSNPNKVYSPFKNMSNTINSIET
GITSTYIFYQWLRSIVKDFSDETGKIDVIDKSSDTLAIVPYIGPLLNIGNDIRHGDFVGAIELAGITA
LLEYVPEFTIPILVGLEVIGGELAREQVEAIVNNALDKRDQKWAEVYNITKAQWWGTIHLQINTRLAH
TYKALSRQANAIKMNMEFQLANYKGNIDDKAKIKNAISETEILLNKSVEQAMKNTEKFMIKLSNSYLT
KEMIPKVQDNLKNFDLETKKTLDKFIKEKEDILGTNLSSSLRRKVSIRLKKNIAFDINDIPFSEFDDL
INQYKKEIEDYEVLNLGAEDGKIKDLSGTTSDINIGSDIELADGRENKAIKIKGSENSTIKIAMNKYL
RFSATDNFSISFWIKHPKPTNLLNKGIEYTLVENFNQRGWKISIQDSKLIWYLRDHNNSIKIVTPDYI
AFNGWNLITITNNRSKGSIVYVNGSKIEEKDISSIWNTEVDDPIIFRLKNNRDTQAFTLLDQFSIYRK
ELNQNEVVKLYNYYFNSNYIRDIWGNPLQYNKKYYLQTQDKPGKGLIREYWSSFGYDYVILSDSKTIT
FPNNIRYGALYNGSKVLIKNSKKLDGLVRNKDFIQLEIDGYNMGISADRFNEDTNYIGTTYGTTHDLT
TDFEIIQRQEKYRNYCQLKTPYNIFHKSGLMSTETSKPTFHDYRDWVYSSAWYFQNYENLNLRKHTKT
NWYFIPKDEGWDED
TeNT-UniProt P04958
SEQ ID NO: 25
MPITINNFRYSDPVNNDTIIMMEPPYCKGLDIYYKAFKITDRIWIVPERYEFGTKPEDFNPPSSLIEG
ASEYYDPNYLRTDSDKDFFLQTMVKLFNRIKNNVAGEALLDKIINAIPYLGNSYSLLDKFDTNSNSVS
FNLLEQDPSGATTKSAMLTNLIIFGPGPVLNKNEVRGIVLRVDNKNYFPCRDGFGSIMQMAFCPEYVP
TFDNVIENITSLTIGKSKYFQDPALLLMHELIHVLHGLYGMQVSSHEIIPSKQEIYMQHTYPISAEEL
FTFGGQDANLISIDIKNDLYEKTLNDYKAIANKLSQVTSCNDPNIDIDSYKQIYQQKYQFDKDSNGQY
IVNEDKFQILYNSIMYGFTEIELGKKFNIKTRLSYFSMNHDPVKIPNLLDDTIYNDTEGFNIESKDLK
SEYKGQNMRVNTNAFRNVDGSGLVSKLIGLCKKIIPPTNIRENLYNRTASLTDLGGELCIKIKNEDLT
FIAEKNSFSEEPFQDEIVSYNTKNKPLNFNYSLDKIIVDYNLQSKITLPNDRTTPVTKGIPYAPEYKS
NAASTIElHNIDDNTIYQYLYAQKSPTTLQRITMTNSVDDALINSTKIYSYFPSVISKVNQGAQGILF
LQWVRDIIDDFTNESSQKTTIDKISDVSTIVPYIGPALNIVKQGYEGNFIGALETTGVVLLLEYIPEI
TLPVIAALSIAESSTQKEKIIKTIDNFLEKRYEKWIEVYKLVKAKWLGTVNTQFQKRSYQMYRSLEYQ
VDAIKKIIDYEYKIYSGPDKEQIADEINNLKNKLEEKAKKAMININIFMRESSRSFLVNQMINEAKKQ
LLEFDTQSKNILMQYIKANSKFIGITELKKLESKINKVFSTPIPFSYSKNLDCWVDNEEDIDVILKKS
TILNLDINNDIISDISGFNSSVITYPDAQLVPGINGKAIHLVNNESSEVIVHKAMDIEYNDMFNNFTV
SFWLRVPKVSASHLEQYGTNEYSIISSMKKHSLSIGSGWSVSLKGNNLIWTLKDSAGEVRQITFRDLP
DKFMAYLANKWVFITITNDRLSSANLYINGVLMGSAEITGLGAIREDNNITLKLDRCNNNNQYVSIDK
FRIFCKALNPKEIEKLYTSYLSITFLRDFWGNPLRYDTEYYLIPVASSSKDVQLKNITDYMYLTNAPS
YTNGKLNIYYRRLYNGLKFIIKRYTPNNEIDSFVKSGDFIKLYVSYNNNEHIVGYPKDGNAFNNLDRI
LRVGYNAPGIPLYKKMEAVKLRDLKTYSVQLKLYDDKNASLGLVGTHNGQIGNDPNRDILIASNWYFN
HLKDKILGCDWYFVPTDEGWTND
Polypeptide sequence of labelled EGF TM polypeptide
SEQ ID NO: 26
*HHHHHHLAETGGSGGSGGSEFVNKQFNYKDPVNGVDIAYIKIPNAGQMQPVKAFKIHNKIWVIPERD
TFTNPEEGDLNPPPEAKQVPVSYYDSTYLSTDNEKDNYLKGVTKLFERIYSTDLGRMLLTSIVRGIPF
WGGSTIDTELKVIDTNCINVIQPDGSYRSEELNLVIIGPSADIIQFECKSFGHEVLNLTRNGYGSTQY
IRFSPDFTFGFEESLEVDTNPLLGAGKFATDPAVTLAHELIHAGHRLYGIAINPNRVFKVNTNAYYEM
SGLEVSFEELRTFGGHDAKFIDSLQENEFRLYYYKKFKDIASTLNKAKSIVGTTASLQYMKNVFKEKY
LLSEDTSGKF3VDKLKFDKLYKMLTEIYTEDNFVKFFKVLNRKTYLNFDKAVFKINIVPKVNYTIYDG
FNLRNTNLAANFNGQNTEINNMNFTKLKNFTGLFEFYKLLCVDGIITSKTKSLIEGRNKALNLQCIKV
NNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKK
VNKATEAAMFLGWVEQLVYDFTDETSEVSTTDKIADITIIIPYIGPALNIGNMLYKDDFVGALIFSGA
VILLEFIPEIAIPVLGTFALVSYIANKVLTVQTIDNALSKRNEKWDEVYKYIVTNWLAKVNTQIDLIR
KKMKEALENQAEATKAIINYQYNQYTEEEKNNIKFNIDDLSSKLNESINKAMININKFLNQCSVSYLM
NSMIPYGVKRLEDFDASLKDALLKYIYDNRGTLIGQVDRLKDKVNNTLSTDIPFQLSKYVDNQRLLST
LEGGGGSGGGGSGGGGSALDNSDPKCPLSHEGYCLNDGVCMYIGTLDRYACNCVVGYVGERCQYRDLK
LAELRGLEAGGSGGGSGLPESGK†
* = HiLyte555; † = HiLyte488
Polupeptide sequence of C. ternatea butelase 1 (plus signal peptide)
SEQ ID NO: 27
MKNPLAILFLIATVVAVVSGIRDDFLRLPSQASKFFQADDNVEGTRWAVLVAGSKGYVNYRHQADVCH
AYQILKKGGLKDENIIVFMYDDIAYNESNPHPGVIINHPYGSDVYKGVPKDYVGEDINPPNFYAVLLA
NKSALTGTGSGKVLDSGPNDHVFIYYTDHGGAGVLGMPSKPYIAASDLNDVLKKKHASGTYKSIVFYV
ESCESGSFMDGLLPEDHNIYVMGASDTGESSWVTYCPLQHPSPPPEYDVCVGDLFSVAWLEDCDVHNL
QTETFQQQYEVVKNKTIVALIEDGTHVVQYGDVGLSKQTLFVYMGTDPANDNNTFTDKNSLGTPRKAV
SQRDADLIHYWEKYRRAPEGSSRKAEAKKQLREVMAHRMHIDNSVKHIGKLLFGIEKGHKMLNNVRPA
GLPVVDDWDCFKTLIRTFETHCGSLSEYGMKHMRSFANLCNAGIRKEQMAEASAQACVSIPDNPWSSL
HAGFSV
Polypeptide sequence of C. ternatea butelase 1
(minus signal peptide)
SEQ ID NO: 28
IRDDFLRLPSQASKFFQADDNVEGTRWAVLVAGSKGYVNYRHQADVCKAYQILKKGGLKDENIIVFMY
DDIAYNESNPHPGVIINHPYGSDVYKGVPKDYVGEDINPPNFYAVLIANKSALTGTGSGKVLDSGPND
HVFIYYTDHGGAGVLGMPSKPYIAASDLNDVLKKKHASGTYKSIVFYVESCESGSMFDGLLPEDHNIY
VMGASDTGESSWVTYCPLQKPSPPPEYDVCVGDLFSVAWLEDCDVHNLQTETFQQQYEVVKNKTIVAL
IEDGTHVVQYGDVGLSKQTLFVYMGTDPANDNNTFTDKNSLGTPRKAVSQRDADLIHYWEKYRRAPEG
SSRKAEAKKQLREVMAHRMHIDNSVKHIGKLLFGIEKGHKMLNNVRPAGLPVVDDWDCFKTLIRTFET
HCGSLSEYGMKHMRSFANLCNAGIRKEQMAEASAQACVSIPDNPWSSLHAGFSV
Peptide with conjugated detectable label and sortase donor site
SEQ ID NO: 29
GGGGK†
† = HiLyte488
Peptide with conjugated detectable label and sortase acceptor site
SEQ ID NO: 30
*HHHHHHLAETGGG
* = HiLyte555
Polypeptide sequence of Staphylococcus aureus Sortase A
SEQ ID NO: 31
MKKWTNRLMTIAGVVLILVAAYLFAKPHIDNYLHDKDKDEKTEQYDKNVKEQASKDKKQQAKPQIPKD
KSKVAGYIEIPDADIKEPVYPGPATPEQLNRGVSFAEENESLDDQNISIAGKTFIDRPNYQFTNLKAA
KKGSMVYFKVGNETRKYKMTSIRDVKPTDVGVLDEQKGKDKQLTLITCDDYNEKTGVWEKRKIFVATE
VK
Polypeptide sequence of Staphylococcus aureus Sortase B
SEQ ID NO: 32
MRMKRFLTIVQILLVVTIIIFGYKIVQTYIEDKQERANYEKLQQKFQMLMSKHQEHVRPQFESLEKIN
KDIVGWIKLSGTSLNYPVLQGKTNHDYLNLDFEREHRRKGSIFMDFRNSLKNLNHNTILYGHHVGDNT
MFDVLEDYLKQSFYEKHKIIEFDNKYGKYQLQVFSAYKTTTKDNYIRTDFENDQDYQQFLDETKRKSV
INSDVNVTVKDRIMTLSTCEDAYSETTKRIVVVAKIIKVS
Polypeptide sequence of Streptococcus pneumoniae Sortase A
SEQ ID NO: 33
MEKLYIHLKNLRKVAVVMLLVFTTFYLLLMFLNQSDNQEIAKNIEKFNDSVIVAKTDNTKADIKEIEK
NIEKVRKIEGGNVERVNQLTSENEKVKENIDLNIEEEIIENSYKSLETTDNFEKLGIIEIPKIDLNLS
IFKGKPFVNTKNRQDTMLYGAVTNKKNQKMGRENYVLASHIISNSNLLFTSINQLEKGDVTTLKDSEY
SYQYTVYNNFIVSKDETWILNDIKDYSILTLYTCYDDSTKLPENRWIRAVLTDIN
Polypeptide sequence of Streptococcus pneumoniae Sortase B
SEQ ID NO: 34
MAKTKKQKRNNLLLGVVFFIGXAVMAYPLVSRLYYRVESNQQIADFDKEKATLDEADIDEPWKLAQAF
NDSLNNVVSGDPWSEEMKKKGRAEYARMLEIHERMGHVEIPAIDVDLPVYAGTAEEVLQQGAGHLEGT
SLPIGGNSTHAVITAHTGLPTAKMFTDLTKLKVGDKFYVHNIKEVMAYQVDQVKVIEPTNFDDLLIVP
GHDYVTLLTCTPYMINTHRLLVRGHRIPYVAEVEEEFIAANKLSHLYRYLFYVAVGLIVILLWIIRRL
RKKKRQSERALKALKEATKEVKVEDE
wherein X is Met or Ile.
Polypeptide sequence of Streptococcus pneumoniae Sortase C
SEQ ID NO: 35
MDNSRRSRKKGTKKKKHPLILLLIFLVGFAVAIYPLVSRYYYRIESNEVIKEFDETVSQMDKAELEER
WRLAQAFNATLKPSEILDPFTEQEKKKGVSEYANMLKVHERIGYVEIPAIDQEIPMYVGTSEDILQKG
AGLLEGASLPVGGKNTHTVITAHRGLPTAELFSQLDKMKKGDIFYLHVLDQVLAYQVDQIVTVEPNDF
EPVLIQHGEDYATLLTCTPYMINSHRLLVRGKRIPYTAPIAERMRAVRERGQFWLWLLLGAMAVILLL
LYRVYRNRRIVKGLEKQLEGRHVKD
Polypeptide sequence of Streptococcus pneumoniae Sortase D
SEQ ID NO: 36
MSRTKLRALLGYLLMLVACLIPIYCFGQMVLQSLGQVKGHATFVKSMTTEMYQEQQNHSLAYNQRIAS
QNRIVDPFLAEGYEVNYQVSDDPDAVYGYLSIPSLEIMEPVYLGADYHHLGMGLAHVDGTPLPMDGTG
IRSVIAGHRAEPSHVFFRHLDQLKVGDALYYDNGQEIVEYQMMDTEIILPSEWEKLESVSSKNIMTLI
TCDPIPTFNKRLLVNFERVAVYQKSDPQTAAVARVAFTKEGQSVSRVATSQWLYPGLVVIAFLGILFV
LWKLARLLRGK
Polypeptide sequence of Streptococcus pyogenes Sortase A
SEQ ID NO: 37
MVKKQKRRKIKSMSWARKLLIAVLLILGLALLFNKPIRNTLIARNSNKYQVTKVSKKQIKKNKEAKST
FDFQAVEPVSTESVLQAQMAAQQLPVIGGIAIPELGINLPIFKGLGNTELIYGAGTMKEEQVMGGENN
YSLASHHIFGITGSSQMLFSPLERAQNGMSIYLTDKEKIYEYIIKDVFTVAPERVDVIDDTAGLKEVT
LVTCTDIEATERIIVKGELKTEYDFDKAPADVLKAFNHSYNQVST
Polypeptide sequence of proteolytically inactive mutant BoNT/A(0)
SEQ ID NO: 38
MPFVNKQFNYKDPVNGVDIAYIKIPNAGQMQPVKAFKIHNKIWVIPERDTFTNPEEGDLNPPPEAKQV
PVSYYDSTYLSTDNSKDNYLKGVTKLFERIYSTDLGRMLLTSIVRGIPFWGGSTIDTELKVIDTNCIN
VIQPDGSYRSEELNLVIIGPSADIIQFECKSFGHEVLNLTRNGYGSTQYIRFSPDFTFGFEESLEVDT
NPLLGAGKFATDPAVTLAHQLIYAGHRLYGIAINPNRVFKVNTNAYYEMSGLEVSFEELRTFGGHDAK
FIDSLQENEFRLYYYNKFKDIASTLNKAKSIVGTTASLQYMKNVFKEKYLLSEDTSGKFSVDKLKFDK
LYKMLTEIYTEDNFVKFFKVLNRKTYLNFDKAVFKINIVPKVNYTIYDGFNLRNTNLAANFNGQNTEI
NNMNFTKLKNFTGLFEFYKLLCVRGIITSKTKSLDKGYNKALNDLCIKVNNWDLFFSPSEDNFTNDLN
KGEEITSDTNIEAAEENISLDLIQQYYLTFNFDNEPENISIENLSSDIIGQLELMPNIERFPNGKKYE
LDKYTMFHYLRAQEFEHGKSRIALTNSVNEALLNPSRVYTFFSSDYVKKVNKATEAAMFLGWVEQLVY
DFTDETSEVSTTDKIADITIIIPYIGPALNIGNMLYKDDFVGALIFSGAVILLEFIPEIAIPVLGTFA
LVSYIANKVLTVQTIDNALSKRNEKWDEVYKYIVTNWLAKVNTQIDLIRKKMKEALENQAEATKAIIN
YQYNQYTEEEKNNINFNIDDLSSKLNESINKAMININKFLNQCSVSYLMNSMIPYGVKRLEDFDASLK
DALLKYIYDNRGTLIGQVDRLKDKVNNTLSTDIPFQLSKYVDNQRLLSTFTEYIKNIINTSILNLRYE
SNHLIDLSRYASKINIGSKVNFDPIDKNQIQLFNLESSKIEVILKNAIVYNSMYENFSTSFWIRIPKY
FNSISLNNEYTIINCMENNSGWKVSLNYGEIIWTLQDTQEIKQRVVFKYSQMINISDYINRWIFVTIT
NNRLNNSKIYINGRLIDQKPISNLGNIHASNNIMFKLDGCRDTHRYIWIKYFNLFDKELNEKEIKDLY
DNQSNSGILKDFWGDYLQYDKPYYMLNLYDPNKYVDVNNVGIRGYMYLKGPRGSVMTTNIYLNSSLYR
GTKFIIKKYASGNKDNIVRNNDRVYINVVVKNKEYRLATNASQAGVEKILSALEIPDVGNLSQVVVMK
SKNDQGITNKCKMNLQDNNGNDIGFIGFHQFNNIAKLVASNWYNRQIERSSRTLGCSWEFIPVDDGWG
ERPL
Nucleotide sequence of full length proteolytically inactive mutant
BoNT/A(0) with dual-labelling SrtA sites
SEQ ID NO: 39
ATGGAGAACCTGTATTTTCAGGGCGGCGGTGGCAGCGGCGGCAGCGGCGGCAGCCCGTTTGTGAACAA
GCAGTTCAACTATAAAGATCCGGTTAATGGTGTGGATATCGCCTATATCAAAATTCCGAATGCAGGTC
AGATGCAGCCGGTTAAAGCCTTTAAAATCCATAACAAAATTTGGGTGATTCCGGAACGTGATACCTTT
ACCAATCCGGAAGAAGGTGATCTGAATCCGCCTCCGGAAGCAAAACAGGTTCCGGTTAGCTATTATGA
TAGCACCTATCTGAGCACCGATAACGAGAAAGATAACTATCTGAAAGGTGTGACCAAACTGTTTGAAC
GCATTTATAGTACCGATCTGGGTCGTATGCTGCTGACCAGCATTGTTCGTGGTATTCCGTTTTGGGGT
GGTAGCACCATTGATACCGAACTGAAAGTTATTGACACCAACTGCATTAATGTGATTCAGCCGGATGG
TAGCTATCGTAGCGAAGAACTGAATCTGGTTATTATTGGTCCGAGCGCAGATATCATTCAGTTTGAAT
GTAAAAGCTTTGGCCACGAAGTTCTGAATCTGACCCGTAATGGTTATGGTAGTACCCAGTATATTCGT
TTCAGTCCGGATTTTACCTTTGGCTTTGAAGAAAGCCTGGAAGTTGATACAAATCCGCTGTTAGGTGC
AGGTAAATTTGCAACCGATCCGGCAGTTACCCTGGCACACCAGCTGATTTATGCCGGTCATCGTCTGT
ATGGTATTGCCATTAATCCGAATCGTGTGTTCAAAGTGAATACCAACGCCTATTATGAAATGAGCGGT
CTGGAAGTGAGTTTTGAAGAACTGCGTACCTTTGGTGGTCATGATGCCAAATTTATCGATAGCCTGCA
AGAAAATGAATTTCGCCTGTACTACTATAACAAATTCAAGGATATTGCGAGCACCCTGAATAAAGCCA
AAAGCATTGTTGGCACCACCGCAAGCCTGCAGTATATGAAAAATGTGTTTAAAGAAAAATATCTGCTG
AGCGAAGATACCAGCGGTAAATTTAGCGTTGACAAACTGAAATTCGATAAACTGTACAAGATGCTGAC
CGAGATTTATACCGAAGATAACTTCGTGAAGTTTTTCAAAGTGCTGAACCGCAAAACCTACCTGAACT
TTGATAAAGCCGTGTTCAAAATCAACATCGTGCCGAAAGTGAACTATACCATCTATGATGGTTTTAAC
CTGCGCAATACCAATCTGGCAGCAAACTTTAATGGTCAGAACACCGAAATCAACAACATGAACTTTAC
CAAACTGAAGAACTTCACCGGTCTGTTCGAATTTTACAAACTGCTGTGTGTTCGTGGCATTATTACCA
GCAAAACCAAAAGTCTGGATAAAGGCTACAATAAAGCCCTGAATGATCTGTGCATTAAGGTGAATAAT
TGGGACCTGTTTTTTAGCCCGAGCGAGGATAATTTCACCAACGATCTGAACAAAGGCGAAGAAATTAC
CAGCGATACCAATATTGAAGCAGCCGAAGAAAACATTAGCCTGGATCTGATTCAGCAGTATTATCTGA
CCTTCAACTTCGATAATGAGCCGGAAAATATCAGCATTGAA&ACCTGAGCAGCGATATTATTGGCCAG
CTGGAACTGATGCCGAATATTGAACGTTTTCCGAACGGCAAAAAATACGAGCTGGATAAATACACCAT
GTTCCATTATCTGCGTGCCCAAGAATTTGAACATGGTAAAAGCCGTATTGCACTGACCAATAGCGTTA
ATGAAGCACTGCTCAACCCGAGCCGTGTTTATACCTTTTTTAGCAGCGATTACGTGAAAAAGGTTAAC
AAAGCAACCGAAGCAGCCATGTTTTTAGGTTGGGTTGAACAGCTGGTTTATGATTTCACCGATGAAAC
CAGCGAAGTTAGCACCACCGATAAAATTGCAGATATTACCATCATCATCCCGTATATCGGTCCGGCAC
TGAATATTGGCAATATGCTGTATAAAGACGATTTTGTGGGTGCCCTGATTTTTAGCGGTGCAGTTATT
CTGCTGGAATTTATTCCGGAAATTGCCATTCCGGTTCTGGGCACCTTTGCACTGGTGAGCTATATTGC
AAATAAAGTTCTGACCGTGCAGACCATCGATAATGCACTGAGCAAACGTAACGAAAAATGGGATGAAG
TGTACAAGTATATCGTGACCAATTGGCTGGCAAAAGTTAACACCCAGATTGACCTGATTCGCAAGAAG
ATGAAAGAAGCACTGGAAAATCAGGCAGAAGCAACCAAAGCCATTATCAACTATCAGTATAACCAGTA
CACCGAAGAAGAGAAAAATAACATCAACTTCAACATCGAGGATCTGTCCAGCAAACTGAACGAAAGCA
TCAACAAAGCCATGATTAACATTAACAAATTTCTGAACCAGTGCAGCGTGAGCTATCTGATGAATAGC
ATGATTCCGTATGGTGTGAAACGTCTGGAAGATTTTGATGCAAGCCTGAAAGATGCCCTGCTGAAATA
TATCTATGATAATCGTGGCACCCTGATTGGTCAGGTTGATCGTCTGAAAGATAAAGTGAACAACACCC
TGAGTACCGATATTCCTTTTCAGCTGAGCAAATATGTGGATAATCAGCGTCTGCTGTCAACCTTTACC
GAATACATTAAGAACATCATCAACACCAGCATTCTGAACCTGCGTTATGAAAGCAATCATCTGATTGA
TCTGAGCCGTTATGCCAGCAAAATCAATATAGGCAGCAAGGTTAACTTCGACCCGATTGACAAAAATC
AGATACAGCTGTTTAATCTGGAAAGCAGCAAAATTGAGGTGATCCTGAAAAACGCCATTGTGTATAAT
AGCATGTACGAGAATTTCTCGACCAGCTTTTGGATTCGTATCCCGAAATACTTTAATAGCATCAGCCT
GAACAACGAGTACACCATTATTAACTGCATGGAAAACAATAGCGGCTGGAAAGTTAGCCTGAATTATG
GCGAAATTATCTGGACCCTGCAGGATACCCAAGAAATCAAACAGCGTGTGGTTTTCAAATACAGCCAG
ATGATTAATATCAGCGACTATATCAACCGCTGGATTTTTGTGACCATTACCAATAATCGCCTGAATAA
CAGCAAGATCTATATTAACGGTCGTCTGATTGACCAGAAACCGATTAGTAATCTGGGTAATATTCATG
CGAGCAACAACATCATGTTTAAACTGGATGGTTGTCGTGATACCCATCGTTATATTTGGATCAAGTAC
TTCAACCTGTTCGATAAAGAGTTGAACGAAAAAGAAATTAAAGACCTGTATGATAACCAGAGCAACAG
CGGTATTCTGAAGGATTTTTGGGGAGATTATCTGCAGTATGACAAACCGTATTATATGCTGAATCTGT
ACGACCCGAATAAATACGTGGATGTGAATAATGTTGGCATCCGTGGTTATATGTACCTGAAAGGTCCG
CGTGGTAGCGTTATGACCACAAACATTTATCTGAATAGCAGCCTGTATCGCGGAACCAAATTCATCAT
TAAAAAGTATGCCAGCGGCAACAAGGATAATATTGTGCGTAATAATGATCGCGTGTACATTAACGTTG
TGGTGAAGAATAAAGAATATCGCCTGGCAACCAATGCAAGCCAGGCAGGCGTTGAAAAAATTCTGAGT
GCCCTGGAAATTCCGGATGTTGGTAATCTGAGCCAGGTTGTTGTGATGAAAAGCAAAAATGATCAGGG
CATCACCAACAAGTGCAAAATGAATCTGCAGGACAATAACGGCAACGATATTGGTTTTATTGGCTTCC
ACCAGTTCAACAATATTGCGAAACTGGTTGCAAGCAATTGGTATAATCGTCAGATTGAACGTAGCAGT
CGTACCCTGGGTTGTAGCTGGGAATTTATCCCTGTGGATGATGGTTGGGGTGAACGTCCGCTGGGCGG
CAGCGGCGGCGGCAGCGGCCTGCCCGAAAGCGGTGGCGGATCTGCTTGGTCTCACCCGCAGTTCGAAA
AAGGTGGTGGTTCTGGTGGTGGTTCTGGTGGTTCTGCTTGGTCTCACCCGCAGTTCGAAAAATAATGA
Polypeptide sequence of full length proteolytically inactive mutant
BoNT/A(0) with dual-labelling SrtA sites
SEQ ID NO: 40
MENLYFQGGGGSGGSGGSPFVNKQFNYKDPVNGVDIAYIKIPNAGQMQPVKAFKIHNKIWVIPERDTF
TNPEEGDLNPPPEAKQVPVSYYDSTYLSTDNEKDNYLKGVTKLFERIYSTDLGRMLLTSIVRGIPFWG
GSTIDTELKVIDTNCINVIQPDGSYRSESLNLVIIGPSADIIQFECKSFGHEVLNLTRNGYGSTQYIR
FSPDFTFGFEESLEVDTNPLLGAGKFATDPAVTLAHQLIYAGHRLYGIAINPNRVFKVNTNAYYEMSG
LEVSFEELRTFGGHDAKFIDSLQENEFRLYYYNKFKDIASTLNKAKSIVGTTASLQYMKNVFKEKYLL
SEDTSGKFSVDKLKFDKLYKMLTEIYTEDNFVKFFKVLNRKTYLNFDKAVFKINIVPKVNYTIYDGFN
LRNTNLAANFNGQNTEINNMNFTKLKNFTGLFEFYKLLCVRGIITSKTKSLDKGYNKALNDLCIKVNN
WDLFFSPSEDNFTNDLNKGEEITSDTNIEAAEENISLDLIQQYYLTFNFDNEPENISIENLSSDIIGQ
LELMPNIERFPNGKKYELDKYTMFHYLRAQEFEHGKSRIALTNSVNEALLNPSRVYTFFSSDYVKKVN
KATEAAMFLGWVEQLVYDFTDETSEVSTTBKIADITIIIPYIGPALNIGNMLYKDDFVGALIFSGAVI
LLEFIPEIAIPVLGTFALVSYIANKVLTVQTIDNALSKRNEKWDEVYKYIVTNWLAKVNTQIDLIRKK
MKEALENQAEATKAIINYQYNQYTEEEKNNINFNIDDLSSKLNESINKAMININKFLNQCSVSYLMNS
MIPYGVKRLEDFDASLKDALLKYIYDNRGTLIGQVDRLKDKVNNTLSTDIPFQLSKYVDNQRLLSTFT
EYIKNIINTSILNLRYESNHLIDLSRYASKINIGSKVNFDPIDKNQIQLFNLESSKIEVILKNAIVYN
SMYENFSTSFWIRIPKYFNSISLNNEYTIINCMENNSGWKVSLNYGEIIWTLQDTQEIKQRVVFKYSQ
MINISDYINRWIFVTITNNRLNNSKIYINGRLIDQKPISNLGNIHASNNIMFKLDGCRDTHRYIWIKY
FNLFDKELNEKEIKDLYDNQSNSGILKDFWGDYLQYDKPYYMLNLYDPNKYVDVNNVGIRGYMYLKGP
RGSVMTTNIYLNSSLYRGTKFIIKKYASGNKDNIVRNNDRVYINVVVKNKEYRLATNASQAGVEKILS
ALEIPDVGNLSQVVVMKSKNDQGITNKCKMNLQDNNGNDIGFIGFHQFNNIAKLVASNWYNRQIERSS
RTLGCSWEFIPVDDGWGERPLGGSGGGSGLPESGGGSAWSHPQFEKGGGSGGGSGGSAWSHPQFEK
Polypeptide sequence of Prochloron didemni PATG
SEQ ID NO: 41
MFSIMITIDYPFTVSLNRDIQVTSTEDYYTLQVTESDPSAWLTFATTPAMDMAFDHLKAGTTTESLVQ
TLAELGGPAAREQFALTLQQLDERGWLSYAVLPLAEAIPMVESAELNLPGNPHWMETGVTLSRFAYQH
PYEGTMVLESPLSKFRVKLLDWRASALLAQLAQPQTLGTIAPPPYLGPETAYQFLNLLWATGFLASDH
EPVSLQLWDFHNLLFHSRSRLGRHDYPGTDLNVDNWSDFPVVKPPMSDRIVPLPRPNLEALMSNDATL
TEAIETRKSVREYDDDNPITIEQLGELLYRAARVTKLLSPEERFGKLWQQNKPVFEEAGVDEGEFSHR
PYPGGGAMYELEIYPVVRLCQGLSQGVYHYDPLNHQLEQIVESKDDIFAVSGSPLASKLGPHVLLVIT
ARFGRLFRLYRSVAYALVLKHVGVLQQNLYLVATNMGLAPCAGGAGDSDAEAQVTGIDYVEESAVGEF
ILGSLASEVESDVVEGEDEIESAGVSASEVESSATKQKVALHPHDLDERIPGLADLHNQTLGDPQITI
VIIDGDPDYTLSCFEGAEVSKVFPYWHEPAEPITPEDYAAFQSIRDQGLKGKEKEEALEAVIPDTKDR
IVLNDHACHVTSTIVGQEHSPVFGIAPNCRVINMPQDAVIRGNYDDVMSPLNLARAIDLALELGANII
HCAFCRPTQTSEGEEILVQAIKKCQDNNVLIVSPTGNNSNESWCLPAVLPGTLAVGAAKVDGTPCHFS
MWGGNNTKEGILAPGEEILGAQPCTEEPVRLTGTSMAAPVMTGISALLMSLQVQQGKPVDAEAVRTAL
LKTAIPCDPEVVEEPERGLRGFVNIPGAMKVLFGQPSVTVSFAGGQATRTEHPGYATVAPASIPSPMA
ERATPAVQAATATEMVIAPSTEPANPATVEASTAFSGNVYALGTIGYDFGDEARRDTFKERMADPYDA
RQMVDYLDRNPDEARSLIWTLNLEGDVIYALDPKGPFATNVYEIFLQMLAGQLEPETSABFIERLSVP
ARRTTRTVELFSGEVMPVVNVPDPRGMYGWNVNALVDAALATVEYEEADEDSLRQGLTAFLNRVYHDL
HNLGQTSRDRALNFTVTNTFQAASTFAQAIASGRQLDTIEVNKSPYCRLNSDCWDVLLTFYDPEKGRR
SRRVFRFTLDWYVLPVTVGSIKSWSLPGKGTVSK
Polypeptide sequence of Saponaria vaccaria PCY1
SEQ ID NO: 42
MATSGFSKPLHYPPVRRDETVVDDYFGVKVADPYRWLEDPNSEETKEFVDNQEKLANSVLEECELIDK
FKQKIIDFVNFPRCGVPFRRANKYFKFYNSGLQAQNVFQMQDDLDGKPEVLYDPNLREGGRSGLSLYS
VSEDAKYFAFGIHSGLTEWVTIKILKTEDRSYLPDTLEWVKFSPAIWTHDNKGFFYCPYPPLKEGEDH
MTRSAVNQEARYHFLGTDQSEDILLWRDLENPAHHLKCQITDDGKYFLLYILDGCDDANKVYCLDLTK
LPNGLESFRGREDSAPFMKLIDSFDASYTAIANDGSVFTFQTNKDAPRKKLVRVDLNNPSVWTDLVPE
SKKDLLESAHAVNENQLILRYLSDVKHVLEIRDLESGALQHRLPIDIGSVDGITARRRDSVVFFKFTS
ILTPGIVYQCDLKNDPTQLKIFRESVVPDFDRSEFEVKQVFVPSKDGTKIPIFIAARKGISLDGSHPC
EMHGYGGFGINMMPTFSASRIVFLKHLGGVFCLANIRGGGEYGEEWHKAGFRDKKQNVFDDFISAAEY
LISSGYTKARRVAIEGGSNGGLLVAACINQRPDLFGCAEANCGVMDMLRFHKFTLGYLWTGDYGCSDK
EEEFKWLIKYSPIHNVRRPWEQPGNEETQYPATMILTADHDDRVVPLHSFKLLATMQHVLCTSLEDSP
QKNPIIARIQRKAAHYGRATMTQIAEVADRYGFMAKALEAPWID
Polypeptide sequence of Galerina marginata POPB
SEQ ID NO: 43
MSSVTWAPGNYPSTRRSDHVDTYQSASKGEVPVPDPYQWLEESTDEVDKWTTAQADLAQSYLDQNADI
QKLAEKFRASRNYAKFSAPTLLDDGHWYWFYNRGLQSQSVLYRSKEPALPDFSKGDDNVGDVFFDPNV
LAADGSAGMVLCKFSPDGKFFAYAVSHLGGDYSTIYVTSTSSPLSQASVAQGVDGRLSDEVKWFKFST
IIWTKDSKGFLYQRYPARERHEGTRSDRNAMMCYHKVGTTQEEDIIVYQDNEHPEWIYGADTSEDGKY
LYLYQFKDTSKKNLLWVAELDEDGVKSGIHWRKVVNEYAADYNIITNHGSLVYIKTNLNAPQYKVITI
DLSKDEPElRDFIPEEKDAKLAQVNCANEEYFVAIYKRNVKDEIYLYSKAGVQLTRLAPDFVGAASIA
NRQKQTHFFLTLSGFNTPGTIARYDFTAPETQRFSILRTTKVNELDPDDFESTQVWYESKDGTKIPMF
IVRHKSTKFDGTAAAIQYGYGGFATSADPFFSPIILTFLQTYGAIFAVPSIRGGGEFGEEWHKGGRRE
TKVNTFDDFIAAAQFLVKNKYAAPGKVAINGASNGGLLVMGSIVRAPEGTFGAAVPEGGYADLLKFHK
FTGGQAWISEYGNPSIPEEFDYIYPLSPVHNVRTDKVMPATLITVNIGDGRVVPMHSFKFIATLQHNV
PQNPHPLLIKIDKSWLGHGMGKPTDKNVKDAADKWGFIARALGLELKTVE
Polypeptide sequence of Oldenlandia affinis Butelase homologue
OaAEP1b (plus signal peptide)
SEQ ID NO: 44
MVRYLAGAVLLLVVLSVAAAVSGARDGDYLHLPSEVSRFFRPQETNDDHGEDSVGTRWAVLIAGSKGY
ANYRHQAGVCHAYQILKRGrGLKDENIVVFMYDDIAYNESNPRPGVIINSPHGSDVYAGVPKDYTGEE
VNAKNFLAAILGNKSAITGGSGKVVDSGPNDHIFIYYTDHGAAGVIGMPSKPYLYADELNDALKKKHA
SGTYKSLVFYLEACESGSMFEGILPEDLNIYALTSTNTTESSWCYYCPAQENPPPPEYWVCLGDLFSV
AWLEDSDVQNSWYETLNQQYHHVDKRISHASHATQYGNLKLGEEGLFVYMGSNPANDNYTSLDGNALT
PSSIVVNQRDADLLHLWEKFRKAPEGSARKEVAQTQIFKAMSKRVHIDSSIKLIGKLLFGIEKCTEIL
NAVRPAGQPLVDDWACLRSLVGTFETHCGSLSEYGMRHTRTIANICNAGISEEQMAEAASQACASIP
Polypeptide sequence of Oldenlandia affinis Butelase homologue
OaAEP1b (minus signal peptide)
SEQ ID NO: 45
ARDGDYLHLPSEVSRFFRPQETNDDHGEDSVGTRWAVLIAGSKGYANYRHQAGVCHAYQILKRGGLKD
ENIVVFMYDDIAYNESNPRPGVIINSPHGSDVYAGVPKDYTGEEVNAKNFLAAILGNKSAITGGSGKV
VDSGPNDHIFIYYTDHGAAGVIGMPSKPYLYADELNDALKKKHASGTYKSLVFYLEACESGSMFEGIL
PEDLNIYALTSTNTTESSWCYYCPAQENPPPPEYNVCLGDLFSVAWLEDSDVQNSWYETLNQQYHHVD
KRISHASHATQYGNLKLGEEGLFVYMGSNPANDNYTSLDGNALTPSSIVVNQRDADLLHLWEKFRKAP
EGSARKEVAQTQIFKAMSHRVHIDSSIKLIGKLLFGIEKCTEILNAVRPAGQPLVDDWACLRSLVGTF
ETHCGSLSEYGMRHTRTIANICNAGISEEQMAEAASQACASIP

### EXAMPLES

### Example 1

Design of Texas Red, eGFP, SNAP and SrtA-Mediated Single and Dual Labelled EGF-Liganded Polypeptide

Several strategies for the labelling of polypeptides were attempted. The aim was to obtain a labelled version of the polypeptide which did not affect its structural characteristics and its ability to traffic into cells and cleave SNARE proteins effectively and in a similar manner to the unlabelled version.

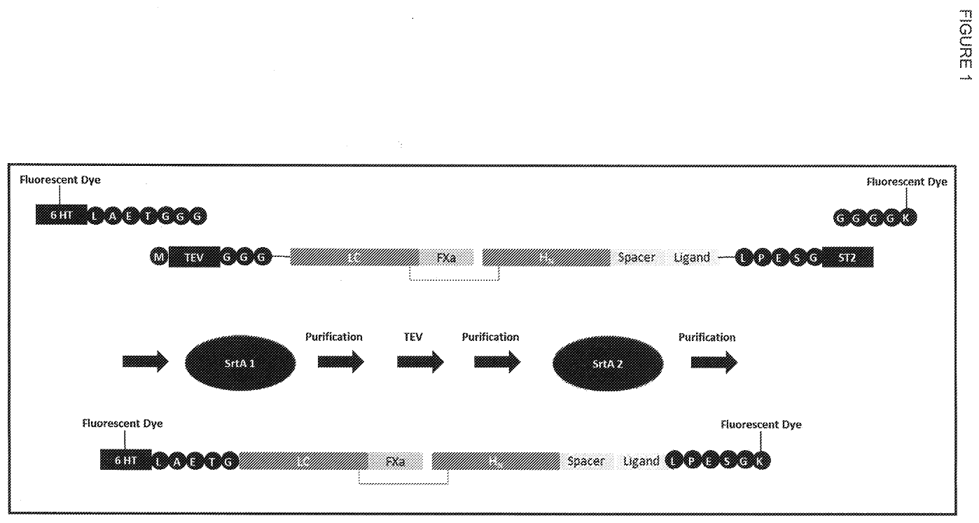

4 different labelling strategies of an EGF-liganded polypeptide (Fonfria, E., S. Donald and V. A. Cadd (2016). “Botulinum neurotoxin A and an engineered derivate targeted secretion inhibitor (TSI) A enter cells via different vesicular compartments.” J Recept Signal Transduct Res 36(1): 79-88) were attempted. Following cloning, when necessary, the polypeptide was recombinantly expressed and purified using standard procedures, as previously published (Masuyer, G., M. Beard, V. A. Cadd, J. A. Chaddock and K. R. Acharya (2011). “Structure and activity of a functional derivative of Clostridium botulinum neurotoxin B.” J Struct Biol 174(1): 52-57, Somm, E., N. Bonnet, A. Martinez, P. M. Marks, V. A. Cadd, M. Elliott, A. Toulotte, S. L. Ferrari, R. Rizzoli, P. S. Huppi, E. Harper, S. Melmed, R. Jones and M. L. Aubert (2012). “A botulinum toxin-derived targeted secretion inhibitor downregulates the GH/IGF1 axis.” J Clin Invest 122(9): 3295-3306). Briefly, the polypeptide was expressed recombinantly in E. coli competent bacteria. The expressed polypeptide was purified using an affinity column followed by anion exchange chromatography, enzymatic activation to generate a di-chain complex and finally a polishing step using hydrophobic interaction.
1. Unmodified EGF-liganded polypeptide, purified as described above was labelled using the Texas Red-X Protein Labelling Kit (Thermo Fisher Scientific) according to the manufacturer's protocol. Successful labelling of the protein was confirmed by confocal microscopy and live imaging. The nucleotide and polypeptide sequences for the polypeptide used for labelling are shown as SEQ ID NOs: 5 and 6, respectively.
2. EGF-liganded polypeptide was tagged at the N-terminal with an enhanced green fluorescent protein (eGFP) by standard cloning procedures. The nucleotide and polypeptide sequences are shown as SEQ ID NOs: 9 and 10, respectively. Protein expression and purification was performed as indicated above. After expression, purification of the eGFP-tagged EGF-liganded polypeptide was attempted unsuccessfully.
3. EGF-liganded polypeptide was tagged at the N-terminal with a SNAP-tag substrate (New England Biolabs) by standard cloning procedures. The nucleotide and polypeptide sequences are shown as SEQ ID NOs: 11 and 12, respectively. Expression and purification of this protein was successful. Labelling of the SNAP-tagged EGF-liganded polypeptide was performed using SNAP-Surface 594 fluorescent substrate (New England Biolabs) according to the manufacturer's protocol. Successful labelling of the protein was confirmed by confocal microscopy and live imaging.
4. Attempts were also made to generate polypeptides containing non-natural amino acids for site-specific labelling. However, these attempts were unsuccessful due to expression and/or purification difficulties.
5. EGF-liganded polypeptide (i.e. a polypeptide having an EGF TM) was tagged with two different Sortase A (SrtA) recognition sites, one at the N-terminus and one at the C-terminus. The use of SrtA allowed conjugation of two fluorophores of different colours on the same protein. The polypeptide was constructed as illustrated in FIG. 1. Two mutated versions of SrtA (Dorr, B. M., H. O. Ham, C. An, E. L. Chaikof and D. R. Liu (2014). “Reprogramming the specificity of sortase enzymes.” Proc Natl Acad Sci USA 111(37): 13343-13348) were chosen (SEQ ID NOs: 14 and 16). These have been shown to be 100% specific for their respective recognition sites. The EGF-liganded polypeptide was cloned with the LPESG recognition site of the first SrtA at the C-terminal, followed by a double StrepTag recognition site (IBA-lifesciences) which allows the initial affinity-mediated purification of the protein. The nucleotide and polypeptide sequences are shown as SEQ ID NOs: 1 and 2, respectively. Separately, a peptide containing a stretch of glycine residues conjugated to a fluorophore of choice was obtained (Eurogentec). The sequence of this peptide was: GGGGK(HF488) (SEQ ID NO: 29). During the SrtA-mediated reaction, the glycine of the LPESG site was cleaved by SrtA (SEQ ID NO: 14) and the stretch of glycines present on the fluorescent peptide recognized by SrtA and used to mediate the conjugation between the polypeptide and the peptide. This generated a fluorescently single-labelled EGF-liganded polypeptide. To note is the fact that the labelled polypeptide no longer possessed the StrepTag and a reverse affinity-mediated purification step was used to select the labelled portion of the polypeptide. For dual-labelling the EGF-liganded polypeptide, a stretch of 3 glycine residues was cloned at the N-terminal site of the polypeptide following the starting codon and a Tobacco Etch Virus (TEV) cleavage recognition site. The TEV site was introduced to help protect the stretch of glycine residues from protein circularization during the initial C-terminal SrtA reaction detailed above. Separately, a peptide containing the LAETG recognition site conjugated to a fluorophore of choice was obtained (Eurogentec). The sequence of this peptide was: HiLyte Fluor™ 555-HHHHHHLAETGGG (SEQ ID NO: 30). In addition, a 6 His-Tag (6HT) was positioned before the LAETG site for ease of protein purification following SrtA reaction (SEQ ID NO: 16). The SrtA reaction was conducted similarly to the C-terminal site and the final dual-labelled EGF-liganded protein was purified using a His affinity purification step. Successful single- and dual-labelling of the protein was confirmed by SDS-PAGE gel electrophoresis, confocal microscopy and live imaging.

Sortase A (SrtA) proteins possessing a C-terminal His Tag were expressed in competent E. coli bacteria and purified using an affinity capture column.

Sortase conjugation of the polypeptide and the fluorescent peptides was performed overnight at 4° C. using a ratio of 1 to 2 to 20 equivalents of polypeptide to SrtA to fluorescent peptide, respectively.

In the present Example, the EGF-liganded polypeptide was conjugated with a HiLyte 555 fluorophore at the C-terminal translocation-ligand portion and a HiLyte 488 fluorophore at the N-terminal light chain portion. The expression of the polypeptide containing the SrtA recognition sites and the two variants of SrtA was successful. Advantageously, by generating a polypeptide capable of being labelled with two different colour fluorophores, the trafficking mechanisms of both the light-chain (containing the non-cytotoxic protease) and the translocation-ligand portions of the protein could be visualised.

### Example 2

### Design of SrtA-Mediated Dual Labelled Nociceptin-Liganded Polypeptide

A polypeptide possessing a nociceptin ligand TM (nociceptin-liganded polypeptide) was generated for dual fluorescent-labelling using the strategy used for the EGF-liganded polypeptide. The design, purification and fluorescent peptides used for the dual-labelling of this polypeptide were exactly the same as for the EGF-liganded polypeptide. Successful dual-labelling of the polypeptide was confirmed by SDS-PAGE gel electrophoresis, confocal microscopy and live imaging. The nucleotide and polypeptide sequences for the polypeptide containing the sortase sites are shown as SEQ ID NOs: 3 and 4, respectively.

Validation of the Labelled Proteins Using SNAP25 Cleavage Assay

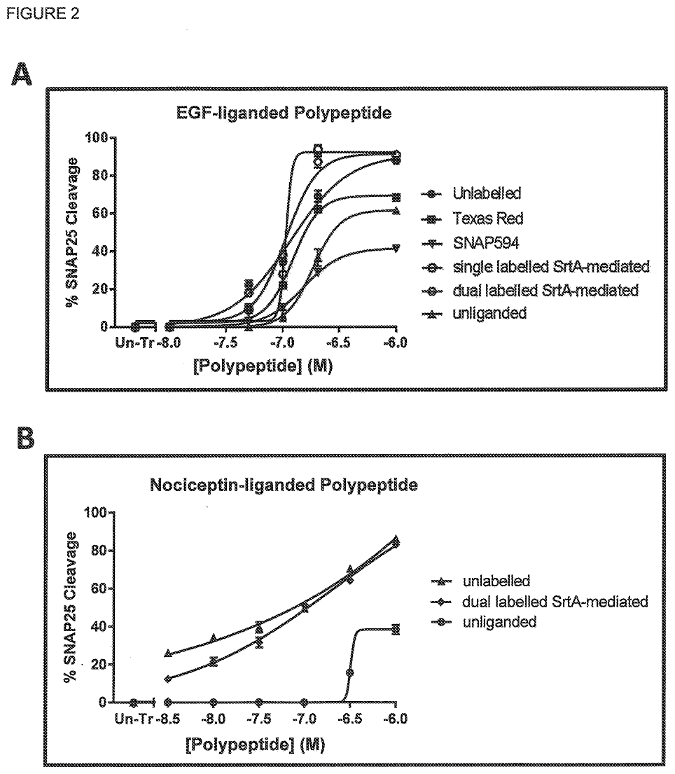

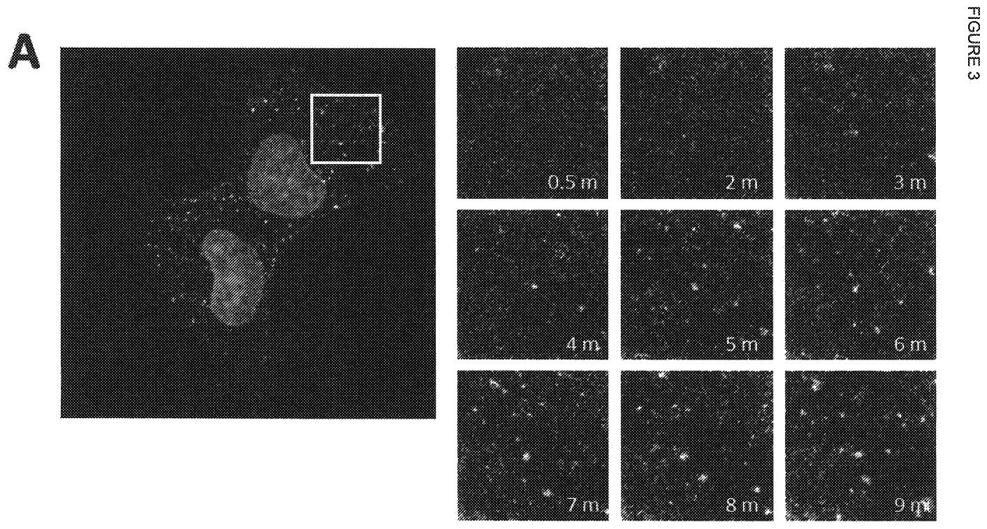

In order to determine that labelling of the liganded polypeptides does not affect their ability to bind to their respective receptors, trafficking into cells and translocation, a SNAP25 cleavage assay was performed to determine the relative potency of the labelled polypeptides compared to the unlabelled versions. A similar potency profile would suggest that the labelled polypeptide is trafficked similarly to the unlabelled version. The SNAP25 cleavage assay was performed as described previously (Fonfria, E., S. Donald and V. A. Cadd (2016). “Botulinum neurotoxin A and an engineered derivate targeted secretion inhibitor (TSI) A enter cells via different vesicular compartments.” J Recept Signal Transduct Res 36(1): 79-88). Briefly, cortical neurons were treated with 3-1000 nM of each labelled and unlabelled protein for 24 hours. Following treatment, cells were harvested in NuPAGE lysis buffer (Thermo Fischer Scientific) supplemented with 0.1M dithiothreitol and 250 units/ml benzonase (Sigma). Lysates were separated by SDS-PAGE and subjected to Western blotting using primary antibodies against SNAP-25 (Sigma). These antibodies enable recognition of both the cleaved and uncleaved portion of SNAP25. Relative potency was determined by the proportion of cleaved SNAP25 versus uncleaved SNAP25 (FIG. 2). FIG. 2A shows the dose response potency of the EGF-liganded polypeptide. In comparison to the unlabelled polypeptide, the Texas Red and SNAP594 labelled versions showed a strong reduction in potency with values similar to the unliganded control polypeptide. In contrast, the SrtA-mediated single and dual-labelled polypeptides showed similar potencies to the unlabelled version demonstrating that this labelling strategy does not affect the protein architecture and its cellular trafficking mechanisms. Similarly, dual-labelling of the nociception-liganded polypeptide did not affect its potency in cortical neurons (FIG. 2B) compared to the unlabelled control polypeptide.

In summary, simple and straightforward tagging techniques such as non-site specific labelling using a Texas Red dye and a SNAP Tag, site specific version were initially trialled. However, although these labelling strategies were successful they were shown to affect the potency of the polypeptides when compared to the unlabelled counterpart suggesting that the addition of several fluorescent molecules, in the case of Texas Red or a SNAP tag affected the trafficking properties of the labelled polypeptide. An attempt at generating an eGFP-tagged EGF-liganded polypeptide was unsuccessful due to the lack of expression of the tagged protein. In stark contrast SNAP25 cleavage assays confirm that the addition of the two fluorophores on the EGF-liganded and nociception-liganded polypeptides did not affect their potencies suggesting that the mechanisms of actions of the labelled polypeptides are similar to their unlabelled counterparts. This was surprising in view of the negative impact SNAP and Texas Red labelling had on potency.

### Example 3

### Visualization of a Dual-Labelled EGF-Liganded Polypeptide in Immortalized Cell Lines

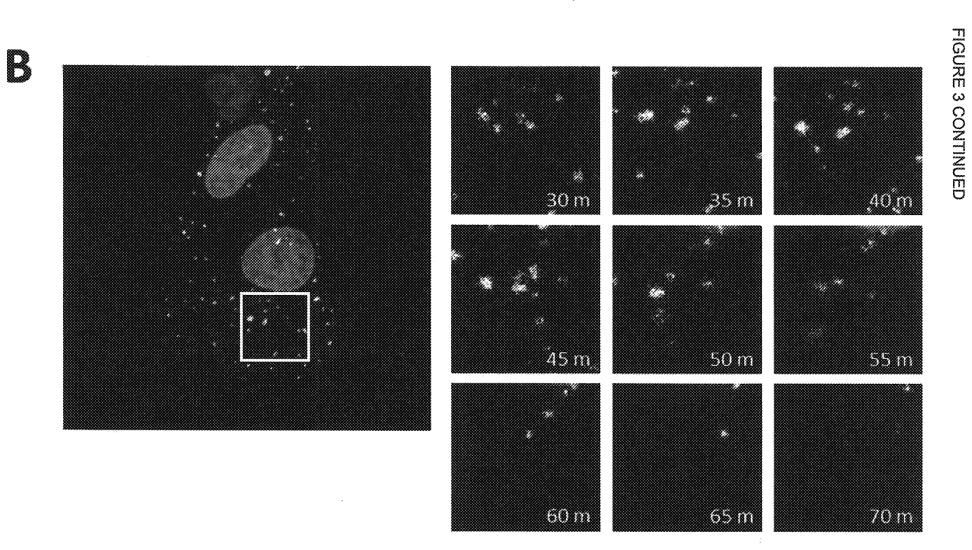

The dual-labelling SrtA-mediated technique was chosen as an optimal strategy for the labelling of polypeptides of the invention. In order to visualize the labelled polypeptide in mammalian cells, 3D live confocal microscopy was performed. Human adenocarcinoma lung cells (A549) were treated with 50 nM dual-labelled EGF-liganded polypeptide and imaged continuously over time using a Zeiss 880 confocal microscope equipped with AiryScan (Zeiss). For these experiments, the EGF-liganded polypeptide was labelled at the N-terminal with a HiLyte 555 fluorophore (AnaSpec) and at the C-terminal with a HiLyte 488 fluorophore (AnaSpec). FIG. 3 shows snapshot images of the dual-coloured agglomerates formed by the EGF-liganded polypeptide during internalization in A549 cells. From FIG. 3A it can be seen that the agglomerates appeared 3 minutes after addition of the polypeptide to the cells and their size and the amount increased over time. In FIG. 3B, the disappearance of the fluorescent agglomerate is shown over time with a total disappearance at 65 minutes after addition of the polypeptide.

The live imaging performed using the dual-labelled EGF-liganded polypeptide clearly validated the labelling technique and the ability to monitor live internalisation and trafficking of the labelled polypeptides.

Having demonstrated that sortase-labelling is advantageous and does not affect potency, this can now be applied to other clostridial neurotoxins, including BoNT serotypes (and derivatives).

### Example 4

Design of SrtA-Mediated Dual-Labelled BoNT/A Polypeptide

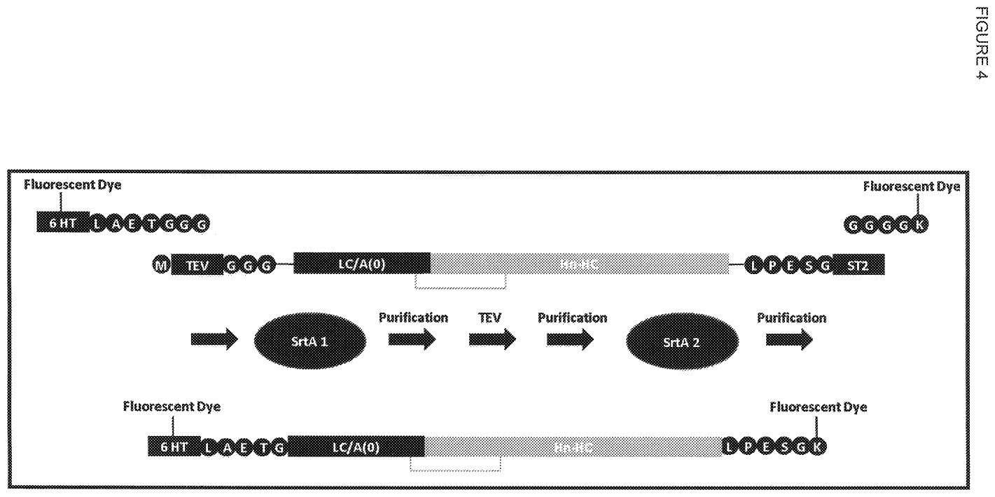

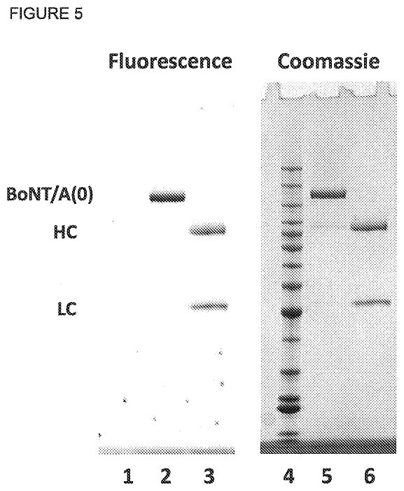

Full length proteolytically inactive mutant BoNT/A(0) (SEQ ID NO: 38) was modified to allow for dual fluorescent-labelling using sortase (see FIG. 4). The dual-labelled polypeptide sequence is shown as SEQ ID NO: 40, while the nucleotide sequence encoding said polypeptide is shown as SEQ ID NO: 39. The design, purification and fluorescent peptides used for the dual-labelling of SEQ ID NO: 40 were the same as for the EGF-liganded polypeptide in Example 1. Successful dual-labelling of the polypeptide was confirmed by SDS-PAGE (FIG. 5). In more detail, by using Coomassie staining, both bands representing the L-chain and H-chain domains of the polypeptide could be visualised, while exposure of the gel to UV light demonstrated (by way of fluorescence) the successful labelling of both the L-chain and H-chain.

### Example 5

Visualization of a Single-Labelled BoNT/A(0) Polypeptide in Primary Cortical Neurons

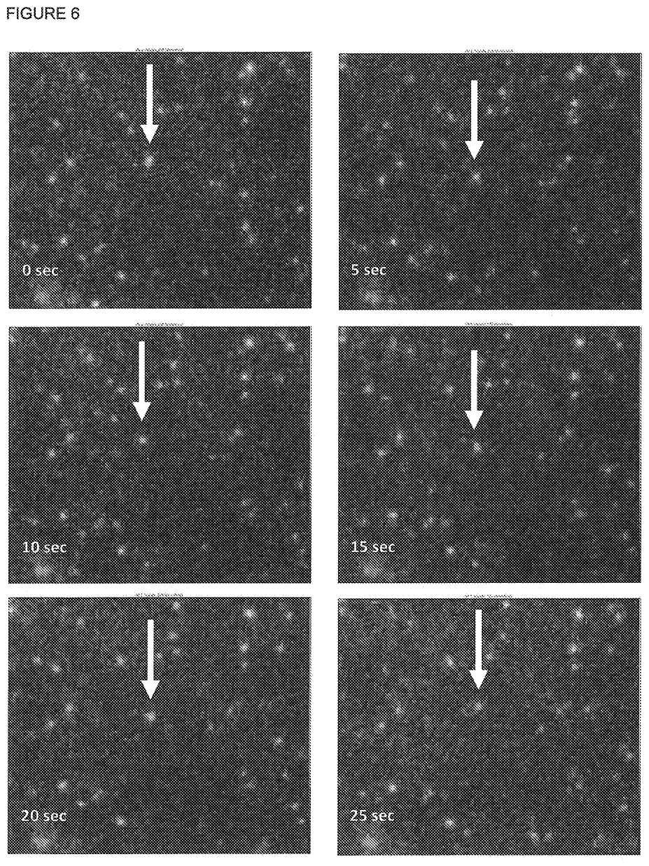

In order to visualize a labelled BoNT/A(0) polypeptide in primary neuronal cells, single molecule live TIRF microscopy was performed in neurons treated therewith. Primary cortical neurons were treated with 1 nM single-labelled BoNT/A(0) polypeptide and imaged continuously over time using a custom made single molecule TIRF microscope. For these experiments, the BoNT/A(0) polypeptide was labelled at the N-terminal with either a HiLyte 555 or HiLyte 488 fluorophore (AnaSpec). FIG. 6 shows timelapse images of the single-coloured molecule of BoNT/A(0) being trafficked into primary cortical neurons. From FIG. 6 it can be seen that the single BoNT/A(0) molecule (white arrow) moves rapidly within the chosen neuronal region. The single molecule live TIRF imaging of a single-labelled BoNT/A(0) polypeptide clearly demonstrates that single molecules of BoNT/A(0) trafficking into neurons can be visualized with specialized, high resolution microscopy techniques.

Having demonstrated that single-labelling of BoNT/A(0) can be visualised at a single molecule level in primary neurons, this method can now be applied to other clostridial neurotoxin serotypes and derivatives, including those having non-cytotoxic protease activity.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A method for preparing a labelled polypeptide, the method comprising:
a. providing a polypeptide comprising:
i. a sortase acceptor site or a sortase donor site;
ii. a non-cytotoxic protease or a proteolytically inactive mutant thereof;
iii. a Targeting Moiety (TM) that is capable of binding to a Binding Site on a target cell; and
iv. a translocation domain;
b. incubating the polypeptide with:
a sortase; and
a labelled substrate comprising a sortase donor site or a sortase acceptor site, respectively, and a conjugated detectable label;
wherein the sortase catalyses:
conjugation between an amino acid of the sortase acceptor site of the polypeptide and an amino acid of the sortase donor site of the labelled substrate; or
conjugation between an amino acid of the sortase acceptor site of the labelled substrate and an amino acid of the sortase donor site of the polypeptide;
thereby labelling the polypeptide; and
c. obtaining the labelled polypeptide.

2. A polypeptide for labelling using a sortase, the polypeptide comprising:
i. a sortase acceptor or donor site;
ii. a non-cytotoxic protease that is capable of cleaving a protein of the exocytic fusion apparatus in a target cell or a proteolytically inactive mutant thereof;
iii. a Targeting Moiety (TM) that is capable of binding to a Binding Site on a target cell; and
iv. a translocation domain that is capable of translocating the non-cytotoxic protease from within an endosome, across the endosomal membrane and into the cytosol of the target cell;
wherein when the polypeptide comprises a sortase donor site, the sortase donor site is located at an N-terminus of the polypeptide, and wherein when the sortase donor site comprises Gₙ or Aₙ, n is at least 2; and
wherein the N-terminal residue of the donor site is the N-terminal residue of the polypeptide; or
wherein the polypeptide comprises one or more amino acid residues N-terminal to the sortase donor site and a cleavable site, which when cleaved exposes the N-terminus of the sortase donor site.

3. The method according to claim 1 or polypeptide according to claim 2, wherein the sortase acceptor or donor site is located C-terminal to the TM or wherein the sortase acceptor or donor site is located N-terminal to the non-cytotoxic protease or proteolytically inactive mutant thereof.

4. The method or polypeptide according to any one of the preceding claims, wherein:
the sortase acceptor site comprises (or consists of) L(A/P/S)X(T/S/A/C)(G/A), NPQTN, YPRTG, IPQTG, VPDTG, or LPXTGS, wherein X is any amino acid, and/or wherein the sortase donor site comprises (or consists of) Gₙ or Aₙ, wherein n is at least 1.

5. The method or polypeptide according to any one of the preceding claims, wherein:
the sortase acceptor site comprises (or consists of) L(A/P/S)X(T/S/A/C)G, wherein X is any amino acid, NPQTN, YPRTG, IPQTG, VPDTG, or LPXTGS, wherein X is any amino acid, and/or wherein the sortase donor site comprises (or consists of) Gₙ, wherein n is at least 1.

6. The method or polypeptide according to any one of the preceding claims, wherein the sortase is Sortase A (SrtA).

7. The method or polypeptide according to any one of the preceding claims, wherein the polypeptide comprises: at least two sortase acceptor sites; at least two sortase donor sites; or at least one sortase acceptor site and at least one sortase donor site.

8. The method or polypeptide according to claim 7, wherein the at least two sites are different, preferably wherein the at least two sites have different amino acid sequences.

9. The method or polypeptide according to claim 7 or 8, wherein:
a first sortase acceptor or donor site is located C-terminal to the TM and a second sortase acceptor or donor site is located N-terminal to the non-cytotoxic protease or proteolytically inactive mutant thereof; or
a first sortase acceptor or donor site is located N-terminal to the non-cytotoxic protease or proteolytically inactive mutant thereof and a second sortase acceptor or donor site is located C-terminal to the TM.

10. The method or polypeptide according to any one of the proceeding claims, wherein the polypeptide comprises a polypeptide sequence having at least 70% sequence identity to SEQ ID NO: 2, 4 or 40.

11. The method or polypeptide according to any one of the proceeding claims, wherein the polypeptide comprises a polypeptide sequence having at least 80% sequence identity to SEQ ID NO: 2, 4 or 40.

12. The method or polypeptide according to any one of the proceeding claims, wherein the polypeptide comprises a polypeptide sequence having at least 90% sequence identity to SEQ ID NO: 2, 4 or 40.

13. The method or polypeptide according to any one of the proceeding claims, wherein the polypeptide comprises (preferably consists of) a polypeptide sequence shown as SEQ ID NO: 2, 4 or 40.

14. A labelled polypeptide, the polypeptide comprising:
i. a detectable label conjugated to the polypeptide;
ii. an amino acid sequence that comprises L(A/P/S)X(T/S/A/C)Gₙ, wherein X is any amino acid and n is at least 1, L(A/P/S)X(T/S/A/C)Aₙ, wherein X is any amino acid and n is at least 1, NPQTN, YPRTG, IPQTG, VPDTG, LPXTGS, wherein X is any amino acid, NPKTG, XPETG, LGATG, IPNTG, IPETG, NSKTA, NPQTG, NAKTN, NPQSS, LPXTX, wherein X is any amino acid, NPX₁TX₂, wherein X, is Lys or Gln and X₂ is Asn, Asp or Gly, X₁PX₂X₃G, wherein X₁ is Leu, Ile, Val or Met, X₂ is any amino acid and X₃ is Ser, Thr or Ala, LPEX₁G, wherein X, is Ala, Cys or Ser, LPXS, LAXT, MPXT, MPXTG, LAXS, NPXT, NPXTG, NAXT, NAXTG, NAXS, NAXSG, LPXP, LPXPG, wherein X is any amino acid, LRXTGₙ or LPAXGₙ, wherein X is any amino acid and n is at least 1;
iii. a non-cytotoxic protease or a proteolytically inactive mutant thereof;
iv. a Targeting Moiety (TM) that is capable of binding to a Binding Site on a target cell; and
v. a translocation domain.

15. The labelled polypeptide according to claim 14, wherein the amino acid sequence that comprises L(A/P/S)X(T/S/A/C)Gₙ, L(A/P/S)X(T/S/A/C)Aₙ, NPQTN, YPRTG, IPQTG, VPDTG, LPXTGS, NPKTG, XPETG, LGATG, IPNTG, IPETG, NSKTA, NPQTG, NAKTN, NPQSS, LPXTX, NPX₁TX₂, X₁PX₂X₃G, LPEX₁G, LPXS, LAXT, MPXT, MPXTG, LAXS, NPXT, NPXTG, NAXT, NAXTG, NAXS, NAXSG, LPXP, LPXPG, LRXTGₙ, or LPAXGₙ wherein X is any amino acid and n is at least 1 is located C-terminal to the TM or wherein the an amino acid sequence that comprises L(A/P/S)X(T/S/A/C)Gₙ, L(A/P/S)X(T/S/A/C)Aₙ, NPQTN, YPRTG, IPQTG, VPDTG, LPXTGS, NPKTG, XPETG, LGATG, IPNTG, IPETG, NSKTA, NPQTG, NAKTN, NPQSS, LPXTX, NPX₁TX₂, X₁PX₂X₃G, LPEX₁G, LPXS, LAXT, MPXT, MPXTG, LAXS, NPXT, NPXTG, NAXT, NAXTG, NAXS, NAXSG, LPXP, LPXPG, wherein X is any amino acid, LRXTGₙ, or LPAXGₙ wherein X is any amino acid and n is at least 1 is located N-terminal to the non-cytotoxic protease or proteolytically inactive mutant thereof.

16. The labelled polypeptide according to claim 14 or 15 comprising a further detectable label conjugated to the polypeptide and a further amino acid sequence that comprises L(A/P/S)X(T/S/A/C)Gₙ, wherein X is any amino acid and n is at least 1, L(A/P/S)X(T/S/A/C)Aₙ, wherein X is any amino acid and n is at least 1, NPQTN, YPRTG, IPQTG, VPDTG, LPXTGS, wherein X is any amino acid, NPKTG, XPETG, LGATG, IPNTG, IPETG, NSKTA, NPQTG, NAKTN, NPQSS, LPXTX, NPX₁TX₂, X₁PX₂X₃G, LPEX₁G, LPXS, LAXT, MPXT, MPXTG, LAXS, NPXT, NPXTG, NAXT, NAXTG, NAXS, NAXSG, LPXP, LPXPG, LRXTGₙ or LPAXGₙ.

17. The labelled polypeptide according to claim 16, wherein the (first) amino acid sequence is different to the further (second) amino acid sequence.

18. The labelled polypeptide according to claim 16 or 17, wherein:
the (first) amino acid sequence is located C-terminal to the TM and the further (second) amino acid sequence is located N-terminal to the non-cytotoxic protease or proteolytically inactive mutant thereof; or
the (first) amino acid sequence is located N-terminal to the non-cytotoxic protease or proteolytically inactive mutant thereof and the further (second) amino acid sequence is located C-terminal to the TM.

19. The labelled polypeptide according to any one of claims 14-18, wherein the polypeptide comprises a polypeptide sequence having at least 70% sequence identity to SEQ ID NO: 2, 4, 26 or 40.

20. The labelled polypeptide according to any one of claims 14-19, wherein the polypeptide comprises a polypeptide sequence having at least 80% sequence identity to SEQ ID NO: 2, 4, 26 or 40.

21. The labelled polypeptide according to any one of claims 14-20, wherein the polypeptide comprises a polypeptide sequence having at least 90% sequence identity to SEQ ID NO: 2, 4, 26 or 40.

22. The labelled polypeptide according to any one of claims 14-21, wherein the polypeptide comprises (preferably consists of) a polypeptide sequence shown as SEQ ID NO: 26.

23. The method, polypeptide or labelled polypeptide according to any one of the preceding claims, wherein the non-cytotoxic protease comprises a clostridial neurotoxin L-chain.

24. The method, polypeptide or labelled polypeptide according to any one of the preceding claims, wherein the translocation domain comprises a clostridial neurotoxin translocation domain.

25. The method, polypeptide or labelled polypeptide according to any one of the preceding claims, wherein the polypeptide lacks a functional H_{C} domain of a clostridial neurotoxin.

26. The method, polypeptide or labelled polypeptide according to any one of claims 1-24, wherein the TM is a clostridial neurotoxin H_{C} peptide.

27. The method, polypeptide or labelled polypeptide according to any one of claims 1-24 or 26, wherein the polypeptide is a clostridial neurotoxin.

28. The method, polypeptide or labelled polypeptide according to any one of claims 1-24 or 26-27, wherein the polypeptide is a botulinum neurotoxin (BoNT).

29. The method, polypeptide or labelled polypeptide according to any one of the preceding claims, wherein the polypeptide comprises a botulinum neurotoxin L-chain or proteolytically inactive mutant thereof.

30. The method, polypeptide or labelled polypeptide according to any one of claims 1-24 or 26-29, wherein the polypeptide comprises of a botulinum neurotoxin H-chain.

31. The method, polypeptide or labelled polypeptide according to any one of claims 1-24 or 26-30, wherein the polypeptide is selected from: BoNT/A, BoNT/B, BoNT/C, BoNT/D, BoNT/E, BoNT/F, BoNT/G, BoNT/X or TeNT.

32. A labelled polypeptide obtainable by the method according to any one of claim 1 or 3-13 or 23-31.

33. The method or labelled polypeptide according to any one of claim 1 or 3-32, wherein the labelled polypeptide does not exhibit reduced potency when compared to an equivalent unlabelled polypeptide.

34. The method or labelled polypeptide according to any one of claim 1 or 3-33, wherein the labelled polypeptide demonstrates similar cell binding, translocation, and SNARE protein cleavage when compared to an equivalent unlabelled polypeptide.

35. The method or labelled polypeptide according to any one of claim 1 or 3-34, wherein the labelled polypeptide demonstrates improved cell binding, translocation, and/or SNARE protein cleavage when compared to an equivalent unlabelled polypeptide.

36. The method or labelled polypeptide according to any one of claim 1 or 3-35, wherein the labelled polypeptide demonstrates improved cell binding, translocation, and SNARE protein cleavage when compared to an equivalent unlabelled polypeptide.

37. A method for assaying a polypeptide, the method comprising:
a. contacting a target cell with the labelled polypeptide according to any one of claims 14-36; and
b. detecting the detectable label.

38. A nucleic acid encoding the polypeptide according to any one of claims 2-13 or 23-31.

39. The nucleic acid according to claim 38, wherein the nucleic acid comprises a nucleic acid sequence having at least 70% sequence identity to SEQ ID NO: 1, 3 or 39.

40. The nucleic acid according to claim 38 or 39, wherein the nucleic acid comprises a nucleic acid sequence having at least 80% sequence identity to SEQ ID NO: 1, 3 or 39.

41. The nucleic acid according to any one of claims 38-40, wherein the nucleic acid comprises a nucleic acid sequence having at least 90% sequence identity to SEQ ID NO: 1, 3 or 39.

42. The nucleic acid according to any one of claims 38-41, wherein the nucleic acid comprises (preferably consists of) a nucleic acid sequence shown as SEQ ID NO: 1, 3 or 39.

43. A method for manufacturing a polypeptide for labelling using a sortase, the method comprising:
a. providing a nucleic acid sequence encoding a polypeptide, wherein the polypeptide comprises:
i. a non-cytotoxic protease or a proteolytically inactive mutant thereof;
ii. a Targeting Moiety (TM) that is capable of binding to a Binding Site on a target cell; and
iii. a translocation domain; and
b. introducing a sortase acceptor or donor site into said nucleic acid, thereby producing a modified nucleic acid that encodes a polypeptide comprising a sortase acceptor or donor site; and
c. optionally expressing the modified nucleic acid in a host cell; and
d. optionally obtaining the expressed polypeptide.

44. The method according to claim 43, wherein the nucleic acid of step a. comprises a nucleic acid sequence having at least 70% sequence identity to SEQ ID NO: 5 or 7.

45. The method according to claim 43 or 44, wherein the nucleic acid of step a. comprises a nucleic acid sequence having at least 80% sequence identity to SEQ ID NO: 5 or 7.

46. The method according to any one of claims 43-45, wherein the nucleic acid of step a. comprises a nucleic acid sequence having at least 90% sequence identity to SEQ ID NO: 5 or 7.

47. The method according to any one of claims 43-46, wherein the nucleic acid of step a. comprises (preferably consists of) a nucleic acid sequence shown as SEQ ID NO: 5 or 7.

48. The method according to any one of claims 43-47, wherein the modified nucleic acid comprises a nucleic acid sequence having at least 70% sequence identity to SEQ ID NO: 1, 3 or 39.

49. The method according to any one of claims 43-48, wherein the modified nucleic acid comprises a nucleic acid sequence having at least 80% sequence identity to SEQ ID NO: 1, 3 or 39.

50. The method according to any one of claims 43-49, wherein the modified nucleic acid comprises a nucleic acid sequence having at least 90% sequence identity to SEQ ID NO: 1, 3 or 39.

51. The method according to any one of claims 43-50, wherein the modified nucleic acid comprises (preferably consists of) a nucleic acid sequence shown as SEQ ID NO: 1, 3 or 39.

52. The method according to any one of claims 43-51, wherein the modified nucleic acid expresses a polypeptide comprising a polypeptide sequence having at least 70% sequence identity to SEQ ID NO: 2, 4, 26 or 40.

53. The method according to any one of claims 43-52, wherein the modified nucleic acid expresses a polypeptide comprising a polypeptide sequence having at least 80% sequence identity to SEQ ID NO: 2, 4, 26 or 40.

54. The method according to any one of claims 43-53, wherein the modified nucleic acid expresses a polypeptide comprising a polypeptide sequence having at least 90% sequence identity to SEQ ID NO: 2, 4, 26 or 40.

55. The method according to any one of claims 43-54, wherein the modified nucleic acid expresses a polypeptide comprising (preferably consisting of) a polypeptide sequence shown as SEQ ID NO: 2, 4, 26 or 40.

56. A method for preparing a labelled polypeptide, the method comprising:
a. providing a polypeptide comprising:
i. a transpeptidase or ligase acceptor site or a transpeptidase or ligase donor site;
ii. a non-cytotoxic protease or a proteolytically inactive mutant thereof;
iii. a Targeting Moiety (TM) that is capable of binding to a Binding Site on a target cell; and
iv. a translocation domain;
b. incubating the polypeptide with:
a transpeptidase or ligase; and
a labelled substrate comprising a transpeptidase or ligase donor site or a transpeptidase or ligase acceptor site, respectively, and a conjugated detectable label;
wherein the transpeptidase or ligase catalyses:
conjugation between an amino acid of the transpeptidase or ligase acceptor site of the polypeptide and an amino acid of the transpeptidase or ligase donor site of the labelled substrate; or
conjugation between an amino acid of the transpeptidase or ligase acceptor site of the labelled substrate and an amino acid of the transpeptidase or ligase donor site of the polypeptide;
thereby labelling the polypeptide; and
c. obtaining the labelled polypeptide.

57. The method according to claim 56, wherein the ligase is butelase, PATG, PCY1 or POPB.

58. The method according to claim 56 or 57, wherein the ligase is butelase, preferably Butelase 1.

59. A polypeptide for labelling using a butelase, the polypeptide comprising:
i. a butelase acceptor or donor site;
ii. a non-cytotoxic protease that is capable of cleaving a protein of the exocytic fusion apparatus in a target cell or a proteolytically inactive mutant thereof;
iii. a Targeting Moiety (TM) that is capable of binding to a Binding Site on a target cell; and
iv. a translocation domain that is capable of translocating the non-cytotoxic protease from within an endosome, across the endosomal membrane and into the cytosol of the target cell;
wherein when the polypeptide comprises a butelase donor site, the butelase donor site is located at an N-terminus of the polypeptide; and
wherein the N-terminal residue of the donor site is the N-terminal residue of the polypeptide; or
wherein the polypeptide comprises one or more amino acid residues N-terminal to the butelase donor site and a cleavable site, which when cleaved exposes the N-terminus of the butelase donor site.

60. A labelled polypeptide, the polypeptide comprising:
i. a detectable label conjugated to the polypeptide;
ii. an amino acid sequence that comprises Asn/Asp-Xaa-(Ile/Leu/Val/Cys), wherein Xaa is any amino acid apart from proline;
iii. a non-cytotoxic protease or a proteolytically inactive mutant thereof;
iv. a Targeting Moiety (TM) that is capable of binding to a Binding Site on a target cell; and
v. a translocation domain.

61. The method, polypeptide or labelled polypeptide according to any one of claims 1-37 or 43-60, wherein the detectable label is a fluorophore.

62. The method, polypeptide or labelled polypeptide according to claim 61, wherein the fluorophore is selected from: HiLyte, AlexaFluor, Atto, Quantum Dots, and Janelia Fluor.

63. The method or labelled polypeptide according to any one of claims 1, 3-37, 43-58 or 60-62, wherein the labelled polypeptide comprises two or more detectable labels.

64. The method or labelled polypeptide according to claim 63, wherein the two or more detectable labels are different fluorophores.

65. The method or polypeptide according to any one of claims 1-13, 23-31, 33-36, 43-55, or 61-64, wherein the sortase acceptor site comprises (or consists of) NPKTG, XPETG, LGATG, IPNTG, IPETG, NSKTA, NPQTG, NAKTN, NPQSS, LPXTX, wherein X is any amino acid, NPX₁TX₂, wherein X₁ is Lys or Gln and X₂ is Asn, Asp or Gly, X₁PX₂X₃G, wherein X, is Leu, Ile, Val or Met, X₂ is any amino acid and X₃ is Ser, Thr or Ala, LPEX₁G, wherein X₁ is Ala, Cys or Ser, LPXS, LAXT, MPXT, MPXTG, LAXS, NPXT, NPXTG, NAXT, NAXTG, NAXS, NAXSG, LPXP, LPXPG, LRXTG or LPAXG wherein X is any amino acid.


SEQUENCE LISTING (123 sequences)

SEQ ID NO: 1 - DNA, 7234 bp, Artificial Sequence
  Nucleotide sequence of EGF-liganded polypeptide with dual-labelling SrtA sites
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatcggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctcaagaaataattttgtttaactttaagaaggagatatacatatgggatccatggagaacctgtattttcagggcggcggtggcagcggcggcagcggcggcagccctttcgttaacaaacagttcaactataaagacccagttaacggtgttgacattgcttacatcaaaatcccgaacgctggccagatgcagccggtaaaggcattcaaaatccacaacaaaatctgggttatcccggaacgtgatacctttactaacccggaagaaggtgacctgaacccgccaccggaagcgaaacaggtgccggtatcttactatgactccacctacctgtctaccgataacgaaaaggacaactacctgaaaggtgttactaaactgttcgagcgtatttactccaccgacctgggccgtatgctgctgactagcatcgttcgcggtatcccgttctggggcggttctaccatcgataccgaactgaaagtaatcgacactaactgcatcaacgttattcagccggacggttcctatcgttccgaagaactgaacctggtgatcatcggcccgtctgctgatatcatccagttcgagtgtaagagctttggtcacgaagttctgaacctcacccgtaacggctacggttccactcagtacatccgtttctctccggacttcaccttcggttttgaagaatccctggaagtagacacgaacccactgctgggcgctggtaaattcgcaactgatcctgcggttaccctggctcacgaactgattcatgcaggccaccgcctgtacggtatcgccatcaatccgaaccgtgtcttcaaagttaacaccaacgcgtattacgagatgtccggtctggaagttagcttcgaagaactgcgtacttttggcggtcacgacgctaaattcatcgactctctgcaagaaaacgagttccgtctgtactactataacaagttcaaagatatcgcatccaccctgaacaaagcgaaatccatcgtgggtaccactgcttctctccagtacatgaagaacgtttttaaagaaaaatacctgctcagcgaagacacctccggcaaattctctgtagacaagttgaaattcgataaactttacaaaatgctgactgaaatttacaccgaagacaacttcgttaagttctttaaagttctgaaccgcaaaacctatctgaacttcgacaaggcagtattcaaaatcaacatcgtgccgaaagttaactacactatctacgatggtttcaacctgcgtaacaccaacctggctgctaattttaacggccagaacacggaaatcaacaacatgaacttcacaaaactgaaaaacttcactggtctgttcgagttttacaagctgctgtgcgtcgacggcatcattacctccaaaactaaatctctgatagaaggtagaaacaaagcgctgaacctgcagtgtatcaaggttaacaactgggatttattcttcagcccgagtgaagacaacttcaccaacgacctgaacaaaggtgaagaaatcacctcagatactaacatcgaagcagccgaagaaaacatctcgctggacctgatccagcagtactacctgacctttaatttcgacaacgagccggaaaacatttctatcgaaaacctgagctctgatatcatcggccagctggaactgatgccgaacatcgaacgtttcccaaacggtaaaaagtacgagctggacaaatataccatgttccactacctgcgcgcgcaggaatttgaacacggcaaatcccgtatcgcactgactaactccgttaacgaagctctgctcaacccgtcccgtgtatacaccttcttctctagcgactacgtgaaaaaggtcaacaaagcgactgaagctgcaatgttcttgggttgggttgaacagcttgtttatgattttaccgacgagacgtccgaagtatctactaccgacaaaattgcggatatcactatcatcatcccgtacatcggtccggctctgaacattggcaacatgctgtacaaagacgacttcgttggcgcactgatcttctccggtgcggtgatcctgctggagttcatcccggaaatcgccatcccggtactgggcacctttgctctggtttcttacattgcaaacaaggttctgactgtacaaaccatcgacaacgcgctgagcaaacgtaacgaaaaatgggatgaagtttacaaatatatcgtgaccaactggctggctaaggttaatactcagatcgacctcatccgcaaaaaaatgaaagaagcactggaaaaccaggcggaagctaccaaggcaatcattaactaccagtacaaccagtacaccgaggaagaaaaaaacaacatcaacttcaacatcgacgatctgtcctctaaactgaacgaatccatcaacaaagctatgatcaacatcaacaagttcctgaaccagtgctctgtaagctatctgatgaactccatgatcccgtacggtgttaaacgtctggaggacttcgatgcgtctctgaaagacgccctgctgaaatacatttacgacaaccgtggcactctgatcggtcaggttgatcgtctgaaggacaaagtgaacaataccttatcgaccgacatcccttttcagctcagtaaatatgtcgataaccaacgccttttgtccactctagaaggcggtggcggtagcggtggcggtggcagcggcggtggcggtagcgcactagacaacagcgaccctaaatgcccactgagtcatgaaggatactgccttaatgatggtgtttgtatgtacataggaacattggaccgttatgcttgcaattgtgtagtgggctatgtcggggaaaggtgtcaatatcgagatctcaagctggcagagttaagagggctagaagcaggcggcagcggcggcggcagcggcctgcccgaaagcggtggcggatctgcttggtctcacccgcagttcgaaaaaggtggtggttctggtggtggttctggtggttctgcttggtctcacccgcagttcgaaaaataatgaaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 2 - protein, 1004 aa, Artificial Sequence
  Polypeptide sequence of EGF-liganded polypeptide with dual-labelling SrtA sites
MENLYFQGGGGSGGSGGSPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVDGIITSKTKSLIEGRNKALNLQCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTLEGGGGSGGGGSGGGGSALDNSDPKCPLSHEGYCLNDGVCMYIGTLDRYACNCVVGYVGERCQYRDLKLAELRGLEAGGSGGGSGLPESGGGSAWSHPQFEKGGGSGGGSGGSAWSHPQFEK

SEQ ID NO: 3 - DNA, 7108 bp, Artificial Sequence
  Nucleotide sequence of nociceptin-liganded polypeptide with dual-labelling SrtA sites
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatcggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctcaagaaataattttgtttaactttaagaaggagatatacatatggagaacctgtattttcagggcggcggtggcagcggcggcagcggcggcagcggcagcatgccttttgtgaacaaacagttcaactataaggatccggttaatggtgtggatatcgcctatatcaaaattccgaatgcaggtcagatgcagccggttaaagcctttaaaatccataacaaaatttgggtgattccggaacgtgatacctttaccaatccggaagaaggtgatctgaatccgcctccggaagcaaaacaggttccggttagctattatgatagcacctatctgagcaccgataacgagaaagataactatctgaaaggtgtgaccaaactgtttgaacgcatttatagtaccgatctgggtcgtatgctgctgaccagcattgttcgtggtattccgttttggggtggtagcaccattgataccgaactgaaagttattgacaccaactgcattaatgtgattcagccggatggtagctatcgtagcgaagaactgaatctggttattattggtccgagcgcagatatcattcagtttgaatgtaaatcctttggccacgaagttctgaatctgacccgtaatggttatggtagtacccagtatattcgtttcagtccggattttacctttggctttgaagaaagcctggaagttgatacaaatccgctgttaggtgcaggtaaatttgcaaccgatccggcagttaccctggcacatgaactgattcatgccggtcatcgtctgtatggtattgcaattaatccgaaccgtgtgttcaaagtgaataccaacgcatattatgaaatgagcggtctggaagtgtcatttgaagaactgcgtacctttggtggtcatgatgccaaatttatcgatagcctgcaagaaaatgaatttcgcctgtactactataacaaattcaaggatattgcgagcaccctgaataaagccaaaagcattgttggcaccaccgcaagcctgcagtatatgaaaaatgtgtttaaagaaaaatatctgctgagcgaagataccagcggtaaatttagcgttgacaaactgaaattcgataaactgtacaagatgctgaccgagatttataccgaagataacttcgtgaagtttttcaaagtgctgaaccgcaaaacctacctgaactttgataaagccgtgttcaaaatcaacatcgtgccgaaagtgaactataccatctatgatggttttaacctgcgcaataccaatctggcagcaaactttaatggtcagaacaccgaaatcaacaacatgaactttaccaaactgaagaacttcaccggtctgttcgaattttacaaactgctgtgtgtggatggcattattaccagcaaaaccaaatccgatgatgacgataaattcggtggttttaccggtgcacgtaaaagcgcacgtaaacgtaaaaatcaggcactggcaggcggtggtggtagcggtggcggtggttcaggtggtggtggctcagcactggttctgcagtgtattaaagttaataactgggacctgttttttagcccgagcgaggataatttcaccaacgatctgaacaaaggcgaagaaattaccagcgataccaatattgaagcagccgaagaaaacattagcctggatctgattcagcagtattatctgaccttcaacttcgataatgagccggaaaatatcagcattgaaaacctgagcagcgatattattggccagctggaactgatgccgaatattgaacgttttccgaacggcaaaaaatacgagctggataaatacaccatgttccattatctgcgtgcccaagaatttgaacatggtaaaagccgtattgcactgaccaatagcgttaatgaagcactgctgaacccgagccgtgtttataccttttttagcagcgattacgtgaaaaaggttaacaaagcaaccgaagcagccatgtttttaggttgggttgaacagctggtttatgatttcaccgatgaaaccagcgaagttagcaccaccgataaaattgcagatattaccatcatcatcccgtatatcggtccggcactgaatattggcaatatgctgtataaagacgattttgtgggtgccctgatctttagcggtgcagttattctgctggaatttattccggaaattgccattccggttctgggcacctttgcactggtgagctatattgcaaataaagttctgaccgtgcagaccatcgataatgcactgagcaaacgtaacgaaaaatgggatgaagtgtacaagtatatcgtgaccaattggctggcaaaagttaacacccagattgacctgattcgcaagaagatgaaagaagcactggaaaaccaggcagaagcaaccaaagccattattaactatcagtacaaccagtacaccgaagaagagaagaataacatcaacttcaacatcgatgatctgagcagcaagctgaatgaaagcatcaacaaagccatgatcaacattaacaaatttctgaatcagtgcagcgtgagctatctgatgaatagcatgattccgtatggtgtgaaacgtctggaagattttgatgcaagcctgaaagatgccctgctgaaatatatctatgataatcgtggcaccctgattggtcaggttgatcgtctgaaagataaagtgaacaacaccctgagtaccgatattccttttcagctgagcaaatatgtggataatcagcgtctgctgagtaccctggatggcggcagcggcggcggcagcggcctgcccgaaagcggtggcggatctgcttggtctcacccgcagttcgaaaaaggtggtggttctggtggtggttctggtggttctgcttggtctcacccgcagttcgaaaaataatgaaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 4 - protein, 948 aa, Artificial Sequence
  Polypeptide sequence of nociceptin-liganded polypeptide with dual-labelling SrtA sites
MENLYFQGGGGSGGSGGSGSMPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVDGIITSKTKSDDDDKFGGFTGARKSARKRKNQALAGGGGSGGGGSGGGGSALVLQCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTLDGGSGGGSGLPESGGGSAWSHPQFEKGGG

SEQ ID NO: 5 - DNA, 7078 bp, Artificial Sequence
  Nucleotide sequence of EGF-liganded polypeptide
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatcggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctcaagaaataattttgtttaactttaagaaggagatatacatatgggatccatggagttcgttaacaaacagttcaactataaagacccagttaacggtgttgacattgcttacatcaaaatcccgaacgctggccagatgcagccggtaaaggcattcaaaatccacaacaaaatctgggttatcccggaacgtgatacctttactaacccggaagaaggtgacctgaacccgccaccggaagcgaaacaggtgccggtatcttactatgactccacctacctgtctaccgataacgaaaaggacaactacctgaaaggtgttactaaactgttcgagcgtatttactccaccgacctgggccgtatgctgctgactagcatcgttcgcggtatcccgttctggggcggttctaccatcgataccgaactgaaagtaatcgacactaactgcatcaacgttattcagccggacggttcctatcgttccgaagaactgaacctggtgatcatcggcccgtctgctgatatcatccagttcgagtgtaagagctttggtcacgaagttctgaacctcacccgtaacggctacggttccactcagtacatccgtttctctccggacttcaccttcggttttgaagaatccctggaagtagacacgaacccactgctgggcgctggtaaattcgcaactgatcctgcggttaccctggctcacgaactgattcatgcaggccaccgcctgtacggtatcgccatcaatccgaaccgtgtcttcaaagttaacaccaacgcgtattacgagatgtccggtctggaagttagcttcgaagaactgcgtacttttggcggtcacgacgctaaattcatcgactctctgcaagaaaacgagttccgtctgtactactataacaagttcaaagatatcgcatccaccctgaacaaagcgaaatccatcgtgggtaccactgcttctctccagtacatgaagaacgtttttaaagaaaaatacctgctcagcgaagacacctccggcaaattctctgtagacaagttgaaattcgataaactttacaaaatgctgactgaaatttacaccgaagacaacttcgttaagttctttaaagttctgaaccgcaaaacctatctgaacttcgacaaggcagtattcaaaatcaacatcgtgccgaaagttaactacactatctacgatggtttcaacctgcgtaacaccaacctggctgctaattttaacggccagaacacggaaatcaacaacatgaacttcacaaaactgaaaaacttcactggtctgttcgagttttacaagctgctgtgcgtcgacggcatcattacctccaaaactaaatctctgatagaaggtagaaacaaagcgctgaacctgcagtgtatcaaggttaacaactgggatttattcttcagcccgagtgaagacaacttcaccaacgacctgaacaaaggtgaagaaatcacctcagatactaacatcgaagcagccgaagaaaacatctcgctggacctgatccagcagtactacctgacctttaatttcgacaacgagccggaaaacatttctatcgaaaacctgagctctgatatcatcggccagctggaactgatgccgaacatcgaacgtttcccaaacggtaaaaagtacgagctggacaaatataccatgttccactacctgcgcgcgcaggaatttgaacacggcaaatcccgtatcgcactgactaactccgttaacgaagctctgctcaacccgtcccgtgtatacaccttcttctctagcgactacgtgaaaaaggtcaacaaagcgactgaagctgcaatgttcttgggttgggttgaacagcttgtttatgattttaccgacgagacgtccgaagtatctactaccgacaaaattgcggatatcactatcatcatcccgtacatcggtccggctctgaacattggcaacatgctgtacaaagacgacttcgttggcgcactgatcttctccggtgcggtgatcctgctggagttcatcccggaaatcgccatcccggtactgggcacctttgctctggtttcttacattgcaaacaaggttctgactgtacaaaccatcgacaacgcgctgagcaaacgtaacgaaaaatgggatgaagtttacaaatatatcgtgaccaactggctggctaaggttaatactcagatcgacctcatccgcaaaaaaatgaaagaagcactggaaaaccaggcggaagctaccaaggcaatcattaactaccagtacaaccagtacaccgaggaagaaaaaaacaacatcaacttcaacatcgacgatctgtcctctaaactgaacgaatccatcaacaaagctatgatcaacatcaacaagttcctgaaccagtgctctgtaagctatctgatgaactccatgatcccgtacggtgttaaacgtctggaggacttcgatgcgtctctgaaagacgccctgctgaaatacatttacgacaaccgtggcactctgatcggtcaggttgatcgtctgaaggacaaagtgaacaataccttatcgaccgacatcccttttcagctcagtaaatatgtcgataaccaacgccttttgtccactctagaaggcggtggcggtagcggtggcggtggcagcggcggtggcggtagcgcactagacaacagcgaccctaaatgcccactgagtcatgaaggatactgccttaatgatggtgtttgtatgtacataggaacattggaccgttatgcttgcaattgtgtagtgggctatgtcggggaaaggtgtcaatatcgagatctcaagctggcagagttaagagggctagaagcacaccatcatcaccaccatcaccatcaccattaatgaaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 6 - protein, 952 aa, Artificial Sequence
  Polypeptide sequence of EGF-liganded polypeptide
MEFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVDGIITSKTKSLIEGRNKALNLQCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTLEGGGGSGGGGSGGGGSALDNSDPKCPLSHEGYCLNDGVCMYIGTLDRYACNCVVGYVGERCQYRDLKLAELRGLEAHHHHHHHHHH

SEQ ID NO: 7 - DNA, 6937 bp, Artificial Sequence
  Nucleotide sequence of nociceptin-liganded polypeptide
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatcggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctcaagaaataattttgtttaactttaagaaggagatatacatatgggcagcatggaatttgtgaacaaacagttcaactataaggatccggttaatggtgtggatatcgcctatatcaaaattccgaatgcaggtcagatgcagccggttaaagcctttaaaatccataacaaaatttgggtgattccggaacgtgatacctttaccaatccggaagaaggtgatctgaatccgcctccggaagcaaaacaggttccggttagctattatgatagcacctatctgagcaccgataacgagaaagataactatctgaaaggtgtgaccaaactgtttgaacgcatttatagtaccgatctgggtcgtatgctgctgaccagcattgttcgtggtattccgttttggggtggtagcaccattgataccgaactgaaagttattgacaccaactgcattaatgtgattcagccggatggtagctatcgtagcgaagaactgaatctggttattattggtccgagcgcagatatcattcagtttgaatgtaaatcctttggccacgaagttctgaatctgacccgtaatggttatggtagtacccagtatattcgtttcagtccggattttacctttggctttgaagaaagcctggaagttgatacaaatccgctgttaggtgcaggtaaatttgcaaccgatccggcagttaccctggcacatgaactgattcatgccggtcatcgtctgtatggtattgcaattaatccgaaccgtgtgttcaaagtgaataccaacgcatattatgaaatgagcggtctggaagtgtcatttgaagaactgcgtacctttggtggtcatgatgccaaatttatcgatagcctgcaagaaaatgaatttcgcctgtactactataacaaattcaaggatattgcgagcaccctgaataaagccaaaagcattgttggcaccaccgcaagcctgcagtatatgaaaaatgtgtttaaagaaaaatatctgctgagcgaagataccagcggtaaatttagcgttgacaaactgaaattcgataaactgtacaagatgctgaccgagatttataccgaagataacttcgtgaagtttttcaaagtgctgaaccgcaaaacctacctgaactttgataaagccgtgttcaaaatcaacatcgtgccgaaagtgaactataccatctatgatggttttaacctgcgcaataccaatctggcagcaaactttaatggtcagaacaccgaaatcaacaacatgaactttaccaaactgaagaacttcaccggtctgttcgaattttacaaactgctgtgtgtggatggcattattaccagcaaaaccaaatccgatgatgacgataaattcggtggttttaccggtgcacgtaaaagcgcacgtaaacgtaaaaatcaggcactggcaggcggtggtggtagcggtggcggtggttcaggtggtggtggctcagcactggttctgcagtgtattaaagttaataactgggacctgttttttagcccgagcgaggataatttcaccaacgatctgaacaaaggcgaagaaattaccagcgataccaatattgaagcagccgaagaaaacattagcctggatctgattcagcagtattatctgaccttcaacttcgataatgagccggaaaatatcagcattgaaaacctgagcagcgatattattggccagctggaactgatgccgaatattgaacgttttccgaacggcaaaaaatacgagctggataaatacaccatgttccattatctgcgtgcccaagaatttgaacatggtaaaagccgtattgcactgaccaatagcgttaatgaagcactgctgaacccgagccgtgtttataccttttttagcagcgattacgtgaaaaaggttaacaaagcaaccgaagcagccatgtttttaggttgggttgaacagctggtttatgatttcaccgatgaaaccagcgaagttagcaccaccgataaaattgcagatattaccatcatcatcccgtatatcggtccggcactgaatattggcaatatgctgtataaagacgattttgtgggtgccctgatctttagcggtgcagttattctgctggaatttattccggaaattgccattccggttctgggcacctttgcactggtgagctatattgcaaataaagttctgaccgtgcagaccatcgataatgcactgagcaaacgtaacgaaaaatgggatgaagtgtacaagtatatcgtgaccaattggctggcaaaagttaacacccagattgacctgattcgcaagaagatgaaagaagcactggaaaaccaggcagaagcaaccaaagccattattaactatcagtacaaccagtacaccgaagaagagaagaataacatcaacttcaacatcgatgatctgagcagcaagctgaatgaaagcatcaacaaagccatgatcaacattaacaaatttctgaatcagtgcagcgtgagctatctgatgaatagcatgattccgtatggtgtgaaacgtctggaagattttgatgcaagcctgaaagatgccctgctgaaatatatctatgataatcgtggcaccctgattggtcaggttgatcgtctgaaagataaagtgaacaacaccctgagtaccgatattccttttcagctgagcaaatatgtggataatcagcgtctgctgagtaccctggatcatcatcaccatcaccactaaaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 8 - protein, 909 aa, Artificial Sequence
  Polypeptide sequence of nociceptin-liganded polypeptide
MGSMEFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVDGIITSKTKSDDDDKFGGFTGARKSARKRKNQALAGGGGSGGGGSGGGGSALVLQCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTLDHHHHHH

SEQ ID NO: 9 - DNA, 7822 bp, Artificial Sequence
  Nucleotide sequence of EGF-liganded polypeptide GFP-tagged
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatcggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctcaagaaataattttgtttaactttaagaaggagatatacatatgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagggcggcagcggcggcggcagcggcggcggatccatggagttcgttaacaaacagttcaactataaagacccagttaacggtgttgacattgcttacatcaaaatcccgaacgctggccagatgcagccggtaaaggcattcaaaatccacaacaaaatctgggttatcccggaacgtgatacctttactaacccggaagaaggtgacctgaacccgccaccggaagcgaaacaggtgccggtatcttactatgactccacctacctgtctaccgataacgaaaaggacaactacctgaaaggtgttactaaactgttcgagcgtatttactccaccgacctgggccgtatgctgctgactagcatcgttcgcggtatcccgttctggggcggttctaccatcgataccgaactgaaagtaatcgacactaactgcatcaacgttattcagccggacggttcctatcgttccgaagaactgaacctggtgatcatcggcccgtctgctgatatcatccagttcgagtgtaagagctttggtcacgaagttctgaacctcacccgtaacggctacggttccactcagtacatccgtttctctccggacttcaccttcggttttgaagaatccctggaagtagacacgaacccactgctgggcgctggtaaattcgcaactgatcctgcggttaccctggctcacgaactgattcatgcaggccaccgcctgtacggtatcgccatcaatccgaaccgtgtcttcaaagttaacaccaacgcgtattacgagatgtccggtctggaagttagcttcgaagaactgcgtacttttggcggtcacgacgctaaattcatcgactctctgcaagaaaacgagttccgtctgtactactataacaagttcaaagatatcgcatccaccctgaacaaagcgaaatccatcgtgggtaccactgcttctctccagtacatgaagaacgtttttaaagaaaaatacctgctcagcgaagacacctccggcaaattctctgtagacaagttgaaattcgataaactttacaaaatgctgactgaaatttacaccgaagacaacttcgttaagttctttaaagttctgaaccgcaaaacctatctgaacttcgacaaggcagtattcaaaatcaacatcgtgccgaaagttaactacactatctacgatggtttcaacctgcgtaacaccaacctggctgctaattttaacggccagaacacggaaatcaacaacatgaacttcacaaaactgaaaaacttcactggtctgttcgagttttacaagctgctgtgcgtcgacggcatcattacctccaaaactaaatctctgatagaaggtagaaacaaagcgctgaacctgcagtgtatcaaggttaacaactgggatttattcttcagcccgagtgaagacaacttcaccaacgacctgaacaaaggtgaagaaatcacctcagatactaacatcgaagcagccgaagaaaacatctcgctggacctgatccagcagtactacctgacctttaatttcgacaacgagccggaaaacatttctatcgaaaacctgagctctgatatcatcggccagctggaactgatgccgaacatcgaacgtttcccaaacggtaaaaagtacgagctggacaaatataccatgttccactacctgcgcgcgcaggaatttgaacacggcaaatcccgtatcgcactgactaactccgttaacgaagctctgctcaacccgtcccgtgtatacaccttcttctctagcgactacgtgaaaaaggtcaacaaagcgactgaagctgcaatgttcttgggttgggttgaacagcttgtttatgattttaccgacgagacgtccgaagtatctactaccgacaaaattgcggatatcactatcatcatcccgtacatcggtccggctctgaacattggcaacatgctgtacaaagacgacttcgttggcgcactgatcttctccggtgcggtgatcctgctggagttcatcccggaaatcgccatcccggtactgggcacctttgctctggtttcttacattgcaaacaaggttctgactgtacaaaccatcgacaacgcgctgagcaaacgtaacgaaaaatgggatgaagtttacaaatatatcgtgaccaactggctggctaaggttaatactcagatcgacctcatccgcaaaaaaatgaaagaagcactggaaaaccaggcggaagctaccaaggcaatcattaactaccagtacaaccagtacaccgaggaagaaaaaaacaacatcaacttcaacatcgacgatctgtcctctaaactgaacgaatccatcaacaaagctatgatcaacatcaacaagttcctgaaccagtgctctgtaagctatctgatgaactccatgatcccgtacggtgttaaacgtctggaggacttcgatgcgtctctgaaagacgccctgctgaaatacatttacgacaaccgtggcactctgatcggtcaggttgatcgtctgaaggacaaagtgaacaataccttatcgaccgacatcccttttcagctcagtaaatatgtcgataaccaacgccttttgtccactctagaaggcggtggcggtagcggtggcggtggcagcggcggtggcggtagcgcactagacaacagcgaccctaaatgcccactaagtcatgaaggatactgccttaatgatggtgtttgtatgtacataggaacattggaccgttatgcttgcaattgtgtagtgggctatgtcggggaaaggtgtcaatatcgagatctcaagctggcagagttaagagggctagaagcacaccatcatcaccaccatcaccatcaccattaatgaaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 10 - protein, 1202 aa, Artificial Sequence
  Polypeptide sequence of EGF-liganded polypeptide GFP-tagged
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKGGSGGGSGGGSMEFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVDGIITSKTKSLIEGRNKALNLQCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTLEGGGGSGGGGSGGGGSALDNSDPKCPLSHEGYCLNDGVCMYIGTLDRYACNCVVGYVGERCQYRDLKLAELRGLEAHHHHHHHHHH

SEQ ID NO: 11 - DNA, 7651 bp, Artificial Sequence
  Nucleotide sequence of EGF-liganded polypeptide SNAP tagged
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatcggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctcaagaaataattttgtttaactttaagaaggagatatacatatgatggacaaagactgcgaaatgaagcgcaccaccctggatagccctctgggcaagctggaactgtctgggtgcgaacagggcctgcaccgtatcatcttcctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctacagccacctggccgccctggccggcaatcccgccgccaccgccgccgtgaaaaccgccctgagcggaaatcccgtgcccattctgatcccctgccaccgggtggtgcagggcgacctggacgtggggggctacgagggcgggctcgccgtgaaagagtggctgctggcccacgagggccacagactgggcaagcctgggctgggtggcggcagcggcggcggcagcggcggcggatccatggagttcgttaacaaacagttcaactataaagacccagttaacggtgttgacattgcttacatcaaaatcccgaacgctggccagatgcagccggtaaaggcattcaaaatccacaacaaaatctgggttatcccggaacgtgatacctttactaacccggaagaaggtgacctgaacccgccaccggaagcgaaacaggtgccggtatcttactatgactccacctacctgtctaccgataacgaaaaggacaactacctgaaaggtgttactaaactgttcgagcgtatttactccaccgacctgggccgtatgctgctgactagcatcgttcgcggtatcccgttctggggcggttctaccatcgataccgaactgaaagtaatcgacactaactgcatcaacgttattcagccggacggttcctatcgttccgaagaactgaacctggtgatcatcggcccgtctgctgatatcatccagttcgagtgtaagagctttggtcacgaagttctgaacctcacccgtaacggctacggttccactcagtacatccgtttctctccggacttcaccttcggttttgaagaatccctggaagtagacacgaacccactgctgggcgctggtaaattcgcaactgatcctgcggttaccctggctcacgaactgattcatgcaggccaccgcctgtacggtatcgccatcaatccgaaccgtgtcttcaaagttaacaccaacgcgtattacgagatgtccggtctggaagttagcttcgaagaactgcgtacttttggcggtcacgacgctaaattcatcgactctctgcaagaaaacgagttccgtctgtactactataacaagttcaaagatatcgcatccaccctgaacaaagcgaaatccatcgtgggtaccactgcttctctccagtacatgaagaacgtttttaaagaaaaatacctgctcagcgaagacacctccggcaaattctctgtagacaagttgaaattcgataaactttacaaaatgctgactgaaatttacaccgaagacaacttcgttaagttctttaaagttctgaaccgcaaaacctatctgaacttcgacaaggcagtattcaaaatcaacatcgtgccgaaagttaactacactatctacgatggtttcaacctgcgtaacaccaacctggctgctaattttaacggccagaacacggaaatcaacaacatgaacttcacaaaactgaaaaacttcactggtctgttcgagttttacaagctgctgtgcgtcgacggcatcattacctccaaaactaaatctctgatagaaggtagaaacaaagcgctgaacctgcagtgtatcaaggttaacaactgggatttattcttcagcccgagtgaagacaacttcaccaacgacctgaacaaaggtgaagaaatcacctcagatactaacatcgaagcagccgaagaaaacatctcgctggacctgatccagcagtactacctgacctttaatttcgacaacgagccggaaaacatttctatcgaaaacctgagctctgatatcatcggccagctggaactgatgccgaacatcgaacgtttcccaaacggtaaaaagtacgagctggacaaatataccatgttccactacctgcgcgcgcaggaatttgaacacggcaaatcccgtatcgcactgactaactccgttaacgaagctctgctcaacccgtcccgtgtatacaccttcttctctagcgactacgtgaaaaaggtcaacaaagcgactgaagctgcaatgttcttgggttgggttgaacagcttgtttatgattttaccgacgagacgtccgaagtatctactaccgacaaaattgcggatatcactatcatcatcccgtacatcggtccggctctgaacattggcaacatgctgtacaaagacgacttcgttggcgcactgatcttctccggtgcggtgatcctgctggagttcatcccggaaatcgccatcccggtactgggcacctttgctctggtttcttacattgcaaacaaggttctgactgtacaaaccatcgacaacgcgctgagcaaacgtaacgaaaaatgggatgaagtttacaaatatatcgtgaccaactggctggctaaggttaatactcagatcgacctcatccgcaaaaaaatgaaagaagcactggaaaaccaggcggaagctaccaaggcaatcattaactaccagtacaaccagtacaccgaggaagaaaaaaacaacatcaacttcaacatcgacgatctgtcctctaaactgaacgaatccatcaacaaagctatgatcaacatcaacaagttcctgaaccagtgctctgtaagctatctgatgaactccatgatcccgtacggtgttaaacgtctggaggacttcgatgcgtctctgaaagacgccctgctgaaatacatttacgacaaccgtggcactctgatcggtcaggttgatcgtctgaaggacaaagtgaacaataccttatcgaccgacatcccttttcagctcagtaaatatgtcgataaccaacgccttttgtccactctagaaggcggtggcggtagcggtggcggtggcagcggcggtggcggtagcgcactagacaacagcgaccctaaatgcccactaagtcatgaaggatactgccttaatgatggtgtttgtatgtacataggaacattggaccgttatgcttgcaattgtgtagtgggctatgtcggggaaaggtgtcaatatcgagatctcaagctggcagagttaagagggctagaagcacaccatcatcaccaccatcaccatcaccattaatgaaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 12 - protein, 1145 aa, Artificial Sequence
  Polypeptide sequence of EGF-liganded polypeptide SNAP tagged
MDKDCEMKRTTLDSPLGKLELSGCEQGLHRIIFLGKGTSAADAVEVPAPAAVLGGPEPLMQATAWLNAYFHQPEAIEEFPVPALHHPVFQQESFTRQVLWKLLKVVKFGEVISYSHLAALAGNPAATAAVKTALSGNPVPILIPCHRVVQGDLDVGGYEGGLAVKEWLLAHEGHRLGKPGLGGGSGGGSGGGSMEFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVDGIITSKTKSLIEGRNKALNLQCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTLEGGGGSGGGGSGGGGSALDNSDPKCPLSHEGYCLNDGVCMYIGTLDRYACNCVVGYVGERCQYRDLKLAELRGLEAHHHHHHHHHH

SEQ ID NO: 13 - DNA, 4683 bp, Artificial Sequence
  Nucleotide sequence of Sortase A (LPESG-targeting)
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatcggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctcaagaaataattttgtttaactttaagaaggagatatcatatgcaggcaaaaccgcagattccgaaagataaaagcaaagtggcaggctatattgaaattccggatgccgatattaaagaaccggtttatccgggtcctgcaacacgtgaacagctggatcgtggtgtttgttttgttgaagaaaatgagagcctggatgatcagaacattagcattaccggtcataccgcaattgatcgtccgaattatcagtttaccaatctgcgtgcagccaaaccgggtagcatggtttatctgaaagttggtaatgaaacccgcatctacaaaatgaccagcattcgtaatgttaaaccgaccgcagttggtgttctggatgaacaaaaaggtaaagataaacagctgaccctggttacctgtgatgattataactttgaaaccggtgtttgggaaacgcgcaaaatctttgttgcaaccgaagttaaacatcaccatcaccaccatcatcatcaccattaaaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 14 - protein, 158 aa, Artificial Sequence
  Polypeptide sequence of Sortase A (LPESG-targeting)
MQAKPQIPKDKSKVAGYIEIPDADIKEPVYPGPATREQLDRGVCFVEENESLDDQNISITGHTAIDRPNYQFTNLRAAKPGSMVYLKVGNETRIYKMTSIRNVKPTAVGVLDEQKGKDKQLTLVTCDDYNFETGVWETRKIFVATEVKHHHHHHHHHH

SEQ ID NO: 15 - DNA, 4684 bp, Artificial Sequence
  Nucleotide sequence of Sortase A (LAETG-targeting)
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatcggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctcaagaaataattttgtttaactttaagaaggagatatacatatgcaggcaaaaccgcagattccgaaagataaaagcaaagtggcaggctatattgaaattccggatgccgatattaaagaaccggtttatccgggtcctgcaacacgtgaacagctgaatcgtggtgtttgttttcacgatgaaaatgagagcctggatgatcagaatattagcattgcaggccatacctttattgatcgtccgaattatcagttcaccaatctgaaagcagcaaaaccgggtagcatggtttatttcaaagttggtaatgaaacccgcatctacaaaatgaccagcattcgtaaagttcatccgaatgcagttggtgttctggatgaacaagaaggcaaagataaacagctgaccctggttacctgtgatgattataacgaagaaaccggtgtttgggaaagccgtaaaatctttgttgcaaccgaagtgaaacatcatcaccaccatcaccatcatcatcactaaaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 16 - protein, 158 aa, Artificial Sequence
  Polypeptide sequence of Sortase A (LAETG-targeting)
MQAKPQIPKDKSKVAGYIEIPDADIKEPVYPGPATREQLNRGVCFHDENESLDDQNISIAGHTFIDRPNYQFTNLKAAKPGSMVYFKVGNETRIYKMTSIRKVHPNAVGVLDEQEGKDKQLTLVTCDDYNEETGVWESRKIFVATEVKHHHHHHHHHH

SEQ ID NO: 17 - protein, 1296 aa, Clostridium botulinum [sequence omitted]

SEQ ID NO: 18 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 19 - protein, 1291 aa, Clostridium botulinum
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 20 - protein, 1276 aa, Clostridium botulinum
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKNSRDDSTCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNSINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVE

SEQ ID NO: 21 - protein, 1251 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLRTKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKIINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTFEDNRGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNCTMNFKNNNGNNIGLLGFKADTVVASTWYYTHMRDHTNSNGCFWNFISEEHGWQEK

SEQ ID NO: 22 - protein, 1278 aa, Clostridium botulinum
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 23 - protein, 1297 aa, Clostridium botulinum
  MISC_FEATURE (7)..(7): Xaa is any amino acid
MPVNIKXFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 24 - protein, 1306 aa, Clostridium botulinum
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICPRNGLLYNAIYRNSKNYLNNIDLEDKKTTSKTNVSYPCSLLNGCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEIEDYEVLNLGAEDGKIKDLSGTTSDINIGSDIELADGRENKAIKIKGSENSTIKIAMNKYLRFSATDNFSISFWIKHPKPTNLLNNGIEYTLVENFNQRGWKISIQDSKLIWYLRDHNNSIKIVTPDYIAFNGWNLITITNNRSKGSIVYVNGSKIEEKDISSIWNTEVDDPIIFRLKNNRDTQAFTLLDQFSIYRKELNQNEVVKLYNYYFNSNYIRDIWGNPLQYNKKYYLQTQDKPGKGLIREYWSSFGYDYVILSDSKTITFPNNIRYGALYNGSKVLIKNSKKLDGLVRNKDFIQLEIDGYNMGISADRFNEDTNYIGTTYGTTHDLTTDFEIIQRQEKYRNYCQLKTPYNIFHKSGLMSTETSKPTFHDYRDWVYSSAWYFQNYENLNLRKHTKTNWYFIPKDEGWDED

SEQ ID NO: 25 - protein, 1315 aa, Clostridium tetani
MPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 26 - protein, 974 aa, Artificial Sequence
  Polypeptide sequence of labelled EGF TM polypeptide
  MISC_FEATURE (1)..(1): HiLyte555 detectable label conjugated to His
  MISC_FEATURE (974)..(974): HiLyte488 detectable label conjugated to Lys
HHHHHHLAETGGSGGSGGSEFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVDGIITSKTKSLIEGRNKALNLQCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTLEGGGGSGGGGSGGGGSALDNSDPKCPLSHEGYCLNDGVCMYIGTLDRYACNCVVGYVGERCQYRDLKLAELRGLEAGGSGGGSGLPESGK

SEQ ID NO: 27 - protein, 482 aa, Clitoria ternatea
MKNPLAILFLIATVVAVVSGIRDDFLRLPSQASKFFQADDNVEGTRWAVLVAGSKGYVNYRHQADVCHAYQILKKGGLKDENIIVFMYDDIAYNESNPHPGVIINHPYGSDVYKGVPKDYVGEDINPPNFYAVLLANKSALTGTGSGKVLDSGPNDHVFIYYTDHGGAGVLGMPSKPYIAASDLNDVLKKKHASGTYKSIVFYVESCESGSMFDGLLPEDHNIYVMGASDTGESSWVTYCPLQHPSPPPEYDVCVGDLFSVAWLEDCDVHNLQTETFQQQYEVVKNKTIVALIEDGTHVVQYGDVGLSKQTLFVYMGTDPANDNNTFTDKNSLGTPRKAVSQRDADLIHYWEKYRRAPEGSSRKAEAKKQLREVMAHRMHIDNSVKHIGKLLFGIEKGHKMLNNVRPAGLPVVDDWDCFKTLIRTFETHCGSLSEYGMKHMRSFANLCNAGIRKEQMAEASAQACVSIPDNPWSSLHAGFSV

SEQ ID NO: 28 - protein, 462 aa, Clitoria ternatea
IRDDFLRLPSQASKFFQADDNVEGTRWAVLVAGSKGYVNYRHQADVCHAYQILKKGGLKDENIIVFMYDDIAYNESNPHPGVIINHPYGSDVYKGVPKDYVGEDINPPNFYAVLLANKSALTGTGSGKVLDSGPNDHVFIYYTDHGGAGVLGMPSKPYIAASDLNDVLKKKHASGTYKSIVFYVESCESGSMFDGLLPEDHNIYVMGASDTGESSWVTYCPLQHPSPPPEYDVCVGDLFSVAWLEDCDVHNLQTETFQQQYEVVKNKTIVALIEDGTHVVQYGDVGLSKQTLFVYMGTDPANDNNTFTDKNSLGTPRKAVSQRDADLIHYWEKYRRAPEGSSRKAEAKKQLREVMAHRMHIDNSVKHIGKLLFGIEKGHKMLNNVRPAGLPVVDDWDCFKTLIRTFETHCGSLSEYGMKHMRSFANLCNAGIRKEQMAEASAQACVSIPDNPWSSLHAGFSV

SEQ ID NO: 29 - protein, 5 aa, Artificial Sequence
  Peptide with conjugated detectable label and sortase donor site
  MISC_FEATURE (5)..(5): HiLyte488 detectable label conjugated to Lys
GGGGK

SEQ ID NO: 30 - protein, 13 aa, Artificial Sequence
  Peptide with conjugated detectable label and sortase acceptor site
  MISC_FEATURE (1)..(1): HiLyte555 detectable label conjugated to His
HHHHHHLAETGGG

SEQ ID NO: 31 - protein, 206 aa, Staphylococcus aureus
MKKWTNRLMTIAGVVLILVAAYLFAKPHIDNYLHDKDKDEKIEQYDKNVKEQASKDKKQQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVK

SEQ ID NO: 32 - protein, 244 aa, Staphylococcus aureus
MRMKRFLTIVQILLVVIIIIFGYKIVQTYIEDKQERANYEKLQQKFQMLMSKHQEHVRPQFESLEKINKDIVGWIKLSGTSLNYPVLQGKTNHDYLNLDFEREHRRKGSIFMDFRNELKNLNHNTILYGHHVGDNTMFDVLEDYLKQSFYEKHKIIEFDNKYGKYQLQVFSAYKTTTKDNYIRTDFENDQDYQQFLDETKRKSVINSDVNVTVKDRIMTLSTCEDAYSETTKRIVVVAKIIKVS

SEQ ID NO: 33 - protein, 260 aa, Streptococcus pneumoniae
MEKLYIHLKNLRKVAVVMLLVFTTFYLLLMFLNQSDNQEIAKNIEKFNDSVIVAKTDNTKADIKEIEKNIEKVRKIEGGNVERVNQLTSENEKVKENIDLNIEEEIIENSYKSLETTDNFEKLGIIEIPKIDLNLSIFKGKPFVNTKNRQDTMLYGAVTNKKNQKMGRENYVLASHIISNSNLLFTSINQLEKGDVITLKDSEYSYQYTVYNNFIVSKDETWILNDIKDYSILTLYTCYDDSTKLPENRVVIRAVLTDIN

SEQ ID NO: 34 - protein, 298 aa, Streptococcus pneumoniae
  MISC_FEATURE (22)..(22): Xaa is Met or Ile
MAKTKKQKRNNLLLGVVFFIGXAVMAYPLVSRLYYRVESNQQIADFDKEKATLDEADIDERMKLAQAFNDSLNNVVSGDPWSEEMKKKGRAEYARMLEIHERMGHVEIPAIDVDLPVYAGTAEEVLQQGAGHLEGTSLPIGGNSTHAVITAHTGLPTAKMFTDLTKLKVGDKFYVHNIKEVMAYQVDQVKVIEPTNFDDLLIVPGHDYVTLLTCTPYMINTHRLLVRGHRIPYVAEVEEEFIAANKLSHLYRYLFYVAVGLIVILLWIIRRLRKKKRQSERALKALKEATKEVKVEDE

SEQ ID NO: 35 - protein, 297 aa, Streptococcus pneumoniae
MDNSRRSRKKGTKKKKHPLILLLIFLVGFAVAIYPLVSRYYYRIESNEVIKEFDETVSQMDKAELEERWRLAQAFNATLKPSEILDPFTEQEKKKGVSEYANMLKVHERIGYVEIPAIDQEIPMYVGTSEDILQKGAGLLEGASLPVGGKNTHTVITAHRGLPTAELFSQLDKMKKGDIFYLHVLDQVLAYQVDQIVTVEPNDFEPVLIQHGEDYATLLTCTPYMINSHRLLVRGKRIPYTAPIAERNRAVRERGQFWLWLLLGAMAVILLLLYRVYRNRRIVKGLEKQLEGRHVKD

SEQ ID NO: 36 - protein, 283 aa, Streptococcus pneumoniae
MSRTKLRALLGYLLMLVACLIPIYCFGQMVLQSLGQVKGHATFVKSMTTEMYQEQQNHSLAYNQRLASQNRIVDPFLAEGYEVNYQVSDDPDAVYGYLSIPSLEIMEPVYLGADYHHLGMGLAHVDGTPLPMDGTGIRSVIAGHRAEPSHVFFRHLDQLKVGDALYYDNGQEIVEYQMMDTEIILPSEWEKLESVSSKNIMTLITCDPIPTFNKRLLVNFERVAVYQKSDPQTAAVARVAFTKEGQSVSRVATSQWLYRGLVVLAFLGILFVLWKLARLLRGK

SEQ ID NO: 37 - protein, 249 aa, Streptococcus pyogenes
MVKKQKRRKIKSMSWARKLLIAVLLILGLALLFNKPIRNTLIARNSNKYQVTKVSKKQIKKNKEAKSTFDFQAVEPVSTESVLQAQMAAQQLPVIGGIAIPELGINLPIFKGLGNTELIYGAGTMKEEQVMGGENNYSLASHHIFGITGSSQMLFSPLERAQNGMSIYLTDKEKIYEYIIKDVFTVAPERVDVIDDTAGLKEVTLVTCTDIEATERIIVKGELKTEYDFDKAPADVLKAFNHSYNQVST

SEQ ID NO: 38 - protein, 1296 aa, Artificial Sequence
  Polypeptide sequence of proteolytically inactive mutant BoNT/A(0
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHQLIYAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 39 - DNA, 4080 bp, Artificial Sequence
  Nucleotide sequence of full length proteolytically inactive mutant BoNT/A(0) with dual labelling SrtA sites
atggagaacctgtattttcagggcggcggtggcagcggcggcagcggcggcagcccgtttgtgaacaagcagttcaactataaagatccggttaatggtgtggatatcgcctatatcaaaattccgaatgcaggtcagatgcagccggttaaagcctttaaaatccataacaaaatttgggtgattccggaacgtgatacctttaccaatccggaagaaggtgatctgaatccgcctccggaagcaaaacaggttccggttagctattatgatagcacctatctgagcaccgataacgagaaagataactatctgaaaggtgtgaccaaactgtttgaacgcatttatagtaccgatctgggtcgtatgctgctgaccagcattgttcgtggtattccgttttggggtggtagcaccattgataccgaactgaaagttattgacaccaactgcattaatgtgattcagccggatggtagctatcgtagcgaagaactgaatctggttattattggtccgagcgcagatatcattcagtttgaatgtaaaagctttggccacgaagttctgaatctgacccgtaatggttatggtagtacccagtatattcgtttcagtccggattttacctttggctttgaagaaagcctggaagttgatacaaatccgctgttaggtgcaggtaaatttgcaaccgatccggcagttaccctggcacaccagctgatttatgccggtcatcgtctgtatggtattgccattaatccgaatcgtgtgttcaaagtgaataccaacgcctattatgaaatgagcggtctggaagtgagttttgaagaactgcgtacctttggtggtcatgatgccaaatttatcgatagcctgcaagaaaatgaatttcgcctgtactactataacaaattcaaggatattgcgagcaccctgaataaagccaaaagcattgttggcaccaccgcaagcctgcagtatatgaaaaatgtgtttaaagaaaaatatctgctgagcgaagataccagcggtaaatttagcgttgacaaactgaaattcgataaactgtacaagatgctgaccgagatttataccgaagataacttcgtgaagtttttcaaagtgctgaaccgcaaaacctacctgaactttgataaagccgtgttcaaaatcaacatcgtgccgaaagtgaactataccatctatgatggttttaacctgcgcaataccaatctggcagcaaactttaatggtcagaacaccgaaatcaacaacatgaactttaccaaactgaagaacttcaccggtctgttcgaattttacaaactgctgtgtgttcgtggcattattaccagcaaaaccaaaagtctggataaaggctacaataaagccctgaatgatctgtgcattaaggtgaataattgggacctgttttttagcccgagcgaggataatttcaccaacgatctgaacaaaggcgaagaaattaccagcgataccaatattgaagcagccgaagaaaacattagcctggatctgattcagcagtattatctgaccttcaacttcgataatgagccggaaaatatcagcattgaaaacctgagcagcgatattattggccagctggaactgatgccgaatattgaacgttttccgaacggcaaaaaatacgagctggataaatacaccatgttccattatctgcgtgcccaagaatttgaacatggtaaaagccgtattgcactgaccaatagcgttaatgaagcactgctgaacccgagccgtgtttataccttttttagcagcgattacgtgaaaaaggttaacaaagcaaccgaagcagccatgtttttaggttgggttgaacagctggtttatgatttcaccgatgaaaccagcgaagttagcaccaccgataaaattgcagatattaccatcatcatcccgtatatcggtccggcactgaatattggcaatatgctgtataaagacgattttgtgggtgccctgatttttagcggtgcagttattctgctggaatttattccggaaattgccattccggttctgggcacctttgcactggtgagctatattgcaaataaagttctgaccgtgcagaccatcgataatgcactgagcaaacgtaacgaaaaatgggatgaagtgtacaagtatatcgtgaccaattggctggcaaaagttaacacccagattgacctgattcgcaagaagatgaaagaagcactggaaaatcaggcagaagcaaccaaagccattatcaactatcagtataaccagtacaccgaagaagagaaaaataacatcaacttcaacatcgacgatctgtccagcaaactgaacgaaagcatcaacaaagccatgattaacattaacaaatttctgaaccagtgcagcgtgagctatctgatgaatagcatgattccgtatggtgtgaaacgtctggaagattttgatgcaagcctgaaagatgccctgctgaaatatatctatgataatcgtggcaccctgattggtcaggttgatcgtctgaaagataaagtgaacaacaccctgagtaccgatattccttttcagctgagcaaatatgtggataatcagcgtctgctgtcaacctttaccgaatacattaagaacatcatcaacaccagcattctgaacctgcgttatgaaagcaatcatctgattgatctgagccgttatgccagcaaaatcaatataggcagcaaggttaacttcgacccgattgacaaaaatcagatacagctgtttaatctggaaagcagcaaaattgaggtgatcctgaaaaacgccattgtgtataatagcatgtacgagaatttctcgaccagcttttggattcgtatcccgaaatactttaatagcatcagcctgaacaacgagtacaccattattaactgcatggaaaacaatagcggctggaaagttagcctgaattatggcgaaattatctggaccctgcaggatacccaagaaatcaaacagcgtgtggttttcaaatacagccagatgattaatatcagcgactatatcaaccgctggatttttgtgaccattaccaataatcgcctgaataacagcaagatctatattaacggtcgtctgattgaccagaaaccgattagtaatctgggtaatattcatgcgagcaacaacatcatgtttaaactggatggttgtcgtgatacccatcgttatatttggatcaagtacttcaacctgttcgataaagagttgaacgaaaaagaaattaaagacctgtatgataaccagagcaacagcggtattctgaaggatttttggggagattatctgcagtatgacaaaccgtattatatgctgaatctgtacgacccgaataaatacgtggatgtgaataatgttggcatccgtggttatatgtacctgaaaggtccgcgtggtagcgttatgaccacaaacatttatctgaatagcagcctgtatcgcggaaccaaattcatcattaaaaagtatgccagcggcaacaaggataatattgtgcgtaataatgatcgcgtgtacattaacgttgtggtgaagaataaagaatatcgcctggcaaccaatgcaagccaggcaggcgttgaaaaaattctgagtgccctggaaattccggatgttggtaatctgagccaggttgttgtgatgaaaagcaaaaatgatcagggcatcaccaacaagtgcaaaatgaatctgcaggacaataacggcaacgatattggttttattggcttccaccagttcaacaatattgcgaaactggttgcaagcaattggtataatcgtcagattgaacgtagcagtcgtaccctgggttgtagctgggaatttatccctgtggatgatggttggggtgaacgtccgctgggcggcagcggcggcggcagcggcctgcccgaaagcggtggcggatctgcttggtctcacccgcagttcgaaaaaggtggtggttctggtggtggttctggtggttctgcttggtctcacccgcagttcgaaaaataatga

SEQ ID NO: 40 - protein, 1358 aa, Artificial Sequence
  Polypeptide sequence of full length proteolytically inactive mutant BoNT/A(0) with dual labelling SrtA sites
MENLYFQGGGGSGGSGGSPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHQLIYAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPLGGSGGGSGLPESGGGSAWSHPQFEKGGGSGGGSGGSAWSHPQFEK

SEQ ID NO: 41 - protein, 1191 aa, Prochloron didemni
MFSIMITIDYPFTVSLNRDIQVTSTEDYYTLQVTESDPSAWLTFATTPAMDMAFDHLKAGTTTESLVQTLAELGGPAAREQFALTLQQLDERGWLSYAVLPLAEAIPMVESAELNLPGNPHWMETGVTLSRFAYQHPYEGTMVLESPLSKFRVKLLDWRASALLAQLAQPQTLGTIAPPPYLGPETAYQFLNLLWATGFLASDHEPVSLQLWDFHNLLFHSRSRLGRHDYPGTDLNVDNWSDFPVVKPPMSDRIVPLPRPNLEALMSNDATLTEAIETRKSVREYDDDNPITIEQLGELLYRAARVTKLLSPEERFGKLWQQNKPVFEEAGVDEGEFSHRPYPGGGAMYELEIYPVVRLCQGLSQGVYHYDPLNHQLEQIVESKDDIFAVSGSPLASKLGPHVLLVITARFGRLFRLYRSVAYALVLKHVGVLQQNLYLVATNMGLAPCAGGAGDSDAFAQVTGIDYVEESAVGEFILGSLASEVESDVVEGEDEIESAGVSASEVESSATKQKVALHPHDLDERIPGLADLHNQTLGDPQITIVIIDGDPDYTLSCFEGAEVSKVFPYWHEPAEPITPEDYAAFQSIRDQGLKGKEKEEALEAVIPDTKDRIVLNDHACHVTSTIVGQEHSPVFGIAPNCRVINMPQDAVIRGNYDDVMSPLNLARAIDLALELGANIIHCAFCRPTQTSEGEEILVQAIKKCQDNNVLIVSPTGNNSNESWCLPAVLPGTLAVGAAKVDGTPCHFSNWGGNNTKEGILAPGEEILGAQPCTEEPVRLTGTSMAAPVMTGISALLMSLQVQQGKPVDAEAVRTALLKTAIPCDPEVVEEPERCLRGFVNIPGAMKVLFGQPSVTVSFAGGQATRTEHPGYATVAPASIPEPMAERATPAVQAATATEMVIAPSTEPANPATVEASTAFSGNVYALGTIGYDFGDEARRDTFKERMADPYDARQMVDYLDRNPDEARSLIWTLNLEGDVIYALDPKGPFATNVYEIFLQMLAGQLEPETSADFIERLSVPARRTTRTVELFSGEVMPVVNVRDPRGMYGWNVNALVDAALATVEYEEADEDSLRQGLTAFLNRVYHDLHNLGQTSRDRALNFTVTNTFQAASTFAQAIASGRQLDTIEVNKSPYCRLNSDCWDVLLTFYDPEHGRRSRRVFRFTLDVVYVLPVTVGSIKSWSLPGKGTVSK

SEQ ID NO: 42 - protein, 724 aa, Saponaria vaccaria
MATSGFSKPLHYPPVRRDETVVDDYFGVKVADPYRWLEDPNSEETKEFVDNQEKLANSVLEECELIDKFKQKIIDFVNFPRCGVPFRRANKYFHFYNSGLQAQNVFQMQDDLDGKPEVLYDPNLREGGRSGLSLYSVSEDAKYFAFGIHSGLTEWVTIKILKTEDRSYLPDTLEWVKFSPAIWTHDNKGFFYCPYPPLKEGEDHMTRSAVNQEARYHFLGTDQSEDILLWRDLENPAHHLKCQITDDGKYFLLYILDGCDDANKVYCLDLTKLPNGLESFRGREDSAPFMKLIDSFDASYTAIANDGSVFTFQTNKDAPRKKLVRVDLNNPSVWTDLVPESKKDLLESAHAVNENQLILRYLSDVKHVLEIRDLESGALQHRLPIDIGSVDGITARRRDSVVFFKFTSILTPGIVYQCDLKNDPTQLKIFRESVVPDFDRSEFEVKQVFVPSKDGTKIPIFIAARKGISLDGSHPCEMHGYGGFGINMMPTFSASRIVFLKHLGGVFCLANIRGGGEYGEEWHKAGFRDKKQNVFDDFISAAEYLISSGYTKARRVAIEGGSNGGLLVAACINQRPDLFGCAEANCGVMDMLRFHKFTLGYLWTGDYGCSDKEEEFKWLIKYSPIHNVRRPWEQPGNEETQYPATMILTADHDDRVVPLHSFKLLATMQHVLCTSLEDSPQKNPIIARIQRKAAHYGRATMTQIAEVADRYGFMAKALEAPWID

SEQ ID NO: 43 - protein, 730 aa, Galerina marginata
MSSVTWAPGNYPSTRRSDHVDTYQSASKGEVPVPDPYQWLEESTDEVDKWTTAQADLAQSYLDQNADIQKLAEKFRASRNYAKFSAPTLLDDGHWYWFYNRGLQSQSVLYRSKEPALPDFSKGDDNVGDVFFDPNVLAADGSAGMVLCKFSPDGKFFAYAVSHLGGDYSTIYVRSTSSPLSQASVAQGVDGRLSDEVKWFKFSTIIWTKDSKGFLYQRYPARERHEGTRSDRNAMMCYHKVGTTQEEDIIVYQDNEHPEWIYGADTSEDGKYLYLYQFKDTSKKNLLWVAELDEDGVKSGIHWRKVVNEYAADYNIITNHGSLVYIKTNLNAPQYKVITIDLSKDEPEIRDFIPEEKDAKLAQVNCANEEYFVAIYKRNVKDEIYLYSKAGVQLTRLAPDFVGAASIANRQKQTHFFLTLSGFNTPGTIARYDFTAPETQRFSILRTTKVNELDPDDFESTQVWYESKDGTKIPMFIVRHKSTKFDGTAAAIQYGYGGFATSADPFFSPIILTFLQTYGAIFAVPSIRGGGEFGEEWHKGGRRETKVNTFDDFIAAAQFLVKNKYAAPGKVAINGASNGGLLVMGSIVRAPEGTFGAAVPEGGVADLLKFHKFTGGQAWISEYGNPSIPEEFDYIYPLSPVHNVRTDKVMPATLITVNIGDGRVVPMHSFKFIATLQHNVPQNPHPLLIKIDKSWLGHGMGKPTDKNVKDAADKWGFIARALGLELKTVE

SEQ ID NO: 44 - protein, 474 aa, Oldenlandia affinis
MVRYLAGAVLLLVVLSVAAAVSGARDGDYLHLPSEVSRFFRPQETNDDHGEDSVGTRWAVLIAGSKGYANYRHQAGVCHAYQILKRGGLKDENIVVFMYDDIAYNESNPRPGVIINSPHGSDVYAGVPKDYTGEEVNAKNFLAAILGNKSAITGGSGKVVDSGPNDHIFIYYTDHGAAGVIGMPSKPYLYADELNDALKKKHASGTYKSLVFYLEACESGSMFEGILPEDLNIYALTSTNTTESSWCYYCPAQENPPPPEYNVCLGDLFSVAWLEDSDVQNSWYETLNQQYHHVDKRISHASHATQYGNLKLGEEGLFVYMGSNPANDNYTSLDGNALTPSSIVVNQRDADLLHLWEKFRKAPEGSARKEVAQTQIFKAMSHRVHIDSSIKLIGKLLFGIEKCTEILNAVRPAGQPLVDDWACLRSLVGTFETHCGSLSEYGMRHTRTIANICNAGISEEQMAEAASQACASIP

SEQ ID NO: 45 - protein, 451 aa, Oldenlandia affinis
ARDGDYLHLPSEVSRFFRPQETNDDHGEDSVGTRWAVLIAGSKGYANYRHQAGVCHAYQILKRGGLKDENIVVFMYDDIAYNESNPRPGVIINSPHGSDVYAGVPKDYTGEEVNAKNFLAAILGNKSAITGGSGKVVDSGPNDHIFIYYTDHGAAGVIGMPSKPYLYADELNDALKKKHASGTYKSLVFYLEACESGSMFEGILPEDLNIYALTSTNTTESSWCYYCPAQENPPPPEYNVCLGDLFSVAWLEDSDVQNSWYETLNQQYHHVDKRISHASHATQYGNLKLGEEGLFVYMGSNPANDNYTSLDGNALTPSSIVVNQRDADLLHLWEKFRKAPEGSARKEVAQTQIFKAMSHRVHIDSSIKLIGKLLFGIEKCTEILNAVRPAGQPLVDDWACLRSLVGTFETHCGSLSEYGMRHTRTIANICNAGISEEQMAEAASQACASIP

SEQ ID NO: 46 - protein, 5 aa, Artificial Sequence
  Sortase Acceptor Site
NPKTG

SEQ ID NO: 47 - protein, 5 aa, Artificial Sequence
  Sortase Acceptor Site
  MISC_FEATURE (1)..(1): Xaa is any amino acid
XPETG

SEQ ID NO: 48 - protein, 5 aa, Artificial Sequence
  Sortase Acceptor Site
LGATG

SEQ ID NO: 49 - protein, 5 aa, Artificial Sequence
  Sortase Acceptor Site
IPNTG

SEQ ID NO: 50 - protein, 5 aa, Artificial Sequence
  Sortase Acceptor Site
IPETG

SEQ ID NO: 51 - protein, 5 aa, Artificial Sequence
  Sortase Acceptor Site
NSKTA

SEQ ID NO: 52 - protein, 5 aa, Artificial Sequence
  Sortase Acceptor Site
NPQTG

SEQ ID NO: 53 - protein, 5 aa, Artificial Sequence
  Sortase Acceptor Site
NAKTN

SEQ ID NO: 54 - protein, 5 aa, Artificial Sequence
  Sortase Acceptor Site
NPQSS

SEQ ID NO: 55 - protein, 5 aa, Artificial Sequence
  Sortase Acceptor Site
  MISC_FEATURE (3)..(3): Xaa is any amino acid
  MISC_FEATURE (5)..(5): Xaa is any amino acid
LPXTX

SEQ ID NO: 56 - protein, 5 aa, Artificial Sequence
  Sortase Acceptor Site
  MISC_FEATURE (3)..(3): Xaa is Lys or Gln
  MISC_FEATURE (5)..(5): Xaa is Asn, Asp or Gly
NPXTX

SEQ ID NO: 57 - protein, 5 aa, Artificial Sequence
  Sortase Acceptor Site
  MISC_FEATURE (1)..(1): Xaa is Leu, Ile, Val or Met
  MISC_FEATURE (3)..(3): Xaa is any amino acid
  MISC_FEATURE (4)..(4): Xaa is Ser, Thr or Ala
XPXXG

SEQ ID NO: 58 - protein, 5 aa, Artificial Sequence
  Artificial Sequence
  MISC_FEATURE (4)..(4): Xaa is Ala, Cys or Ser
LPEXG

SEQ ID NO: 59 - protein, 5 aa, Artificial Sequence
  Sortase Acceptor Site
  MISC_FEATURE (2)..(2): Xaa is Ala, Pro or Ser
  MISC_FEATURE (3)..(3): Xaa is any amino acid
  MISC_FEATURE (4)..(4): Xaa is Thr, Ser, Ala or Cys
  MISC_FEATURE (5)..(5): Xaa is n number of Gly
LXXXX

SEQ ID NO: 60 - protein, 5 aa, Artificial Sequence
  Sortase Acceptor Site
  MISC_FEATURE (2)..(2): Xaa is Ala, Pro or Ser
  MISC_FEATURE (3)..(3): Xaa is any amino acid
  MISC_FEATURE (4)..(4): Xaa is Thr, Ser, Ala or Cys
  MISC_FEATURE (5)..(5): Xaa is n number of Ala
LXXXX

SEQ ID NO: 61 - protein, 5 aa, Artificial Sequence
  Sortase Acceptor Site
NPQTN

SEQ ID NO: 62 - protein, 5 aa, Artificial Sequence
  Sortase Acceptor Site
YPRTG

SEQ ID NO: 63 - protein, 5 aa, Artificial Sequence
  Sortase Acceptor Site
IPQTG

SEQ ID NO: 64 - protein, 5 aa, Artificial Sequence
  Sortase Acceptor Site
VPDTG

SEQ ID NO: 65 - protein, 6 aa, Artificial Sequence
  Sortase Acceptor Site
  MISC_FEATURE (3)..(3): Xaa is any amino acid
LPXTGS

SEQ ID NO: 66 - protein, 4 aa, Artificial Sequence
  Sortase Acceptor Site
  MISC_FEATURE (3)..(3): Xaa is any amino acid
LPXS

SEQ ID NO: 67 - protein, 4 aa, Artificial Sequence
  Sortase Acceptor Site
  MISC_FEATURE (3)..(3): Xaa is any amino acid
LAXT

SEQ ID NO: 68 - protein, 4 aa, Artificial Sequence
  Sortase Acceptor Site
  MISC_FEATURE (3)..(3): Xaa is any amino acid
MPXT

SEQ ID NO: 69 - protein, 5 aa, Artificial Sequence
  Sortase Acceptor Site
  MISC_FEATURE (3)..(3): Xaa is any amino acid
MPXTG

SEQ ID NO: 70 - protein, 4 aa, Artificial Sequence
  Sortase Acceptor Site
  MISC_FEATURE (3)..(3): Xaa is any amino acid
LAXS

SEQ ID NO: 71 - protein, 4 aa, Artificial Sequence
  Sortase Acceptor Site
  MISC_FEATURE (3)..(3): Xaa is any amino acid
NPXT

SEQ ID NO: 72 - protein, 5 aa, Artificial Sequence
  Sortase Acceptor Site
  MISC_FEATURE (3)..(3): Xaa is any amino acid
NPXTG

SEQ ID NO: 73 - protein, 4 aa, Artificial Sequence
  Sortase Acceptor Site
  MISC_FEATURE (3)..(3): Xaa is any amino acid
NAXT

SEQ ID NO: 74 - protein, 5 aa, Artificial Sequence
  Sortase Acceptor Site
  MISC_FEATURE (3)..(3): Xaa is any amino acid
NAXTG

SEQ ID NO: 75 - protein, 4 aa, Artificial Sequence
  Sortase Acceptor Site
  MISC_FEATURE (3)..(3): Xaa is any amino acid
NAXS

SEQ ID NO: 76 - protein, 5 aa, Artificial Sequence
  Sortase Acceptor Site
  MISC_FEATURE (3)..(3): Xaa is any amino acid
NAXSG

SEQ ID NO: 77 - protein, 4 aa, Artificial Sequence
  Sortase Acceptor Site
  MISC_FEATURE (3)..(3): Xaa is any amino acid
LPXP

SEQ ID NO: 78 - protein, 5 aa, Artificial Sequence
  Sortase Acceptor Site
  MISC_FEATURE (3)..(3): Xaa is any amino acid
LPXPG

SEQ ID NO: 79 - protein, 7 aa, Artificial Sequence
  Leucine-Based Motif
  MISC_FEATURE (1)..(1): Xaa is any amino acid
  MISC_FEATURE (3)..(5): Xaa is any amino acid
XDXXXLL

SEQ ID NO: 80 - protein, 7 aa, Artificial Sequence
  Leucine-Based Motif
  MISC_FEATURE (1)..(1): Xaa is any amino acid
  MISC_FEATURE (3)..(5): Xaa is any amino acid
XEXXXLL

SEQ ID NO: 81 - protein, 7 aa, Artificial Sequence
  Leucine-Based Motif
  MISC_FEATURE (1)..(1): Xaa is any amino acid
  MISC_FEATURE (3)..(5): Xaa is any amino acid
XEXXXIL

SEQ ID NO: 82 - protein, 7 aa, Artificial Sequence
  Leucine-Based Motif
  MISC_FEATURE (1)..(1): Xaa is any amino acid
  MISC_FEATURE (3)..(5): Xaa is any amino acid
XEXXXLM

SEQ ID NO: 83 - protein, 4 aa, Artificial Sequence
  Tyrosine-Based Motif
  MISC_FEATURE (2)..(3): Xaa is any amino acid
  MISC_FEATURE (4)..(4): Xaa is a hydrophobic amino acid
YXXX

SEQ ID NO: 84 - protein, 5 aa, Artificial Sequence
  Enterokinase Cleavage Site
DDDDK

SEQ ID NO: 85 - protein, 4 aa, Artificial Sequence
  Factor Xa Cleavage Site
IEGR

SEQ ID NO: 86 - protein, 4 aa, Artificial Sequence
  Factor Xa Cleavage Site
IDGR

SEQ ID NO: 87 - protein, 7 aa, Artificial Sequence
  TEV Cleavage Site
ENLYFQG

SEQ ID NO: 88 - protein, 6 aa, Artificial Sequence
  Thrombin Cleavage Site
LVPRGS

SEQ ID NO: 89 - protein, 8 aa, Artificial Sequence
  PreScission Cleavage Site
LEVLFQGP

SEQ ID NO: 90 - protein, 10 aa, Artificial Sequence
  ADAM17 Cleavage Site
PLAQAVRSSS

SEQ ID NO: 91 - protein, 10 aa, Artificial Sequence
  Human Airway Trypsin-Like Protease (HAT) Cleavage Site
SKGRSLIGRV

SEQ ID NO: 92 - protein, 6 aa, Artificial Sequence
  Elastase Cleavage Site
MEAVTY

SEQ ID NO: 93 - protein, 5 aa, Artificial Sequence
  Furin Cleavage Site
  MISC_FEATURE (2)..(2): Xaa is any amino acid
RXRKR

SEQ ID NO: 94 - protein, 4 aa, Artificial Sequence
  Granzyme Cleavage Site
IEPD

SEQ ID NO: 95 - protein, 4 aa, Artificial Sequence
  Caspase 2 Cleavage Site
DVAD

SEQ ID NO: 96 - protein, 4 aa, Artificial Sequence
  Caspase 3 Cleavage Site
DMQD

SEQ ID NO: 97 - protein, 4 aa, Artificial Sequence
  Caspase 4 Cleavage Site
LEVD

SEQ ID NO: 98 - protein, 4 aa, Artificial Sequence
  Caspase 7 Cleavage Site
DEVD

SEQ ID NO: 99 - protein, 4 aa, Artificial Sequence
  Caspase 9 Cleavage Site
LEHD

SEQ ID NO: 100 - protein, 4 aa, Artificial Sequence
  Caspase 10 Cleavage Site
IEHD

SEQ ID NO: 101 - protein, 23 aa, Influenza virus
GLFGAIAGFIENGWEGMIDGWYG

SEQ ID NO: 102 - protein, 5 aa, Artificial Sequence
  Sortase Acceptor Site
  MISC_FEATURE (3)..(3): Xaa is any amino acid
  MISC_FEATURE (5)..(5): Xaa is n number of Ala
LPXTX

SEQ ID NO: 103 - protein, 5 aa, Artificial Sequence
  Sortase Acceptor Site
  MISC_FEATURE (3)..(3): Xaa is any amino acid
  MISC_FEATURE (5)..(5): Xaa is n number of Gly
LPXSX

SEQ ID NO: 104 - protein, 5 aa, Artificial Sequence
  Sortase Acceptor Site
  MISC_FEATURE (3)..(3): Xaa is any amino acid
  MISC_FEATURE (5)..(5): Xaa is n number of Gly
LAXTX

SEQ ID NO: 105 - protein, 5 aa, Artificial Sequence
  Sortase Acceptor Site
  MISC_FEATURE (3)..(3): Xaa is any amino acid
  MISC_FEATURE (5)..(5): Xaa is n number of Gly
LPXTX

SEQ ID NO: 106 - protein, 5 aa, Artificial Sequence
  Sortase Acceptor Site
  MISC_FEATURE (4)..(4): Xaa is any amino acid
  MISC_FEATURE (5)..(5): Xaa is n number of Gly
LPAXX

SEQ ID NO: 107 - protein, 5 aa, Artificial Sequence
  Sortase Acceptor Site
  MISC_FEATURE (3)..(3): Xaa is any amino acid
  MISC_FEATURE (5)..(5): Xaa is n number of Gly
LPXCX

SEQ ID NO: 108 - protein, 5 aa, Artificial Sequence
  Sortase Acceptor Site
  MISC_FEATURE (3)..(3): Xaa is any amino acid
  MISC_FEATURE (5)..(5): Xaa is n number of Gly
LAXSX

SEQ ID NO: 109 - protein, 5 aa, Artificial Sequence
  Sortase Acceptor Site
  MISC_FEATURE (3)..(3): Xaa is any amino acid
  MISC_FEATURE (5)..(5): Xaa is n number of Gly
LPXAX

SEQ ID NO: 110 - protein, 5 aa, Artificial Sequence
  Sortase Acceptor Site
  MISC_FEATURE (3)..(3): Xaa is any amino acid
  MISC_FEATURE (5)..(5): Xaa is n number of Gly
LSXTX

SEQ ID NO: 111 - protein, 5 aa, Artificial Sequence
  Sortase Acceptor Site
  MISC_FEATURE (3)..(3): Xaa is any amino acid
  MISC_FEATURE (5)..(5): Xaa is n number of Gly
LRXTX

SEQ ID NO: 112 - protein, 5 aa, Artificial Sequence
  Sortase Acceptor Site
LPESG

SEQ ID NO: 113 - protein, 5 aa, Artificial Sequence
  Sortase Acceptor Site
LAETG

SEQ ID NO: 114 - protein, 5 aa, Artificial Sequence
  Sortase Acceptor Site
  MISC_FEATURE (3)..(3): Xaa is any amino acid
LPXTA

SEQ ID NO: 115 - protein, 5 aa, Artificial Sequence
  Sortase Acceptor Site
  MISC_FEATURE (3)..(3): Xaa is any amino acid
LPXSG

SEQ ID NO: 116 - protein, 5 aa, Artificial Sequence
  Sortase Acceptor Site
  MISC_FEATURE (3)..(3): Xaa is any amino acid
LAXTG

SEQ ID NO: 117 - protein, 5 aa, Artificial Sequence
  Sortase Acceptor Site
  MISC_FEATURE (3)..(3): Xaa is any amino acid
LPXTG

SEQ ID NO: 118 - protein, 5 aa, Artificial Sequence
  Sortase Acceptor Site
  MISC_FEATURE (4)..(4): Xaa is any amino acid
LPAXG

SEQ ID NO: 119 - protein, 5 aa, Artificial Sequence
  Sortase Acceptor Site
  MISC_FEATURE (3)..(3): Xaa is any amino acid
LPXCG

SEQ ID NO: 120 - protein, 5 aa, Artificial Sequence
  Sortase Acceptor Site
  MISC_FEATURE (3)..(3): Xaa is any amino acid
LAXSG

SEQ ID NO: 121 - protein, 5 aa, Artificial Sequence
  Sortase Acceptor Site
  MISC_FEATURE (3)..(3): Xaa is any amino acid
LPXAG

SEQ ID NO: 122 - protein, 5 aa, Artificial Sequence
  Sortase Acceptor Site
  MISC_FEATURE (3)..(3): Xaa is any amino acid
LSXTG

SEQ ID NO: 123 - protein, 5 aa, Artificial Sequence
  Sortase Acceptor Site
  MISC_FEATURE (3)..(3): Xaa is any amino acid
LRXTG